SEQUENCE LISTING (102 sequences)

SEQ ID NO: 1 - protein, 108 aa, ARTIFICIAL SEQUENCE
  Synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 2 - protein, 121 aa, ARTIFICIAL SEQUENCE
  Synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 3 - protein, 8 aa, ARTIFICIAL SEQUENCE
  Synthetic construct
DYKDDDDK

SEQ ID NO: 4 - protein, 6 aa, ARTIFICIAL SEQUENCE
  Synthetic construct
HHHHHH

SEQ ID NO: 5 - protein, 9 aa, ARTIFICIAL SEQUENCE
  Synthetic construct
YPYDVPDYA

SEQ ID NO: 6 - protein, 184 aa, ARTIFICIAL SEQUENCE
  Synthetic construct
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 7 - protein, 201 aa, ARTIFICIAL SEQUENCE
  Synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 8 - protein, 214 aa, ARTIFICIAL SEQUENCE
  Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 683 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 10 - protein, 123 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSS

SEQ ID NO: 11 - protein, 9 aa, ARTIFICIAL SEQUENCE
  Synthetic construct
YTFSSHYVH

SEQ ID NO: 12 - protein, 13 aa, ARTIFICIAL SEQUENCE
  Synthetic construct
GIIDPSGGSTSYA

SEQ ID NO: 13 - protein, 18 aa, ARTIFICIAL SEQUENCE
  Synthetic construct
CARGRYDYGDYLGWFDPW

SEQ ID NO: 14 - protein, 114 aa, ARTIFICIAL SEQUENCE
  Synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 15 - protein, 9 aa, ARTIFICIAL SEQUENCE
  Synthetic construct
HTFTSYFMH

SEQ ID NO: 16 - protein, 13 aa, ARTIFICIAL SEQUENCE
  Synthetic construct
GIINPSDDYANYA

SEQ ID NO: 17 - protein, 9 aa, ARTIFICIAL SEQUENCE
  Synthetic construct
CATAIFDYW

SEQ ID NO: 18 - protein, 107 aa, ARTIFICIAL SEQUENCE [sequence omitted]

SEQ ID NO: 19 - protein, 11 aa, ARTIFICIAL SEQUENCE
  Synthetic construct
RASQSISSYLN

SEQ ID NO: 20 - protein, 7 aa, ARTIFICIAL SEQUENCE
  Synthetic construct
AASSLQS

SEQ ID NO: 21 - protein, 11 aa, ARTIFICIAL SEQUENCE [sequence omitted]

SEQ ID NO: 22 - protein, 5 aa, ARTIFICIAL SEQUENCE
  Synthetic construct
GGGGS

SEQ ID NO: 23 - protein, 4 aa, ARTIFICIAL SEQUENCE
  Synthetic construct
GGGS

SEQ ID NO: 24 - protein, 683 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 25 - protein, 114 aa, ARTIFICIAL SEQUENCE
  Synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 26 - protein, 683 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 27 - protein, 683 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 28 - protein, 304 aa, ARTIFICIAL SEQUENCE
  Synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 29 - protein, 370 aa, ARTIFICIAL SEQUENCE
  Synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 30 - protein, 365 aa, ARTIFICIAL SEQUENCE
  Synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 31 - protein, 335 aa, ARTIFICIAL SEQUENCE
  Synthetic construct
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTV

SEQ ID NO: 32 - protein, 216 aa, ARTIFICIAL SEQUENCE
  Synthetic construct
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 33 - protein, 252 aa, ARTIFICIAL SEQUENCE
  Synthetic construct
MGNSCYNIVATLLLVLNFERTRSLQDPCSNCPAGTFDNNRNQICSPCPPNSFSSAGGQRTCDICRQCKGVFRTRKECSSTSNAECDCTPGFHCLGAGCSMCEQDCKQGQLKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 34 - protein, 254 aa, ARTIFICIAL SEQUENCE
  Synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 35 - protein, 351 aa, ARTIFICIAL SEQUENCE
  Synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 36 - protein, 276 aa, ARTIFICIAL SEQUENCE
  Synthetic construct
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 37 - protein, 1225 aa, ARTIFICIAL SEQUENCE
  Synthetic construct
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 38 - protein, 9 aa, Artificial sequence
  Synthetic construct
  X (4)..(4): X=T or S
  X (5)..(5): X=N or S
  X (6)..(6): X=Y or H
  X (8)..(8): X=M or V
YTFXXXYXH

SEQ ID NO: 39 - protein, 13 aa, Artificial sequence
  Synthetic construct
  X (2)..(2): X=V or I
  X (7)..(7): X=G or D
  X (9)..(9): X=G, Y or S
  X (11)..(11): X=N or S
GXIDPSXGXTXYA

SEQ ID NO: 40 - protein, 16 aa, Artificial sequence
  Synthetic construct
  X (8)..(8): X=G or S
  X (16)..(16): X=P or G
ARGRYDYXDYLGWFDX

SEQ ID NO: 41 - protein, 16 aa, ARTIFICIAL SEQUENCE
  Synthetic construct
ARGRYDYGDYLGWFDP

SEQ ID NO: 42 - protein, 7 aa, ARTIFICIAL SEQUENCE
  Synthetic construct
ATAIFDY

SEQ ID NO: 43 - protein, 9 aa, ARTIFICIAL SEQUENCE
  Synthetic construct
QQSYSTPLT

SEQ ID NO: 44 - protein, 9 aa, ARTIFICIAL SEQUENCE
  Synthetic construct
YTFTNYYMH

SEQ ID NO: 45 - protein, 13 aa, ARTIFICIAL SEQUENCE
  Synthetic construct
GVIDPSGGYTNYA

SEQ ID NO: 46 - protein, 16 aa, ARTIFICIAL SEQUENCE
  Synthetic construct
ARGRYDYGDYLGWFDG

SEQ ID NO: 47 - protein, 9 aa, ARTIFICIAL SEQUENCE
  Synthetic construct
YTFTSYYMH

SEQ ID NO: 48 - protein, 13 aa, ARTIFICIAL SEQUENCE
  Synthetic construct
GVIDPSGGSTNYA

SEQ ID NO: 49 - protein, 16 aa, ARTIFICIAL SEQUENCE
  Synthetic construct
ARGRYDYSDYLGWFDP

SEQ ID NO: 50 - protein, 13 aa, ARTIFICIAL SEQUENCE
  Synthetic construct
GVIDPSDGGTNYA

SEQ ID NO: 51 - protein, 454 aa, ARTIFICIAL SEQUENCE
  Synthetic construct
GTQVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 123 aa, ARTIFICIAL SEQUENCE
  Synthetic construct
QVQLVQSGAEVKKPGASVKVSCKASGYTFTNYYMHWVRQAPGQGLEWMGVIDPSGGYTNYAQKFQGRVTMTRDTSTSTVYMELSSLRSEDTAVYYCARGRYDYGDYLGWFDGWGQGTLVTVSS

SEQ ID NO: 53 - protein, 123 aa, ARTIFICIAL SEQUENCE
  Synthetic construct
QVQLVQSGAEVKKPGASVKVSCKASGYTFTSYYMHWVRQAPGQGLEWMGVIDPSGGSTNYAQKFQGRVTMTRDTSTSTVYMELSSLRSEDTAVYYCARGRYDYGDYLGWFDGWGQGTLVTVSS

SEQ ID NO: 54 - protein, 123 aa, ARTIFICIAL SEQUENCE
  Synthetic construct
QVQLVQSGAEVKKPGASVKVSCKASGYTFSSHYVHWVRQAPGQGLEWMGVIDPSGGYTNYAQKFQGRVTMTRDTSTSTVYMELSSLRSEDTAVYYCARGRYDYSDYLGWFDPWGQGTLVTVSS

SEQ ID NO: 55 - protein, 123 aa, ARTIFICIAL SEQUENCE
  Synthetic construct
QVQLVQSGAEVKKPGASVKVSCKASGYTFSSHYVHWVRQAPGQGLEWMGVIDPSDGGTNYAQKFQGRVTMTRDTSTSTVYMELSSLRSEDTAVYYCARGRYDYGDYLGWFDPWGQGTLVTVSS

SEQ ID NO: 56 - protein, 123 aa, ARTIFICIAL SEQUENCE
  Synthetic construct
QVQLVQSGAEVKKPGASVKVSCKASGYTFTNYYMHWVRQAPGQGLEWMGVIDPSDGGTNYAQKFQGRVTMTRDTSTSTVYMELSSLRSEDMAVYYCARGRYDYGDYLGWFDPWGQGTLVTVSS

SEQ ID NO: 57 - protein, 123 aa, ARTIFICIAL SEQUENCE
  Synthetic construct
QVQLVQSGAEVKKPGASVKVSCKASGYTFTNYYMHWVRQAPGQGLEWMGVIDPSDGGTNYAQKFQGRVTMTRDTSTSTVYMELSSLRSEDTAVYYCARGRYDYGDYLGWFDPWGQGTLVTVSS

SEQ ID NO: 58 - protein, 452 aa, ARTIFICIAL SEQUENCE
  Synthetic construct
QVQLVQSGAEVKKPGASVKVSCKASGYTFTNYYMHWVRQAPGQGLEWMGVIDPSGGYTNYAQKFQGRVTMTRDTSTSTVYMELSSLRSEDTAVYYCARGRYDYGDYLGWFDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 452 aa, ARTIFICIAL SEQUENCE
  Synthetic construct
QVQLVQSGAEVKKPGASVKVSCKASGYTFTSYYMHWVRQAPGQGLEWMGVIDPSGGSTNYAQKFQGRVTMTRDTSTSTVYMELSSLRSEDTAVYYCARGRYDYGDYLGWFDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 452 aa, ARTIFICIAL SEQUENCE; identical to SEQ ID NO: 59
  Synthetic construct

SEQ ID NO: 61 - protein, 452 aa, ARTIFICIAL SEQUENCE
  Synthetic construct
QVQLVQSGAEVKKPGASVKVSCKASGYTFSSHYVHWVRQAPGQGLEWMGVIDPSDGGTNYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 452 aa, ARTIFICIAL SEQUENCE
  Synthetic construct
QVQLVQSGAEVKKPGASVKVSCKASGYTFTNYYMHWVRQAPGQGLEWMGVIDPSDGGTNYAQKFQGRVTMTRDTSTSTVYMELSSLRSEDMAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 452 aa, ARTIFICIAL SEQUENCE
  Synthetic construct
QVQLVQSGAEVKKPGASVKVSCKASGYTFTNYYMHWVRQAPGQGLEWMGVIDPSDGGTNYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 445 aa, ARTIFICIAL SEQUENCE
  Synthetic construct
GTQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 683 aa, ARTIFICIAL SEQUENCE
  Synthetic construct
QVQLVQSGAEVKKPGASVKVSCKASGYTFTSYYMHWVRQAPGQGLEWMGVIDPSGGSTNYAQKFQGRVTMTRDTSTSTVYMELSSLRSEDTAVYYCARGRYDYGDYLGWFDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 66 - protein, 683 aa, ARTIFICIAL SEQUENCE
  Synthetic construct
QVQLVQSGAEVKKPGASVKVSCKASGYTFTNYYMHWVRQAPGQGLEWMGVIDPSGGYTNYAQKFQGRVTMTRDTSTSTVYMELSSLRSEDTAVYYCARGRYDYGDYLGWFDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 67 - protein, 693 aa, ARTIFICIAL SEQUENCE
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 68 - protein, 683 aa, ARTIFICIAL SEQUENCE
  Synthetic construct
QVQLVQSGAEVKKPGASVKVSCKASGYTFTSYYMHWVRQAPGQGLEWMGVIDPSGGSTNYAQKFQGRVTMTRDTSTSTVYMELSSLRSEDTAVYYCARGRYDYGDYLGWFDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 69 - protein, 9 aa, ARTIFICIAL SEQUENCE
  Synthetic construct
GTFTSYSVS

SEQ ID NO: 70 - protein, 13 aa, ARTIFICIAL SEQUENCE
  Synthetic construct
GGIVPIFGTADYA

SEQ ID NO: 71 - protein, 12 aa, ARTIFICIAL SEQUENCE
  Synthetic construct
ARGYSYGQTFDY

SEQ ID NO: 72 - protein, 119 aa, ARTIFICIAL SEQUENCE
  Synthetic construct
QVQLVQSGAEVKKPGSSVKVSCKASGGTFTSYSVSWVRQAPGQGLEWMGGIVPIFGTADYAQKFQGRVTITADESTSTAYMELSSLRSEDTAVYYCARGYSYGQTFDYWGQGTLVTVSS

SEQ ID NO: 73 - protein, 448 aa, ARTIFICIAL SEQUENCE
  Synthetic construct
QVQLVQSGAEVKKPGSSVKVSCKASGGTFTSYSVSWVRQAPGQGLEWMGGIVPIFGTADYAQKFQGRVTITADESTSTAYMELSSLRSEDTAVYYCARGYSYGQTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 689 aa, ARTIFICIAL SEQUENCE
  Synthetic construct
GQVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVQSGAEVKKPGSSVKVSCKASGGTFTSYSVSWVRQAPGQGLEWMGGIVPIFGTADYAQKFQGRVTITADESTSTAYMELSSLRSEDTAVYYCARGYSYGQTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 75 - protein, 9 aa, ARTIFICIAL SEQUENCE
  Synthetic construct
HTFTNYYMH

SEQ ID NO: 76 - protein, 13 aa, ARTIFICIAL SEQUENCE
  Synthetic construct
GVINPNGGDTSYA

SEQ ID NO: 77 - protein, 17 aa, ARTIFICIAL SEQUENCE
  Synthetic construct
ARDRAWDYGGNVRAFDI

SEQ ID NO: 78 - protein, 124 aa, ARTIFICIAL SEQUENCE
  Synthetic construct
QVQLVQSGAEVKKPGASVKVSCKASGHTFTNYYMHWVRQAPGQGLEWMGVINPNGGDTSYAQKFQGRVTMTRDTSTSTVYMELSSLRSEDTAVYYCARDRAWDYGGNVRAFDIWGQGTMVTVSS

SEQ ID NO: 79 - protein, 453 aa, ARTIFICIAL SEQUENCE
  Synthetic construct
QVQLVQSGAEVKKPGASVKVSCKASGHTFTNYYMHWVRQAPGQGLEWMGVINPNGGDTSYAQKFQGRVTMTRDTSTSTVYMELSSLRSEDTAVYYCARDRAWDYGGNVRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 112 aa, ARTIFICIAL SEQUENCE
  Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGGGTKVEIK

SEQ ID NO: 81 - protein, 219 aa, ARTIFICIAL SEQUENCE
  Synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 183 aa, ARTIFICIAL SEQUENCE
  Synthetic construct
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 83 - DNA, 321 bp, ARTIFICIAL SEQUENCE
  Synthetic construct
gatatccagatgacacagtctccatcctctctgagtgctagtgtgggtgaccgcgttacgataacctgccgcgcaagtcagtccatatcatcatatctgaactggtatcagcaaaaacccggcaaagcacctaaactattgatctacgccgcatcgtcacttcagagtggtgtgccgagtcgttttagtggttccggaagcggtaccgattttaccctgactattagcagcctccagcccgaagatttcgcgacctactattgccagcagagctactcgacgccactaactttcggcggagggaccaaggtggagatcaaa

SEQ ID NO: 84 - DNA, 2049 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtatcatcgatccgtctggtggttctacctcttacgcacagaaatttcaaggtcgcgtgaccatgacccgagatacctccacgagtactgtgtacatggaattgtcgtccctgcgctccgaagacacggccgtgtattactgtgcgagaggacggtatgactacggtgactacttggggtggttcgacccctggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 85 - DNA, 2049 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtatcatcgatccgtctggtggttctacctcttacgcacagaaatttcaaggtcgcgtgaccatgacccgagatacctccacgagtactgtgtacatggaattgtcgtccctgcgctccgaagacacggccgtgtattactgtgcgagaggacggtatgactacggtgactacttggggtggttcgacccctggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 86 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtatcatcgatccgtctggtggttctacctcttacgcacagaaatttcaaggtcgcgtgaccatgacccgagatacctccacgagtactgtgtacatggaattgtcgtccctgcgctccgaagacacggccgtgtattactgtgcgagaggacggtatgactacggtgactacttggggtggttcgacccctggggccagggaaccctggtcaccgtctcctca

SEQ ID NO: 87 - DNA, 342 bp, ARTIFICIAL SEQUENCE
  Synthetic construct
caggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccccggtcaccgtctcctca

SEQ ID NO: 88 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacaccttcaccaactactacatgcactgggtgcggcaagcgccgggtcagggcttggaatggatgggtgttatcgatccgtctggtggttacaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacggttggggccagggaaccctggtcaccgtctcctca

SEQ ID NO: 89 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacacgttcacgtcatattacatgcattgggtgcggcaagcgccgggtcagggcttggaatggatgggtgttatcgatccgtctggtggttctaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacgggtggggccagggaaccctggtcaccgtctcctca

SEQ ID NO: 90 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtgttatcgatccgtctggtggttacaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacagtgactacttggggtggttcgacccctggggccagggaaccctggtcaccgtctcctca

SEQ ID NO: 91 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtgttatcgatccgtctgatggtggtaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacccctggggccagggaaccctggtcaccgtctcctca

SEQ ID NO: 92 - DNA, 369 bp, ARTIFICIAL SEQUENCE
  Synthetic construct
caggtacagctggtgcaatctggtgcggaggtgaagaaaccgggtgccagtgtgaaagttagttgtaaggcaagcggttacaccttcaccaactactacatgcactgggtgcggcaagcgccgggtcagggcttggaatggatgggtgttatcgatccgtctgatggtggtaccaactacgcacagaaatttcaaggtcgcgtgaccatgacccgagatacctccacgagtactgtgtacatggaattgtcgtccctgcgctccgaagacatggccgtgtattactgtgcaagaggacggtatgactacggtgactacttggggtggttcgacccctggggccagggaaccctggtcaccgtctcctca

SEQ ID NO: 93 - protein, 369 aa, ARTIFICIAL SEQUENCE
  Synthetic construct
CAGGTACAGCTGGTGCAATCTGGTGCGGAGGTGAAGAAACCGGGTGCCAGTGTGAAAGTTAGTTGTAAGGCAAGCGGTTACACCTTCACCAACTACTACATGCACTGGGTGCGGCAAGCGCCGGGTCAGGGCTTGGAATGGATGGGTGTTATCGATCCGTCTGATGGTGGTACCAACTACGCACAGAAATTTCAAGGTCGCGTGACCATGACCCGAGATACCTCCACGAGTACTGTGTACATGGAATTGTCGTCCCTGCGCTCCGAAGACACGGCCGTGTATTACTGTGCAAGAGGACGGTATGACTACGGTGACTACTTGGGGTGGTTCGACCCCTGGGGCCAGGGAACCCTGGTCACCGTCTCCTCA

SEQ ID NO: 94 - DNA, 2049 bp, ARTIFICIAL SEQUENCE
  Synthetic construct
caggtacagctggtgcaatctggtgcggaggtgaagaaaccgggtgccagtgtgaaagttagttgtaaggcaagcggttacacgttcacgtcatattacatgcattgggtgcggcaagcgccgggtcagggcttggaatggatgggtgttatcgatccgtctggtggttctaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacgggtggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 95 - DNA, 2049 bp, ARTIFICIAL SEQUENCE
  Synthetic construct
caggtacagctggtgcaatctggtgcggaggtgaagaaaccgggtgccagtgtgaaagttagttgtaaggcaagcggttacaccttcaccaactactacatgcactgggtgcggcaagcgccgggtcagggcttggaatggatgggtgttatcgatccgtctggtggttacaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacggttggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 96 - DNA, 357 bp, ARTIFICIAL SEQUENCE
  Synthetic construct
caagtgcagcttgttcagagcggagctgaagtgaagaaacccggttctagtgttaaagtgtcatgtaaagccagtggcggtaccttcacctcttactctgtttcttgggtgcgccaggctccgggccagggcctggagtggatgggtggtatcgttccaatcttcggtaccgcagattatgcccaaaaatttcaaggaagagtgacgataaccgctgatgaatctacaagcactgcatatatggaattgtcgagtttacggtccgaggatacggccgtgtattactgtgcgagaggatacagctatggtcagacctttgactactggggccagggaaccctggtcaccgtctcctca

SEQ ID NO: 97 - DNA, 2079 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtatcatcgatccgtctggtggttctacctcttacgcacagaaatttcaaggtcgcgtgaccatgacccgagatacctccacgagtactgtgtacatggaattgtcgtccctgcgctccgaagacacggccgtgtattactgtgcgagaggacggtatgactacggtgactacttggggtggttcgacccctggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggcggagggagtggaggtggaggcagtggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 98 - DNA, 372 bp, ARTIFICIAL SEQUENCE
  Synthetic construct
caggtacagctggtgcaatctggtgcggaggtgaagaaaccgggtgccagtgtgaaagttagttgtaaggcaagcggtcacaccttcaccaactactacatgcactgggtgcggcaagcgccgggtcagggcttggaatggatgggtgttatcaacccgaacggtggtgatacctcttacgcacagaaatttcaaggtcgcgtgaccatgacccgagatacctccacgagtactgtgtacatggaattgtcgtccctgcgctccgaagacacggccgtgtattactgtgcgagagatcgggcctgggactacggtggtaacgtgcgtgcttttgatatctggggccaagggacaatggtcaccgtctcttca

SEQ ID NO: 99 - DNA, 2049 bp, ARTIFICIAL SEQUENCE
  Synthetic construct
caggtacagctggtgcaatctggtgcggaggtgaagaaaccgggtgccagtgtgaaagttagttgtaaggcaagcggttacacgttcacgtcatattacatgcattgggtgcggcaagcgccgggtcagggcttggaatggatgggtgttatcgatccgtctggtggttctaccaactacgcacagaaatttcaaggtcgcgtgaccatgacccgagatacctccacgagtactgtgtacatggaattgtcgtccctgcgctccgaagacacggccgtgtattactgtgcaagaggacggtatgactacggtgactacttggggtggttcgacgggtggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaggtacagctggtgcaatctggtgcggaggtgaagaaaccgggtgccagtgtgaaagttagttgtaaggcaagcggtcacaccttcacctcttacttcatgcactgggtgcggcaagcgccgggtcagggcttggaatggatgggtatcatcaacccgtctgatgattacgcaaactacgcacagaaatttcaaggtcgcgtgaccatgacccgagatacctccacgagtactgtgtacatggaattgtcgtccctgcgctccgaagacacggccgtgtattactgtgccacagcgatctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 100 - DNA, 2064 bp, ARTIFICIAL SEQUENCE
  Synthetic construct
caggtacagctggtgcaatctggtgcggaggtgaagaaaccgggtgccagtgtgaaagttagttgtaaggcaagcggttacaccttctcttctcactacgttcactgggtgcggcaagcgccgggtcagggcttggaatggatgggtatcatcgatccgtctggtggttctacctcttacgcacagaaatttcaaggtcgcgtgaccatgacccgagatacctccacgagtactgtgtacatggaattgtcgtccctgcgctccgaagacacggccgtgtattactgtgcgagaggacggtatgactacggtgactacttggggtggttcgacccctggggccagggaaccctggtcaccgtctcctcagctagcaccaagggtccgagcgtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcactgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacaaaagagcctctccctgtctccgggtggaggcggaagcggaggtggcggatcccaagtgcagcttgttcagagcggagctgaagtgaagaaacccggttctagtgttaaagtgtcatgtaaagccagtggcggtaccttcacctcttactctgtttcttgggtgcgccaggctccgggccagggcctggagtggatgggtggtatcgttccaatcttcggtaccgcagattatgcccaaaaatttcaaggaagagtgacgataaccgctgatgaatctacaagcactgcatatatggaattgtcgagtttacggtccgaggatacggccgtgtattactgtgcgagaggatacagctatggtcagacctttgactactggggccagggaaccctggtcaccgtctcctcagctagcactaaaggacccagtgtatttcctttggcccctagcagcaaatctacatctggcggcacagccgctctgggttgtctggtgaaagactactttcctgaacccgtgactgtttcatggaacagtggggcactcacgagcggagtgcatactttccccgctgtgcttcagagttctggactctattcgcttagctctgtcgtaaccgtccctagttccagcctgggcacccaaacatatatatgcaacgttaaccataaaccctcaaatacaaaggttgataagaaggtggagccgaagagttgtgacaagacccacacc

SEQ ID NO: 101 - protein, 443 aa, Artificial sequence
  Synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 452 aa, Artificial sequence
  Synthetic construct
QVQLVQSGAEVKKPGASVKVSCKASGYTFSSHYVHWVRQAPGQGLEWMGIIDPSGGSTSYAQKFQGRVTMTRDTSTSTVYMELSSLRSEDTAVYYCARGRYDYGDYLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2020/0109195 A1
(43) Pub. Date: Apr. 9, 2020

(54) COMPOSITIONS AND METHODS FOR ENHANCING THE KILLING OF TARGET CELLS BY NK CELLS

(71) Applicant: COMPASS THERAPEUTICS LLC, Cambridge, MA (US)

(72) Inventors: Jennifer Watkins, Cambridge, MA (US); Michael March Schmidt, Cambridge, MA (US); Monia Draghi, Cambridge, MA (US); Amanda Frank Oliphant, Cambridge, MA (US); Sara Marie Halmos, Cambridge, MA (US); Thomas Joseph Schuetz, Cambridge, MA (US); Jason Michael Lajoie, Cambridge, MA (US); Allison Nelson, Cambridge, MA (US)

(73) Assignee: COMPASS THERAPEUTICS LLC, Cambridge, MA (US)

(21) Appl. No.: 16/418,166

(22) Filed: May 21, 2019

Related U.S. Application Data
(60) Provisional application No. 62/674,289, filed on May 21, 2018.
(60) Provisional application No. 62/674,279, filed on May 21, 2018.
(60) Provisional application No. 62/674,286, filed on May 21, 2018.
(60) Provisional application No. 62/728,542, filed on Sep. 7, 2018.
(60) Provisional application No. 62/731,030, filed on Sep. 13, 2018.
(60) Provisional application No. 62/731,045, filed on Sep. 13, 2018.
(60) Provisional application No. 62/731,047, filed on Sep. 13, 2018.
(60) Provisional application No. 62/756,012, filed on Nov. 5, 2018.
(60) Provisional application No. 62/760,473, filed on Nov. 13, 2018.
(60) Provisional application No. 62/760,670, filed on Nov. 13, 2018.
(60) Provisional application No. 62/760,644, filed on Nov. 13, 2018.
(60) Provisional application No. 62/767,786, filed on Nov. 15, 2018.
(60) Provisional application No. 62/767,792, filed on Nov. 15, 2018.
(60) Provisional application No. 62/767,831, filed on Nov. 15, 2018.
(60) Provisional application No. 62/789,946, filed on Jan. 8, 2019.
(60) Provisional application No. 62/789,943, filed on Jan. 8, 2019.
(60) Provisional application No. 62/789,947, filed on Jan. 8, 2019.
(60) Provisional application No. 62/817,450, filed on Mar. 12, 2019.
(60) Provisional application No. 62/817,442, filed on Mar. 12, 2019.
(60) Provisional application No. 62/817,467, filed on Mar. 12, 2019.
(60) Provisional application No. 62/822,243, filed on Mar. 22, 2019.
(60) Provisional application No. 62/822,420, filed on Mar. 22, 2019.
(60) Provisional application No. 62/830,417, filed on Apr. 6, 2019.
(60) Provisional application No. 62/830,420, filed on Apr. 6, 2019.

(51) Int. Cl. C07K 16/28; C07K 16/32; A61P 35/00

(52) U.S. Cl. CPC C07K 16/2803; C07K 16/2878; C07K 16/32; A61P 35/00; C07K 2317/565; A61K 2039/505; C07K 2317/41; C07K 2317/72; C07K 2317/732; C07K 2317/92; C07K 2317/31

(57) ABSTRACT

The present disclosure provides immunotherapeutic compositions and methods for enhancing an immune response and for treating cancer or inflammatory conditions mediated by autoreactive B cells in a subject. In some aspects, multispecific antigen-binding constructs are provided that recognize at least one tumor antigen or B-lineage cell antigen and NKp30 and/or another activating NK receptor. In some aspects, multispecific antigen-binding constructs are provided that recognize at least two tumor antigens or two antigens expressed by B-lineage cells, NKp30, and another activating NK receptor. The multispecific antigen-binding constructs and methods disclosed herein can be used for the treatment of cancer, even a cancer characterized by a CD16 deficient microenvironment and/or characterized by target cells (e.g., cancer cells) having a low level of expression of the tumor antigen.

### CROSS-REFERENCE RELATED APPLICATIONS

This application claims benefit under 35 U.S.C. § 119(e) to U.S. Provisional Patent Application No. 62/674,289, filed on May 21, 2018; U.S. Provisional Patent Application No. 62/674,279, filed on May 21, 2018; U.S. Provisional Patent Application No. 62/674,286, filed on May 21, 2018; U.S. Provisional Patent Application No. 62/728,542, filed on Sep. 7, 2018; U.S. Provisional Patent Application No. 62/731,030, filed on Sep. 13, 2018; U.S. Provisional Patent Application No. 62/731,045, filed on Sep. 13, 2018; U.S. Provisional Patent Application No. 62/731,047, filed on Sep. 13, 2018; U.S. Provisional Patent Application No. 62/756,012, filed on Nov. 5, 2018; U.S. Provisional Patent Application No. 62/760,473, filed on Nov. 13, 2018; U.S. Provisional Patent Application No. 62/760,670, filed on Nov. 13, 2018; U.S. Provisional Patent Application No. 62/760,644, filed on Nov. 13, 2018; U.S. Provisional Patent Application No. 62/767,786, filed on Nov. 15, 2018; U.S. Provisional Patent Application No. 62/767,792, filed on Nov. 15, 2018; U.S. Provisional Patent Application No. 62/767,831, filed on Nov. 15, 2018; U.S. Provisional Patent Application No. 62/789,946, filed on Jan. 8, 2019; U.S. Provisional Patent Application No. 62/789,943, filed on Jan. 8, 2019; U.S. Provisional Patent Application No. 62/789,947, filed on Jan. 8, 2019; U.S. Provisional Patent Application No. 62/817,450, filed on Mar. 12, 2019; U.S. Provisional Patent Application No. 62/817,442, filed on Mar. 12, 2019; U.S. Provisional Patent Application No. 62/817,467, filed on Mar. 12, 2019; U.S. Provisional Patent Application No. 62/822,243, filed on Mar. 22, 2019; U.S. Provisional Patent Application No. 62/822,420, filed on Mar. 22, 2019; U.S. Provisional Patent Application No. 62/830,417, filed on Apr. 6, 2019; and U.S. Provisional Patent Application No. 62/830,420, filed on Apr. 6, 2019, the contents of each of which are incorporated herein in their entireties.

### BACKGROUND

Cancer is one of the leading causes of death, accounting for almost one in six deaths worldwide in 2015, according to the American Association for Cancer Research (AACR) Cancer Progress Report of 2017. The molecular mechanisms involved in cancer are highly complex. In some circumstances, immune cells, such as T cells, natural killer cells, and macrophages, exhibit anti-tumor activity and can effectively control the development and/or progression of tumors. The immune cells recognize tumor antigens (tumor-specific or tumor-associated antigens) and eliminate cells expressing the antigens. However, tumors also constitute highly suppressive microenvironments and can downregulate the function of infiltrating immune cells. For example, tumor cells can downregulate the level of tumor-specific or tumor-associated antigens and escape cell death by the infiltrating immune cells. As a result, the patient's immune system may not recognize cancer cells as foreign or may not be strong enough to destroy the cancer cells.

A number of treatments for cancer are currently available, including surgery, radiation, chemotherapy, hormone therapy, immunotherapy, targeted therapy, stem cell transplants, and precision medicine. Immunotherapeutic approaches, in particular, have been developed in recent years to utilize the patient's endogenous immune system cells (e.g., T cells, natural killer cells, and macrophages) to inhibit tumor formation and progression. CD16 is a target for certain immunotherapies. For example, margetuximab is an Fc-optimized monoclonal antibody that recognizes human epidermal growth factor receptor 2 (HER2) expressed on a number of tumor cells and targets CD16a on immune effector cells (e.g., NK cells). In addition, bispecific antibody fragments that recognize CD16 and CD19 have been developed to target immunotherapeutic drugs to B cell lymphomas. Another immunotherapy in development involves multispecific binding proteins that bind NKG2D, a tumor antigen, and CD16. However, certain patients have cancers in which the levels of CD16 are present at decreased levels on NK cells as compared to control NK cells.

The existing immuno-oncology therapies are still somewhat ineffective for the majority of patients and tumors. When tumor cells downregulate expression of tumor antigens, for example, an immunotherapy directed to the tumor antigen can prove ineffective. Monoclonal antibodies targeting tumor-associated antigens have failed to show effectiveness against low antigen-expressing tumors. Therefore, patients with low antigen expression levels are not eligible for certain available immunotherapies.

Additionally, inflammatory conditions (e.g., inflammatory conditions mediated in whole or in part by auto-reactive B cells) are common. B cells (including plasma cells) have multiple roles in such conditions, including secretion of autoantibodies, presentation of autoantigen, secretion of inflammatory cytokines, modulation of antigen processing and presentation, and generation of ectopic germinal centers. Although depletion of B cells has been proposed, global B-cell depletion eliminates both protective and pathogenic B cells.

### SUMMARY OF THE DISCLOSURE

The present disclosure is based, at least in part, on the discovery of multispecific antigen-binding constructs that are capable of enhancing an immune response (e.g., enhancing the activity of NK cells) against cancer cells. While the disclosure is not bound by any particular theory or mechanism of action, the constructs described herein, when bound to one or more tumor or B-lineage cell antigens expressed by a cancer cell or B-lineage cell, and/or one or more additional NK cell receptors, are capable of agonizing NKp30 activity on adjacent immune effector cells and thereby enhancing an immune response (e.g., increasing NK effector function, T cell proliferation, IFNγ production and secretion, antibody-dependent cell-mediated cytotoxicity (ADCC), and/or the cytolytic activity of T cells) toward the target cells to which the constructs are bound. For example, multispecific antigen-binding constructs are provided that agonize NKp30 function and/or one or more additional NK cell receptors and enhance the immune response with respect to tumor cells that express one or more tumor antigens (e.g., B cell maturation antigen (BCMA) or HER2).

The present multispecific antigen-binding constructs are marked by one or more exemplary features. While the disclosure is not bound by any particular theory or mechanism of action, first, the antigen-binding unit(s) that bind(s) to the one or more tumor antigens or B-lineage cell antigens, such as BCMA, can inhibit the interaction between the target antigen and its cognate ligand or receptor. In some embodiments, signaling via the tumor or B-lineage cell antigen can enhance the proliferation and/or viability of the cancer cell that expresses the antigen. Second, though such activity is not required for function of the described constructs, the constructs can also be effector function enabled (e.g., they can comprise an effector function enabled Fc region of an antibody), which supports antibody-dependent cellular cytotoxicity (ADCC). Third, the constructs are structured such that they can bind simultaneously to target cells and NK cells, and, fourth, the constructs are capable of triggering NK cell activation via engagement of NKp30, inhibiting the effect of soluble NKp30 ligands and increasing pro-inflammatory cytokines by NK cells, e.g., in the tumor microenvironment, as well as, in some embodiments, engaging one or more additional NK cell receptors.

Accordingly, in one aspect, the disclosure provides multispecific (e.g., bispecific, trispecific, tetraspecific) antigen-binding constructs that include at least two linked antigen-binding units, wherein a first antigen-binding unit specifically binds a tumor or B-lineage cell antigen, and wherein a second antigen-binding unit specifically binds an NKp30 antigen.

In another aspect, the disclosure features a multispecific antigen-binding construct comprising at least three linked antigen-binding units, wherein a first antigen-binding unit specifically binds a first tumor or B-lineage cell antigen, a second antigen-binding unit specifically binds an NKp30 antigen, and a third antigen-binding unit binds to a second tumor or B-lineage cell antigen or an NK receptor (an activating NK cell receptor).

In some embodiments of any of the constructs described herein, the third antigen-binding unit binds to a second tumor or B-lineage cell antigen.

In some embodiments of any of the constructs described herein, the third antigen-binding unit binds to an NK receptor.

In some embodiments of any of the constructs described herein, the NK receptor is NKp30, e.g., the construct is bivalent for NKp30. In some embodiments of any of the constructs described herein, the NK receptor is NKG2D. In some embodiments of any of the constructs described herein, the NK receptor is NKp46 or NKp44. In some embodiments of any of the constructs described herein, the NK receptor is any other NK cell activating receptor described herein or known in the art.

In some embodiments of any of the constructs described herein, the construct comprises two antigen-binding units that bind to the first tumor or B-lineage cell antigen.

In some embodiments of any of the constructs described herein, the first tumor or B-lineage cell antigen and the second tumor or B-lineage cell antigen are the same antigen. For example, a construct described herein can be at least bivalent for a single target antigen, e.g., BCMA.

In yet another aspect, the disclosure features a multispecific antigen-binding construct comprising at least three linked antigen-binding units, wherein a first antigen-binding unit specifically binds a first tumor or B-lineage cell antigen, a second antigen-binding unit specifically binds an NKp30 antigen, and a third antigen-binding unit binds to a second tumor or B-lineage cell antigen.

In another aspect, the disclosure features a multispecific antigen-binding construct comprising at least three linked antigen-binding units, wherein a first antigen-binding unit specifically binds a first tumor or B-lineage cell antigen, a second antigen-binding unit specifically binds an NKp30 antigen, and a third antigen-binding unit binds to an NK receptor.

In another aspect, the disclosure provides a multispecific antigen-binding construct comprising at least four linked antigen-binding units, wherein (i) a first antigen-binding unit specifically binds a first tumor or B-lineage cell antigen, a second antigen-binding unit specifically binds a first NK cell activating receptor, a third antigen-binding unit binds a second NK cell activating receptor, and a fourth antigen-binding unit binds to a second tumor or B-lineage cell antigen, and wherein (ii) the first NK receptor is NKp30 and the second NK receptor is not NKp30. For example, the second NK receptor can be NKG2D, 2B4, NK-46, CD226, CD137, or NKp44.

In some embodiments of any of the constructs described herein, the first tumor or B-lineage cell antigen and the second tumor or B-lineage cell antigen are the same antigen.

In some embodiments of any of the constructs described herein, the first tumor antigen or the second tumor antigen is 1GH-IGK, 43-9F, 5T4, 791Tgp72, acyclophilin C-associated protein, alpha-fetoprotein (AFP), α-actinin-4, A3, antigen specific for A33 antibody, ART-4, B7, Ba 733, BAGE, BCMA, BCR-ABL, beta-catenin, beta-HCG, BrE3-antigen, BCA225, BTAA, CA125, CA 15-3\CA 27.29\BCAA, CA195, CA242, CA-50, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEA (CEACAMS), CEACAM6, c-Met, DAM, E2A-PRL, EGFR, EGFRvIII, EGP-1 (TROP-2), EGP-2, ELF2-M, Ep-CAM, fibroblast growth factor (FGF), FGF-5, Flt-1, Flt-3, folate receptor, G250 antigen, Ga733VEpCAM, GAGE, gp100, GRO-β, H4-RET, HLA-DR, HM1.24, human chorionic gonadotropin (HCG) and its subunits, HER2/neu, HMGB-1, hypoxia inducible factor (HIF-1), HSP70-2M, HST-2, HTgp-175, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, mCRP, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pme117 prostatic acid phosphatase, PSA, PRAME, PSMA, P1GF, ILGF, ILGF-1R, IL-6, IL-25, RCAS1, RS5, RAGE, RANTES, Ras, T101, SAGE, S100, survivin, survivin-2B, SDDCAG16, TA-90\Mac2 binding protein, TAAL6, TAC, TAG-72, TLP, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, 17-1A-antigen, complement factors C3, C3a, C3b, C5a, C5, an angiogenesis marker, bc1-2, bc1-6, or K-ras.

In some embodiments of any of the constructs described herein, the first or second target antigen expressed by B-lineage cells are selected from the group consisting of BCMA, CD19, CD20, CD21, CD22, CD23, CD24, CD27, CD38, CD40, CD72, CD78, CD79a, CD79b, CD80, CD84, CD86, CD126, CD138, CD319, TAC. In some embodiments of any of the constructs described herein, one or both of the first tumor or B-lineage cell antigen and second tumor or B-lineage cell antigen is B cell maturation antigen (BCMA).

In some embodiments of any of the constructs described herein, one or more of the antigen-binding units is/are an antibody or an antigen-binding portion thereof.

In some embodiments of any of the constructs described herein, each of the antigen-binding units are an antibody or an antigen-binding portion thereof. In some embodiments of any of the constructs described herein, the antibody or antigen-binding portion thereof of the first antigen-binding unit is a monoclonal antibody or antigen-binding portion thereof.

In some embodiments of any of the constructs described herein, the antibody or antigen-binding portion thereof of any one of the antigen-binding units is a humanized antibody, a fully human antibody, or an antigen-binding portion of either.

In some embodiments of any of the constructs described herein, the antibody or antigen-binding portion thereof of any one of the antigen-binding units is a scFv or Fab antibody fragment.

In some embodiments of any of the constructs described herein, one or more of the antigen-binding units is/are a non-antibody scaffold protein.

In some embodiments of any of the constructs described herein, one or more of the antigen-binding units is/are a polypeptide, small molecule, or an aptamer.

In some embodiments, any of the constructs described herein further comprise an additional antigen-binding unit that binds specifically to a molecule expressed by an effector immune cell. The molecule expressed by the effector immune cell can be, e.g., CD16, CD16a, CD16b, CD32a, CD64, or CD89.

In some embodiments of any of the constructs described herein, the binding of one antigen-binding unit to its target does not block the binding of another antigen-binding unit to its target.

In some embodiments of any of the constructs described herein, the first antigen-binding unit and second antigen-binding unit bind to their respective targets and both antigen-binding units remain bound concurrently.

In some embodiments of any of the constructs described herein, all antigen-binding units bind to their respective targets and remain bound concurrently.

In some embodiments of any of the constructs described herein, binding of a first antigen-binding unit and one or more additional antigen-binding units to their respective targets can bridge a first cell and a tumor cell or B-lineage cell together.

In some embodiments of any of the constructs described herein, the bridging of the first cell and the tumor cell or B-lineage cell is determined by flow cytometry and/or fluorescence plate reader.

In some embodiments of any of the constructs described herein, one or both of the first antigen-binding unit and the fourth antigen-binding unit inhibits the target antigen function.

In some embodiments of any of the constructs described herein, the construct is a multispecific antibody.

In some embodiments of any of the constructs described herein, the construct comprises a common light chain.

In some embodiments of any of the constructs described herein, the construct is tetravalent.

In some embodiments of any of the constructs described herein, the construct is at least bivalent for at least one tumor or B-lineage cell antigen.

In some embodiments of any of the constructs described herein, the construct does not comprise an Fc domain.

In some embodiments of any of the constructs described herein, one or more antigen-binding units comprise a heavy chain comprising one or more immunoglobulin Fc modifications in an Fc domain.

In some embodiments of any of the constructs described herein, the immunoglobulin Fc domain of the heavy chain comprises one or more amino acid mutations that promote heterodimerization of the one or more antigen-binding units.

In some embodiments of any of the constructs described herein, the mutation is present in a CH3 domain of the heavy chain.

In some embodiments of any of the constructs described herein, the multispecific antigen-binding construct is produced in a quadroma cell.

In some embodiments of any of the constructs described herein, the construct comprises one or more immunoglobulin constant region modifications.

In some embodiments of any of the constructs described herein, the immunoglobulin constant region comprises one or more amino acid mutations that promote heterodimerization of antibodies.

In some embodiments of any of the constructs described herein, one or more mutations is present in the light chain constant region of one antigen-binding unit and one or more mutations is present in the heavy chain constant region of another antigen-binding unit.

In some embodiments of any of the constructs described herein, the Fc domain has increased effector function.

In some embodiments of any of the constructs described herein, the Fc domain has decreased effector function.

In some embodiments of any of the constructs described herein, the Fc domain enhances half-life of the construct.

In some embodiments of any of the constructs described herein, one or both of the first tumor antigen and second tumor antigen is/are a tumor-specific antigen.

In some embodiments of any of the constructs described herein, the first antigen-binding unit and the second antigen-binding unit are linked by at least one amino linker amino acid sequence.

In some embodiments of any of the constructs described herein, the linker amino acid sequence comprises GGGGSx (SEQ ID NO: 22), wherein x is an integer between and including 1 to 6.

In some embodiments of any of the constructs described herein, the construct comprises heavy chain CDR1, CDR2 and CDR3 sequences set forth in SEQ ID NOs: 11, 12, and 13, respectively.

In some embodiments of any of the constructs described herein, the construct comprises the heavy chain variable region sequence set forth in SEQ ID NO: 10.

In some embodiments of any of the constructs described herein, the construct comprises heavy chain CDR1, CDR2 and CDR3 sequences set forth in SEQ ID NOs: 15, 16, and 17, respectively.

In some embodiments of any of the constructs described herein, the construct comprises the heavy chain variable region sequence set forth in SEQ ID NOs: 14, 25, or 72 or a heavy chain variable sequence having at least 90% identity to the sequence of SEQ ID NOs: 14, 25, or 72.

In some embodiments of any of the constructs described herein, the construct comprises light chain CDR1, CDR2 and CDR3 sequences set forth in SEQ ID NOs: 19, 20, and 21, respectively.

In some embodiments of any of the constructs described herein, the construct comprises the light chain variable region sequence set forth in SEQ ID NO: 18 or a light chain variable sequence having at least 90% identity to the sequence of SEQ ID NO: 18.

In some embodiments of any of the constructs described herein, the construct comprises the heavy chain sequence set forth in SEQ ID NO: 9 or 24 and the light chain sequence set forth in SEQ ID NO: 8.

In some embodiments, the multispecific antigen-binding construct comprises heavy chain CDR1, CDR2 and CDR3 sequences set forth in either SEQ ID NOs: 38, 39, and 40, respectively; SEQ ID NOs: 11, 12, and 41, respectively; SEQ ID NOs: 44, 45, and 46, respectively; SEQ ID NOs: 47, 48, and 46, respectively; SEQ ID NOs: 11, 45, and 49, respectively; SEQ ID NOs: 11, 50, and 41, respectively; or SEQ ID NOs: 44, 50, and 41, respectively. Optionally, the construct comprises the heavy chain variable region sequence of any one of SEQ ID NO: 10, SEQ ID NO: 52, SEQ ID NO: 53, SEQ ID NO: 54, SEQ ID NO: 55, SEQ ID NO: 56, and SEQ ID NO: 57 or a heavy chain variable sequence having at least 90% identity to the sequence of any one of SEQ ID NO: 10, SEQ ID NO: 52, SEQ ID NO: 53, SEQ ID NO: 54, SEQ ID NO: 56, and SEQ ID NO: 57. By way of example, the construct can comprise (a) a heavy chain CDR1, CDR2 and CDR3 sequences set forth in SEQ ID NOs: 15, 16, and 42, (b) a heavy chain CDR1, CDR2 and CDR3 sequences set forth in SEQ ID NOs: 69, 70, and 71, or (c) a heavy chain CDR1, CDR2 and CDR3 sequences set forth in SEQ ID Nos: 75, 76, and 77, respectively. The construct can comprise light chain CDR1, CDR2 and CDR3 sequences set forth in SEQ ID NOs: 19, 20, and 43, respectively.

Also provided herein is a construct comprising a heavy chain having an amino acid sequence of any one of SEQ ID NO: 9, SEQ ID NO: 24, SEQ ID NO: 26, SEQ ID NO: 27, SEQ ID NO: 65, SEQ ID NO: 66, SEQ ID NO: 67, SEQ ID NO: 68, and SEQ ID NO: 74, and a light chain having an amino acid sequence of SEQ ID NO: 8.

In some embodiments of any of the constructs described herein, the second antigen-binding unit that specifically binds a NKp30 antigen optionally binds to an epitope on human NKp30 comprising at least one of residues I50, S82, or L113 of SEQ ID NO: 7. Optionally, the epitope comprises residue I50 of SEQ ID NO: 7. Optionally, the epitope comprises residue S82 of SEQ ID NO: 7. Optionally, the epitope comprises residue L113 of SEQ ID NO: 7. By way of example, the epitope optionally comprises residues (i) I50 and S82 of SEQ ID NO: 7; (ii) I50 and SL113 of SEQ ID NO: 7; (iii) S82 and L113 of SEQ ID NO: 7 or (iv) I50, S82, and L113 of SEQ ID NO: 7. The second antigen-bindng unit or construct optionally binds NKp30 with an affinity (K_{D}) of about 1×10⁻⁶ or less.

In some embodiments of any of the constructs described herein, the second antigen-binding unit or construct optionally binds to a ligand-binding region of human NKp30. In some aspects, the NKp30 ligand is BAG6, B7-H6, or Gal-3. Optionally, mutation of residues I50, S82, or L113 of SEQ ID NO:7, or any combination thereof, to alanine, results in loss of binding to human NKp30. The epitope optionally is a non-linear epitope.

Optionally, the second antigen-binding unit or construct competes for binding to the epitope on human NKp30 with an antibody or antigen-binding portion thereof having heavy and light chain CDRs selected from the group consisting of: (a) heavy chain CDR1, CDR2 and CDR3 sequences set forth in SEQ ID NOs: 15, 16, and 42, respectively, and light chain CDR1, CDR2 and CDR3 sequences set forth in SEQ ID NOs: 19, 20 and 43, respectively; (b) heavy chain CDR1, CDR2 and CDR3 sequences set forth in SEQ ID NOs: 69, 70 and 71, respectively, and light chain CDR1, CDR2 and CDR3 sequences set forth in SEQ ID NOs: 19, 20 and 43, respectively; and (c) heavy chain CDR1, CDR2 and CDR3 sequences set forth in SEQ ID NOs: 75, 76, and 77, respectively, and light chain CDR1, CDR2 and CDR3 sequences of SEQ ID NO: 80. Optionally, the second antigen-binding unit or construct competes for binding to the epitope on human NKp30 with an antibody or antigen-binding portion thereof having a heavy chain variable region amino acid sequence of SEQ ID NO: 14, SEQ ID NO: 25, or SEQ ID NO: 72; and a light chain variable region amino acid sequence of SEQ ID NO: 18; or a heavy chain variable region amino acid sequence of SEQ ID NO: 78 and a light chain variable region amino acid sequence of SEQ ID NO: 80. Optionally, the second antigen-binding unit or construct competes for binding to the epitope on human NKp30 with an antibody or antigen-binding portion thereof having (i) a heavy chain variable region having an amino acid sequence that is at least 90% identical to the amino acid sequence of SEQ ID NO: 14, SEQ ID NO: 25, or SEQ ID NO:72; and a light chain variable region having an amino acid sequence that is at least 90% identical to the amino acid sequence of SEQ ID NO: 18 or (ii) a heavy chain variable region having an amino acid sequence that is at least 90% identical to the amino acid sequence of SEQ ID NO: 78 and a light chain variable region having an amino acid sequence that is at least 90% identical to the amino acid sequence of SEQ ID NO: 80.

In some embodiments of any of the constructs described herein, the second antigen-binding unit binds to an epitope of human NKp30, wherein the epitope is within or overlapping with amino acids 50-113 of SEQ ID NO: 7, and wherein one or more of substitutions at I50, S82, and L113 disrupts binding of the antibody or antigen-binding portion to human NKp30. Optionally, the epitope is a conformational epitope. Optionally, the epitope is a linear epitope.

Provided herein is an isolated monoclonal antibody or antigen-binding fragment thereof that specifically binds human BCMA, wherein the antibody or antigen-binding fragment thereof comprises a heavy chain variable region comprising a CDRH1 of SEQ ID NO: 38 (YTFX₁X₂X₃YX₄H, wherein X₁ is T or S, X₂ is N or S, X₃ is Y or H, and X₄ is M or V); a CDRH2 of SEQ ID NO: 39 (GX₅IDPSX₆GX₇TX₈YA, wherein X₅ is V or I, X₆ is G or D, X₇ is G, Y or S, and X₈ is N or S); and a CDRH3 of SEQ ID NO: 40 (ARGRYDYX₉DYLGWFDX₁₀, wherein X₉ is G or S, X₁₀ is P or G). The isolated monoclonal antibody or antigen-binding fragment thereof optionally comprises CDRH1, CDRH2, and CDRH3 as set forth in either SEQ ID NO: 11, SEQ ID NO: 12, and SEQ ID NO: 41, respectively; SEQ ID NO: 44, SEQ ID NO: 45, and SEQ ID NO: 46, respectively; SEQ ID NO: 47, SEQ ID NO: 48, and SEQ ID NO: 46, respectively; SEQ ID NO: 11, SEQ ID NO: 45, and SEQ ID NO: 49, respectively; SEQ ID NO: 11, SEQ ID NO: 50, and SEQ ID NO: 41, respectively; SEQ ID NO: 44, SEQ ID NO: 50, and SEQ ID NO: 41, respectively. Optionally, antibody or antigen-binding fragment thereof further comprises a CDRL1 of SEQ ID 19, a CDRL2 of SEQ ID NO: 20, and a CDRL3 of SEQ ID NO: 43. By way of example, an antibody or antigen-binding fragment thereof as described can comprise a heavy chain variable sequence that is at least 90% identical to the amino acid sequence of either SEQ ID NO: 10, SEQ ID NO: 52, SEQ ID NO: 53, SEQ ID NO: 54, SEQ ID NO: 55, SEQ ID NO: 56, or SEQ ID NO: 57. By way of further example, the antibody or antigen-binding fragment thereof optionally comprises a light chain variable sequence that is at least 90% identical to the amino acid sequence of SEQ ID NO: 18. Thus, the antibody or antigen-binding fragment thereof can comprise a heavy chain variable sequence selected from any one of SEQ ID NO: 10, SEQ ID NO: 52, SEQ ID NO: 53, SEQ ID NO: 54, SEQ ID NO: 55, SEQ ID NO: 56, and SEQ ID NO: 57, and a light chain variable sequence of SEQ ID NO: 18.

Provided herein is an isolated monoclonal antibody or antigen-binding fragment thereof, that specifically binds to human BCMA, wherein, when bound to human BCMA, the antibody or antigen-binding fragment thereof binds to at least one of the amino acid residues bound by an anti-human BCMA antibody comprising the heavy chain variable region sequence of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and a light chain variable sequence of SEQ ID NO: 18.

Also provided is an isolated monoclonal antibody or antigen-binding fragment thereof that specifically binds human NKp30, wherein the antibody or antigen-binding fragment thereof comprises a heavy chain variable region comprising (a) a CDRH1 of SEQ ID NO: 15, a CDRH2 of SEQ ID NO: 16, and a CDRH3 of SEQ ID NO: 42; (b) a CDRH1 of SEQ ID NO: 69, a CDRH2 of SEQ ID NO: 70, and a CDRH3 of SEQ ID NO: 71, or (c) a CDRH1 of SEQ ID NO:75, a CDRH2 of SEQ ID NO: 76, and a CDRH3 of SEQ ID NO: 77. Optionally, the antibody or antigen-binding fragment thereof comprises a light chain variable region comprising a CDRL1 of SEQ ID NO: 19, a CDRL2 of SEQ ID NO: 20, and a CDRL3 of SEQ ID NO: 43. Optionally, the antibody or antigen-binding fragment thereof comprises a heavy chain variable sequence that is at least 90% identical to the amino acid sequence of SEQ ID NO: 14, SEQ ID NO: 25, or SEQ ID NO: 72. Optionally, the antibody or antigen-binding fragment thereof comprises a light chain variable sequence that is at least 90% identical to the amino acid sequence of SEQ ID NO: 18. Optionally, the antibody or antigen-binding fragment thereof comprises a heavy chain variable sequence that is at least 90% identical to the amino acid sequence of SEQ ID NO: 78. Optionally, the antibody or antigen-binding fragment thereof comprises a light chain variable sequence that is at least 90% identical to the amino acid sequence of SEQ ID NO: 80. Thus, provided herein is an antibody or antigen-binding fragment thereof comprising (i) the heavy chain variable region sequence of SEQ ID NO: 14, SEQ ID NO: 25, or SEQ ID NO: 72, and the light chain variable region sequence of SEQ ID NO: 18, or (ii) the heavy chain variable region sequence of SEQ ID NO: 78 and the light chain variable region sequence of SEQ ID NO: 80.

Further provided is an isolated monoclonal antibody or antigen-binding fragment thereof that specifically binds to human NKp30, wherein, when bound to human NKp30, the antibody or antigen-binding fragment thereof cross blocks the binding of an anti-human NKp30 antibody comprising (i) the heavy chain variable region sequence of SEQ ID NO: 14, SEQ ID NO: 25, or SEQ ID NO: 72, and the light chain variable region sequence of SEQ ID NO: 18, or (ii) the heavy chain variable region sequence of SEQ ID NO: 78 and the light chain variable region sequence of SEQ ID NO: 80.

In some embodiments of these aspects and all such aspects described herein, the first and/or second tumor antigen is associated with a carcinoma, sarcoma, myeloma, leukemia, lymphoma, or combination thereof. Optionally, in some embodiments, the first and/or second tumor antigen is an epithelial cancer antigen, a prostate specific cancer antigen (PSA) or prostate specific membrane antigen (PSMA), a bladder cancer antigen, a lung cancer (e.g., small cell lung) antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, a colorectal cancer antigen, a lymphoma (e.g., non-Hodgkin's lymphoma or Hodgkin's lymphoma) antigen, a B-cell lymphoma cancer antigen, a leukemia antigen, a myeloma (e.g., multiple myeloma or plasma cell myeloma) antigen, an acute lymphoblastic leukemia antigen, a chronic myeloid leukemia antigen, or an acute myelogenous leukemia antigen. In certain embodiments, the tumor antigen is BCMA.

In certain embodiments, the first antigen-binding unit of the multispecific antigen-binding construct binds a first antigen expressed by the B cell, such as an autoreactive B cell or plasma cell. Optionally, the first antigen is a B cell-specific, B cell-restricted, or B cell-enriched antigen. Optionally, the first antigen is selected from the group consisting of CD19, CD20 and BCMA. In certain embodiments, the third antigen-binding unit of the multispecific antigen-binding construct binds to a second antigen expressed by a B cell. Optionally, the third antigen-binding unit binds to an NK receptor (e.g., NKp30, NKG2D, NKp46, or NKp44). Optionally, the construct comprises two antigen-binding units that bind to the first target antigen expressed by a B-lineage cell. Optionally, the first target antigen expressed by a B cell and the second target antigen expressed by a B cell are the same antigen.

Provided herein is a multispecific antigen-binding construct comprising at least three linked antigen-binding units, wherein a first antigen-binding unit specifically binds a first target antigen expressed by a B-lineage cell, a second antigen-binding unit specifically binds an NKp30 antigen, and a third antigen-binding unit binds to an antigen expressed by a second B-lineage cell antigen or to an NK receptor. Optionally, the first target antigen expressed by a B-lineage cell is B cell maturation antigen (BCMA). Optionally, the second target antigen expressed by a B-lineage cell is selected from the group consisting of BCMA, CD1c, CD5, CD10, CD19, CD20, CD21, CD22, CD23, CD24, CD27, CD34, CD38, CD40, CD72, CD78, CD79a, CD79b, CD80, CD84, CD86, CD126, CD138, CD319, TAC, GPRCSD (G protein-coupled receptor class C group 5 member D), SLAMF7 (CS1), and IL7/3R. The B-lineage cell is selected from the group consisting of a pro-B cell, a pre-B cell, a transitional B cell, a follicular B cell, a marginal zone B cell, a germinal center B cell, a plasma cell, and a memory B cell. In certain embodiments, the first antigen-binding unit inhibits the function of the first target antigen expressed by the B-lineage cell.

In some embodiments, the first antigen-binding unit is a non-antibody scaffold protein. In certain embodiments, the non-antibody scaffold protein is an anticalin. In other embodiments, the first antigen-binding unit is a polypeptide, small molecule, or an aptamer. Optionally, the first antigen-binding unit is an antibody or antigen-binding fragment thereof. The antibody or antigen-binding portion thereof of the first antigen-binding unit is optionally a monoclonal antibody or antigen-binding portion thereof. The antibody or antigen-binding portion thereof of the first antigen-binding unit can be a humanized antibody, a fully human antibody, or an antigen-binding portion of either. The antibody or antigen-binding portion thereof of the first antigen-binding unit is optionally an scFv or Fab antibody fragment.

In some embodiments, the second antigen-binding unit is a non-antibody scaffold protein. In certain embodiments, the non-antibody scaffold protein is an anticalin. In other embodiments, the second antigen-binding unit is a polypeptide, small molecule, or an aptamer. Optionally, the second antigen-binding unit is an antibody or antigen-binding fragment thereof. The second antigen-binding unit is optionally a monoclonal antibody or antigen-binding portion thereof. The second antigen-binding unit can be a humanized antibody or a fully human antibody, or an antigen-binding portion of either. The antibody or antigen-binding portion thereof of the second antigen-binding unit is optionally a scFv or Fab antibody fragment.

In some embodiments, the third antigen-binding unit is a non-antibody scaffold protein. In certain embodiments, the non-antibody scaffold protein is an anticalin. In other embodiments, the third antigen-binding unit is a polypeptide, small molecule, or an aptamer. Optionally, the third antigen-binding unit is an antibody or antigen-binding fragment thereof. The third antigen-binding unit is optionally a monoclonal antibody or antigen-binding portion thereof. The third antigen-binding unit can be a humanized antibody or a fully human antibody, or an antigen-binding portion of either. The antibody or antigen-binding portion thereof of the third antigen-binding unit is optionally a scFv or Fab antibody fragment.

In some embodiments, the fourth antigen-binding unit is a non-antibody scaffold protein. In certain embodiments, the non-antibody scaffold protein is an anticalin. In other embodiments, the fourth antigen-binding unit is a polypeptide, small molecule, or an aptamer. Optionally, the fourth antigen-binding unit is an antibody or antigen-binding fragment thereof. The fourth antigen-binding unit is optionally a monoclonal antibody or antigen-binding portion thereof. The fourth antigen-binding unit can be a humanized antibody or a fully human antibody, or an antigen-binding portion of either. The antibody or antigen-binding portion thereof of the fourth antigen-binding unit is optionally a scFv or Fab antibody fragment. The antigen-binding construct optionally further comprises an additional antigen-binding unit that binds specifically to a molecule expressed by an effector immune cell. In certain embodiments, the molecule expressed by the effector immune cell is CD16, CD16a, CD16b, CD32a, CD64, or CD89.

In some embodiments, the antigen-binding construct further comprises a third antigen-binding unit that binds specifically to a second tumor or B-lineage cell antigen.

The antigen-binding construct further comprises a third antigen-binding unit, in some embodiments, that binds specifically to another activating NK cell receptor, in addition to NKp30, i.e., that is not an antigen found on NKp30. Non-limiting examples of such activating NK cell receptors include NKp46, NKp44, 2B4, CD226, NKG2D, CD137, CD16a, and CD2. The antigen-binding construct, in some embodiments, further comprises a third antigen-binding unit that binds to a second tumor or B-lineage cell antigen and a fourth antigen-binding unit specifically binding to another activating NK cell receptor, in addition to NKp30, i.e., that is not an antigen found on NKp30. Non-limiting examples of such activating NK cell receptors include NKp46, NKp44, 2B4, CD226, NKG2D, CD137, CD16a, and CD2.

The two or more antigen-binding units disclosed herein can be the same or different structure within the same antigen-binding construct. For example, all antigen-binding units can be antibodies or antigen-binding portions thereof, or a subset can be antibodies or antigen-binding portions thereof.

In some embodiments, the binding of one antigen-binding unit to its target does not block the binding of other antigen-binding unit(s) to their target(s). Optionally, the two or more antigen-binding units bind to their respective targets and all antigen-binding units remain bound concurrently. In some embodiments, binding of one antigen-binding unit and binding of another antigen-binding unit to their respective targets can bridge a first cell, such as an immune cell, and a tumor cell together. Optionally, the bridging of the first cell and the tumor cell is determined by flow cytometry and/or fluorescence plate reader. In certain embodiments, the first antigen-binding unit inhibits the tumor antigen function.

The disclosure provides, in some embodiments, that the multispecific antigen-binding construct is a bispecific antibody, a trispecific antibody, or a tetraspecific antibody. Optionally, the construct comprises a common light chain. In certain embodiments, the construct is tetravalent. Optionally, the construct is at least bivalent for the tumor or B-lineage cell antigen and/or at least bivalent for the NKp30 antigen. In some embodiments, the construct does not comprise an Fc domain. In other embodiments, the first antigen-binding unit or second antigen-binding unit, or both, comprises a heavy chain comprising one or more immunoglobulin Fc modifications. In some embodiments, the immunoglobulin Fc domain of the heavy chain comprises one or more amino acid mutations that promote heterodimerization of the first and second antigen-binding units. Optionally, the mutation is present in a CH3 domain of the heavy chain. In some embodiments, the multispecific antigen-binding construct is produced in a quadroma cell. In certain embodiments, the construct comprises one or more immunoglobulin constant region modifications. In some embodiments, the immunoglobulin constant region comprises one or more amino acid mutations that promote heterodimerization of antibodies. Optionally, one or more mutations is present in the light chain constant region of one antigen-binding unit and one or more mutations is present in the heavy chain constant region of another antigen-binding unit. In some embodiments, the bispecific antibody is selected from the group consisting of a bispecific IgG, bispecific antibody fragment, bispecific fusion protein, appended IgG, and bispecific antibody conjugate. In some embodiments, the Fc region has reduced effector function or enhances the half-life of the construct. In certain embodiments, the first antigen-binding unit and the second antigen-binding unit are linked by at least one amino linker amino acid sequence. Optionally, the linker amino acid sequence comprises GGGGSX (SEQ ID NO: 22), wherein x is an integer between and including 1 to 6.

In some embodiments, the multispecific antigen-binding construct comprises heavy chain CDR1, CDR2 and CDR3 sequences set forth in SEQ ID NOs: 11, 12, and 13, respectively. Optionally, the construct comprises the heavy chain variable region sequence set forth in SEQ ID NO: 10. In some embodiments, the multispecific antigen-binding construct comprises heavy chain CDR1, CDR2 and CDR3 sequences set forth in SEQ ID NOs: 15, 16, and 17, respectively. Optionally, the construct comprises the heavy chain variable region sequence set forth in SEQ ID NO: 14 or 25. In some embodiments, the construct comprises light chain CDR1, CDR2 and CDR3 sequences set forth in SEQ ID NOs: 19, 20, and 21, respectively. Optionally, the construct comprises the light chain variable region sequence set forth in SEQ ID NO: 18. In some embodiments, the constructs optionally comprise the heavy chain sequence set forth in SEQ ID NO: 9 or 24 and the light chain sequence set forth in SEQ ID NO: 8.

In embodiments of any of the constructs described herein, the second antigen-binding unit is optionally capable of agonizing NKp30. In some embodiments, the constructs described herein are capable of agonizing NKp30 expressed by an immune effector cell when the constructs are bound to a cell (e.g., a cancer cell or B-lineage cell) expressing the target antigen.

The disclosure provides, in some embodiments, that the multispecific antigen-binding construct is a multispecific antibody. Optionally, the construct comprises a common light chain. In certain embodiments, the construct is tetravalent. In some embodiments, the construct is bivalent for the NKp30 antigen, monovalent for a first tumor or B-lineage cell antigen, and monovalent for a second tumor or B-lineage cell antigen. In some embodiments, the construct is bivalent for the NKp30 antigen, monovalent for a second activating NK receptor, and monovalent for a tumor or B-lineage cell antigen. In some embodiments, the construct is monovalent for the NKp30 antigen, monovalent for a second activating NK receptor, monovalent for a first tumor or B-lineage cell antigen, and monovalent for a second tumor or B-lineage cell antigen. In some embodiments, the construct does not comprise an Fc domain. In other embodiments, at least one antigen-binding unit comprises a heavy chain comprising one or more immunoglobulin Fc modifications. In some embodiments, the immunoglobulin Fc domain of the heavy chain comprises one or more amino acid mutations that promote heterodimerization of the antigen-binding units. Optionally, the mutation is present in a CH3 domain of the heavy chain. In some embodiments, the multispecific antigen-binding construct is produced in a quadroma cell. In certain embodiments, the construct comprises one or more immunoglobulin constant region modifications. In some embodiments, the immunoglobulin constant region comprises one or more amino acid mutations that promote heterodimerization of antibodies. Optionally, one or more mutations is present in the light chain constant region of one antigen-binding unit and one or more mutations is present in the heavy chain constant region of another antigen-binding unit. In some embodiments, the multispecific antibody is selected from the group consisting of a multispecific IgG, multispecific antibody fragment, multispecific fusion protein, appended IgG, and multispecific antibody conjugate. In some embodiments, the Fc region has reduced effector function or enhances the half-life of the construct. In certain embodiments, one or more antigen-binding units are linked by at least one amino linker amino acid sequence. Optionally, the linker amino acid sequence comprises GGGGSX (SEQ ID NO: 22), wherein x is an integer between and including 1 to 6.

The disclosure also provides an isolated monoclonal antibody, or antigen-binding fragment thereof, that specifically binds to human BCMA, wherein, when bound to human BCMA, the antibody, or antigen-binding fragment thereof, binds to at last one of the amino acid residues bound by an anti-human BCMA antibody comprising the heavy chain variable region sequence depicted in SEQ ID NO: 10 and the light chain variable region sequence depicted in SEQ ID NO: 18. In another aspect, the disclosure features an isolated monoclonal antibody, or antigen-binding fragment thereof, that specifically binds to human BCMA, wherein, when bound to human BCMA, the antibody, or antigen-binding fragment thereof, cross blocks the binding of an anti-human BCMA antibody comprising the heavy chain variable region sequence depicted in SEQ ID NO: 10 and the light chain variable region sequence depicted in SEQ ID NO: 18. In some embodiments, an isolated antibody, or antigen-binding fragment thereof, is provided that specifically binds to human BCMA, wherein the antibody or antigen-binding fragment thereof comprises heavy chain CDR1, CDR2 and CDR3 sequences set forth in SEQ ID NOs: 11, 12, and 13, respectively. Optionally, the antibody or antigen-binding fragment thereof comprises light chain CDR1, CDR2 and CDR3 sequences set forth in SEQ ID NOs: 19, 20, and 21, respectively. In certain embodiments, the antibody or antigen-binding fragment thereof comprises an amino acid sequence that is at least 90% identical to the amino acid sequence set forth in SEQ ID NO: 10. In some embodiments, the antibody or antigen-binding fragment thereof comprises an amino acid sequence that is at least 90% identical to the amino acid sequence set forth in SEQ ID NO: 18. Optionally, the antibody or antigen-binding fragment thereof comprises a heavy chain comprising the amino acid sequence set forth in SEQ ID NO: 10 and a light chain comprising the amino acid sequence set forth in SEQ ID NO: 18.

Also provided is an isolated monoclonal antibody, or antigen-binding fragment thereof, that specifically binds to human NKp30, wherein, when bound to human NKp30, the antibody or antigen-binding fragment thereof binds to at least one of the amino acid residues bound by an anti-human NKp30 antibody comprising the heavy chain variable region sequence set forth in SEQ ID NO: 14 or 25 and the light chain variable region sequence set forth in SEQ ID NO: 18. The disclosure also provides an isolated monoclonal antibody, or antigen-binding fragment thereof, that specifically binds to human NKp30, wherein, when bound to human NKp30, the antibody or antigen-binding fragment thereof cross blocks the binding of an anti-human NKp30 antibody comprising the heavy chain variable region sequence set forth in SEQ ID NO: 14 or 25 and the light chain variable region sequence set forth in SEQ ID NO: 18. In some embodiments, an isolated antibody, or antigen-binding fragment thereof, is provided that comprises heavy chain CDR1, CDR2 and CDR3 sequences set forth in SEQ ID NOs: 15, 16, and 17, respectively. In some embodiments, the antibody or antigen-binding fragment thereof comprises light chain CDR1, CDR2 and CDR3 sequences set forth in SEQ ID NOs: 19, 20, and 21, respectively. In certain embodiments, the antibody or antigen-binding fragment thereof comprises an amino acid sequence that is at least 90% identical to the amino acid sequence set forth in SEQ ID NO: 14 or 25. In other embodiments, the antibody or antigen-binding fragment thereof comprises an amino acid sequence that is at least 90% identical to the amino acid sequence set forth in SEQ ID NO: 18. Optionally, the antibody or antigen-binding fragment thereof comprises a heavy chain comprising the amino acid sequence set forth in SEQ ID NO: 14 or 25 and a light chain comprising the amino acid sequence set forth in SEQ ID NO: 18.

Also provided herein is an isolated antibody or antigen-binding portion thereof that binds to an epitope of human NKp30, wherein the epitope is within or overlapping with amino acids 50-113 of SEQ ID NO: 7, and wherein one or more of substitutions at I50, S82, and L113 disrupts binding of the antibody or antigen-binding portion to human NKp30. Optionally, the epitope is a conformational epitope. Optionally, the epitope is a linear epitope.

Also provided herein is an isolated monoclonal antibody or antigen-binding portion thereof that specifically binds human NKp30, wherein the antibody or antigen binding portion binds to an epitope on human NKp30 comprising at least one of residues I50, S82, or L113 of SEQ ID NO: 7. Optionally, the epitope comprises residue I50 of SEQ ID NO: 7. Optionally, the epitope comprises residue S82 of SEQ ID NO: 7. Optionally, the epitope comprises residue L113 of SEQ ID NO: 7. By way of further example, the epitope optionally comprises residues (i) I50 and S82 of SEQ ID NO: 7; (ii) I50 and SL113 of SEQ ID NO: 7; (iii) S82 and L113 of SEQ ID NO: 7 or (iv) I50, S82, and L113 of SEQ ID NO: 7. Optionally, the antibody or antigen binding portion thereof binds to an epitope comprising a sequence of one or more amino acid residues corresponding to amino acid positions 50-113 of SEQ ID NO: 7. In certain embodiments, the antibody or antigen binding portion thereof binds to an epitope comprising 3, 4, 5, 6, 7, 8, 9, 10, 11, 12, 13, 14, 15, 16, 17, 18, 19, or 20 amino acid residues corresponding to amino acid positions 50-113 of SEQ ID NO: 7. The isolated monoclonal antibody or antigen-binding portion thereof optionally binds NKp30 with an affinity (K_{D}) of about 1×10⁻⁶ or less.

The antibody or antigen-binding portion thereof optionally binds to a ligand-binding region of the human NKp30. In some aspects, the NKp30 ligand is BAG6, B7-H6, or Gal-3. Optionally, mutation of residues I50, S82, or L113 of SEQ ID NO:7, or any combination thereof, to alanine, results in reduced binding to human NKp30, as compared to binding in the absence of the mutation. Such reduced binding can be a complete loss of detectable binding. The antibody or antigen-binding portion thereof optionally binds an epitope located within amino acid residues 50-113 of SEQ ID NO:7. The epitope optionally is a non-linear epitope. Optionally, the antibody or antigen-binding portion thereof competes for binding to the epitope on human NKp30 with an antibody or antigen-binding portion thereof having heavy and light chain CDRs selected from the group consisting of: (a) heavy chain CDR1, CDR2 and CDR3 sequences set forth in SEQ ID NOs: 15, 16, and 42, respectively, and light chain CDR1, CDR2 and CDR3 sequences set forth in SEQ ID NOs: 19, 20 and 43, respectively; (b) heavy chain CDR1, CDR2 and CDR3 sequences set forth in SEQ ID NOs: 69, 70 and 71, respectively, and light chain CDR1, CDR2 and CDR3 sequences set forth in SEQ ID NOs: 19, 20 and 43, respectively; and (c) heavy chain CDR1, CDR2 and CDR3 sequences set forth in SEQ ID NOs: 75, 76, and 77, respectively, and light chain CDR1, CDR2 and CDR3 sequences of SEQ ID NO: 80. Optionally, the antibody or antigen-binding portion thereof competes for binding to the epitope on human NKp30 with an antibody or antigen-binding portion thereof having a heavy chain variable region amino acid sequence of SEQ ID NO: 14, SEQ ID NO: 25, or SEQ ID NO: 72; and a light chain variable region amino acid sequence of SEQ ID NO: 18; or a heavy chain variable region amino acid sequence of SEQ ID NO: 78 and a light chain variable region amino acid sequence of SEQ ID NO: 80. Optionally, the antibody or antigen-binding portion thereof competes for binding to the epitope on human NKp30 with an antibody or antigen-binding portion thereof comprising (i) a heavy chain variable region having an amino acid sequence that is at least 90% identical to the amino acid sequence of SEQ ID NO: 14, SEQ ID NO: 25, or SEQ ID NO:72; and a light chain variable region having an amino acid sequence that is at least 90% identical to the amino acid sequence of SEQ ID NO: 18 or (ii) a heavy chain variable region having an amino acid sequence that is at least 90% identical to the amino acid sequence of SEQ ID NO: 78 and a light chain variable region having an amino acid sequence that is at least 90% identical to the amino acid sequence of SEQ ID NO: 80.

The disclosure further provides compositions that include a multispecific antigen-binding construct (e.g., a bispecific antigen-binding construct, a trispecific antigen-binding construct, or a tetraspecific antigen-binding construct) or an isolated antibody or antigen-binding fragment thereof disclosed herein and a pharmaceutically acceptable carrier. The antigen-binding construct optionally further comprises a third antigen-binding unit that specifically binds to a molecule expressed by an effector immune cell. In some embodiments, the composition can include more than one multispecific antigen-binding construct disclosed herein.

The disclosure also provides nucleic acids encoding the multispecific antigen-binding construct or the isolated antibody or antigen-binding portion thereof described herein. Also provided are vectors comprising the nucleic acids (optionally with an expression control sequence) and cells comprising the vectors or nucleic acids. Methods for producing a polypeptide are provided, including culturing such cells under conditions for the expression of one or more polypeptides from the vector by the cell. The methods optionally include isolating the one or more polypeptides from the cell or media in which the cell is cultured. Also provided are protein conjugate molecules that comprise a heterologous moiety conjugated to the multispecific antigen-binding construct or the isolated antibody, or antigen-binding fragment thereof, described herein. Optionally, the heterologous moiety is a therapeutic agent, a toxin, a drug, or a radioactive moiety. The disclosure also provides methods for treating a cancer in a subject, comprising administering to the subject having cancer an effective amount of the multispecific antigen-binding construct; the isolated antibody, or antigen-binding fragment thereof; the pharmaceutical composition; or the protein conjugate molecule described herein.

The disclosure is also based, in part, on the discovery that the multispecific antigen-binding constructs described herein are capable of enhancing an immune response to a cancer or tumor in a CD16-engagement independent manner. CD16 (FCγRIII) binds to the Fc portion of IgG antibodies, such as the Fc portion of IgG1 isotype antibodies. CD16A is a transmembrane protein that co-localizes with CD3ζ and Fc-εRI-γ on NK cells. The binding of such Fc regions to CD16A results in cytokine production by NK cells, and increased cytotoxic effector activity (ADCC) of these cells towards target cells, such as cancer cells. Many tumor-directed monoclonal antibody therapies rely on CD16A engagement for their activity, which can present challenges for their use in cancer patients. For example, in humans, NK cells are composed of two distinct populations: CD56dim/CD16pos and CD56bright/CD16neg. In healthy individuals, the CD16neg population represents 5-15% of the total NK cell population. However, in some cancer patients the proportion of CD16neg NK cells is greatly increased (e.g., up to 50%).

In addition, the tumor micro-environment has been shown to affect the phenotype of CD56dim/CD16pos NK cells by either inducing shedding of CD16A from the surface of the cells (activity mediated by ADAM17 enzyme) or TGFβ can promote conversion from CD16Apos to CD16neg NK cells. In addition, due to CD16A polymorphism, some individuals have mutations in CD16A that dramatically impair ADCC mediated by monoclonal antibody therapy. Thus, tumor-directed therapies that can maintain their anti-tumor activity even in the absence of CD16A engagement are greatly desired and needed. The instantly described multispecific antigen-binding constructs, which are capable of overcoming CD16A deficiency, fulfill that need.

The disclosure is also based, in part, on the discovery that the multispecific antigen-binding constructs described herein are capable of enhancing an immune response (e.g., an NK cell-mediated response) to target cells that express low levels of a target antigen (i.e., the tumor antigen to which the multispecific constructs bind). Tumor antigen-directed monoclonal antibody therapies, such as HERCEPTIN® (Genentech, South San Francisco, Calif.) and RITUXAN® (Biogen, Cambridge, Mass.), have exhibited reduced efficacy towards tumors that express low levels of the tumor antigen. In fact, patients bearing tumors with low level expression of tumor antigen can be ineligible for treatment with antibody therapies that target the antigen. Also established is that tumors can escape such antibody therapies by, among other things, downregulating the expression of the tumor antigen to which the antibody therapies bind (see, e.g., Sugimoto et al. (2009) Biochem. Biophys. Res. Commun. 390(1):48-53; Bodogai et al. (2013) Cancer Res. 73(7):1-12). While the disclosure is not bound by any particular theory or mechanism of action, the multispecific constructs described herein overcome these limitations of antibody therapies by lowering the threshold of NK cell activation and potently redirecting NK cell killing of tumor cells expressing high, medium, and low levels of a tumor antigen.

Thus, the disclosure further provides methods for treating cancer in a subject, including the step of administering to the subject having cancer an effective amount of a multispecific antigen-binding construct or antibody or antigen-binding portion thereof described herein, or a protein conjugate or a composition including a multispecific antigen-binding construct or antibody or antigen-binding portion thereof described herein. Optionally, the method of treating cancer comprises administering to the subject with cancer an effective amount of the multispecific antigen-binding construct or antibody or antigen-binding portion thereof or a protein conjugate or a composition including a multispecific antigen-binding construct or antibody or antigen-binding portion, wherein the subject has a cancer that comprises a CD16 deficient (including CD16 low) microenvironment (e.g., cancer that has a population of infiltrating NK cells that have less than 50% expression of CD16 as compared to a control NK cell or cancer that has a population of infiltrating NK cells in which at least 10% of the infiltrating NK cells have reduced expression of CD16 as compared to a control NK cell). In some embodiments, the CD16 deficient microenvironment is associated with hematopoietic stem cell transplantation to the subject. The subject can be a human or other mammal. The multispecific antigen-binding construct or antibody or antigen-binding portion thereof can enhance the subject's immune response by agonizing NKp30 function. For example, the multispecific antigen-binding construct or antibody or antigen-binding portion thereof can enhance NK cell mediated cancer cell lysis. The methods can include detecting CD16 in the microenvironment. Optionally, the CD16 deficient microenvironment is detected by detecting the level of CD16a or CD16b (e.g., in a biopsy or sample of tissue). In some embodiments of any of the methods described herein, the CD16A genotype of the subject (e.g., a human cancer patient) can be determined prior to administering the multispecific antibody. For example, a test can be run to determine the subject's genotype with respect to CD16A 158 polymorphism. See, e.g., Xu et al. (2016) Med. Sci. Monitor. 22:2086-2096.

Optionally, the method of treating cancer comprises administering to a subject with cancer an effective amount of the multispecific antigen-binding construct or an effective amount of antibody or antigen-binding portion thereof, wherein the subject has a cancer that comprises a low level of the tumor antigen (e.g., less than about 100,000 copies per cancer cell). The subject can be a human or other mammal. The multispecific antigen-binding construct or antibody or antigen-binding portion thereof can enhance the subject's immune response by agonizing NKp30 function. The multispecific antigen-binding construct or antibody or antigen-binding portion thereof effectively lowers the threshold for natural killer (NK) cell activation and redirects NK cell killing of target cells (e.g., tumor cells) expressing even low levels of the tumor antigen. The multispecific antigen-binding construct or antibody or antigen-binding portion thereof retains IFNγ production and ADCC function even with low tumor antigen expressing cells.

The cancer is optionally selected from the group consisting of a hematological cancer, neurological cancer, breast cancer, prostate cancer, skin cancer, lung cancer, bladder cancer, kidney cancer, brain cancer, head and neck cancer, gastrointestinal cancer, liver cancer, pancreatic cancer, genitourinary cancer, bone cancer, and vascular cancer. The methods for treating cancer can further include the step of administering a second anticancer therapy to the subject. In certain embodiments, the anticancer therapy is chemotherapy, immunotherapy, hormone therapy, cytokine therapy, radiotherapy, cryotherapy, or surgical therapy. The multispecific antigen-binding construct or composition is administered, for example, subcutaneously, intravenously, intradermally, intraperitoneally, orally, intramuscularly, or intracranially.

The disclosure also provides methods of enhancing an immune response in a subject in need thereof, comprising administering to the subject (e.g., a human or other mammal) a therapeutically effective amount of a multispecific antigen-binding construct, an effective amount of or antibody or antigen-binding portion thereof described herein, or an effective amount of a composition that includes the disclosed multispecific antigen-binding construct or antibody or antigen-binding portion thereof. The enhanced immune response includes one or more of enhanced T cell function, enhanced NK cell function, enhanced macrophage function, cytokine production, and/or ADCC function. The multispecific antigen-binding construct, antibody or fragment, or composition is administered subcutaneously, intravenously, intradermally, intraperitoneally, orally, intramuscularly, or intracranially.

Also provided herein are methods for activating or sustaining activation of an NK cell that has reduced expression of CD16 as compared to the expression of CD16 by a control NK cell. The methods include contacting the NK cell with an effective amount of a multispecific antigen-binding construct, antibody or antigen-binding portion thereof, or composition comprising either described herein. The contacting of the NK cell with the multispecific antigen-binding construct or antibody or antigen-binding portion thereof can be in vitro or in vivo. In some embodiments, the methods are used to activate or sustain activation of an NK cell in a subject having cancer. The activation or sustained activation of the NK cell can occur in a CD16 deficient microenvironment. Optionally, the tumor antigen to which the multispecific antigen-binding construct specifically binds is BCMA or HER2. The contacted NK cell can be a tumor infiltrating NK cell. Optionally, the CD16 expression of the NK cell is less than the CD16 expression of a control NK cell (e.g., less than 50% expression of CD16 as compared to a control NK cell). Optionally, the CD16 deficient microenvironment comprises a population of tumor infiltrating NK cells in which at least 10% of the infiltrating NK cells have reduced expression of CD16 as compared to a control NK cell. The NK cell can have a CD16 copy number less than 150,000. In some embodiments, the NK cell does not express CD16.

Also provided herein is a method for enhancing the killing of a target cell by an NK cell. The method includes contacting the target cell with an effective amount of a multispecific antigen-binding construct, antibody or antigen-binding portion thereof, or composition described herein in the presence of the NK cell. Optionally, the target cell expresses a low level of tumor antigen (e.g., a copy number of less than about 100,000). More specifically the target cell can be a cancer cell and the antigen is a tumor antigen. Optionally, the cancer cell is in a subject, and the method comprises administering to the subject an effective amount of a multispecific antigen-binding construct, antibody or fragment, or composition to enhance killing of the cancer cell by the NK cell.

In yet another aspect, the disclosure features a method for depleting target cells from a subject, the method comprising administering to a subject in need thereof a multispecific antigen-binding construct as described herein in an amount sufficient to deplete one or more target cells expressing a target antigen, wherein the multispecific binding construct comprises at least two linked antigen-binding units, wherein a first antigen-binding unit specifically binds the target antigen, and wherein a second antigen-binding unit specifically binds an NKp30 antigen. In some embodiments, the multispecific binding construct comprises a third antigen-binding unit. In some embodiments, the multispecific binding construct comprises a third antigen-binding unit and a fourth antigen-binding unit. In some embodiments, the target antigen is a tumor antigen, such as any of the tumor antigens described herein or known in the art. In some embodiments, the target cells are B cells, such as autoreactive B cells (and, in such cases, the target antigen is a B cell-specific, B cell-restricted, or B cell-enriched antigen, such as CD20 or BCMA). In some embodiments, the subject is a human. In some embodiments, the subject is afflicted with an autoimmune condition. In some embodiments, the autoimmune condition is mediated, in whole or in part, by autoreactive B cells. In some embodiments, the subject being depleted of target cells has or is at risk for red-cell aplasia. The red-cell aplasia can be due, for example, to ABO blood-group incompatibility following an allogenic stem-cell transplant. In some such embodiments, the subject has blood group O and the allogenic stem-cell transplant was from a blood group A donor. In those embodiments where the subject being depleted of target cells has or is at risk for red-cell aplasia, the target cells are, for example, target plasma B cells. Accordingly, in some embodiments, the target antigen is an antigen found on plasma B cells but not on other B cells, for example.

In another aspect, the disclosure features a method of treating a subject with an autoreactive-B cell inflammatory condition, comprising administering to the subject an effective amount of a multispecific antigen-binding construct or antibody or antigen-binding portion thereof disclosed herein or a pharmaceutical composition thereof. An effective amount of a multispecific antigen-binding construct, antibody or fragment thereof, or pharmaceutical composition thereof optionally reduces bone marrow levels of plasma cells expressing IgG, IgM, or IgA. The autoreactive-B cell inflammatory condition includes autoimmune disease mediated by autoreactive B cells. Examples of autoreactive-B cell inflammatory conditions include, but are not limited to, myasthenia gravis, light-chain amyloidosis, pemphigus vulgaris, and immune thrombocytopenia.

Also provided herein is a method for treating a subject having an inflammatory condition (e.g., an autoimmune condition) that is mediated in whole or in part by autoreactive B cells, the method comprising administering to the subject a multispecific antigen-binding construct described herein in an amount sufficient to deplete one or more autoreactive B cells expressing a target antigen, wherein the multispecific binding construct comprises a first antigen-binding unit that specifically binds the target antigen, a second antigen-binding unit specifically binds an NKp30 antigen, and a third antigen-binding unit. In some embodiments, the multispecific antigen-binding construct comprises a fourth antigen-binding unit. In some embodiments, the target antigen is a B cell-specific, B cell-restricted, or B cell-enriched antigen, such as CD20, CD19, or BCMA. In some embodiments, the autoimmune disease mediated in whole or in part by autoreactive B cells can be one selected from the group consisting of systemic lupus erythematosus (SLE), Sjogren's syndrome, Type 1 diabetes, Addison disease, Pernicious anemia, autoimmune hepatitis, multiple sclerosis, rheumatoid arthritis, myasthenia gravis, primary biliary cholangitis, Goodpasture's disease, primary membranous nephropathy, ovarian insufficiency, and autoimmune orchitis. Other such autoimmune diseases are known in the art and described in, e.g., Ludwig et al. (2017) Frontiers in Immunol 8: Article 603 and Hofmann et al. (2018) Frontiers in Immunol 9: Article 835. In some embodiments, such multispecific constructs can be combined with other therapies for inflammatory disorders (e.g., autoimmune diseases), such as corticosteroids, DMARDs, or anti-cytokine therapies (e.g., anti-TNFα antibody, a TNFα receptor trap, an anti-IL 17 antibody, or an anti-IL 23 antibody).

Also provided herein are a multispecific antigen-binding construct, an isolated monoclonal antibody or antigen-binding fragment thereof, a pharmaceutical composition, or a protein conjugate, as described herein, for use in enhancing an immune response against a cancer cell in a subject. The immune response optionally is an NK cell-mediated immune response. The cancer cell optionally is a hematological cancer cell, a lymphoma cell, a myeloma cell, a leukemia cell, a neurological cancer cell, a breast cancer cell, a prostate cancer cell, a skin cancer cell, a lung cancer cell, a bladder cancer cell, a kidney cancer cell, a head and neck cancer cell, a gastrointestinal cancer cell, a colorectal cancer cell, a liver cancer cell, a pancreatic cancer cell, a genitourinary cancer cell, a bone cancer cell, and a vascular cancer cell. In some aspects, the cancer cell expresses the first tumor antigen. In some aspects, the cancer cell expresses the first tumor antigen and the second tumor antigen.

The disclosure also provides are a multispecific antigen-binding construct, an isolated monoclonal antibody or antigen-binding fragment thereof, a pharmaceutical composition, or a protein conjugate, as described herein, for use in enhancing an immune response against a B cell in a subject. The immune response optionally is an NK cell-mediated immune response. The B cell optionally is an autoreactive B cell or plasma cell. In some aspects, the B cell expresses the first B cell antigen. In some aspects, the B cell expresses the first B cell antigen and the second B cell antigen.

The disclosure further provides a multispecific antigen-binding construct, an isolated monoclonal antibody or antigen-binding fragment thereof, a pharmaceutical composition, or a protein conjugate, as described herein, for use in treating cancer in a subject.

The disclosure also provides a multispecific antigen-binding construct, an isolated monoclonal antibody or antigen-binding fragment thereof, a pharmaceutical composition, or a protein conjugate, as described herein, for use in treating or delaying progression of a cancer or reducing or inhibiting tumor growth in a subject in need thereof.

Optionally, the cancer is a hematological cancer, lymphoma, myeloma, leukemia, neurological cancer, breast cancer, prostate cancer, skin cancer, lung cancer, bladder cancer, kidney cancer, head and neck cancer, gastrointestinal cancer, colorectal cancer, liver cancer, pancreatic cancer, genitourinary cancer, bone cancer, or a vascular cancer. In some aspects, the cancer comprises cancer cells expressing the first tumor antigen. In some aspects, the cancer comprises cancer cells expressing the first tumor antigen and the second tumor antigen.

In embodiments of any of the uses described herein, the multispecific antigen-binding constructs, isolated monoclonal antibody or antigen-binding fragment thereof, the pharmaceutical composition, or the protein conjugate enhances the subject's immune response by agonizing NKp30 function.

In embodiments of any of the uses described herein, the multispecific antigen-binding constructs, isolated monoclonal antibody or antigen-binding fragment thereof, the pharmaceutical composition, or the protein conjugate enhances natural killer (NK) cell mediated cancer cell lysis and/or γδ T cell function.

In embodiments of any of the uses described herein, the multispecific antigen-binding constructs, isolated monoclonal antibody or antigen-binding fragment thereof, the pharmaceutical composition, or the protein conjugate enhances γδ T cell-mediated cytotoxicity or cytokine production.

In embodiments of any of the uses described herein, the use further comprises administering an anticancer therapy to the subject. Optionally, the anticancer therapy is chemotherapy, immunotherapy, hormone therapy, cell therapy, cytokine therapy, radiotherapy, cryotherapy, or surgical therapy. The anticancer therapy is optionally administered prior to, concurrently with, or after treatment with the multispecific antigen-binding construct or isolated monoclonal antibody or antigen-binding fragment thereof. The anticancer therapy optionally is an immunotherapy, and the cancer in the subject is refractory to the immunotherapy in the absence of treatment with the multispecific antigen-binding constructs, isolated monoclonal antibody or antigen-binding fragment thereof, the pharmaceutical composition, or the protein conjugate. Optionally, the multispecific antigen-binding constructs, isolated monoclonal antibody or antigen-binding fragment thereof, the pharmaceutical composition, or the protein conjugate is administered subcutaneously, intravenously, intradermally, intraperitoneally, orally, intramuscularly, or intracranially.

In some embodiments of any of the uses described herein, the cancer comprises a CD16 deficient tumor microenvironment or the cancer cells are present in a CD16 deficient tumor microenvironment. Optionally, the use further comprises detecting the expression level of CD16 in the cancer of the subject. Detecting the CD16 optionally comprises detecting the level of CD16a or CD16b. In some aspects, the CD16 deficient microenvironment is associated with hematopoietic stem cell transplantation to the subject. Optionally, the CD16 deficient microenvironment comprises a population of infiltrating NK cells, and the infiltrating NK cells have less than 50% expression of CD16 as compared to a control NK cell. In some aspects, the infiltrating NK cells have less than 30%, less than 20%, or less than 10% expression of CD16 as compared to a control NK cell. Optionally, the CD16 deficient microenvironment comprises a population of infiltrating NK cells, and at least 10% of the infiltrating NK cells have reduced expression of CD16 as compared to a control NK cell. In some aspects, at least 20%, at least 30%, or at least 40% of the infiltrating NK cells have reduced expression of CD16 as compared to a control NK cell.

In some embodiments of any of the uses described herein, the cancer comprises cells expressing a low level of tumor antigen. Optionally, the level of the tumor antigen is less than 100,000 tumor antigen copies per cancer cell. In some aspects, the level of the tumor antigen is less than 90,000, less than 75,000, less than 50,000, or less than 40,000 tumor antigen copies per cancer cell. The uses optionally further comprise detecting the level of tumor antigen of one or more cancer cells of the subject.

The disclosure further provides a multispecific antigen-binding construct, an isolated monoclonal antibody or antigen-binding fragment thereof, a pharmaceutical composition, or a protein conjugate, as described herein, for use in treating a subject with an autoimmune disease. Optionally, the autoimmune disease is mediated in whole or in part by autoreactive B cells. The subject optionally is being depleted of target cells and has or is at risk of red-cell aplasia. In some aspects, the autoimmune disease is selected from the group consisting of systemic lupus erythematosus (SLE), Sjogren's syndrome, Type 1 diabetes, Addison disease, Pernicious anemia, autoimmune hepatitis, multiple sclerosis, rheumatoid arthritis, myasthenia gravis, primary biliary cholangitis, Goodpasture's disease, primary membranous nephropathy, ovarian insufficiency, and autoimmune orchitis.

In some embodiments of any of the uses described herein, the multispecific antigen-binding construct enhances the subject's immune response by agonizing NKp30 function.

In some embodiments of any of the uses described herein, the multispecific antigen-binding construct enhances NK cell mediated lysis of B cells.

In some embodiments of any of the uses described herein, the use further comprises administering an agent to the subject, wherein the agent is selected from the group consisting of a corticosteroid, a DMARD, and anti-cytokine therapy. Optionally, the anti-cytokine therapy is an anti-TNFα antibody, a TNFα receptor trap, an anti-IL 17 antibody, or an anti-IL 23 antibody.

In some embodiments of any of the uses described herein, the multispecific antigen-binding construct, isolated monoclonal antibody or antigen-binding fragment thereof, the pharmaceutical composition, or the protein conjugate is administered subcutaneously, intravenously, intradermally, intraperitoneally, orally, intramuscularly, or intracranially.

The disclosure further provides a multispecific antigen-binding construct, an isolated monoclonal antibody or antigen-binding fragment thereof, a pharmaceutical composition, or a protein conjugate, as described herein, for use in activating or sustaining activation of a natural killer (NK) cell in a subject, wherein the NK cell has reduced expression of CD16 as compared to a control NK cell. Optionally, the subject has cancer. In some aspects, the cancer expresses one or both of the tumor antigens to which the construct specifically binds. The contacted NK cell optionally is a tumor infiltrating NK cell. Optionally, the activation or sustained activation of the NK cell occurs in a tumor microenvironment. The CD16 expression of the NK cell optionally is less than 50% of the CD16 expression of the control NK cell. In some aspects, the CD16 expression of the NK cell is less than 40%, less than 30%, less than 20%, less than 10%, less than 5%, or less than 1% of the CD16 expression of the control NK cell. Optionally, the tumor microenvironment comprises a population of infiltrating NK cells, and wherein at least 10% of the infiltrating NK cells have reduced expression of CD16 as compared to a control NK cell. In some aspects, at least 20%, at least 30%, or at least 40% of the infiltrating NK cells have reduced expression of CD16 as compared to a control NK cell. In some aspects, the NK cell does not express CD16.

The disclosure further provides a multispecific antigen-binding construct, an isolated monoclonal antibody or antigen-binding fragment thereof, a pharmaceutical composition, or a protein conjugate, as described herein, for use in enhancing the killing of a cancer cell by an NK cell in a subject, wherein the cancer cell expresses a tumor antigen to which the construct or isolated monoclonal antibody or antigen-binding fragment thereof binds at a tumor antigen copy number of less than 100,000. The cancer cell optionally expresses the tumor antigen at a copy number of less than 90,000 tumor antigen copies per cancer cell. In some aspects, the cancer cell expresses the tumor antigen at a copy number of less than 75,000, less than 50,000, or less than 40,000 tumor antigen copies per cancer cell. In some aspects, the use further comprises detecting expression of the tumor antigen by one or more cancer cells of the subject.

The disclosure also provides a multispecific antigen-binding construct, an isolated monoclonal antibody or antigen-binding fragment thereof, a pharmaceutical composition, or a protein conjugate, as described herein, for use in treating an autoreactive-B cell inflammatory condition in a subject. Optionally, the effective amount of a multispecific antigen-binding construct or pharmaceutical composition reduces bone marrow levels of plasma cells expressing IgG, IgM, or IgA. The autoreactive-B cell inflammatory condition optionally is an autoimmune disease mediated by autoreactive B cells. In some aspects, the autoreactive-B cell inflammatory condition is a disease selected from myasthenia gravis, light-chain amyloidosis, pemphigus vulgaris, and immune thrombocytopenia. In some aspects, the autoimmune disease mediated by autoreactive B cells is selected from the group consisting of systemic lupus erythematosus (SLE), Sjogren's syndrome, Type 1 diabetes, Addison disease, Pernicious anemia, autoimmune hepatitis, multiple sclerosis, rheumatoid arthritis, myasthenia gravis, primary biliary cholangitis, Goodpasture's disease, primary membranous nephropathy, ovarian insufficiency, and autoimmune orchitis. For example, the autoimmune disease mediated by autoreactive B cells is myasthenia gravis.

In some embodiments of any of the uses described herein, the use further comprises administering to the subject an anti-inflammatory agent. Optionally, the anti-inflammatory agent is selected from the group consisting of a corticosteroid, DMARDs, or as anti-cytokine agent. Optionally, the subject is a human.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is an illustrative representation of an exemplary antibody format (e.g., as described in Kontermann and Brinkmann (2015) Drug Discovery Today 20(7):838-47).

FIG. 2A and FIG. 2B are line graphs depicting the enhancement of H929 tumor cell killing by NK cells in the presence of exemplary BCMA-NKp30 multispecific constructs. The X-axis shows the concentration of construct or antibody. The Y-axis shows the % specific lysis of H929 cells by NK cells. FIG. 2A shows the results with two glycosylated multispecific constructs (Construct 1 (squares) and Construct 2 (diamonds), which differ from each other only in the amino acid sequence of the anti-NKp30 Fab) that were tested alongside an IgG1 isotype control (solid circles) and an anti-BCMA IgG1 antibody (hatched circles). The constructs and the anti-BCMA antibody are capable of binding to CD16a. FIG. 2B shows the results with versions of Construct 1 and Construct 2, which lack glycosylation (N297A mutation) and, thus, are unable to bind to CD16a. The aglycosylated versions of Construct 1 and Construct 2 (open squares and open diamonds; also referred to herein as Construct 3 and Construct 4, respectively) were tested alongside an aglycosylated version of the IgG1 isotype control (open circles) and anti-BCMA IgG1 antibody (hatched circles).

FIG. 3A and FIG. 3B are line graphs showing that the BCMA-NKp30 multispecific construct (Construct #1) enhanced ADCC in the presence of CD16A expression. The concentration of the antibodies is shown on the X-axis, and the percentage of specific lysis is shown on the Y-axis. FIG. 3A shows results using MM.1S tumor cells, and FIG. 3B shows results using RPMI-8226 tumor cells. When cells of the CD16A-expressing NK cell line were used as effector cells, the BCMA-NKp30 multispecific construct (open circles) demonstrated increased ADCC compared to the BCMA monoclonal antibody (hatched circles) or IgG1 isotype control (solid circles).

FIG. 4A, FIG. 4B, and FIG. 4C are line graphs showing that the present BCMA-NKp30 multispecific constructs induced tumor cell killing in a CD16-independent manner. The concentration of the constructs or antibodies is shown on the X-axis, and the percentage of specific lysis is shown on the Y-axis. FIG. 4A and FIG. 4C show results using H929 tumor cells, which express the tumor antigen BCMA at high levels, and FIG. 4B shows results using RPMI-8226 tumor cells, which express BCMA at low levels. When CD16A-negative NK cell lines (KHYG-1 in FIG. 4A and FIG. 4C, NK cell line in FIG. 4B) were used as effector cells, the BCMA-NKp30 multispecific constructs (squares and diamonds) induced tumor-cell killing by CD16-negative NK cells compared to the BCMA monoclonal antibody (hatched circles in FIG. 4B and FIG. 4C), IgG1 isotype control (solid circles in FIG. 4B), or CD16A-BCMA bispecific construct (open circles in FIG. 4C).

FIG. 5A, FIG. 5B, and FIG. 5C are line graphs showing that the present BCMA-NKp30 multispecific constructs exhibit superior activity by dual targeting of NKp30 and CD16. The concentration of the constructs or antibodies is shown on the X-axis in FIG. 5A, FIG. 5B, and FIG. 5C. The amount (in pg/mL) of IFNγ produced is shown on the Y-axis in FIG. 5A and FIG. 5B, and the percentage of specific lysis is shown on the y axis in FIG. 5C. FIG. 5A shows results using H929 tumor cells, which express BCMA at high levels, and FIG. 5B and FIG. 5C show results using MM.1S tumor cells, which express BCMA at low levels. The NKp30-BCMA multispecific construct (solid squares) exhibited superior activity to an NKp30-BCMA Fc-null construct (open squares in FIG. 5A), the BCMA monoclonal antibody (solid circles in FIG. 5A, FIG. 5B, and FIG. 5C), a Her2 IgG1 isotype control (triangles in FIG. 5A), a CD16-BCMA bispecific construct (open circles in FIG. 5B and FIG. 5C), and an NKp30 null-BCMA Fc null construct (inverted triangles in FIG. 5B and FIG. 5C).

FIG. 6A and FIG. 6B are line graphs showing that exemplary glycosylated multispecific constructs that bind BCMA and NKp30 (Construct 1 (square) and Construct 2 (diamond)) enhance NK-cell activation and ADCC activity against high BCMA-expressing tumor cells. The multispecific constructs, which differ only in the amino acid sequence of the anti-NKp30 Fab portion, exhibit superior ADCC activity (FIG. 6A) and IFNγ production (FIG. 6B) against high BCMA-expressing tumor cells, when tested alongside an IgG1 isotype control (solid circle) and an anti-BCMA IgG1 antibody (hatched dark). The X-axis shows the concentration of the construct or antibody, and the Y-axis shows the % specific lysis of H929 cells by NK cells (FIG. 6A) or the concentration of IFNγ released (FIG. 6B).

FIG. 7A and FIG. 7B are line graphs showing that exemplary glycosylated multispecific constructs that bind BCMA and NKp30 (Construct 1 (square) and Construct 2 (diamond)) enhance NK-cell activation and ADCC activity against moderate BCMA-expressing tumor cells. The multispecific constructs exhibit superior ADCC activity (FIG. 7A) and IFNγ production (FIG. 7B) against moderate BCMA-expressing tumor cells, when tested alongside an IgG1 isotype control (solid circle) and an anti-BCMA IgG1 antibody (hatched circle). The X-axis shows the concentration of the construct or antibody, and the Y-axis shows the % specific lysis of MM1.S cells by NK cells (FIG. 7A) or the concentration of IFNγ released (FIG. 7B).

FIG. 8A and FIG. 8B are line graphs showing that exemplary glycosylated multispecific constructs that bind BCMA and NKp30 (Construct 1 (square) and Construct 2 (diamond)) enhance NK-cell activation and ADCC activity against low BCMA-expressing tumor cells. The multispecific constructs exhibit superior ADCC activity (FIG. 8A) and IFNγ production (FIG. 8B) against low BCMA-expressing tumor cells, when tested alongside an IgG1 isotype control (solid circle) and an anti-BCMA IgG1 antibody (hatched circle). The X-axis shows the concentration of the construct or antibody, and the Y-axis shows the % specific lysis of RPMI-8226 cells by NK cells (FIG. 8A) or the concentration of IFNγ released (FIG. 8B).

FIG. 9 is a bar graph depicting the effect of NKp30-BCMA multispecific antigen-binding constructs on NK cell activation in the presence or absence of H929 cancer cells. Effects for constructs 1, 3Z, 4Z, 2Z (Construct 2Z is also referred to herein as Construct 9), 5Z, and anti-BCMA IgG1 are shown from left to right. The Y-axis represents IFNγ production, which is expressed in units of pg/mL.

FIG. 10 is a line graph depicting the effect of NKp30-BCMA multispecific antigen-binding constructs on NK cell activation in the presence (NK+H929+ construct 1 (square) and NK+H929+ construct 2 (diamond)) or absence (open circle depicts NK+ construct 1 and hatched circle depicts NK+ construct 2) of H929 cancer cells. The Y-axis represents the percentage of CD69+NK cells.

FIG. 11 is a line graph depicting the effect of NKp30-BCMA multispecific antigen-binding constructs on NK cell activation in the presence (NK+H929+ construct 1 (square) and NK+H929+ construct 2 (diamond)) or absence (open circle depicts NK+ construct 1 and solid circle depicts NK+ construct 2) of H929 cancer cells. The Y-axis represents the percentage of CD137+NK cells.

FIG. 12 is a line graph depicting the effect of NKp30-BCMA multispecific antigen-binding constructs (Construct 1 (square) and Construct 2 (diamond)) on NK cell activation in the absence of prior stimulation with cytokines IL-2 and IL-15. The effect of anti-BCMA IgG1 (circle) is shown for comparison. The Y-axis represents IFNγ production, which is expressed in units of pg/mL. The X-axis represents the concentration of the constructs or antibody in nM.

FIG. 13 is a line graph depicting the effect of NKp30-BCMA multispecific antigen-binding constructs on NK cell activation in the absence of prior stimulation with cytokines IL-2 and IL-15. Data are shown for Construct 1 (square), Construct 2 (diamond), and anti-BCMA IgG1 (circle). The Y-axis represents the percentage of CD69+NK cells. The X-axis represents the concentration of the constructs or antibody in nM.

FIG. 14 is a line graph depicting the effect of NKp30-BCMA multispecific antigen-binding constructs on NK cell activation in the absence of prior stimulation with cytokines IL-2 and IL-15. The Y-axis represents the percentage of CD137+NK cells. Data are shown for Construct 1 (square), Construct 2 (diamond), and anti-BCMA IgG1 (circle). The X-axis represents the concentration of the constructs or antibody in nM.

FIG. 15A, FIG. 15B, and FIG. 15C show that the NKp30-BCMA multispecific antigen-binding construct is a more potent inducer of target cell lysis than elotuzumab and daratumumab. FIG. 15A reflects the surface expression of tumor antigens BCMA, CD38, and CS1 on MIMI S tumor cells. The multispecific construct (square) exhibited increased IFNγ production (FIG. 15B) and superior ADCC activity (FIG. 15C) against tumor cells, when tested alongside an anti-BCMA IgG1 antibody (circle), daratumumab (inverted triangle), and elotuzumab (diamond). The X-axis shows the concentration of the construct or antibody, and the Y-axis shows the concentration of IFNγ released (FIG. 15B) or the % specific lysis of MM.1S tumor cells by NK cells (FIG. 15C).

FIG. 16 is an illustrative representation of an exemplary embodiment of a multispecific antigen-binding construct described herein, where the construct specifically binds: NKp30, a first tumor antigen, a second tumor antigen, and a second NK activating receptor, such as NKp46.

FIG. 17A, FIG. 17B, and FIG. 17C are line graphs showing that the present Her2-NKp30 multispecific constructs exhibit superior activity by targeting of NKp30 and CD16. The concentration of the constructs or antibodies is shown on the X-axis in FIG. 17A, FIG. 17B, and FIG. 17C. The amount (in pg/mL) of IFNγ produced is shown on the Y-axis in FIG. 17B and FIG. 17C, and the percentage of specific lysis is shown on the Y axis in FIG. 17A. FIG. 17A shows results using Her2+ SKBR3 tumor cells, and FIG. 17B and FIG. 17C show results using SW480 tumor cells and T47D tumor cells, which express Her2 at low and intermediate to low expression levels, respectively. The NKp30-Her2 multispecific construct (Construct A (open diamonds) and Construct B (squares)) exhibited superior activity (determined as lower EC50 values) to a monoclonal anti-Her2 IgG1 antibody (trastuzumab, circles) or a variant of the anti-Her2 IgG1 antibody that lacked glycosylation (solid diamonds). Likewise, such Constructs were more potent at inducing IFNγ expression by NK cells as compared to a number of anti-Her2 IgG1 monoclonal antibodies FIG. 17A).

FIG. 18 is a flow cytometry plot showing human Her2 expression levels by various cultured tumor cells.

FIG. 19 demonstrates that multispecific antigen-binding construct 1, targeting both NKp30 and BCMA, depletes plasma cells in the bone marrow of cynomolgus macaques. Adult cynomolgus monkeys (n=2) received a single intravenous injection of construct 1 at 30.25 mg/kg. The number of immunoglobulin-secreting cells in the bone marrow of treated monkeys was measured by ELISA over time using assays specific for IgM, IgG, and IgA. A strong decrease in bone marrow plasma cells (>80%) at 2 weeks post-treatment was observed, followed by a rebound 3 weeks later in both treated animals.

FIG. 20A and FIG. 20B demonstrate that multispecific antigen-binding Construct 1, targeting both NKp30 and BCMA, induces a decrease of serum IgM in plasma of treated cynomolgus macaques. Using ELISA, the level of plasma IgM in the peripheral blood of treated monkeys was measured over time (FIG. 20A). This assay was specific for cynomolgus IgM, and a standard cynomolgus IgM was used to calculate the IgM concentration in blood of treated monkeys. A strong decrease in plasma IgM starting was observed 5 weeks post-treatment (FIG. 20B). Data is representative of 3 independent experiments. This level of serum IgM depletion in monkeys is similar to what has been observed after six courses of plasmapheresis in human (See, for example, JT Guptill et al., Autoimmunity (2016) 49(7): 472-479), and after Fc/FcRn blockade in humans (See, for example, Ulrichts, 2017).

FIG. 21A and FIG. 21B demonstrate that multispecific antigen-binding Construct 1, targeting both NKp30 and BCMA induces in vivo NK-cell expansion in treated cynomolgus monkeys. As indicated in FIG. 21A, NK cells expanded in the blood of treated monkeys with a maximum peak at about 14 days post-treatment. NK cells also expanded in bone marrow of treated monkeys (FIG. 21B), although this expansion was less for monkey B6016 than for AK749J.

FIG. 22A and FIG. 22B demonstrate that multispecific antigen-binding Construct 1, targeting both NKp30 and BCMA induces NK-cell activation in monkey AK749J. As indicated by CD69 expression, NK cells were activated in the blood (FIG. 22A) and bone marrow (FIG. 22B) of cynomolgus monkey AK749J. Monkey B6016 had high CD69 expression (>70%) before treatment and maintained a high CD69 expression during the course of treatment.

FIG. 23 demonstrates that multispecific antigen-binding Construct 1, targeting both NKp30 and BCMA, displays a typical IgG1-like pharmacokinetic profile. Plasma was collected after a single IV injection of Construct 1, and levels of Construct 1 in blood of treated monkeys was measured using an antigen-specific ELISA. Using a two-phase decay model, it was estimated that the β-phase half-life was ˜16 days for both monkeys.

FIG. 24A, FIG. 24B, and FIG. 24C show line graphs demonstrating that multispecific antigen-binding Construct 1 targeting both NKp30 and BCMA (square) promotes higher production of IFNγ (FIG. 24A), TNFα (FIG. 24B), and Rantes (FIG. 24C) by NK cells only in the presence of tumor cells and has superior activity to an IgG1 monoclonal antibody against BCMA alone (circle). The X-axis shows the concentration of the construct or antibody, and the Y-axis shows the concentration of the various cytokines released in pg/ml.

FIG. 25A and FIG. 25B demonstrate that multispecific antigen-binding Construct 1 targeting both NKp30 and BCMA induces activation and cytotoxicity towards multiple myeloma tumor cells by peripheral NK Cells from myeloma patients. FIG. 25A shows that healthy donors and multiple myeloma patients express comparable levels of NKp30 and CD16A on PBMCs. FIG. 25B shows that, compared to Trastuzumab as an isotype control or no antibody, multispecific antigen-binding Construct 1 leads to an increase in activated NK cells isolated from patients with multiple myeloma, as well as increased cytotoxic activity towards multiple myeloma cells.

FIG. 26A and FIG. 26B demonstrate that multispecific antigen-binding Construct 1 targeting both NKp30 and BCMA induces NK-cell killing of autologous myeloma cells from bone marrow of a multiple myeloma patient. FIG. 26A shows that plasma cells in the bone marrow of a newly diagnosed multiple myeloma patient are expressing BCMA, and NK cells are expressing high levels of NKp30 and CD16A. As shown in FIG. 26B, when bone marrow cells were tested in the presence of Construct 1 or Construct 3, an aglycosylated (Fc null) version of Construct 1, or BCMA-IgG1 mAb, death of malignant bone marrow cells was observed over no antibody control at a higher extent with construct 1 and its aglycosylated version, construct 3, as compared to a BCMA-IgG1 monoclonal control.

FIG. 27 demonstrates that multispecific antigen-binding Construct 5, a variant of multispecific antigen-binding Construct 1 having an affinity matured BCMA arm, has improved binding in the presence of the soluble BCMA ligand, APRIL. As shown in the line graph on the left, Construct 1 does not bind to H929 BCMA positive tumor cells in the presence of 100 ng/ml APRIL. However, multispecific antigen-binding Construct 5 (as shown in the line graph on the right), a variant of multispecific antigen-binding Construct 1 having an affinity matured BCMA arm, can bind to BCMA positive tumor cells, even in the presence of soluble APRIL.

FIG. 28A and FIG. 28B demonstrate that the activity of multispecific antigen-binding Construct 5, a variant of multispecific antigen-binding Construct 1 having an affinity matured BCMA arm, is not impacted by the presence of soluble APRIL and BAFF. Primary NK cells were cultured with BCMAP^{pos} multiple myeloma tumor cell lines U266 (FIG. 28A) or MM1R (FIG. 28B) in the presence of various concentrations of Construct 5, with or without the soluble BCMA ligands, April and BAFF. IFNγ secreted by NK cells was measured in the supernatants after 48 hours.

FIG. 29 demonstrates that multispecific antigen-binding Construct 5, a variant of multispecific antigen-binding Construct 1 having an affinity matured BCMA arm, shows activity in the presence of high levels of soluble BCMA. Primary NK cells were incubated with H929 cancer cells in the absence (hatched circle) or presence (open circle) of soluble BCMA (50 ng/ml) and various concentrations of Construct 5. Secretion of IFNγ by NK cells was measured.

FIG. 30 demonstrates that multispecific antigen-binding Construct 5, a variant of multispecific antigen-binding Construct 1 having an affinity matured BCMA arm, retained activity towards BCMA^{low} target cells. Primary NK cells were cultured with BCMAP^{pos} tumor cell lines expressing high and low BCMA copies/cell in the presence of construct 5. IFN-γ released by NK cells in the supernatant was measured by ELISA at 48 hours. The data show that Construct 5 shows selectivity towards BCMA^{high} expressing tumor cells and retained activity towards BCMA^{low} expressing tumor cells, but did not show activity towards tumor cells (e.g., Raji) expressing less than 2,000 BCMA copies/cell.

FIG. 31 demonstrates that multispecific antigen-binding Construct 5, a variant of multispecific antigen-binding Construct 1 having an affinity matured BCMA arm, shows selectivity towards higher expressing BCMA tumor cells and retained activity in low BCMA expressing cells. Primary NK cells were cultured with BCMA positive tumor cell lines expressing high and low BCMA copies/cell in the presence of Construct 5. IFN-γ released by NK cells in the supernatant was measured by ELISA at 48 hours. Construct 5 showed activity even at picomolar concentrations in inducing IFNγ production by NK cells co-cultured with a range of cell lines varying in levels of BCMA expression.

FIG. 32 demonstrates that multispecific antigen-binding Construct 5 (square), a variant of multispecific antigen-binding Construct 1 having an affinity matured BCMA arm, activates NK cells in the presence of lymphoma cells expressing low copies/cells of BCMA, whereas BCMA-IgG1 monoclonal (circle) does not show any activity. Primary NK cells were cultured with JeKo-1 tumor cells (mantle cell lymphoma) in the presence of various concentrations of construct 5 or BCMA-IgG1. IFNγ released by NK cells in the supernatant was measured by ELISA at 48 hours.

FIG. 33 demonstrates that the proliferative signal induced by multispecific antigen-binding Construct 5 (solid square), a variant of multispecific antigen-binding construct 1 having an affinity matured BCMA arm, is mainly dependent on NKp30 engagement. Primary NK cells were labeled with CELLTRACE™ Violet (CTV) and incubated with H929 cancer cells in the presence of IL-2 and (i) Construct 5, (ii) a bispecific that binds BCMA, CD16 (through the Fc), but not NKp30 [NKp30 null xBCMA IgG1 (open square)], or (iii) a bispecific that binds BCMA, but not CD16 or NKp30 [NKp30 null xBCMA Fc null (inverted triangle)]. NK cell proliferation was measured by flow cytometry assessing CTV dilution after 5 days. Construct 5 induces NK cells proliferation at higher extent than bispecifics that bind CD16A but not NKp30 [NKp30 null×BCMA (IgG1)], or those that do not bind CD16A and NKp30 [NKp30 null×BCMA Fc null] showing that construct 5's proliferative signal is mainly dependent on NKp30 engagement.

FIG. 34 demonstrates that afucosylation improves activity of multispecific antigen-binding Construct 1 (square). Primary NK cells were cultured with H929 tumor cells in the presence of various concentrations of antibodies and CD107 expression on NK cells, as well as intracellular IFNγ were measured by flow cytometry. These data demonstrate that using construct 7 (inverted triangle), an afucosylated Fc of the multispecific antigen-binding construct 1, improves activity.

FIG. 35A and FIG. 35B demonstrate that NKp30 is expressed on γδ T cells in the bone marrow of multiple myeloma patients. Bone marrow (BM) cells were stained, and frequencies of γδ T cells in the BM of four multiple myeloma patients, as well as expression of NKp30 by γδ T cells were assessed via flow cytometry, as shown in FIG. 35A. FIG. 35B shows the frequency of TCR γδ T cells in the bone marrow aspirates of each patient. These data indicate that NKp30 expressing γδ T cells in multiple myeloma patients can also be activated using the multispecific antigen-binding constructs targeting NKp30 and BCMA described herein.

FIG. 36 demonstrates that multispecific antigen-binding Construct 5, a variant of multispecific antigen-binding Construct 1 having an affinity matured BCMA arm, blocks proliferation of BCMA positive tumor cells, MM.1R, even in the presence of 100 ng/ml APRIL and 1 ng/ml of BAFF and in the absence of NK cells. Proliferation of MM.1R tumor cells was monitored over a time period of 100 hours by fluorescence imaging using an INCUCYTE® Live Cell analysis system (Essen Instruments, Inc., Ann Arbor, Mich.).

FIG. 37 demonstrates that multispecific antigen-binding Construct 5, a variant of multispecific antigen-binding Construct 1 having an affinity matured BCMA arm, induced proliferation of NK cells to a higher extent than a monoclonal BCMA-IgG1 antibody, as measured by dilution of CELLTRACE′ Violet (Thermo Fisher, Waltham, Mass.).

FIG. 38 demonstrates that multispecific antigen-binding Construct 5, a variant of multispecific antigen-binding Construct 1 having an affinity matured BCMA arm, induced potent killing of BCMA positive tumor cells, MM.1R, by NK cells in the presence of 100 ng/ml APRIL and 1 ng/ml of BAFF. Killing of MM.1R tumor cells by NK cells was monitored over a time period of four hours by fluorescence imaging using an INCUCYTE® Live Cell analysis system.

FIG. 39 demonstrates that multispecific antigen-binding Construct 1, targeting both NKp30 and BCMA, induces potent killing of BCMA tumor cells expressing a wide range of antigen expression, compared to a monoclonal BCMA-IgG1 antibody.

FIG. 40 demonstrates that multispecific antigen-binding Construct 8, an aglycosylated (Fc null) variant of multispecific antigen-binding Construct 5, shows activity even in the absence of CD16A engagement.

FIG. 41 demonstrates that multispecific antigen-binding Construct 5, a variant of multispecific antigen-binding Construct 1 having an affinity matured BCMA arm, induces NK cell killing of BCMA-low JeKo-1 tumor cells, but not BCMA-negative HL-60 tumor cells.

FIG. 42 shows cytotoxicity results using multispecific antigen-binding constructs targeting Her2 and NKp30. The multispecific antigen binding constructs targeting Her2 and NKp30, Constructs C and D, exhibited superior activity to an anti-Her2 monoclonal antibody (trastuzumab).

FIG. 43 shows phenotypic and functional assessment of expanded γδ T-cells. As shown, roughly 40% of the expanded γδ T-cells are positive for NKp30 expression.

FIG. 44 demonstrates that γδ T-cell specific cytotoxicity of target U266 cells shows a dose-dependent effect in the presence of Construct 5.

FIG. 45 shows that there is a noticeable decrease in target U266 cells, after 30 minutes in the presence of 10 pM Construct 5, demonstrating that engagement of NKp30 on γδ T-cells by Construct 5 promotes target cell killing.

FIG. 46 demonstrates that engagement of NKp30 on γδ T-cells by Construct 5 increases γδ T-cell proliferation.

FIG. 47A shows a Table with a partial amino acid sequence of NKp30 where residues that comprise an epitope bound by mAb8, mAb10, and mAb11 are indicated in bold and underlined text. FIG. 47B depicts X-ray crystallography images of human NKp30, with residues I50, S82, and L113 shown as spheres (left panel) and X-ray crystallography images of human NKp30 bound to B7-H6 with residues I50, S82, and L113 shown as spheres (right panel).

### DETAILED DESCRIPTION

The following description recites various aspects and embodiments of the disclosed compositions and methods. No particular embodiment is intended to define the scope of the compositions and methods. Rather, the embodiments merely provide non-limiting examples that are at least included within the scope of the disclosed compositions and methods. The description is to be read from the perspective of one of ordinary skill in the art; therefore, information well known to the skilled artisan is not necessarily included. It is also to be understood that as used herein, the singular forms a, and, and the include plural references unless the context clearly dictates otherwise.

### Multispecific Antigen-Binding Constructs

Provided herein are multispecific antigen-binding constructs comprising at least two linked antigen-binding units. The first antigen-binding unit specifically targets a tumor cell by binding a tumor antigen or specifically targets a B-lineage cell by binding a target antigen expressed by a B-lineage cell. The second antigen-binding unit specifically binds a NKp30 antigen. The constructs are useful in a variety of methods including targeting and killing tumor cells or B cells (including B-lineage cells, such as plasma cells or autoreactive B cells) in subjects with cancer or autoimmune diseases.

The immune system has the capability of recognizing and eliminating tumor cells;

however, tumor cells can use multiple strategies to evade the immune system. Blockade of immune checkpoints is one of the approaches to activating or reactivating therapeutic antitumor immunity. Another approach to inhibit cancer formation or progression is by enhancing the function of various effector molecules and interactions involved in native immune responses. The present disclosure provides multispecific antigen-binding constructs that enhance the native immune response. The immune response is enhanced by the constructs even in conditions in which the subject's NK cells exhibit a reduced level of CD16 and/or the subject's tumor cells exhibit a reduced level of tumor antigen expression. As used herein, “enhancing an immune response” refers to an increase in an immune response that occurs in the presence of the multispecific antigen-binding construct, but not in the absence thereof. Such an enhancement can refer to an increase in one or more NK or T cell functions (e.g., cytotoxic activity, cytokine production) and the like, as described elsewhere herein.

The disclosure provides, in some aspects, multispecific (e.g., bispecific, trispecific, and tetraspecific) antigen-binding constructs and compositions thereof that include at least two linked antigen-binding units, wherein a first antigen-binding unit specifically binds a tumor antigen or an antigen expressed by B-lineage cells, and wherein a second antigen-binding unit specifically binds an NKp30 antigen.

In some embodiments of these aspects and all such aspects described herein, the multispecific antigen-binding constructs include at least three linked antigen-binding units, wherein a first antigen-binding unit specifically binds a first tumor antigen or an antigen expressed by B-lineage cells, a second antigen-binding unit specifically binds a second tumor antigen or an antigen expressed by B-lineage cells, and a third antigen-binding unit specifically binds an NKp30 antigen.

In some embodiments of these aspects and all such aspects described herein, the multispecific antigen-binding constructs include at least three linked antigen-binding units, wherein a first antigen-binding unit specifically binds a tumor antigen or an antigen expressed by B-lineage cells, a second antigen-binding unit specifically binds an NKp30 antigen, and a third antigen-binding unit specifically binds an NK receptor. In some such embodiments, the third antigen-binding unit also binds NKp30. In some such embodiments, the third antigen-binding unit binds a different activating NK receptor, i.e., does not bind NKp30.

In some embodiments of these aspects and all such aspects described herein, the multispecific antigen-binding constructs include at least four linked antigen-binding units, wherein a first antigen-binding unit specifically binds a first tumor antigen or an antigen expressed by B-lineage cells, a second antigen-binding unit specifically binds a second tumor antigen or an antigen expressed by B-lineage cells, a third antigen-binding unit specifically binds an NKp30 antigen, and a fourth antigen-binding unit specifically binds an activating NK receptor. In some such embodiments, the fourth antigen-binding unit also binds NKp30. In some such embodiments, the fourth antigen-binding unit binds a different activating NK receptor, i.e., does not bind NKp30. In those embodiments where the fourth antigen-binding unit binds a different activating NK receptor, the receptor is selected from NKp46, NKp44, 2B4, CD226, NKG2D, CD137, CD16a, and CD2. Exemplary human amino acid sequences for these NK activating receptors can be found as SEQ ID NOs: 28-36.

The multispecific antigen-binding constructs described herein can target one or more tumor antigens, which include (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

In some embodiments, the first and/or second tumor antigen is a tumor-specific antigen (TSA). As used herein, a “TSA” is an antigen that is unique to tumor cells and does not occur on other cells in the body. In some embodiments, the first and/or second tumor antigen is a tumor-associated antigen (TAA). A “TAA,” as used herein, is not unique to a tumor cell and instead is also expressed on a normal cell under conditions that fail to induce a state of immunologic tolerance to the antigen. The expression of the antigen on the tumor can occur under conditions that enable the immune system to respond to the antigen. In some embodiments, a TAA is expressed on normal cells during fetal development when the immune system is immature and unable to respond or is normally present at extremely low levels on normal cells, but which is expressed at much higher levels on tumor cells.

In certain embodiments, the TAA is determined by sequencing a patient's tumor cells and identifying mutated proteins only found in the tumor. These antigens are referred to as “neoantigens.” Once a neoantigen has been identified, therapeutic antibodies can be produced against it and used in the methods described herein.

In some aspects, the tumor antigen is an epithelial cancer antigen, a prostate specific cancer antigen (PSA) or prostate specific membrane antigen (PSMA), a bladder cancer antigen, a lung cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, a colorectal cancer antigen, a lymphoma antigen, a B-cell lymphoma cancer antigen, a leukemia antigen, a myeloma antigen, an acute lymphoblastic leukemia antigen, a chronic myeloid leukemia antigen, or an acute myelogenous leukemia antigen.

The B cell maturation antigen (BCMA) is an example of a tumor antigen that can be targeted by the disclosed multispecific antigen-binding constructs described herein. BCMA is a cell surface receptor of the TNF receptor superfamily and is normally expressed on mature B cells. BCMA naturally occurs in various isoforms. By way of example, human BCMA comprises the amino acid sequence of SEQ ID NO: 6. BCMA is also known as BCM and tumor necrosis factor receptor superfamily 17 (TNFRSF17). BCMA binds to its ligand B cell activating factor (BAFF), which is a member of the TNF family and is expressed by T cells and dendritic cells for the purpose of B cell costimulation. BCMA is detected on certain cancer cells, for example, multiple myeloma cells.

Human epidermal growth factor 2 (HER2) is another example of a tumor antigen that can be targeted by the disclosed multispecific antigen-binding constructs described herein. HER2 is a membrane bound tyrosine kinase receptor encoded by the erbB2 gene. HER2 is also known as HER2/neu, EGFR2, CD340, and ERBB2. HER2 is detected on certain cancer cells, for example, breast cancer cells, ovarian cancer cells, stomach cancer cells, lung cancer cells, and uterine cancer cells. By way of example, human HER2 isoform b comprises the amino acid sequence of SEQ ID NO: 37.

Additional tumor antigens that can be targeted by the disclosed multispecific antigen-binding constructs include, but are not limited to, 1GH-IGK, 43-9F, 5T4, 791Tgp72, 9D7, acyclophilin C-associated protein, alpha-fetoprotein (AFP), α-actinin-4, A3, antigen specific for A33 antibody, ART-4, B7, Ba 733, BAGE, BCR-ABL, beta-catenin, beta-HCG, BrE3-antigen, BCA225, BING-4, BRCA1/2, BTAA, CA125, CA 15-3\CA 27.29\BCAA, CA195, CA242, CA-50, calcium activated chloride channel 2, CAGE, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CML6/6, CO-029, CTLA4, CXCR4, CXCR7, CXCL12, cyclin B, HIF-1a, colon-specific antigen-p (CSAp), CEA (CEACAMS), CEACAM6, c-Met, DAM, E2A-PRL, EGFR, EGFRvIII, EGP-1 (TROP-2), EGP-2, ELF2-M, Ep-CAM, EphA3, fibroblast growth factor (FGF), FGF-5, fibronectin, Flt-1, Flt-3, folate receptor, G250 antigen, Ga733VEpCAM, GAGE, gp100, GRO-β, H4-RET, HLA-DR, HM1.24, human chorionic gonadotropin (HCG) and its subunits, HMGB-1, hypoxia inducible factor (HIF-1), HSP70-2M, HST-2, HTgp-175, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, immature laminin receptor, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MC1R, mCRP, MCP-1, mesothelin, MIP-1A, MIP-1B, MIF, MG7-Ag, MOV18, MUC1, MUC2, MUC3, MUC4, MUCSac, MUC13, MUC16, MUM-1/2, MUM-3, MYL-RAR, NB/70K, Nm23H1, NuMA, NCA66, NCA95, NCA90, NY-ESO-1, P polypeptide, p15, p16, p53, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pme117 prostatic acid phosphatase, PSA, PRAME, PSMA, P1GF, ILGF, ILGF-1R, IL-6, IL-25, RCAS1, RS5, RAGE, RANTES, Ras, T101, SAGE, SAP-1, 5100, SSX-2, survivin, survivin-2B, SDDCAG16, TA-90\Mac2 binding protein, TAAL6, TAC, TAG-72, TGF-βRII, Ig TCR, TLP, telomerase, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, XAGE, 17-1A-antigen, complement factors C3, C3a, C3b, C5a, C5, an angiogenesis marker, bc1-2, bc1-6, and K-ras, an oncogene marker and an oncogene product (see, e.g., Sensi et al. (2006) Clin. Cancer Res. 12:5023-32; Parmiani et al. (2007) J. Immunol. 178:1975-79; Novellino et al. (2005) Cancer Immunol. Immunother. 54:187-207).

In some embodiments, the tumor antigen is a viral antigen derived from a virus associated with a human chronic disease or cancer (such as cervical cancer). For example, in some embodiments, the viral antigen is derived from Epstein-Barr virus (EBV), HPV antigens E6 and/or E7, hepatitis C virus (HCV), hepatitis B virus (HBV), or cytomegalovirus (CMV).

Notably, tumor antigen expression can be downregulated by the tumor cells, so as to evade targeting by NK cells, in particular NK cells infiltrating a tumor. The present multispecific antigen-binding constructs have the capacity to enhance the killing of target cells by NK cells even when the target cells have a low tumor antigen expression level. Additionally, the multispecific antigen-binding constructs retain the ability to promote IFNγ production and ADCC functions even in the presence of low tumor antigen-expressing tumor cells.

Natural cytotoxicity receptors (NCRs) also referred to herein as “NK activating receptors,” such as NKp30, are activating natural killer (NK) cell receptors that typically belong to the immunoglobulin (Ig) superfamily. Signaling through such NK receptors leads to strong NK cell activation resulting in increased intracellular Ca++ levels, triggering of cytotoxicity, and cytokine and lymphokine release, and activation of NK cytotoxicity against many types of target cells, including tumor cells. Non-limiting examples of additional NK activating receptors contemplated for use as targets in embodiments of the multispecific antigen-binding constructs described herein include NKp46, NKp44, 2B4, CD226, NKG2D, CD137, CD16a, and CD2. Exemplary human amino acid sequences for NKp30 and these additional NK activating receptors can be found as SEQ ID NOs: 7, and 28-36.

NKp30 (also known as CD337) is expressed on NK cells and a subset of T cells, such as effector γδ T cells. NKp30 is activated by extracellular ligands, including BAG6, B7-H6 (NCR3LG1), and Galectin-3 (Gal-3), and stimulates NK cell cytotoxicity against neighboring cells expressing such ligands. Thus, it is believed that NKp30 controls NK cell cytotoxicity toward tumor cells. The amino acid sequence of human NKp30 comprises, for example, SEQ ID NO: 7, although other naturally occurring variants exist.

NKp46 (also known as CD335) is selectively expressed by both resting and activated NK cells. Upon activation, NKp46 stimulates NK cell cytotoxicity against neighboring cells expressing its ligands. The amino acid sequence of human NKp46 comprises, for example, SEQ ID NO: 28, although other naturally occurring variants exist.

NKp44 (also known as CD336) is selectively expressed by activated NK cells and by in vitro cultured (i.e., activated) TCRg/d lymphoid cells. NKp44 is activated by NKp44L, a novel isoform of the mixed-lineage leukemia-5 protein. The amino acid sequence of human NKp44 comprises, for example, SEQ ID NO: 36, although other naturally occurring variants exist.

NKG2D (also known as CD314) is expressed in natural killer (NK) cells, CD8+ alpha-beta and gamma-delta T-cells, on essentially all CD56+CD3− NK cells from freshly isolated PBMCs, and in interferon-producing killer dendritic cells (IKDCs). NKG2D is activated by binding to various cellular stress-inducible ligands displayed at the surface of autologous tumor cells and virus-infected cells and stimulates NK cell cytotoxicity against cells expressing such ligands, such as lectin. The amino acid sequence of human NKG2D comprises, for example, SEQ ID NO: 32, although other naturally occurring variants exist.

2B4 (also known as CD244) is a heterophilic receptor of the signaling lymphocytic activation molecule (SLAM) family and its ligand is CD48. 2B4 acts as costimulator in NK activation by enhancing signals by other NK receptors, such as NCR3 and NCR1. The amino acid sequence of human 2B44 comprises, for example, SEQ ID NOs: 29 and 30, although other naturally occurring variants exist.

CD137 (also known as 41BB) is expressed on activated T cells, as well as dendritic cells, B cells, follicular dendritic cells, natural killer cells, granulocytes and cells of blood vessel walls at sites of inflammation, and its ligand is TNFSF9/4-1BBL. The amino acid sequence of human CD137 comprises, for example, SEQ ID NO: 33, although other naturally occurring variants exist.

CD226 (also known as DNAM1) is expressed on subsets of natural killer (NK) and T cells, and its ligands are CD155 or CD112. The amino acid sequence of human CD226 comprises, for example, SEQ ID NO: 31, although other naturally occurring variants exist.

CD16a (also known as FcRIIIa) is expressed on natural killer cells, neutrophil polymorphonuclear leukocytes, monocytes and macrophages, and is a receptor for the Fc region of Ig, and binds complexed or aggregated IgG, as well as monomeric IgG. The amino acid sequence of human CD16a comprises, for example, SEQ ID NO: 34, although other naturally occurring variants exist.

CD2 (also known as LFA2) is expressed on natural killer cells and T cells, and is a receptor for LFA3/CD48 and CD48. The amino acid sequence of human CD2 comprises, for example, SEQ ID NO: 35, although other naturally occurring variants exist.

Accordingly, in some embodiments of the multispecific antigen-binding constructs described herein, at least one antigen-binding unit binds specifically to NKp30. In some embodiments of the multispecific antigen-binding constructs described herein, an antigen-binding unit binds specifically to NKp46. In some embodiments of the multispecific antigen-binding constructs described herein, an antigen-binding unit binds specifically to NKp44. In some embodiments of the multispecific antigen-binding constructs described herein, an antigen-binding unit binds specifically to NKG2D. In some embodiments of the multispecific antigen-binding constructs described herein, an antigen-binding unit binds specifically to 2B4. In some embodiments of the multispecific antigen-binding constructs described herein, an antigen-binding unit binds specifically to CD226. In some embodiments of the multispecific antigen-binding constructs described herein, an antigen-binding unit binds specifically to CD137. In some embodiments of the multispecific antigen-binding constructs described herein, an antigen-binding unit binds specifically to CD16a. In some embodiments of the multispecific antigen-binding constructs described herein, an antigen-binding unit binds specifically to CD2.

The present disclosure provides compositions and methods for enhancing an immune response by agonizing certain immune response effector molecules and interactions. The present compositions and methods can be used to enhance an immune response in a subject, even when the subject has a cancer or condition that comprises a CD16 deficient microenvironment. CD16 is an Fc receptor present on NK cells, neutrophil polymorphonuclear leukocytes, monocytes, and macrophages and is also known as FcγRIII because it binds to the Fc receptor on IgG antibodies. CD16 has been identified as CD16a and CD16b Fc receptors, which participate in signal transduction. CD16a is present on certain NK cells and induces cytokine production and cytotoxic effector activity via antibody-dependent cellular cytotoxicity (ADCC). Downregulation of CD16a has been shown to occur after cytokine activation and target cell stimulation.

Mediating tumor cell killing in the absence of CD16 expression is important. First, NK cells are composed of two distinct populations: CD56dim/CD16-positive and CD56bright/CD16-negative. In healthy individuals, CD16-negative represents 5-15% of the total NK population. However, in some cancer patients the proportion of CD16-negative NK cells is greatly increased. Those cell populations can be identified by flow cytometry. In addition, the tumor microenvironment has been shown to affect the phenotype of CD56dim/CD16pos NK cells by either inducing shedding of CD16a from the surface of the cells (activity mediated by ADAM17 enzyme) or downregulating its expression on the surface of the cells (activity mediated by TGFβ and others). The level of CD16a expression on NK cells can be measured using flow cytometry. Further, due to CD16a polymorphism, some individuals have a mutation in CD16a that dramatically impaired ADCC mediated by monoclonal antibodies. Patients can be genotyped. In addition, in allogenic hematopoietic stem-cell transplants (HCSTs), the presence of a mismatch between NK inhibitory receptors and HLA allows donor NK cells to kill recipient immune cells, therefore eliminating residual malignant cells. Recently, it was shown that the first NK cells that are reconstituted after transplant present an NKp30+/CD16− phenotype. Therefore, it is of great importance to mediate tumor cell killing in the absence of CD16 expression.

Optionally, the present compositions and methods for enhancing an immune response involve subjects in which the NK cells have less than 50% expression of CD16 as compared to a control NK cell. Optionally, the subject's CD16 deficient microenvironment includes a population of tumor infiltrating NK cells in which at least 10% of the infiltrating NK cells have reduced expression of CD16 as compared to a control NK cell. The NK cell can have a CD16 copy number less than 150,000. In some embodiments, the NK cell does not express CD16.

Multispecific antigen-binding constructs that include at least two linked antigen-binding units that recognize specific target antigens are provided. Thus, the term “multispecific antigen-binding construct” as used herein includes bispecific, trispecific, tetraspecific, and multispecific antigen-binding constructs.

A multispecific antigen-binding construct can be a single multifunctional polypeptide, small molecule, or aptamer, or it can be a multimeric complex of two or more molecules that are covalently or non-covalently associated with one another. Multispecific antigen-binding constructs include antibodies (or antigen-binding fragments thereof) that can be linked to or co-expressed with another functional molecule, e.g., another peptide, protein, and/or aptamer. For example, an antibody or fragment thereof can be functionally linked (e.g., by chemical coupling, genetic fusion, non-covalent association or otherwise) to one or more other molecular entities, such as a protein or fragment thereof to produce a multispecific antigen-binding construct with a second binding specificity. In certain embodiments, an antibody or antigen-binding fragment thereof is functionally linked to one or more antibody or antigen-binding fragment thereof having a different binding specificity to produce a multispecific antigen-binding construct. Each antibody or antigen-binding portion thereof of the construct can have one or more antigen-binding specificities.

As used herein, an antigen-binding unit refers to a domain, region, or the like, of the multispecific antigen-binding construct that forms an area of the construct that binds to an antigen. A first antigen-binding unit forms a separate binding area of the multispecific antigen-binding construct from a second antigen-binding unit of the construct, each unit forming a separate region of antigen-binding. Generally, one unit (first unit) is distinct from the other unit (second unit) in its antigen-binding. For example, one antigen-binding unit of the construct is monovalent for and binds to a tumor or B-lineage cell antigen (e.g., BCMA) whereas the other antigen-binding unit of the construct is monovalent for and binds to NKp30. Further, by way of example, one antigen-binding unit of the construct binds to a tumor or B-lineage cell antigen whereas the other arm of the construct binds to NKp30 or a related molecule (cross-reacts with two antigens due to, e.g., similarity in structure). See, e.g., U.S. Pat. No. 9,845,356.

Optionally, and in some embodiments, the multispecific construct is tetravalent. For example, in some embodiments, one antigen-binding unit is bivalent for a tumor or B-lineage cell antigen, each binding the same epitope on the tumor or B-lineage cell antigen, whereas the other antigen-binding unit is bivalent for NKp30, each binding the same epitope on NKp30. In some embodiments, the multispecific construct is tetravalent, wherein one antigen-binding unit is bivalent for a tumor or B-lineage cell antigen, each binding two different epitopes on the tumor or B-lineage cell antigen. In some embodiments, the multispecific construct is tetravalent, wherein one antigen-binding unit is bivalent for NKp30, each binding two different epitopes on NKp30. In some embodiments, the multispecific construct is tetravalent, wherein one antigen-binding unit is bivalent for a tumor or B-lineage cell antigen, each binding two different but overlapping epitopes on the tumor or B-lineage cell antigen. In some embodiments, the multispecific construct is tetravalent, wherein one antigen-binding unit is bivalent for NKp30, each binding two different but overlapping epitopes of NKp30. In some embodiments, the multispecific construct is tetravalent, wherein one antigen-binding unit is monovalent for a first tumor antigen, one antigen-binding unit is monovalent for a second tumor or B-lineage cell antigen, and one antigen-binding unit is bivalent for NKp30, each binding the same epitope on NKp30. In some embodiments, the multispecific construct is tetravalent, wherein one antigen-binding unit is monovalent for a tumor or B-lineage cell antigen, one antigen-binding unit is bivalent for NKp30, each binding the same epitope on NKp30, and one antigen-binding unit is monovalent for a second NK receptor. In some embodiments, the multispecific construct is tetravalent, wherein one antigen-binding unit is monovalent for a first tumor or B-lineage cell antigen, one antigen-binding unit is monovalent for a second tumor or B-lineage cell antigen, one antigen-binding unit is monovalent for NKp30, and one antigen-binding unit is monovalent for a second NK receptor.

The term valency, when used to describe an antigen-binding construct or protein, refers to the number of recognition (binding) sites in the antigen-binding construct or protein, regardless of whether those different recognition or binding sites bind to the same epitope. Each recognition site specifically recognizes, and is therefore capable of binding, one epitope (binding site) on an antigen. When an antigen-binding protein comprises more than one recognition site (e.g., when an antigen-binding protein is an IgG, which has two recognition sites in its variable regions), each recognition site can specifically recognize the same epitope on the same antigen, or different epitopes, whether on the same or different antigens. Multivalency can increase the avidity, i.e., the strength of binding between a receptor-binding unit or construct and the pertinent antigen or target receptor. Avidity is related to both the affinity between an epitope or antigenic determinant and its binding site on the antigen-binding unit, and the actual number of pertinent binding sites present on the antigen-binding unit.

In some embodiments, the multispecific antigen-binding constructs comprise a first antigen-binding unit that specifically binds a tumor or B-lineage cell antigen, and a second antigen-binding unit that specifically binds NKp30. With regard to the binding of an antigen-binding unit to a target molecule, the terms specific binding, specifically binds to, specific for, selectively binds, selective for, and the like, as related to a particular target antigen or molecule (e.g., a polypeptide target) or an epitope on a particular target antigen or molecule mean binding that is measurably different from a non-specific or non-selective interaction. Specific binding can be measured, for example, by determining binding of a target molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

The term epitope, as used herein, means a component of an antigen capable of specific binding to an antigen-binding construct or unit. Epitopes frequently consist of surface-accessible amino acid residues and/or sugar side chains and can have specific three-dimensional structural characteristics, as well as specific charge characteristics, and can be contiguous or noncontiguous. Conformational and non-conformational epitopes are distinguished in that the binding to the former, but not the latter, is lost in the presence of denaturing solvents. An epitope can comprise amino acid residues that are directly involved in the binding, and other amino acid residues, which are not directly involved in the binding. The epitope to which an antigen-binding protein binds can be determined using known techniques for epitope determination such as, for example, testing for antigen-binding protein binding to antigen variants with different point mutations, e.g., “epitope mapping,” and/or using X-ray crystallography techniques.

In some embodiments, at least one antigen-binding unit has a K_{D} of at least 1×10⁻⁷M, at least 1×10⁻⁸M, at least 1×10⁻⁹M, at least 1×10⁻¹⁰ M, at least 1×10⁻¹¹M, at least 1×10⁻¹²M, or at least 1×10⁻¹³M. In some embodiments, the antigen-binding units of the construct have the same or similar K_{D}.

The term K_{D} (M), as used herein, refers to the dissociation equilibrium constant of a particular antigen-binding unit/antigen interaction. K_{D}=k_{d}/kₐ. The term k_{d} (sec⁻¹), as used herein, refers to the dissociation rate constant of a particular antigen-binding unit/antigen interaction. This value is also referred to as the k_{off} value. The term kₐ (M⁻¹×sec⁻¹), as used herein, refers to the association rate constant of a particular antigen-binding unit/antigen interaction. This value is also referred to as the kₒₙ value.

In some embodiments, the binding of one antigen-binding unit (e.g., the first antigen-binding unit) of the multispecific antigen-binding construct to its target does not block or sterically hinder the binding of the other antigen-binding unit(s) (e.g., the second antigen-binding unit) to its target. For example, upon the binding of a first antigen-binding unit to a tumor or B-lineage cell antigen, the second or subsequent antigen-binding unit is free to bind an NKp30 antigen. Thus, in some embodiments, the first antigen-binding unit and second antigen-binding unit bind to their respective targets concurrently.

In some embodiments, binding of the first antigen-binding unit and the second antigen-binding unit to their respective targets bridges the immune cell and the second cell together, bringing the two cells in close proximity. As used herein, bridge refers to the joining of two cell types (e.g., one immune cell that expresses NKp30 and a second cell that expresses a tumor or B-lineage cell antigen) or bringing of the two cells together in close proximity, such that the two cells need not be in physical contact. Thus, the multispecific antigen-binding construct acts as a connector (e.g., a bridge) to the two cells, each one expressing either NKp30 and/or a second NK activating receptor or one or more tumor or B-lineage cell antigens.

Methods for determining whether two cells are bridged by a construct of the present disclosure are known in the art. For example, in some embodiments, the bridging of the immune cell and the second cell is determined by, e.g., flow cytometry, FRET, immunoprecipitation, microscopy, or fluorescence plate reader.

As described herein, the constructs of the present disclosure are capable of binding to one or more immune cells that express NKp30 and/or a second NK activating receptor and one or more cells that express one or more tumor or B-lineage cell antigens. The type of immune cell depends on the context of the disease to be treated, and the particular type of immune cell can be determined by one of skill in the art depending on the disorder under consideration. In some embodiments, the immune cell is a T cell, including a CD8+ T cell and CD4+ cell, including an effector γδ T cell. In some embodiments, the immune cell is a natural killer (NK) cell. In some embodiments, the immune cell is a B cell. In some embodiments, the immune cell is a macrophage.

In some embodiments, the constructs are capable of binding one or more tumor cells (e.g., a solid or non-solid tumor cell). As used herein, tumor cell is sometimes used interchangeably with cancer cell, but also encompasses non-malignant (non-cancerous) cells exhibiting increased proliferation as compared to a normal cell. In some embodiments, the tumor cell is a cancer cell that can be treated by enhancing NKp30 function, while bridging the immune cell and the cell expressing a tumor antigen (e.g., BCMA-expressing tumor cell).

By way of example, the tumor cell is from a cancer selected from the group consisting of a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, liver cancer, pancreatic cancer, a genitourinary cancer, a bone cancer, renal cancer, and a vascular cancer. Optionally, the tumor cell (and specific tumor antigens associated with such tumor cells, but not exclusively) is from a cancer selected from the group consisting of Kaposi's sarcoma, leukemia, acute lymphocytic leukemia (etv6, aml1, cyclophilin b), acute myelocytic leukemia, myeloblasts promyelocyte myelomonocytic monocytic erythroleukemia, chronic leukemia, chronic myelocytic (granulocytic) leukemia, chronic lymphocytic leukemia (cyclophilin b), mantle cell lymphoma, primary central nervous system lymphoma, Burkitt's lymphoma, marginal zone B cell lymphoma (Ig-idiotype), Polycythemia vera Lymphoma, Hodgkin's disease (Imp-1, EBNA-1), non-Hodgkin's disease, mycloma (MUC family, p21ras), multiple myeloma, Waldenstrom's macroglobulinemia, heavy chain disease, solid tumors, sarcoma, carcinoma, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colon carcinoma (p21ras, HER2/neu, c-erbB-2, MUC family), pancreatic cancer, breast cancer (MUC family, HER2/neu, c-erbB-2), ovarian cancer, prostate cancer (Prostate Specific Antigen (PSA) and its antigenic epitopes PSA-1, PSA-2, and PSA-3, PSMA, HER2/neu, c-erbB-2, ga733 glycoprotein), squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma (HER2/neu, c-erbB-2), hepatoma, hepatocellular cancer (α-fetoprotein), bile duct carcinoma, choriocarcinoma, seminoma, embryonal carcinoma, Wilm's tumor, cervical cancer, uterine cancer, testicular tumor (NY-ESO-1), lung carcinoma, small cell lung carcinoma, non-small cell lung carcinoma (HER2/neu, c-erbB-2), bladder carcinoma, epithelial carcinoma, glioma (E-cadherin, α-catenin, β-catenin, γ-catenin, p120ctn), astrocytoma, medulloblastoma, craniopharyngioma, ependymoma, pinealoma, hemangioblastoma, acoustic neuroma, oligodendroglioma, menangioma, melanoma (p5 protein, gp75, oncofetal antigen, GM2 and GD2 gangliosides, Melan-A/MART-1, cdc27, MAGE-3, p21ras, gp100), neuroblastoma, retinoblastoma, nasopharyngeal carcinoma (Imp-1, EBNA-1), esophageal carcinoma, basal cell carcinoma, biliary tract cancer (p21ras), bladder cancer (p21ras), bone cancer, brain and central nervous system (CNS) cancer, cervical carcinoma (p53, p21ras), choriocarcinoma (CEA), colorectal cancers (colorectal associated antigen (CRC)-0017-1A/GA733, APC), connective tissue cancer, cancer of the digestive system, endometrial cancer, esophageal cancer, eye cancer, head and neck cancer, gastric cancer (HER2/neu, c-erbB-2, ga733 glycoprotein), epithelial cell cancer (cyclophilin b), intraepithelial neoplasm, kidney cancer, larynx cancer, liver cancer, lung cancer (small cell, large cell) (CEA, MAGE-3, NY-ESO-1), oral cavity cancer (for example lip, tongue, mouth, and pharynx cancers), ovarian cancer (MUC family, HER2/neu, c-erbB-2), pancreatic cancer, rectal cancer, cancer of the respiratory system, skin cancer, thyroid cancer, and cancer of the urinary system.

In some constructs, the constructs are capable of binding one or more B-lineage cells. As used herein B-lineage cells include pro-B cells, pre-B cells, transitional B cells, follicular B cells, marginal zone B cells, germinal center B cells, plasma cells, and memory B cells.

Specifically provided is a multispecific antigen-binding construct comprising at least two linked antigen-binding units, wherein a first antigen-binding unit specifically binds a first target antigen expressed by a B-lineage cell and wherein a second antigen-binding unit specifically binds a NKp30 antigen. For example, the first target antigen can be a B-lineage cell maturation antigen (BCMA). Optionally, the multispecific antigen-binding construct further comprises a third antigen-binding unit that binds to a second target antigen expressed by a B-lineage cell.

In addition to BCMA, target antigens expressed by a B-lineage cell include, for example, CD1c, CD5, CD10, CD19, CD20, CD21, CD22, CD23, CD24, CD27, CD34, CD38, CD40, CD72, CD78, CD79a, CD79b, CD80, CD84, CD86, CD126, CD138, CD319, TAC, GPRC5D (G protein-coupled receptor class C group 5 member D), SLAMF7 (CS1), and IL7/3R.

### Antibodies and Antigen-Binding Portions Thereof

As described herein, the disclosed multispecific antigen-binding constructs include bispecific, trispecific, tetraspecific, or multispecific antibodies or antigen-binding fragments thereof.

The constructs described herein can, in various aspects and embodiments, comprise one or more antibodies and/or antigen-binding portions thereof. For example, an antigen-binding unit can comprise a variable heavy and/or variable light chain, or complementarity determining regions thereof, of a given antibody to NKp30 or a given antibody to a tumor antigen, such as BCMA or HER2. Accordingly, in some embodiments of any of the aspects described herein, the first antigen-binding unit, second antigen-binding unit, third antigen-binding unit, or any combination thereof, can comprise an antibody or an antigen-binding portion thereof. In some embodiments of any of the aspects described herein, the first antigen-binding unit, second antigen-binding unit, third antigen-binding unit, or any combination thereof is an antibody or an antigen-binding portion thereof.

The term immunoglobulin or antibody, as used herein, refers to a class of structurally related proteins generally comprising two pairs of polypeptide chains: one pair of light (L) chains and one pair of heavy (H) chains. In an intact immunoglobulin, all four of these chains are interconnected by disulfide bonds. The structure of immunoglobulins has been well characterized. See, e.g., Paul (2013) Fundamental Immunology 7th ed., Ch. 5, Lippincott Williams & Wilkins, Philadelphia, Pa. Briefly, each heavy chain typically comprises a heavy chain variable region (V_{H}) and a heavy chain constant region (CH). The heavy chain constant region typically comprises three domains, C_{H1}, C_{H2}, and C_{H3}. Each light chain typically comprises a light chain variable region (V_{L}) and a light chain constant region. The light chain constant region typically comprises one domain, abbreviated C_{L}.

An antibody, as used herein, can refer to intact antibodies (e.g., intact immunoglobulins). However, terms antigen-binding portions and antigen-binding fragments can be used interchangeably with an intact antibody. Antigen-binding fragments comprise at least one antigen-binding domain. One example of an antigen-binding domain is an antigen-binding domain formed by a V_{H}-V_{L} dimer. Antibodies and antigen-binding fragments can be described by the antigen to which they specifically bind. For example, an NKp30 antibody, or anti-NKp30 antibody, is an antibody that specifically binds to NKp30.

The V_{H} and V_{L} regions can be further subdivided into regions of hypervariability (hypervariable regions (HVRs), also called complementarity determining regions (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen-binding and confer antigen specificity and binding affinity to the antibody. (See Kabat et al. (1991) Sequences of Proteins of Immunological Interest 5th ed., Public Health Service, National Institutes of Health, Bethesda, Md. and Chothia, C. et al. (1987) J. Mol. Biol. 196:901-917; which are incorporated by reference herein in their entireties.) The three heavy chain CDRs can be referred to as CDRH1, CDRH2, and CDRH3, and the three light chain CDRs can be referred to as CDRL1, CDRL2, and CDRL3.

The system described by Kabat, also referred to as “numbered according to Kabat,” “Kabat numbering”, “Kabat definitions”, and “Kabat labeling,” provides an unambiguous residue numbering system applicable to any variable domain of an antibody, and provides precise residue boundaries defining the three CDRs of each chain. (Kabat et al., Sequences of Proteins of Immunological Interest, National Institutes of Health, Bethesda, Md. (1987) and (1991), the contents of which are incorporated by reference in their entirety. These CDRs are referred to as Kabat CDRs and comprise about residues 24-34 (CDR1), 50-56 (CDR2) and 89-97 (CDR3) in the light chain variable domain, and 31-35 (CDR1), 50-65 (CDR2) and 95-102 (CDR3) in the heavy chain variable domain. When the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. The “EU index as in Kabat” refers to the residue numbering of the human IgG1 EU antibody.

Other CDR numbering systems are also used in the art. Chothia and coworkers found that certain sub-portions within Kabat CDRs adopt nearly identical peptide backbone conformations, despite having great diversity at the level of amino acid sequence. (Chothia et al. (1987) J. Mol. Biol. 196: 901-917; and Chothia et al. (1989) Nature 342: 877-883). These sub-portions were designated as L1, L2, and L3 or H1, H2, and H3 where the “L” and the “H” designates the light chain and the heavy chains regions, respectively. These CDRs can be referred to as “Chothia CDRs,” “Chothia numbering,” or “numbered according to Chothia,” and comprise about residues 24-34 (CDR1), 52-56 (CDR2) and 89-97 (CDR3) in the light chain variable domain, and 26-32 (CDR1), 50-56 or 52-56 (CDR2), and 95-102 (CDR3) in the heavy chain variable domain. Mol. Biol. 196:901-917 (1987).

The system described by MacCallum, also referred to as “numbered according to MacCallum,” or “MacCallum numbering” comprises about residues 30-36 (CDR1), 46-55 (CDR2) and 89-96 (CDR3) in the light chain variable domain, and 30-35 (CDR1), 47-58 (CDR2) and 93-101 (CDR3) in the heavy chain variable domain. MacCallum et al. ((1996) J. Mol. Biol. 262(5):732-745).

The system described by AbM, also referred to as “numbering according to AbM,” or “AbM numbering” comprises about residues 24-34 (CDR1), 50-56 (CDR2) and 89-97 (CDR3) in the light chain variable domain, and 26-35 (CDR1), 50-58 (CDR2) and 95-102 (CDR3) in the heavy chain variable domain.

The IMGT (INTERNATIONAL IMMUNOGENETICS INFORMATION SYSTEM) numbering of variable regions can also be used, which is the numbering of the residues in an immunoglobulin variable heavy or light chain according to the methods of the IMGT, as described in Lefranc, M.-P., “The IMGT unique numbering for immunoglobulins, T cell Receptors and Ig-like domains”, The Immunologist, 7, 132-136 (1999), and is expressly incorporated herein in its entirety by reference. As used herein, “IMGT sequence numbering” or “numbered according to IMTG,” refers to numbering of the sequence encoding a variable region according to the IMGT. For the heavy chain variable domain, when numbered according to IMGT, the hypervariable region ranges from amino acid positions 27 to 38 for CDR1, amino acid positions 56 to 65 for CDR2, and amino acid positions 105 to 117 for CDR3. For the light chain variable domain, when numbered according to IMGT, the hypervariable region ranges from amino acid positions 27 to 38 for CDR1, amino acid positions 56 to 65 for CDR2, and amino acid positions 105 to 117 for CDR3.

In some embodiments of the constructs and antigen-binding units described herein, the CDRs recited herein comprise about residues 24-34 (CDR1), 49-56 (CDR2) and 89-97 (CDR3) in the light chain variable domain, and 27-35 (CDR1), 49-60 (CDR2) and 93-102 (CDR3) in the heavy chain variable domain, when numbered according to Chothia numbering. In some embodiments, CDR2 in the light chain variable domain can comprise amino acids 49-56, when numbered according to Chothia numbering.

Methods of generating and screening for an antibody or antibody fragment against a desired target are well-known in the art. Methods of further modifying antibodies or antibody fragments for enhanced properties (e.g., enhanced affinity, chimerization, humanization) as well as generating antigen-binding fragments, as described herein, are also well-known in the art.

The term chimeric antibody or antibody fragment refers to an antibody or antibody fragment in which a component of the heavy and/or light chain is derived from a particular source or species, while the remainder of the heavy and/or light chain is derived from a different source or species.

Humanized forms of non-human antibodies or antibody fragments are chimeric antibodies or antibody fragments that contain minimal sequence derived from the non-human antibody or antibody fragment. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues of the donor antibody. Humanized antibodies or antibody fragments can also comprise residues that are not found in either the recipient antibody or the donor antibody. Such modifications can be made to further refine antibody function. (See Jones et al. (1986) Nature 321:522-525; Riechmann et al. (1988) Nature, 332:323-329; and Presta, (1992) Curr. Op. Struct. Biol., 2:593-596).

A human antibody or antibody fragment is one that possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies or antibody fragments specifically exclude humanized antibodies or antibody fragments.

In some embodiments, an antibody molecule comprises a diabody and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy chain variable domain sequences and two light chain variable domain sequence, thereby forming two antigen-binding sites, such as Fab, Fab′, F(ab′)2, Fc, Fd, Fd′, Fv, single chain antibodies (scFv, for example), single variable domain antibodies, diabodies (Dab) (bivalent and bispecific), and chimeric (e.g., humanized) antibodies, which can be produced by the modification of whole antibodies or those synthesized de novo using recombinant DNA technologies. These functional antibody fragments retain the ability to selectively bind with their respective antigen. Antibodies and antibody fragments can be from any class of antibodies including, but not limited to, IgG, IgA, IgM, IgD, and IgE, and from any subclass (e.g., IgG1, IgG2, IgG3, IgG4, IgA1, and IgA2) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

Antigen-binding fragments or antigen-binding portions of an antibody molecule are well known in the art, and include, for example, (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); or (viii) a single domain antibody. These antibody fragments can be obtained using conventional techniques known to those skilled in the art, and the fragments can be screened for utility in the same manner as are intact antibodies.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies can be any known in the art or any future single domain antibodies. Single domain antibodies can be derived from any species including, but not limited to mouse, human, camel, llama, fish, shark, goat, rabbit, and bovine. Single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca, and guanaco) or other species besides Camelidae.

In some embodiments, the multispecific antigen-binding construct comprises a bispecific antibody, having specificity for at least two antigens but optionally having more than two binding sites. A bispecific antibody molecule is characterized by a first immunoglobulin variable domain sequence that has binding specificity for a first antigen (e.g., BCMA or other tumor or B-lineage cell antigen) and a second immunoglobulin variable domain sequence that has binding specificity for a second antigen (e.g., NKp30). In some embodiments, a bispecific antibody molecule comprises a scFv or fragment thereof having binding specificity for a first antigen and a scFv or fragment thereof having binding specificity for a second antigen (See, e.g., Kontermann and Brinkmann (2015) Drug Discovery Today 20(7):838-47).

Various bispecific antibody formats are known in the art, including, for example, a bispecific IgG, a bispecific antibody fragment, a bispecific fusion protein, an appended IgG, and a bispecific antibody conjugate, described herein. Exemplary bispecific formats that can be used in the context of the present disclosure include, without limitation, e.g., scFv-based or diabody bispecific formats, IgG-scFv fusions, dual variable domain (DVD)-1g, Quadroma, knobs-into-holes, common light chain (e.g., common light chain with knobs-into-holes, etc.), CrossMab, CrossFab, (SEED)body, leucine zipper, Duobody, lgG1/lgG2, dual acting Fab (DAF)-1gG, and Mab² bispecific formats (see, e.g., Klein et al. (2012) mAbs 4:6, 1-11, and references cited therein, for a review of the foregoing formats; see also Spiess et al. (2015) Mol. Immunol. 67:95-106). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency, and geometry. (See, e.g., Kazane et al. (2013) J. Am. Chem. Soc. 135(1):340-6). In some embodiments, the multispecific antigen-binding constructs disclosed herein comprise antibodies that comprise a common light chain. Non-limiting examples of amino acid sequences of common light chains used in the constructs described herein include SEQ ID NO: 8 and SEQ ID NO: 81.

In certain embodiments, the antibody or antigen-binding fragment thereof of an antigen-binding unit comprises known antibodies or the CDRs of known NKp30 antibodies, for example, anti-NKp30 antibodies described in U.S. Pat. No. 7,517,966, the contents and sequences of which are herein incorporated by reference in their entirety. Optionally, the NKp30 antibody is a humanized version of the antibody produced by the hybridoma having Collection Nationale De Cultures De Micro-organismes (CNCM) Registration Number 1-2576.

In certain embodiments, the antibody or antigen-binding fragment thereof of an antigen-binding unit comprises known NKp46 antibodies or the CDRs of known NKp46 antibodies, for example, anti-NKp46 antibodies described in any of WO2018138032, WO2017114694, WO2016207278, WO2016207273, WO2015197593, WO2015197598, WO2015197582, US20150376274, US20180207290, and WO2018047154, the contents and sequences of each of which are herein incorporated by reference in their entireties.

In certain embodiments, the antibody or antigen-binding fragment thereof of an antigen-binding unit comprises known antibodies or the CDRs of known NKG2D antibodies, for example, anti-NKG2D antibodies described in any of WO2018148447, WO2018035330, WO2010017103, and U.S. Pat. No. 9,273,136, the contents and sequences of each of which are herein incorporated by reference in their entireties.

In certain embodiments, the antibody or antigen-binding fragment thereof of an antigen-binding unit comprises known antibodies or the CDRs of known CD137 antibodies, for example, anti-CD137 antibodies described in any of WO2017205745, US20160244528, WO2016134358, US20170226215, and WO2018191502, the contents and sequences of each of which are herein incorporated by reference in their entireties.

In certain embodiments, the antibody or antigen-binding fragment thereof of the first antigen-binding unit comprises known antibodies or the CDRs of known antibodies to a tumor antigen. By way of example, trademarked and non-proprietary names of exemplary tumor antigen-targeting antibodies currently approved for use in cancer therapy by the U.S. Food and Drug Administration and/or the European Medicines Agency include, but are not limited to: LEMTRADA® (alemtuzumab, SanofiGenzyme; see e.g., Keating et al. (2002) Blood 99(10):3556-3561; Hillmen et al. (2007) J. Clin. Oncol. 25(35):5616-5623); AVASTIN® (bevacizumab, Genentech; see e.g., Ferrara et al. (2005) Biochem. Biophys. Res. Commun. 333(2):328-335); ADCETRIS® (brentuximab vedotin, Seattle Genetics; see e.g., Younes et al. (2010) N. Engl. J. Med. 363(19): 1812-1821; Senter et al. (2012) Nat. Biotechnol. 30(7):631-637); REMOVAB® (catumaxomab, Fresenius Biotech; see e.g., Seimetz (2011) J. Cancer 2:309-316); ERBITUX® (cetuximab, Eli Lilly; see e.g., Wong (2005) Clin. Ther. 27(6):684-694); ZEVALIN® (ibritumomab tiuxetan, Spectrum Pharmaceuticals; see e.g., Witzig et al. (2002) J. Clin. Oncol. 20(10):2453-2463; Marcus (2005) Semin. Oncol. 32(1 Suppl 1:S36-43); VECTIBIX® (panitumumab, Amgen; see e.g., Chu (2006) Clin. Colorectal Cancer 6(1):13; Van Cutsem et al. (2007) J. Clin. Oncol. 25(13):1658-1664); PERJETA® (pertuzumab, Genentech; Baselga et al. (2012) N. Engl. J. Med. 366(2):109-119; Agus et al. (2005) J. Clin. Oncol. 23(11):2534-2543); RITUXAN® (rituximab, Biogen; see e.g., Feugier (2015) Future Oncol. 11(9):1327-1342); ARZERRA® (ofatumumab, Novartis; see e.g., Teeling et al. (2006) J. Immunol. 177(1):362-371; Teeling et al. (2004) Blood 104(6):1793-1800); GAZVYA® (obinutuzumab, Genentech; see e.g., Mossner et al. (2010) Blood 115(22):4393-4402; Golay et al. (2013) Blood 122(20):3482-3491; Goede et al. (2014) N. Engl. J. Med. 370(12):1101-1110; Reddy et al. (2017) Rheumatology (Oxford) 56(7):1227-1237); OCREVUS® (ocrelizumab, Genentech; see e.g., Reichert (2017) MAbs 9(2):167-181; Montalban (2016) N. Engl. J. Med. 376(3):209-220); PORTRAZZA® (necitumumab, Eli Lilly; see e.g., Dienstmann and Tabernero (2010) Curr. Opin. Investig. Drugs 11(12):1434-1441); HERCEPTIN® (trastuzumab, Genentech; see e.g., Slamon et al. (2001) N. Engl. J. Med. 344(11):783-792; Maximiano et al. (2016) BioDrugs 30(2):75-86); KADCYLA® (ado-trastuzumab emtansine, Genentech; see e.g., Verma et al. (2012) N. Engl. J. Med. 367(19):1783-1791; Krop et al. (2014) 15(7):689-699); DARZALEX® (daratumumab, Janssen; see e.g., de Weers et al. (2011) J. Immunol. 186(3):1840-1848; Lonial et al. (2016) Lancet 387(10027):1551-1560); UNITUXIN® (dinutuximab, United Therapeutics; see e.g., Dhillon (2015) Drugs 75(8):923-927); and LARTRUVO® (olaratumab, Eli Lilly; see e.g., Tap et al. (2016) Lancet 388(10043):488-497; Shirley (2017) Drugs 77(1):107-112).

Additional exemplary tumor antigen-targeting antibodies include, but are not limited to, I-131-BC8 (alternatively known as Iomab-B, Actinium Pharmaceuticals), talacotuzumab (alternatively known as JNJ-56022473, Janssen), vadastuximab talirine (Seattle Genetics), ublituximab (TG Therapeutics), moxetumomab pasudotox (AstraZeneca/MedImmune), XMAB-5574 (alternatively known as MOR208, Xencor), oportuzumab monatox (Viventia Bio), margetuximab (MacroGenics), MM-302 (Merrimack Pharmaceuticals), sacituzumab govitecan (Immunomedics, Inc.), glembatumumab vedotin (Celldex Therapeutics), andecaliximab (Gilead Sciences), depatuxizumab mafodotin (AbbVie), tremelimumab (AstraZeneca/MedImmune), racotumomab (Recombio SL), anetumab ravtansine (Bayer), mirvetuximab sorvtansine (ImmunoGen), and carotuximab (TRACON Pharma), all of which are further described in Reichert (2017) MAbs 9(2):167-181, which is incorporated herein by reference in its entirety.

In certain embodiments, the antibody or antigen-binding fragment thereof of the first antigen-binding unit comprises a heavy chain variable region comprising a CDRH1, a CDRH2, and a CDRH3 of SEQ ID NO: 2; and a light chain variable region comprising a CDRL1, a CDRL2, and a CDRL3 of SEQ ID NO: 1, optionally with one or more conservative amino acid substitutions. A conservative amino acid substitution is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain embodiments, a string of amino acids can be replaced with a structurally similar string that differs in order and/or composition of side chain family members. Alternatively, in certain embodiments, mutations may be introduced randomly along all or part of a coding sequence, such as by saturation mutagenesis, and the resultant mutants can be incorporated into an antigen-binding unit as described herein and screened for their ability to bind to the desired target.

In some aspects, the antibody or antigen-binding fragment thereof of the first antigen-binding unit comprises a heavy chain comprising an amino acid sequence that is at least 90% identical to SEQ ID NO: 2 and a light chain comprising an amino acid sequence that is at least 90% identical to SEQ ID NO: 1. In some aspects, the multispecific antigen-binding construct comprises at least one heavy chain comprising an amino acid sequence that is at least 90% identical to SEQ ID NO: 2. In certain aspects, the multispecific antigen-binding construct comprises at least one heavy chain comprising an amino acid sequence of SEQ ID NO: 2. In some aspects, the multispecific antigen-binding construct comprises at least one light chain comprising an amino acid sequence that is at least 90% identical to SEQ ID NO: 1. Optionally, the multispecific antigen-binding construct comprises at least one light chain comprising an amino acid sequence of SEQ ID NO: 1. Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. As used herein, at least 90% identity includes at least 91%, at least 92%, at least 93%, at least 94%, at least 95%, at least 96%, at least 97%, at least 98%, and at least 99% identical, and every percentage between 90% and 100%, inclusively.

In certain embodiments, the antibody or antigen-binding fragment thereof of an antigen-binding unit comprises a CDRH1, a CDRH2, a CDRH3, a CDRL1, a CDRL2, and a CDRL3 of an NKp30 antibody described in U.S. Pat. No. 7,517,966. In some embodiments, the antibody or antigen-binding fragment thereof of an antigen-binding unit comprises a heavy chain comprising an amino acid sequence that is at least 90% identical to the heavy chain of an antibody described in U.S. Pat. No. 7,517,966 and a light chain comprising an amino acid sequence that is at least 90% identical to the light chain of an antibody described in U.S. Pat. No. 7,517,966. In certain embodiments, the antibody or antigen-binding fragment thereof of an antigen-binding unit comprises a CDRH1, a CDRH2, a CDRH3, a CDRL1, a CDRL2, and a CDRL3 of an NKp46 antibody described in any of WO2018138032, WO2017114694, WO2016207278, WO2016207273, WO2015197593, WO2015197598, WO2015197582, US20150376274, US20180207290, and WO2018047154. In some embodiments, the antibody or antigen-binding fragment thereof of an antigen-binding unit comprises a heavy chain comprising an amino acid sequence that is at least 90% identical to the heavy chain of an antibody described in any of WO2018138032, WO2017114694, WO2016207278, WO2016207273, WO2015197593, WO2015197598, WO2015197582, US20150376274, US20180207290, and WO2018047154 and a light chain comprising an amino acid sequence that is at least 90% identical to the light chain of an antibody described in any of WO2018138032, WO2017114694, WO2016207278, WO2016207273, WO2015197593, WO2015197598, WO2015197582, US20150376274, US20180207290, and WO2018047154.

In certain embodiments, the antibody or antigen-binding fragment thereof of an antigen-binding unit comprises a CDRH1, a CDRH2, a CDRH3, a CDRL1, a CDRL2, and a CDRL3 of an NKG2D antibody described in any of WO2018148447, WO2018035330, WO2010017103, and U.S. Pat. No. 9,273,136. In some embodiments, the antibody or antigen-binding fragment thereof of an antigen-binding unit comprises a heavy chain comprising an amino acid sequence that is at least 90% identical to the heavy chain of an antibody described in any of WO2018148447, WO2018035330, WO2010017103, and U.S. Pat. No. 9,273,136 and a light chain comprising an amino acid sequence that is at least 90% identical to the light chain of an antibody described in any of WO2018148447, WO2018035330, WO2010017103, and U.S. Pat. No. 9,273,136.

In certain embodiments, the antibody or antigen-binding fragment thereof of an antigen-binding unit comprises a CDRH1, a CDRH2, a CDRH3, a CDRL1, a CDRL2, and a CDRL3 of a CD137 antibody described in any of WO2017205745, US20160244528, WO2016134358, US20170226215, and WO2018191502. In some embodiments, the antibody or antigen-binding fragment thereof of an antigen-binding unit comprises a heavy chain comprising an amino acid sequence that is at least 90% identical to the heavy chain of an antibody described in any of WO2017205745, US20160244528, WO2016134358, US20170226215, and WO2018191502 and a light chain comprising an amino acid sequence that is at least 90% identical to the light chain of an antibody described in any of WO2017205745, US20160244528, WO2016134358, US20170226215, and WO2018191502.

In some embodiments, the multispecific antigen-binding construct comprises at least one copy of a heavy chain that includes both CDRH sequences for the tumor antigen, such as BCMA or HER2, as well as CDRH sequences for NKp30.

Also, provided herein, in some aspects, are novel antibodies and antigen-binding fragments thereof that can be used in the multispecific antigen-binding constructs described herein. In other words, one or more antigen-binding units of the multispecific antigen-binding constructs described herein can comprise heavy and/or light chain CDRs, heavy and/or light chain variable regions, and/or full-length heavy and/or light chains derived or selected from any of the novel antibodies described herein.

### BCMA Binding Antibodies and Antigen Binding Portions Thereof

Accordingly, provided herein is a novel antibody or antigen-binding portion thereof that specifically binds human BCMA, wherein the antibody or antigen-binding portion thereof comprises a heavy chain variable region comprising (i) a CDRH1 comprising SEQ ID NO: 38 (YTFX₁X₂X₃YX₄H, where X₁ is T or S, X₂ is N or S, X₃ is Y or H, and X₄ is M or V); (ii) a CDRH2 comprising SEQ ID NO: 39 (GX₅IDPSX₆GX₇TX₈YA, where X₅ is V or I, X₆ is G or D, X₇ is G, Y or S, and X₈ is N or S); and (iii) a CDRH3 comprising SEQ ID NO: 40 (ARGRYDYX₉DYLGWFDX₁₀, where X₉ is G or S, X₁₀ is P or G). In some embodiments, the antibody or antigen-binding portion thereof that specifically binds human BCMA further comprises a light chain variable region comprising (i) a CDRL1 comprising SEQ ID NO: 19; (ii) a CDRL2 comprising SEQ ID NO: 20; and (iii) a CDRL3 comprising SEQ ID NO: 43. Representative examples of antibodies having such heavy and light chain CDRs are mAb 1, mAb2, mAb3, mAb4, mAb5, mAb6, and mAb7.

Thus, provided herein, in some aspects, is an antibody or antigen-binding portion thereof that specifically binds human BCMA comprising a heavy chain CDR1 of SEQ ID NO: 11, a heavy chain CDR2 of SEQ ID NO: 12, and a heavy chain CDR3 of SEQ ID NO: 41. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb1.

In some aspects, provided herein is an antibody or antigen-binding portion thereof that specifically binds human BCMA comprising a heavy chain CDR1 of SEQ ID NO: 44, a heavy chain CDR2 of SEQ ID NO: 45, and a heavy chain CDR3 of SEQ ID NO: 46. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb2.

In some aspects, provided herein is an antibody or antigen-binding portion thereof that specifically binds human BCMA comprising a heavy chain CDR1 of SEQ ID NO: 47, a heavy chain CDR2 of SEQ ID NO: 48, and a heavy chain CDR3 of SEQ ID NO: 46. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb3.

In some aspects, provided herein is an antibody or antigen-binding portion thereof that specifically binds human BCMA comprising a heavy chain CDR1 of SEQ ID NO: 11, a heavy chain CDR2 of SEQ ID NO: 45, and a heavy chain CDR3 of SEQ ID NO: 49. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb4.

In some aspects, provided herein is an antibody or antigen-binding portion thereof that specifically binds human BCMA comprising a heavy chain CDR1 of SEQ ID NO: 11, a heavy chain CDR2 of SEQ ID NO: 50, and a heavy chain CDR3 of SEQ ID NO: 41. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb5.

In some aspects, provided herein is an antibody or antigen-binding portion thereof that specifically binds human BCMA comprising a heavy chain CDR1 of SEQ ID NO: 44, a heavy chain CDR2 of SEQ ID NO: 50, and a heavy chain CDR3 of SEQ ID NO: 41. In some embodiments, the antibody or antigen-binding portion thereof that specifically binds human BCMA further comprises a light chain variable region comprising a light chain CDR1 of SEQ ID NO: 19, a light chain CDR2 of SEQ ID NO: 20, and a light chain CDR3 of SEQ ID NO: 43. Representative antibodies having such CDRs are mAb6 and mAb7.

In some aspects and embodiments of the constructs, antibodies, and antigen-binding portions thereof described herein, the antibody or antigen-binding portion thereof that specifically binds human BCMA comprises a heavy chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to any one of SEQ ID NO: 10, SEQ ID NO: 52, SEQ ID NO: 53, SEQ ID NO: 54, SEQ ID NO: 55, SEQ ID NO: 56, and SEQ ID NO: 57. In some embodiments, the antibody or antigen-binding portion thereof that specifically binds human BCMA comprises a light chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 18. In some such embodiments, the heavy chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from any one of SEQ ID NO: 10, SEQ ID NO: 52, SEQ ID NO: 53, SEQ ID NO: 54, SEQ ID NO: 55, SEQ ID NO: 56, and SEQ ID NO: 57. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 18.

In some embodiments, the CDRs of the constructs, antibodies, and antigen-binding portions thereof that specifically bind human BCMA recited herein comprise about residues 24-34 (CDR1), 50-56 (CDR2) and 89-97 (CDR3) in the light chain variable domain, and 27-35 (CDR1), 49-60 (CDR2) and 93-102 (CDR3) in the heavy chain variable domain, when numbered according to Chothia numbering. In some embodiments, CDR2 in the light chain variable domain can comprise amino acids 49-56, when numbered according to Chothia numbering.

The disclosure also provides, in some embodiments, an antibody or antigen-binding portion thereof that specifically binds human BCMA that comprises three heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and three light chain CDRs of the light chain variable region of SEQ ID NO: 18.

The disclosure also provides, in some embodiments, an antibody or antigen-binding portion thereof that specifically binds human BCMA that comprises heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and light chain CDRs of the light chain variable region of SEQ ID NO: 18, wherein the heavy and light chain CDR residues are numbered according to Kabat.

The disclosure also provides, in some embodiments, an antibody or antigen-binding portion thereof that specifically binds human BCMA that comprises heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and light chain CDRs of the light chain variable region of SEQ ID NO: 18, wherein the heavy and light chain CDR residues are numbered according to Chothia.

The disclosure also provides, in some embodiments, an antibody or antigen-binding portion thereof that specifically binds human BCMA that comprises heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and light chain CDRs of the light chain variable region of SEQ ID NO: 18, wherein the heavy and light chain CDR residues are numbered according to MacCallum.

The disclosure also provides, in some embodiments, an antibody or antigen-binding portion thereof that specifically binds human BCMA that comprises heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and light chain CDRs of the light chain variable region of SEQ ID NO: 18, wherein the heavy and light chain CDR residues are numbered according to AbM.

The disclosure also provides, in some embodiments, an antibody or antigen-binding portion thereof that specifically binds human BCMA that comprises heavy chain CDRs of the heavy chain variable regions of any one of SEQ ID NO: 10, SEQ ID NO: 52, SEQ ID NO: 53, SEQ ID NO: 54, SEQ ID NO: 55, SEQ ID NO: 56, and SEQ ID NO: 57, and light chain CDRs of the light chain variable region of SEQ ID NO: 18, wherein the heavy and light chain CDR residues are numbered according to IMGT.

In some aspects and embodiments of the constructs, antibodies, and antigen-binding portions thereof described herein, the antibody or antigen-binding portion thereof that specifically binds human BCMA comprises a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to any one of SEQ ID NO: 51, SEQ ID NO: 58, SEQ ID NO: 59, SEQ ID NO: 60, SEQ ID NO: 61, SEQ ID NO: 62, SEQ ID NO: 63, and SEQ ID NO: 102. In some embodiments, the antibody or antigen-binding portion thereof that specifically binds human BCMA comprises a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some such embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from any one of SEQ ID NO: 51, SEQ ID NO: 58, SEQ ID NO: 59, SEQ ID NO: 60, SEQ ID NO: 61, SEQ ID NO: 62, SEQ ID NO: 63, and SEQ ID NO: 102. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

The disclosure also provides an isolated monoclonal antibody, or antigen-binding fragment thereof, that specifically binds to human BCMA, wherein, when bound to human BCMA, the antibody, or antigen-binding fragment thereof, binds to at last one of the amino acid residues bound by an anti-human BCMA antibody comprising the heavy chain variable region sequence depicted in any one of SEQ ID NO: 10, SEQ ID NO: 52, SEQ ID NO: 53, SEQ ID NO: 54, SEQ ID NO: 55, SEQ ID NO: 56, and SEQ ID NO: 57, and the light chain variable region sequence depicted in SEQ ID NO: 18. In another aspect, the disclosure features an isolated monoclonal antibody, or antigen-binding fragment thereof, that specifically binds to human BCMA, wherein, when bound to human BCMA, the antibody, or antigen-binding fragment thereof, cross blocks the binding of an anti-human BCMA antibody comprising the heavy chain variable region sequence depicted in any one of SEQ ID NO: 10, SEQ ID NO: 52, SEQ ID NO: 53, SEQ ID NO: 54, SEQ ID NO: 55, SEQ ID NO: 56, and SEQ ID NO: 57, and the light chain variable region sequence depicted in SEQ ID NO: 18.

### NKp30 Binding Antibodies and Antigen-Binding Portions Thereof

Also provided herein, in some aspects is a novel antibody or antigen-binding portion thereof that specifically binds human NKp30, wherein the antibody or antigen-binding portion thereof comprises a heavy chain variable region comprising a heavy chain CDR1 of SEQ ID NO: 15, a heavy chain CDR2 of SEQ ID NO: 16, and a heavy chain CDR3 of SEQ ID NO: 42. In some embodiments, the antibody or antigen-binding portion thereof that specifically binds human NKp30 further comprises a light chain variable region comprising a light chain CDR1 of SEQ ID NO: 19, a light chain CDR2 of SEQ ID NO: 20, and a light chain CDR3 of SEQ ID NO: 43. Representative antibodies having such CDRs are mAb8 and mAb9.

In some aspects and embodiments of the constructs described herein, the antibody or antigen-binding portion thereof that specifically binds human NKp30 comprises a heavy chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 14 or SEQ ID NO: 25 and/or a light chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 18. In some such embodiments, the heavy chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 14 or SEQ ID NO: 25. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 18.

In some embodiments, the CDRs of the antibody or antigen-binding portion thereof that specifically binds human NKp30 recited herein comprise about residues 24-34 (CDR1), 50-56 (CDR2) and 89-97 (CDR3) in the light chain variable domain, and 27-35 (CDR1), 49-60 (CDR2) and 93-102 (CDR3) in the heavy chain variable domain, when numbered according to Chothia numbering. In some embodiments, CDR2 in the light chain variable domain can comprise amino acids 49-56, when numbered according to Chothia numbering.

The disclosure also provides, in some embodiments, an antibody or antigen-binding portion thereof that specifically binds NKp30 that comprises the three heavy chain CDRs of the heavy chain variable regions of SEQ ID NO: 14 or 25, and the three light chain CDRs of the light chain variable region of SEQ ID NO: 18.

The disclosure also provides, in some embodiments, an antibody or antigen-binding portion thereof that specifically binds NKp30 that comprises heavy chain CDRs of the heavy chain variable regions of SEQ ID NO: 14 or 25, and light chain CDRs of the light chain variable region of SEQ ID NO: 18, wherein the heavy and light chain CDR residues are numbered according to Kabat.

The disclosure also provides, in some embodiments, an antibody or antigen-binding portion thereof that specifically binds NKp30 that comprises heavy chain CDRs of the heavy chain variable regions of SEQ ID NO: 14 or 25, and light chain CDRs of the light chain variable region of SEQ ID NO: 18, wherein the heavy and light chain CDR residues are numbered according to Chothia.

The disclosure also provides, in some embodiments, an antibody or antigen-binding portion thereof that specifically binds NKp30 that comprises heavy chain CDRs of the heavy chain variable regions of SEQ ID NO: 14 or 25, and light chain CDRs of the light chain variable region of SEQ ID NO: 18, wherein the heavy and light chain CDR residues are numbered according to MacCallum.

The disclosure also provides, in some embodiments, an antibody or antigen-binding portion thereof that specifically binds NKp30 that comprises heavy chain CDRs of the heavy chain variable regions of SEQ ID NO: 14 or 25, and light chain CDRs of the light chain variable region of SEQ ID NO: 18, wherein the heavy and light chain CDR residues are numbered according to AbM.

The disclosure also provides, in some embodiments, an antibody or antigen-binding portion thereof that specifically binds NKp30 that comprises heavy chain CDRs of the heavy chain variable regions of SEQ ID NO: 14 or 25, and light chain CDRs of the light chain variable region of SEQ ID NO: 18, wherein the heavy and light chain CDR residues are numbered according to IMGT.

In some aspects and embodiments of the constructs, antibodies, and antigen-binding portions thereof described herein, the antibody or antigen-binding portion thereof that specifically binds human NKp30 comprises a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 64 or SEQ ID NO: 101. In some embodiments, the antibody or antigen-binding portion thereof that specifically binds human NKp30 comprises a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some such embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 64 or SEQ ID NO: 101. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Provided herein, in some aspects, is a novel antibody or antigen-binding portion thereof that specifically binds human NKp30, wherein the antibody or antigen-binding portion thereof comprises a heavy chain variable region comprising a heavy chain CDR1 of SEQ ID NO: 69, a heavy chain CDR2 of SEQ ID NO: 70, and a heavy chain CDR3 of SEQ ID NO: 71. In some embodiments, the antibody or antigen-binding portion thereof that specifically binds human NKp30 further comprises a light chain variable region comprising a light chain CDR1 of SEQ ID NO: 19, a light chain CDR2 of SEQ ID NO: 20, and a light chain CDR3 of SEQ ID NO: 43. A representative antibody having such CDRs is mAb10.

In some aspects and embodiments of the constructs described herein, the antibody or antigen-binding portion thereof that specifically binds human NKp30 comprises a heavy chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 72 and/or a light chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 18. In some such embodiments, the heavy chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 72. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 18.

In some embodiments, the CDRs of the antibody or antigen-binding portion thereof that specifically binds human NKp30 recited herein comprise about residues 24-34 (CDR1), 50-56 (CDR2) and 89-97 (CDR3) in the light chain variable domain, and 27-35 (CDR1), 49-60 (CDR2) and 93-102 (CDR3) in the heavy chain variable domain, when numbered according to Chothia numbering. In some embodiments, CDR2 in the light chain variable domain can comprise amino acids 49-56, when numbered according to Chothia numbering.

The disclosure also provides, in some embodiments, an antibody or antigen-binding portion thereof that specifically binds NKp30 that comprises the three heavy chain CDRs of the heavy chain variable region of SEQ ID NO: 72, and the three light chain CDRs of the light chain variable region of SEQ ID NO: 18.

The disclosure also provides, in some embodiments, an antibody or antigen-binding portion thereof that specifically binds NKp30 that comprises heavy chain CDRs of the heavy chain variable region of SEQ ID NO: 72, and light chain CDRs of the light chain variable region of SEQ ID NO: 18, wherein the heavy and light chain CDR residues are numbered according to Kabat.

The disclosure also provides, in some embodiments, an antibody or antigen-binding portion thereof that specifically binds NKp30 that comprises heavy chain CDRs of the heavy chain variable region of SEQ ID NO: 72, and light chain CDRs of the light chain variable region of SEQ ID NO: 18, wherein the heavy and light chain CDR residues are numbered according to Chothia.

The disclosure also provides, in some embodiments, an antibody or antigen-binding portion thereof that specifically binds NKp30 that comprises heavy chain CDRs of the heavy chain variable region of SEQ ID NO: 72, and light chain CDRs of the light chain variable region of SEQ ID NO: 18, wherein the heavy and light chain CDR residues are numbered according to MacCallum.

The disclosure also provides, in some embodiments, an antibody or antigen-binding portion thereof that specifically binds NKp30 that comprises heavy chain CDRs of the heavy chain variable region of SEQ ID NO: 72, and light chain CDRs of the light chain variable region of SEQ ID NO: 18, wherein the heavy and light chain CDR residues are numbered according to AbM.

The disclosure also provides, in some embodiments, an antibody or antigen-binding portion thereof that specifically binds NKp30 that comprises heavy chain CDRs of the heavy chain variable region of SEQ ID NO: 72, and light chain CDRs of the light chain variable region of SEQ ID NO: 18, wherein the heavy and light chain CDR residues are numbered according to IMGT.

In some aspects and embodiments of the constructs, antibodies, and antigen-binding portions thereof described herein, the antibody or antigen-binding portion thereof that specifically binds human NKp30 comprises a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 73. In some embodiments, the antibody or antigen-binding portion thereof that specifically binds human NKp30 comprises a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some such embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 73. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Also provided is an isolated monoclonal antibody, or antigen-binding fragment thereof, that specifically binds to human NKp30, wherein, when bound to human NKp30, the antibody or antigen-binding fragment thereof binds to at least one of the amino acid residues bound by an anti-human NKp30 antibody comprising the heavy chain variable region sequence set forth in SEQ ID NO: 72 and the light chain variable region sequence set forth in SEQ ID NO: 18. The disclosure also provides an isolated monoclonal antibody, or antigen-binding fragment thereof, that specifically binds to human NKp30, wherein, when bound to human NKp30, the antibody or antigen-binding fragment thereof cross blocks the binding of an anti-human NKp30 antibody comprising the heavy chain variable region sequence set forth in SEQ ID NO: 72 and the light chain variable region sequence set forth in SEQ ID NO: 18.

Provided herein, in some aspects, is a novel antibody or antigen-binding portion thereof that specifically binds human NKp30, wherein the antibody or antigen-binding portion thereof comprises a heavy chain variable region comprising a heavy chain CDR1 of SEQ ID NO: 75, a heavy chain CDR2 of SEQ ID NO: 76, and a heavy chain CDR3 of SEQ ID NO: 77. In some embodiments, the antibody or antigen-binding portion thereof that specifically binds human NKp30 further comprises a light chain variable region comprising a light chain CDR1, a light chain CDR2, and a light chain CDR3 of SEQ ID NO: 80. A representative antibody having such CDRs is mAb11.

In some aspects and embodiments of the constructs described herein, the antibody or antigen-binding portion thereof that specifically binds human NKp30 comprises a heavy chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 78 and/or a light chain variable region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 80. In some such embodiments, the heavy chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 78. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 80.

In some embodiments, the CDRs of the antibody or antigen-binding portion thereof that specifically binds human NKp30 recited herein comprise about residues 24-34 (CDR1), 50-56 (CDR2) and 89-97 (CDR3) in the light chain variable domain, and 27-35 (CDR1), 49-60 (CDR2) and 93-102 (CDR3) in the heavy chain variable domain, when numbered according to Chothia numbering. In some embodiments, CDR2 in the light chain variable domain can comprise amino acids 49-56, when numbered according to Chothia numbering.

The disclosure also provides, in some embodiments, an antibody or antigen-binding portion thereof that specifically binds NKp30 that comprises the three heavy chain CDRs of the heavy chain variable region of SEQ ID NO: 78, and the three light chain CDRs of the light chain variable region of SEQ ID NO: 80.

The disclosure also provides, in some embodiments, an antibody or antigen-binding portion thereof that specifically binds NKp30 that comprises heavy chain CDRs of the heavy chain variable region of SEQ ID NO: 78, and light chain CDRs of the light chain variable region of SEQ ID NO: 80, wherein the heavy and light chain CDR residues are numbered according to Kabat.

The disclosure also provides, in some embodiments, an antibody or antigen-binding portion thereof that specifically binds NKp30 that comprises heavy chain CDRs of the heavy chain variable region of SEQ ID NO: 78, and light chain CDRs of the light chain variable region of SEQ ID NO: 80, wherein the heavy and light chain CDR residues are numbered according to Chothia.

The disclosure also provides, in some embodiments, an antibody or antigen-binding portion thereof that specifically binds NKp30 that comprises heavy chain CDRs of the heavy chain variable region of SEQ ID NO: 78, and light chain CDRs of the light chain variable region of SEQ ID NO: 80, wherein the heavy and light chain CDR residues are numbered according to MacCallum.

The disclosure also provides, in some embodiments, an antibody or antigen-binding portion thereof that specifically binds NKp30 that comprises heavy chain CDRs of the heavy chain variable region of SEQ ID NO: 78, and light chain CDRs of the light chain variable region of SEQ ID NO: 80, wherein the heavy and light chain CDR residues are numbered according to AbM.

The disclosure also provides, in some embodiments, an antibody or antigen-binding portion thereof that specifically binds NKp30 that comprises heavy chain CDRs of the heavy chain variable region of SEQ ID NO: 78, and light chain CDRs of the light chain of SEQ ID NO: 80, wherein the heavy and light chain CDR residues are numbered according to IMGT.

In some aspects and embodiments of the constructs, antibodies, and antigen-binding portions thereof described herein, the antibody or antigen-binding portion thereof that specifically binds human NKp30 comprises a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 79. In some embodiments, the antibody or antigen-binding portion thereof that specifically binds human NKp30 comprises a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 81. In some such embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 79. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 81.

Provided herein is an isolated monoclonal antibody, or antigen-binding fragment thereof, that specifically binds to human NKp30, wherein, when bound to human NKp30, the antibody or antigen-binding fragment thereof binds to at least one of the amino acid residues bound by an anti-human NKp30 antibody comprising the heavy chain variable region sequence set forth in SEQ ID NO: 78 and the light chain variable region sequence set forth in SEQ ID NO: 80. The disclosure also provides an isolated monoclonal antibody, or antigen-binding fragment thereof, that specifically binds to human NKp30, wherein, when bound to human NKp30, the antibody or antigen-binding fragment thereof cross blocks the binding of an anti-human NKp30 antibody comprising the heavy chain variable region sequence set forth in SEQ ID NO: 78 and the light chain variable region sequence set forth in SEQ ID NO: 80.

### NKp30 Epitope Binding

While the disclosure is not bound by any particular theory or mechanism of action, as demonstrated herein, the superior and multifaceted properties of the multispecific antigen-binding constructs described herein are believed to derive, in part, from their ability to bind and agonize NKp30. The novel multispecific antigen-binding constructs described herein engage NKp30 on immune effector cells, such as NK cells and γδ T cells, resulting in activation of such immune effector cells and consequent production of inflammatory cytokines, enhancement of cytotoxic activity, and immune effector cell proliferation, e.g., in the tumor microenvironment. As used herein, the term “tumor microenvironment” (alternatively “cancer microenvironment”; abbreviated “TME”) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

Accordingly, epitope mapping analyses were performed to identify residues important for binding to NKp30 and functional activity of the multispecific antigen-binding constructs, antigen-binding units, and antibodies described herein. As used herein, the term “epitope” or “antigenic determinant” refers to a determinant or site on an antigen (e.g., NKp30) to which an antigen-binding protein (e.g., an antigen-binding unit, an immunoglobulin, antibody, or antigen-binding portion thereof) specifically binds. The epitopes of protein antigens can be demarcated into “linear epitopes” and “conformational epitopes.” As used herein, the term “linear epitope” refers to an epitope formed from a contiguous, linear sequence of linked amino acids. Linear epitopes of protein antigens are typically retained upon exposure to chemical denaturants (e.g., acids, bases, solvents, cross-linking reagents, chaotropic agents, disulfide bond reducing agents) or physical denaturants (e.g., thermal heat, radioactivity, or mechanical shear or stress). In some embodiments, an epitope is non-linear, also referred to as an interrupted epitope. As used herein, the term “conformational epitope” or “non-linear epitope” refers to an epitope formed from noncontiguous amino acids juxtaposed by tertiary folding of a polypeptide. Conformational epitopes are typically lost upon treatment with denaturing agents. An epitope typically includes at least 3, 4, 5, 6, 7, 8, 9, 10, 11, 12, 13, 14 or 15 amino acids in a unique spatial conformation. In some embodiments, an epitope includes fewer than 20, 19, 18, 17, 16, 15, 14, 13, 12, 11, 10, 9, 8, 7, 6, 5, 4, or 3 amino acids in a unique spatial conformation. Generally, an antigen-binding unit, antibody, or antigen-binding portion thereof that is specific for a particular target molecule preferentially recognizes and bind to a specific epitope on the target molecule within a complex mixture of proteins and/or macromolecules. In some embodiments, an epitope does not include all amino acids of the extracellular domain of human NKp30.

Domain(s) or region(s) containing residues that are in contact with or are buried by an antigen-binding protein, e.g., a multi specific antigen-binding construct, an antigen-binding unit, an immunoglobulin, antibody or antigen-binding portion thereof, can be identified by mutating specific residues in NKp30 (e.g., a wild-type NKp30 of SEQ ID NO: 7) and determining whether the antigen-binding protein can bind the mutated or variant NKp30 protein. By making a number of individual mutations, residues that play a direct role in binding or that are in sufficiently close proximity to the antigen-binding protein such that a mutation can affect binding between the antigen-binding protein and antigen can be identified. From a knowledge of these amino acids, the domain(s) or region(s) of the antigen that contain residues in contact with the antigen binding protein or covered by the antibody can be elucidated. Such a domain can include the binding epitope of an antigen-binding protein. One specific example of this general approach utilizes an alanine/arginine scanning protocol (see, e.g., Nanevicz, T., et al., 1995, J. Biol. Chem., 270:37, 21619-21625 and Zupnick, A., et al., 2006, J. Biol. Chem., 281:29, 20464-20473). In general, arginine is substituted (typically individually) for an amino acid in the wild-type polypeptide because it is charged and bulky, and thus can disrupt binding between an antigen-binding protein and an antigen in the region of the antigen where the mutation is introduced. Arginines that exist in the wild-type antigen are replaced with alanine. A variety of such individual mutants are obtained, and the collected binding results analyzed to determine what residues affect binding.

Thus, also encompassed by the compositions and methods described herein are antigen-binding proteins (e.g., a multispecific antigen-binding construct, an antigen-binding unit, an immunoglobulin, antibody or antigen-binding portion thereof) that bind to an epitope on NKp30 that comprises all or a portion of an epitope recognized by the particular antibodies described herein (e.g., the same or an overlapping region or a region between or spanning the region). Such antigen-binding proteins antibodies can be identified using routine techniques known in the art, including, for example, competitive binding assays.

As demonstrated herein, multiple novel NKp30 antibodies, namely mAb8, mAb9, mAb10, and mAb11, have been developed that specifically bind NKp30 and agonize NKp30 function and activity, and can be used in the multispecific antigen-binding constructs described herein. To determine which amino acid residues within NKp30 are critical for the binding of mAb8, mAb10, and mAb11 to human NKp30, a combined alanine and arginine scanning mutagenesis analysis was performed to identify residues important for antibody binding to NKp30. NKp30 mutants were generated with single point mutations at surface-exposed amino acid residues on the face of NKp30. These His-tagged NKp30 mutants were then tested for their ability to bind to mAb8, mAb10, and mAb11, as measured by Octet.

An alteration (for example a reduction or increase) in binding between an antigen-binding protein and a variant NKp30, as used herein, means that there is a change in binding affinity (e.g., as measured by known methods such as Biacore testing or Octet, as described below in the examples), EC₅₀, and/or a change (for example a reduction) in the total binding capacity of the antigen-binding protein). A significant alteration in binding indicates that the mutated residue is directly involved in binding to the antigen-binding protein or is in close proximity to the binding protein when the binding protein is bound to antigen.

In some embodiments, a significant reduction in binding means that the binding affinity, EC50, and/or capacity between an antigen-binding protein and a mutant NKp30 antigen is reduced by greater than 10%, greater than 20%, greater than 40%, greater than 50%, greater than 55%, greater than 60%, greater than 65%, greater than 70%, greater than 75%, greater than 80%, greater than 85%, greater than 90% or greater than 95% relative to binding between the antigen binding protein and a wild type NKp30 (e.g., shown in SEQ ID NO: 7). In certain embodiments, binding is reduced below detectable limits. In some embodiments, a significant reduction in binding is evidenced when binding of an antigen-binding protein to a variant NKp30 protein is less than 50% (for example, less than 40%, 35%, 30%, 25%, 20%, 15% or 10%) of the binding observed between the antigen binding protein and a wild-type NKp30 protein (for example, the protein of SEQ ID NO: 7. Such binding measurements can be made using a variety of binding assays known in the art. A specific example of one such assay is described in Example 37.

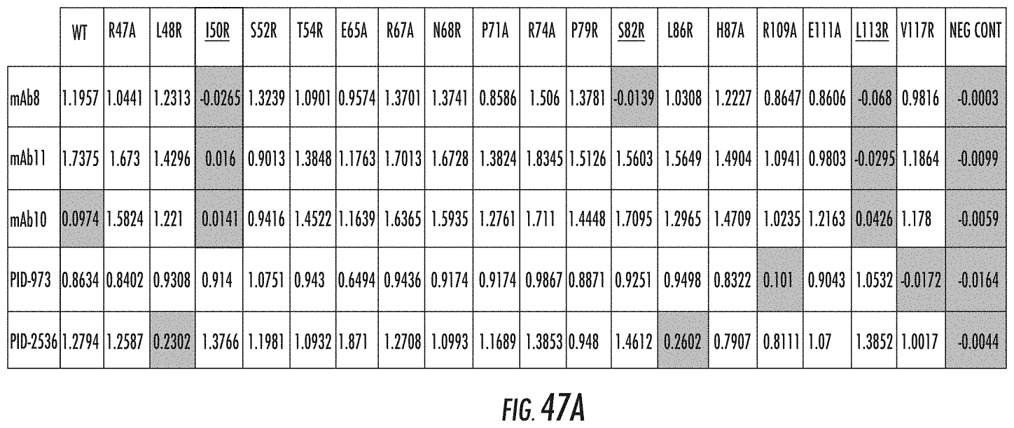

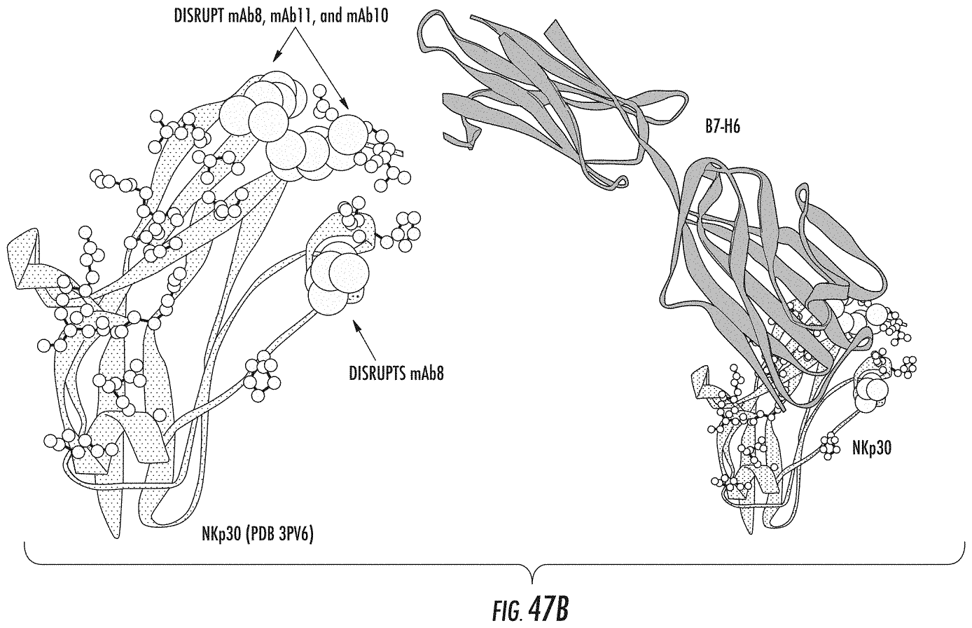

For mAb8, 150, S82, and L113 were identified as important amino acid residues for binding to NKp30, as mutating these residues resulted in a loss of binding to NKp30. For mAb10 and mAb11, I50 and L113 were identified as important amino acid residues for binding to NKp30, as mutating these residues resulted in a loss of binding to NKp30. FIG. 47A shows a Table with a partial amino acid sequence of NKp30, comprising surface-exposed amino acid residues on the face of NKp30, where residues comprising an epitope bound by mAb8, mAb10, and mAb11 are indicated in bold and underlined text. FIG. 47B depicts X-ray crystallography images of human NKp30, with residues I50, S82, and L113 shown as spheres (left panel) and X-ray crystallography images of human NKp30 bound to B7-H6 with residues I50, S82, and L113 shown as spheres (right panel). These figures indicate that the NKp30 antigen-binding proteins described herein bind to a conformational or non-linear epitope of NKp30, thereby blocking ligand binding.

Thus, provided herein are multispecific antigen-binding constructs, antigen-binding units, and isolated antibodies or antigen-binding portions thereof that specifically bind to at least one of the amino acid residues I50, S82, and L113 of SEQ ID NO: 7, and wherein the multispecific antigen-binding constructs, antigen-binding units, and isolated antibodies or antigen-binding portions thereof block binding of NKp30 to an NKp30 ligand. In some embodiments of these aspects and all such aspects described herein, the NKp30 ligand is selected from BAG6, B7-H6, and Gal-3. In some embodiments of these aspects and all such aspects described herein, the NKp30 ligand is B7-H6.

In some embodiments of these aspects and all such aspects described herein, the multispecific antigen-binding constructs, antigen-binding units, and isolated antibodies or antigen-binding portions thereof specifically binds to an epitope of human NKp30 comprising residue I50 of SEQ ID NO: 7. In some embodiments, the multispecific antigen-binding constructs, antigen-binding units, and isolated antibodies or antigen-binding portions thereof specifically binds to an epitope of human NKp30 comprising residue S82 of SEQ ID NO: 7. In some embodiments, the multispecific antigen-binding constructs, antigen-binding units, and isolated antibodies or antigen-binding portions thereof specifically binds to an epitope of human NKp30 comprising residue L113 of SEQ ID NO: 7. In some embodiments, the multispecific antigen-binding constructs, antigen-binding units, and isolated antibodies or antigen-binding portions thereof specifically binds to an epitope of human NKp30 comprising residue 150 and L113 of SEQ ID NO: 7. In some embodiments, the multispecific antigen-binding constructs, antigen-binding units, and isolated antibodies or antigen-binding portions thereof specifically binds to an epitope of human NKp30 comprising residues I50 and S82 of SEQ ID NO: 7. In some embodiments, the multispecific antigen-binding constructs, antigen-binding units, and isolated antibodies or antigen-binding portions thereof specifically binds to an epitope of human NKp30 comprising residues S82 and L113 of SEQ ID NO: 7. In some embodiments, the multispecific antigen-binding constructs, antigen-binding units, and isolated antibodies or antigen-binding portions thereof specifically binds to an epitope of human NKp30 comprising residues I50, S82, and L113 of SEQ ID NO: 7.

In some embodiments of these aspects and all such aspects described herein, the multispecific antigen-binding constructs, antigen-binding units, and isolated antibodies or antigen-binding portions thereof bind to an epitope of human NKp30 and compete with mAb8 for binding to the epitope of human NKp30. In some embodiments of these aspects and all such aspects described herein, the multispecific antigen-binding constructs, antigen-binding units, and isolated antibodies or antigen-binding portions thereof bind to an epitope of human NKp30 and compete with mAb10 for binding to the epitope of human NKp30. In some embodiments of these aspects and all such aspects described herein, the multispecific antigen-binding constructs, antigen-binding units, and isolated antibodies or antigen-binding portions thereof bind to an epitope of human NKp30 and compete with mAb11 for binding to the epitope of human NKp30. In some such embodiments, the multispecific antigen-binding constructs, antigen-binding units, and isolated antibodies or antigen-binding portions thereof bind to and agonize NKp30. In some such embodiments, the multispecific antigen-binding constructs, antigen-binding units, and isolated antibodies or antigen-binding portions thereof provided by the disclosure bind to and agonize NKp30 and co-stimulate activation of immune effector cells, such as NK cells and/or γδ T cells.

The present disclosure provides multispecific antigen-binding constructs, antigen-binding units, and isolated antibodies or antigen-binding portions thereof that compete for binding to an epitope on NKp30 that comprises all or a portion of an epitope recognized by one or more particular reference antibodies described herein (e.g., mAb8, mAb10, or mAb11). In some embodiments, the provides multispecific antigen-binding constructs, antigen-binding units, and isolated antibodies or antigen-binding portions thereof bind to an epitope of human NKp30 and compete with a reference antibody (e.g., mAb8, mAb10, or mAb11) for binding to the epitope of human NKp30 and bind to human NKp30 with an equilibrium dissociation constant K_{D} of 1×10⁻⁶ or less. In some embodiments, one or more mutations to the epitope inhibit, reduce, or block binding to both the multispecific antigen-binding constructs, antigen-binding units, and isolated antibodies or antigen-binding portions thereof and a reference antibody (e.g., mAb8, mAb 10, or mAb11). In some embodiments, the reference antibody is the mAb8 antibody, described herein.

In some embodiments, the multispecific antigen-binding constructs, antigen-binding units, and isolated antibodies or antigen-binding portions thereof provided by the disclosure can be assessed through x-ray crystallographic analysis of a crystal structure comprising an antibody bound to NKp30, or a fragment or portion thereof. In some embodiments, the epitopes that are bound by the antibodies provided by the disclosure are identified by determining the residues on the human NKp30 antigen that reside or are located within 4 angstroms (Å) of an antibody paratope residue, e.g., mAb8, mAb10, or mAb11.

In some embodiments, multispecific antigen-binding constructs, antigen-binding units, and isolated antibodies or antigen-binding portions thereof are provided that exhibit significantly lower binding for a variant NKp30 protein in which a residue in a wild-type NKp30 protein (e.g., SEQ ID NO: 7 is substituted with arginine or alanine. In some embodiments, binding of an antigen-binding protein (e.g., multispecific antigen-binding constructs, antigen-binding units, and isolated antibodies or antigen-binding portions thereof) is significantly reduced for a variant NKp30 protein having any one or more (e.g., 1, 2, 3) of the following mutations: I50R, S82R, and L113R as compared to a wild-type NKp30 protein (e.g., SEQ ID NO: 7). In some embodiments, binding of an antigen-binding protein is significantly reduced for a variant NKp30 protein having one or more (e.g., 1, 2, 3) substitutions at the following positions: 50, 82, and 113, as shown in FIG. 47A, as compared to a wild-type NKp30 protein (e.g., SEQ ID NO: 7). In some embodiments, the reduction in binding is observed as a change in EC50. In some embodiments, the change in EC50 is an increase in the numerical value of the EC50 (and thus is a decrease in binding). In some embodiments, for an amino acid to be part of an NKp30 epitope, the binding is reduced by at least 10%, for example, reductions of at least any of the following amounts: at least 20%, at least 30%, at least 40%, at least 50%, at least 60%, at least 70%, at least 80%, at least 90%, at least 95%, at least 98%, at least 99%, or at least 100% can, in some embodiments, indicate that a residue is part of the epitope.

Also provided herein, in some aspects, is a multispecific antigen-binding construct, antigen-binding unit, antibody or antigen-binding portion thereof, that binds to an epitope of human NKp30, wherein the epitope is within or overlapping with amino acids 50-113 of SEQ ID NO: 7, and wherein one or more substitutions at amino acid residues I50, S82, and L113 disrupts binding of the antibody or antigen-binding portion to human NKp30. In some embodiments, the epitope is a conformational epitope. In some embodiments, the epitope is a linear epitope.

Although the variant forms listed are referenced with respect to the wild-type sequence shown in SEQ ID NO: 7, it will be appreciated that in an allelic variant of NKp30 the amino acid at the indicated position could differ. Antigen-binding proteins showing significantly lower binding for such allelic forms of NKp30 are also contemplated. Accordingly, in some embodiments, any of the above embodiments can be compared to an allelic sequence, rather than purely the wild-type sequence of SEQ ID NO: 7.

As noted above, amino acid residues directly involved in binding or covered by an antigen-binding protein can be identified from scanning results. These residues can thus provide an indication of the domains or regions of SEQ ID NO: 7 that contain the binding region(s) to which antigen-binding proteins bind. As can be seen from the results summarized in Example 37, in some embodiments, an antigen-binding protein binds to a domain containing at least one of amino acids: 50, 82, and 113 of SEQ ID NO: 7.

In some embodiments, the antigen-binding protein (e.g., multispecific antigen-binding constructs, antigen-binding units, and isolated antibodies or antigen-binding portions thereof) binds to a region containing at least one of amino acids 50, 82, and 113 of SEQ ID NO: 7. In some embodiments, more than one of the identified residues are part of the region that is bound by the antigen-binding protein. In some embodiments, the antigen-binding protein competes with mAb8.

In some embodiments, the antigen-binding protein (e.g., multispecific antigen-binding constructs, antigen-binding units, and isolated antibodies or antigen-binding portions thereof) binds to a region containing at least 150 of SEQ ID NO: 7. In some embodiments, the antigen-binding protein competes with one or more of mAb8, mb10, and mAb11.

In some embodiments, the antigen-binding protein (e.g., multispecific antigen-binding constructs, antigen-binding units, and isolated antibodies or antigen-binding portions thereof) binds to a region containing at least S82 of SEQ ID NO: 7. In some embodiments, the antigen-binding protein competes with mAb8.

In some embodiments, the antigen-binding protein (e.g., multispecific antigen-binding constructs, antigen-binding units, and isolated antibodies or antigen-binding portions thereof) binds to a region containing at least L113 of SEQ ID NO: 7. In some embodiments, the antigen-binding protein competes with one or more of mAb8, mb10, and mAb11.

In some embodiments, the antigen-binding protein (e.g., multispecific antigen-binding constructs, antigen-binding units, and isolated antibodies or antigen-binding portions thereof) binds to a region containing at least I50 and S82 of SEQ ID NO: 7. In some embodiments, the antigen-binding protein competes with mAb8.

In some embodiments, the antigen-binding protein (e.g., multispecific antigen-binding constructs, antigen-binding units, and isolated antibodies or antigen-binding portions thereof) binds to a region containing at least I50 and L113 of SEQ ID NO: 7. In some embodiments, the antigen-binding protein competes with one or more of mAb8, mb10, and mAb11.

In some embodiments, the antigen-binding protein (e.g., multispecific antigen-binding constructs, antigen-binding units, and isolated antibodies or antigen-binding portions thereof) binds to a region containing at least S82 and L113 of SEQ ID NO: 7. In some embodiments, the antigen-binding protein competes with mAb8.

In some embodiments, the antigen-binding protein (e.g., multispecific antigen-binding constructs, antigen-binding units, and isolated antibodies or antigen-binding portions thereof) binds to a region containing at least I50, S82, and L113 of SEQ ID NO: 7. In some embodiments, the antigen-binding protein competes with mAb8.

In another aspect, antigen-binding proteins (e.g., multispecific antigen-binding constructs, antigen-binding units, and isolated antibodies or antigen-binding portions thereof) are provided that compete with one of the exemplified antibodies or antigen-binding portions thereof to the epitope described herein for specific binding to NKp30. Such antigen-binding proteins can also bind to the same epitope as one of the herein exemplified antigen-binding proteins (e.g., multispecific antigen-binding constructs, antigen-binding units, and isolated antibodies or antigen-binding portions thereof), or an overlapping epitope. Antigen-binding proteins that compete with or bind to the same NKp30 epitope as the exemplified antigen-binding proteins (e.g., mAb8, mAb10, mAb11, or any one of Constructs1-10) are expected to show similar functional properties. Thus, as a specific example, the antigen-binding proteins that are provided herein include those that compete with an antibody or multispecific antigen-binding construct

(a) having all 6 of the CDRs described herein for mAb8, mAb10, and mAb11;
(b) a VH and a VL described herein for mAb8, mAb10, mAb11, or any one of Constructs1-10;
(c) two light chains and two heavy chains as specified for mAb8, mAb10, mAb11, or any one of Constructs1-10.

### Multispecific Antigen-Binding Constructs

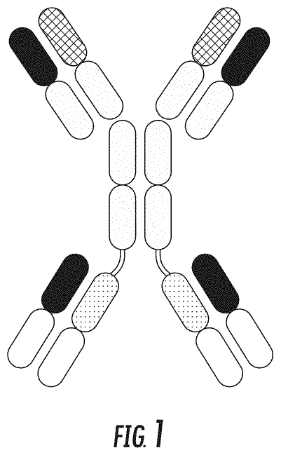

Also provided herein, in some aspects, are multispecific antigen-binding constructs that specifically bind NKp30 and BCMA for use in the compositions and methods described herein. For example, Construct 1 is a multispecific antigen-binding construct that specifically binds human BCMA and human NKp30. The construct contains an anti-BCMA IgG1 antibody (mAb1) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 1, the structure for which is represented by the illustration of FIG. 1, comprises the heavy chain sequence depicted in SEQ ID NO: 9 and the light chain sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 9 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 9. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 2 comprises an anti-BCMA IgG1 antibody in which the heavy chain of the antibody is a fusion protein further comprising at its C-terminus the heavy chain variable region of an anti-NKp30 antibody (mAb9), which is connected to the Fc region of the anti-BCMA antibody by way of a poly-GGGS (SEQ ID NO:22) linker. The light chains for the anti-BCMA portion and the anti-NKp30 portion of the construct are identical. Construct 2, the structure for which is represented by the illustration of FIG. 1, comprises the same anti-BCMA IgG1 antibody portion as Construct 1, and the same light chain as Construct 1, but differs by the variable region sequence (SEQ ID NO: 25) of the anti-NKp30 antibody portion of the construct.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 24 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 24. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Constructs 3 and 4 are aglycosylated versions of Constructs 1 and 2 (also referred to as “Construct 1 aglyco” or “Construct 2 aglyco”) in which the Fc portion of the heavy chain of each construct contains the N297A amino acid substitution (numbered according to EU numbering). For example, Construct 3 comprises a heavy chain having the amino acid sequence depicted in SEQ ID NO: 26 and a light chain having the amino acid sequence depicted in SEQ ID NO: 8. Construct 4 comprises a heavy chain having the amino acid sequence depicted in SEQ ID NO: 27 and a light chain having the amino acid sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 26 or SEQ ID NO: 27 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 26 or SEQ ID NO: 27. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 5 comprises an affinity matured anti-BCMA IgG1 antibody (mAb3) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 5 comprises a heavy chain having the amino acid sequence depicted in SEQ ID NO: 65 and a light chain having the amino acid sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 65 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 65. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 6 comprises an affinity matured anti-BCMA IgG1 antibody (mAb2) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 6 comprises a heavy chain having the amino acid sequence depicted in SEQ ID NO: 66 and a light chain having the amino acid sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 66 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 66. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 7 is an afucosylated construct that comprises an anti-BCMA IgG1 antibody (mAb1) in which the heavy chain of the antibody is a fusion protein further comprising at its C-terminus the heavy chain variable region of an anti-NKp30 antibody (mAb8), which is connected to the Fc region of the anti-BCMA antibody by way of an extended poly-GGGS (SEQ ID NO:22) linker. The light chains for the anti-BCMA portion and the anti-NKp30 portion of the construct are identical. Construct 7 comprises a heavy chain having the amino acid sequence depicted in SEQ ID NO: 67 and a light chain having the amino acid sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 67 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 67. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 8 is an aglycosylated version of Construct 5 that comprises an affinity matured anti-BCMA IgG1 antibody (mAb3) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker, and in which the Fc portion of the heavy chain contains the N297A amino acid substitution (numbered according to EU numbering). The light chains for the anti-BCMA portion and the anti-NKp30 portion of the construct are identical. Construct 8 comprises a heavy chain having the amino acid sequence depicted in SEQ ID NO: 68 and a light chain having the amino acid sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 68 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 68. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 9 (also referred to herein as Construct 2Z) is a multispecific antigen-binding construct that specifically binds human BCMA and human NKp30. The construct contains an anti-BCMA IgG1 antibody (mAb1) in which the heavy chain of the antibody is a fusion protein further comprising at its C-terminus the heavy chain variable region of an anti-NKp30 antibody (mAb10), which is connected to the Fc region of the anti-BCMA antibody by way of a poly-GGGS (SEQ ID NO:22) linker. The light chains for the anti-BCMA portion and the anti-NKp30 portion of the construct are identical. Construct 9 comprises the heavy chain sequence depicted in SEQ ID NO: 74 and the light chain sequence depicted in SEQ ID NO: 8.

Accordingly, in some aspects, provided herein is a multispecific antigen-binding construct that specifically binds BCMA and NKp30 that comprises (i) a heavy chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 74 and (ii) a light chain region comprising an amino acid sequence that is at least 90% identical (e.g., at least 90%, at least 91%, at least 92%, at least 93%, at least 94%, at least 95%, at least 96%, at least 97%, at least 98%, or at least 99% identical) to SEQ ID NO: 8. In some embodiments, the heavy chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 74. In some embodiments, the light chain region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 8.

Construct 10 is an afucosylated construct that comprises an affinity matured anti-BCMA IgG1 antibody (mAb3) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 10 comprises a heavy chain having the amino acid sequence depicted in SEQ ID NO: 65 and a light chain having the amino acid sequence depicted in SEQ ID NO: 8.

Various formats and methods are known in the art that can be used to generate the multivalent and/or multispecific constructs described herein, such as multivalent and/or multispecific antibody formats of both asymmetric and symmetric architectures. Non-limiting examples of such formats include (i) Fc-less bispecific antibody formats, such as tandem single-chain variable fragments (scFv2, taFv) and triplebodies, including bi-specific T cell engager (BiTE) and bispecific killer cell engagers (BiKE) molecules; bispecific single-domain antibody fusion proteins comprising single-domain antibodies, such as VH or VL domains, VHH, VNAR and Nanobodies; diabodies and diabody derivatives, including tandem diabody and dual-affinity retargeting (DART) proteins; Fab fusion proteins; and other Fc-less fusion proteins, through the use of heterodimerizing peptides or miniantibodies from various proteins, e.g., leucine zippers with a coiled coil structure; (ii) bispecific IgGs with asymmetric architecture, such as asymmetric IgGs with heavy and light chains from two different antibodies; bispecific IgGs with an asymmetric Fc region using knobs-into-holes approaches, electrostatic interactions (steering) to avoid homodimerization of CH3 domains, preferential heavy chain heterodimerization by introducing charge pairs into the hinge region of IgG1 and IgG2, strand-exchange engineered domain (SEED) heterodimers, and bispecific engagement by antibodies based on the T cell receptor (BEAT) technologies; asymmetric Fc and CH3 fusion proteins; (iii) bispecific antibodies with a symmetric architecture, such as appended IgGs by fusion of scFvs, fusion of domain antibodies and scaffold proteins, fusion of Fab arms, and fusion of additional variable heavy and light chain domains; modified IgG molecules; symmetric Fc- and CH3-based bispecific antibodies; and bispecific antibodies using immunoglobulin-derived homodimerization domains. See, for example, “The making of bispecific antibodies,” Brinkmann and Kontermann, MABS 2017, Vol. 9:2, pp. 182-212, the contents of which are herein incorporated by reference in its entirety. See also, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligo-valent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; string of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573; 5,932,448; 5,959,083; 5,989,830; 6,005,079; 6,239,259; 6,294,353; 6,333,396; 6,476,198; 6,511,663; 6,670,453; 6,743,896; 6,809,185; 6,833,441; 7,129,330; 7,183,076; 7,521,056; 7,527,787; 7,534,866; 7,612,181; US Patent Publication Nos. US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1; European Pat. No. EP346087A2; and PCT Publication Nos. WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1.

In some embodiments, the multispecific antigen-binding construct of the present disclosure selectively binds a tumor or B-lineage cell antigen (e.g., BCMA) and NKp30. The antigen-binding units can be generated by standard techniques, as disclosed herein. In some embodiments, any known antibodies against a tumor or B-lineage cell antigen or NKp30 can be used to generate a bispecific antibody according to the present disclosure, or the multispecific antigen-binding constructs can be generated using antibodies known in the art.

In some embodiments, the multispecific antigen-binding construct (e.g., a trispecific antigen-binding construct) further comprises a third antigen-binding unit that binds specifically to a molecule expressed by an effector immune cell. Optionally, the third antigen-binding unit is an immunoglobulin Fc domain. The molecule expressed by the effector immune cell can be, for example, CD16, CD32a, CD64, or CD89. Optionally, the multispecific antigen-binding construct does not comprise an immunoglobulin Fc domain.

In some embodiments, the multispecific antigen-binding construct (e.g., a trispecific antigen-binding construct) further comprises a third antigen-binding unit that binds specifically to a second tumor or B-lineage cell antigen. In such embodiments, the trispecific antigen-binding construct is monovalent for a first tumor or B-lineage cell antigen, monovalent for a second tumor or B-lineage cell antigen, and bivalent for NKp30.

In some embodiments, the multispecific antigen-binding construct (e.g., a trispecific antigen-binding construct) further comprises a third antigen-binding unit that binds specifically to a second NK activating receptor. In some such embodiments, the trispecific antigen-binding construct is monovalent for a tumor or B-lineage cell antigen, bivalent for NKp30, and monovalent for a second NK activating receptor.

In some embodiments, the multispecific antigen-binding construct (e.g., a tetraspecific antigen-binding construct) further comprises a third antigen-binding unit that binds specifically to a second NK activating receptor, and a fourth antigen-binding unit that binds specifically to a second tumor or B-lineage cell antigen. In such embodiments, the tetraspecific antigen-binding construct is monovalent for a tumor or B-lineage cell antigen, monovalent for a second tumor or B-lineage cell antigen, monovalent for NKp30, and monovalent for a second NK activating receptor.

Non-limiting examples of additional NK activating receptors contemplated for use as targets in embodiments of the multispecific antigen-binding constructs described herein include NKp46, 2B4, CD226, NKG2D, CD137, CD16a, and CD2. Exemplary human amino acid sequences for these NK activating receptors can be found as SEQ ID NOs: 27-35.

Accordingly, in some embodiments of the multispecific antigen-binding constructs described herein, an antigen-binding unit binds specifically to NKp46.

In some embodiments of the multispecific antigen-binding constructs described herein, an antigen-binding unit binds specifically to 2B4.

In some embodiments of the multispecific antigen-binding constructs described herein, an antigen-binding unit binds specifically to CD226.

In some embodiments of the multispecific antigen-binding constructs described herein, an antigen-binding unit binds specifically to NKG2D.

In some embodiments of the multispecific antigen-binding constructs described herein, an antigen-binding unit binds specifically to CD137.

In some embodiments of the multispecific antigen-binding constructs described herein, an antigen-binding unit binds specifically to CD16a.

In some embodiments of the multispecific antigen-binding constructs described herein, an antigen-binding unit binds specifically to CD2.

In certain embodiments, the first antigen-binding unit and the second antigen-binding unit are linked by at least one amino linker amino acid sequence. Optionally, the linker amino acid sequence comprises GGGGSX (SEQ ID NO: 22), wherein x is an integer between and including 1 to 6.

The first antigen-binding unit or second antigen-binding unit, or both, can comprise a heavy chain comprising one or more immunoglobulin Fc modifications. Similarly, third or subsequent antigen-binding units, or both, also can comprise a heavy chain comprising one or more immunoglobulin Fc modifications. In some embodiments, the immunoglobulin Fc domain of the heavy chain comprises one or more amino acid mutations that, e.g., promote heterodimerization of the first and second antigen-binding unit, promote serum half-life, and/or modify effector function. In some embodiments, the mutation is present in a CH3 domain of the heavy chain (See, e.g., Xu et al. (2015) mAbs 7(1): 231-42).

While traditional Fc fusion proteins and antibodies are examples of unguided interaction pairs, a variety of engineered Fc domains have been designed as asymmetric interaction pairs (Spiess et al. (2015) Molecular Immunology 67(2A): 95-106) to promote heterodimerization, e.g., of a first antigen-binding unit and a second antigen-binding unit. Various methods are known in the art that increase desired pairing of Fc-containing polypeptide chains in a single cell line to produce a preferred asymmetric fusion protein at acceptable yields (see, for example, Klein et al. (2012) mAbs 4:653-663; and Spiess et al. (2015) Molecular Immunology 67(2PartA): 95-106). Methods to obtain desired pairing of Fc-containing polypeptides include, but are not limited to, charge-based pairing (electrostatic steering), “knobs-into-holes” steric pairing, SEEDbody pairing, and leucine zipper-based pairing (See, for example, Ridgway et al. (1996) Protein Eng. 9:617-621; Merchant et al. (1998) Nat. Biotech. 16:677-681; Davis et al. (2010) Protein Eng. Des. Sel. 23:195-202; Gunasekaran et al. (2010) J. Biol. Chem. 285:19637-19646; Wranik et al. (2012) J. Biol. Chem. 287:43331-43339; U.S. Pat. No. 5,932,448; and PCT Publication Nos. WO 1993/011162, WO 2009/089004, and WO 2011/034605).

For example, one means by which interaction between specific polypeptides can be promoted is by engineering protuberance-into-cavity (knob-into-holes) complementary regions such as described in U.S. Pat. Nos. 7,183,076 and 5,731,168; and PCT Publication No. WO 2016/164089. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction is represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation. The electrostatic steering effect on heterodimer formation and homodimer discouragement can be further enhanced by mutation of additional charge residues which may or may not be paired with an oppositely charged residue in the second chain including, for example, Arg355 and Lys360 (See, e.g., PCT Publication No. WO 2016/164089).

Thus, in some embodiments, the multispecific antigen-binding constructs described herein can comprise a constant domain of an immunoglobulin, including, for example, the Fc portion of an immunoglobulin. For example, a first antigen-binding unit can comprise an amino acid sequence that is derived from an Fc domain of an IgG (IgG1, IgG2, IgG3, or IgG4), IgA (IgA1 or IgA2), IgE, or IgM immunoglobulin. Optionally, a second antigen-binding unit and/or subsequent antigen-binding units can comprise an amino acid sequence that is derived from an Fc domain of an IgG (IgG1, lgG2, lgG3, or IgG4), IgA (IgA1 or IgA2), IgE, or IgM. Such immunoglobulin domains can comprise one or more amino acid modifications (e.g., deletions, additions, and/or substitutions) that promote heterodimer formation. In some embodiments, a multispecific antigen-binding construct is of the IgG1 isotype. In some embodiments, a multispecific antigen-binding construct is of the IgG1 isotype and comprises a substitution. In some embodiments, a multispecific antigen-binding construct is of the IgG2 isotype. In some embodiments, a multispecific antigen-binding construct is of the IgG3 isotype. In some embodiments, a multispecific antigen-binding construct is of the IgG4 isotype. In some embodiments, a multispecific antigen-binding construct is of the IgG4 isotype and comprises a substitution. In some embodiments, the substitution is at Ser228 when numbered according to EU numbering. In some embodiments, the substitution at Ser228 is S228P. In some embodiments, a first antigen-binding unit and a second antigen-binding unit comprise Fc domains derived from the same immunoglobulin class and subtype. In some embodiments, a first and second antigen-binding unit comprise Fc domains derived from different immunoglobulin classes or subtypes. Similarly, a first and/or a second antigen-binding unit (e.g., an asymmetric pair or an unguided interaction pair) optionally comprise a modified constant domain of an immunoglobulin, including, for example, one or more amino acid modifications (e.g., deletions, additions, and/or substitutions) that promote heterodimer formation. One or more subsequent antigen-binding units are optionally from the same class and subtype or different than the first and/or second antigen-binding units. Methods of generating Fc modifications having the desired heterodimer formation are known in the art.

In some embodiments, the Fc domain can be modified to enhance serum half-life of the multispecific antigen-binding construct disclosed herein. Fc domain comprising one or more mutations which enhance or diminish antibody binding to the Fc receptor, e.g., at acidic pH as compared to neutral pH, are known in the art. For example, the constructs disclosed herein can comprise one or more mutations in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations can result in an increase in serum half-life of the construct when administered to an animal. Methods of modifying the Fc domain for desired characteristics, such as enhanced serum half-life are known in the art.

In some embodiments, the constructs described herein comprise an altered heavy chain constant region that has reduced (or no) effector function relative to its corresponding unaltered constant region. Effector functions involving the constant region of the constructs described herein can be modulated by altering properties of the constant or Fc region. Altered effector functions include, for example, a modulation in one or more of the following activities: ADCC, complement-dependent cytotoxicity (CDC), apoptosis, binding to one or more Fc-receptors, and pro-inflammatory responses. Modulation refers to an increase, decrease, or elimination of an effector function activity exhibited by a subject antibody or antigen-binding fragment thereof containing an altered constant region as compared to the activity of the unaltered form of the constant region. In particular embodiments, modulation includes situations in which an activity is abolished or completely absent.

An altered constant region with altered FcR binding affinity and/or ADCC activity and/or altered CDC activity is a polypeptide that has either an enhanced or diminished FcR binding activity and/or ADCC activity and/or CDC activity compared to the unaltered form of the constant region. An altered constant region that displays increased binding to an FcR binds at least one FcR with greater affinity than the unaltered polypeptide. An altered constant region which displays decreased binding to an FcR binds at least one FcR with lower affinity than the unaltered form of the constant region. Such variants that display decreased binding to an FcR can possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity can exhibit either increased or reduced ADCC and/or CDC activity compared to the unaltered constant region. For example, in some embodiments, the antibody or antigen-binding fragment thereof comprising an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. A multispecific antigen-binding construct described herein comprising an altered constant region displaying reduced ADCC and/or CDC can exhibit reduced or no ADCC and/or CDC activity.

In some embodiments, the multispecific antigen-binding constructs described herein exhibit reduced or no effector function. In some embodiments, the multispecific antigen-binding constructs comprise a hybrid constant region, or a portion thereof, such as a G2/G4 hybrid constant region (See e.g., Burton et al. (1992) Adv. Immun. 51:1-18; Canfield et al. (1991) J. Exp. Med. 173:1483-1491; and Mueller et al. (1997) Mol. Immunol. 34(6):441-452).

In some embodiments, the multispecific antigen-binding constructs described herein exhibit increased effector function. Methods for enhancing or increasing effector function, including substitutions that can be made in immunoglobulin Fc regions, are known in the art. Examples of such substitutions can be found in Wang et al. (2018) Protein Cell 9(1)63-73 (See, for example, Table 1), the contents of which are herein incorporated by reference in their entireties.

In some embodiments, modulating effector function of the multispecific antigen-binding constructs described herein include changes in glycosylation. A summary of the importance of the oligosaccharides found on human IgGs with their degree of effector function is described in Raju T S., BioProcess International April 2003. 44-53. According to Wright and Morrison, the microheterogeneity of human IgG oligosaccharides can affect biological functions such as CDC and ADCC, binding to various Fc receptors, and binding to C1q protein (Wright A. & Morrison SL. TIBTECH 1997, 15 26-32). It is also documented that glycosylation patterns of antibodies can differ depending on the producing cell and the cell culture conditions (Raju, T S. BioProcess International April 2003. 44-53). Such differences can lead to changes in both effector function and pharmacokinetics (Israel et al. Immunology. 1996; 89(4):573-578; Newkirk et al. P. Clin. Exp. 1996; 106(2):259-64). Differences in effector function may be related to the IgGs ability to bind to the Fcγ receptors (FcγRs) on the effector cells. It has also been shown that IgG, with variants in amino acid sequence that have improved binding to FcγR, can exhibit up to 100% enhanced ADCC using human effector cells (Shields et al. J Biol. Chem. 2001 276(9):6591-604). While these variants include changes in amino acids not found at the binding interface, both the nature of the sugar component as well as its structural pattern also contribute to the differences observed. In addition, it has been shown that the presence or absence of fucose in the oligosaccharide component of an IgG can improve binding and ADCC (Shields et al. J Biol. Chem. 2002; 277(30):26733-40). An IgG that lacked a fucosylated carbohydrate linked to Asn297 exhibited normal receptor binding to the Fcγ receptor. In contrast, binding to the FcγRIIA receptor was improved 50% and accompanied by enhanced ADCC, especially at lower antibody concentrations. Accordingly, in some embodiments, methods for enhancing or increasing effector function include expressing or producing the multispecific antigen-binding constructs described herein in genetically engineered CHO cells having altered glyosylation or fucosylation activity. In some embodiments, the multispecific antigen-binding constructs described herein lack fucosylation or are afucosylated. Such genetically engineered CHO cells can have, for example, decreased or no α1,6-fucosyltransferase activity, such that the resulting multispecific antigen-binding construct comprises a Fc region, such that the complex N-glycoside-linked sugar chains bound to the Fc region through N-acetylglucosamine of the reducing terminal of the sugar chains do not contain any, or contain reduced amounts, of sugar chains with a fucose bound to the N-acetylglucosamines. Non-limiting examples of such engineered cells that can be used with the multispecific antigen-binding constructs described herein include those described in WO 00/61739, U.S. Pat. Nos. 6,946,292, 7,214,775, 7,425,446, 7,708,992, and 7,737,325, the contents of each of which are herein incorporated by reference in their entireties.

In some embodiments, methods for enhancing or increasing effector function include expressing or producing the multispecific antigen-binding constructs described herein in genetically engineered or modified CHO cells having altered glycosyltransferase enzyme (GnnU) activity, which bisects oligosaccharides that have been implicated in ADCC activity.

In some embodiments, the multispecific antigen-binding constructs contain an altered constant region exhibiting enhanced or reduced complement dependent cytotoxicity (CDC). Modulated CDC activity can be achieved by introducing one or more amino acid substitutions, insertions, or deletions in an Fc region of the antibody. See, e.g., U.S. Pat. No. 6,194,551.

Antibodies or fragments thereof can be further selected for binding to more than one species. For example, antibodies or fragments that bind both mouse and human can be selected by screening with both mouse and human target cells.

The constructs and antigen-binding units described herein can comprise, in part, scaffold domains, proteins, or portions, e.g., molecules which do not provide target receptor-binding activity, but which can provide a portion or domain of the construct which provides spatial organization, structural support, a means of linking of multiple receptor-binding units, or other desired characteristics, e.g., improved half-life. Various scaffold technologies and compositions are known in the art and can be readily linked or conjugated to the antigen-binding units described herein. The scaffold domain, protein, or portion can be derived from an antibody or not derived from an antibody. Such scaffold proteins, and domains thereof, are, generally, obtained through combinatorial chemistry-based adaptation of preexisting antigen-binding proteins.

Non-antibody protein scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. These non-antibody scaffolds and the underlying proteins or peptides on which they are based or from which they have been derived are reviewed by, e.g., Simeon and Chen, Protein Cell 9(1): 3-14 (2018); Vazquez-Lombardi et al., Drug Discovery Today 20: 1271-1283 (2015), and by Binz et al., Nature Biotechnol. 23: 1257-1268 (2005), the contents of each of which are herein incorporated by reference in their entireties. Advantages of using non-antibody scaffolds include increased affinity, target neutralization, and stability. Various non-antibody scaffolds also can overcome some of the limitations of antibody scaffolds, e.g., in terms of tissue penetration, smaller size, and thermostability. Some non-antibody scaffolds can also permit easier construction, not being hindered, for example, by the light chain association issue when bispecific constructs are desired. Methods of constructing constructs on a non-antibody scaffold are known to those of ordinary skill in the art. While not formally on an antibody scaffold, such constructs often include antibody binding domains, whether in the form of single-domain antibodies, scFvs or other antibody binding-domain variants that provide specific target-binding capabilities.

Accordingly, in some embodiments of any of the aspects described herein, a construct can comprise a non-antibody scaffold protein. In some embodiments of any of the aspects described herein, at least one of the receptor-binding units can comprise a non-antibody scaffold protein. One of skill in the art would appreciate that the scaffold portion of a non-antibody scaffold protein can include, in some embodiments, e.g., an adnectin scaffold or a portion derived from human tenth fibronectin type III domain (10Fn3); an anticalin scaffold derived from human lipocalin (e.g., such as those described in, e.g., WO2015/104406); an avimer scaffold or a protein fragment derived from the A-domain of low density-related protein (LRP) and/or very low density lipoprotein receptor (VLDLR); a fynomer scaffold or portion of the SH3 domain of FYN tyrosine kinase; a kunitz domain scaffold or portion of Kunitz-type protease inhibitors, such as a human trypsin inhibitor, aprotinin (bovine pancreatic trypsin inhibitor), Alzheimer's amyloid precursor protein, and tissue factor pathway inhibitor; a knottin scaffold (cysteine knot miniproteins), such as one based on a trypsin inhibitor from E. elaterium; an affibody scaffold or all or part of the Z domain of S. aureus protein A; a β-Hairpin mimetic scaffold; a Designed ankyrin repeat protein (DARPin) scaffold or artificial protein scaffolds based on ankyrin repeat (AR) proteins; or any scaffold derived or based on human transferrin, human CTLA-4, human crystallin, and human ubiquitin. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al. (2005) TRENDS Biotechnol. 23(10):514-522.

The disclosure also provides nucleic acids encoding the multispecific antigen-binding construct or the isolated antibody, or antigen-binding portion thereof described herein. Non-limiting examples of nucleic acid sequences encoding the multispecific antigen-binding constructs or isolated antibodies, or antigen-binding portions thereof, described herein include SEQ ID NOs: 84-100. Also provided are vectors for the multispecific antigen-binding construct or the isolated antibody, or antigen-binding fragment thereof described herein comprising the nucleic acids (optionally with an expression control sequence) and cells comprising the vectors or nucleic acids. Methods for producing a polypeptide are provided, including culturing such cells under conditions for the expression of one or more polypeptides from the vector by the cell. The methods optionally include isolating the one or more polypeptides from the cell or media in which the cell is cultured. Also provided are protein conjugate molecules that comprise a heterologous moiety conjugated to the multispecific antigen-binding construct or the isolated antibody, or antigen-binding fragment thereof, described herein. Optionally, the heterologous moiety is a therapeutic agent, a toxin, a drug, or a radioactive moiety.

### Methods for Producing the Multispecific Antigen-Binding Constructs

Provided herein are methods for producing any of the multispecific antigen-binding constructs described herein. The methods for producing the disclosed constructs include methods for preparing an antibody and antigen-binding fragments thereof. Such methods are well-known in the art and include, e.g., immunizing a subject (e.g., a non-human mammal) with an appropriate immunogen. To generate an antibody that binds to a tumor antigen or an activating NK receptor, such as NKp30, NKp46, NKp44, or NKG2D, for example, a skilled artisan immunizes a suitable subject (e.g., a non-human mammal such as a rat, a mouse, a gerbil, a hamster, a dog, a cat, a pig, a goat, a horse, or a non-human primate) with full-length tumor antigen or polypeptide or a variant or fragment thereof of an NK activating receptor. Antigenic fragments of a polypeptide (tumor antigen or NK activating receptor) can be selected to generate antibodies based on known structural features of the polypeptide. For example, regions within a tumor antigen or NK activating receptor, based on receptor/ligand interface information available in the art, can be used to design a suitable antigenic fragment to generate antibodies having desirable properties. Resulting antibodies or antigen-binding constructs can then be screened for desired binding properties (e.g., binding affinities for the tumor antigen and NK activating receptor and capacity to bridge cells on which the tumor antigen and NK activating receptor are expressed).

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; and chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. (See also Bieg et al. (1999) Autoimmunity 31(1):15-24; see also, e.g., Lodmell et al. (2000) Vaccine 18:1059-1066; Johnson et al. (1999) J. Med. Chem. 42:4640-4649; Baldridge et al. (1999) Methods 19:103-107; and Gupta et al. (1995) Vaccine 13(14): 1263-1276).

In some embodiments, the methods include preparing a hybridoma cell line that secretes a monoclonal antibody that binds to the immunogen. For example, a suitable mammal such as a laboratory mouse is immunized with a polypeptide (e.g., a tumor antigen or NK activating receptor) or antigenic fragment as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter, such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybridoma cells are then screened for secretion of the desired antibodies, e.g., an antibody that binds to the desired antigen.

In some embodiments, an antibody specific for a tumor antigen or NK activating receptor is selected from a non-immune biased library as described in, e.g., U.S. Pat. No. 6,300,064 and Schoonbroodt et al. (2005) Nucleic Acids Res 33(9):e81.

In some embodiments, the methods described herein involve, or can be used in conjunction with, e.g., phage display technologies, bacterial display, yeast surface display, eukaryotic viral display, mammalian cell display, and cell-free (e.g., ribosomal display) antibody screening techniques (See, e.g., Etz et al. (2001) J. Bacteriol. 183:6924-6935; Cornelis (2000) Curr. Opin. Biotechnol. 11:450-454; Klemm et al. (2000) Microbiology 146:3025-3032; Kieke et al. (1997) Protein Eng. 10:1303-1310; Yeung et al. (2002) Biotechnol. Prog. 18:212-220; Boder et al. (2000) Methods Enzymology 328:430-444; Grabherr et al. (2001) Comb. Chem. High Throughput Screen 4:185-192; Michael et al. (1995) Gene Ther. 2:660-668; Pereboev et al. (2001) J. Virol. 75:7107-7113; Schaffitzel et al. (1999) J. Immunol. Methods 231:119-135; and Hanes et al. (2000) Nat. Biotechnol. 18:1287-1292).

Methods for identifying antibodies using various phage display methods are known in the art. In phage display methods, functional antibody domains are displayed on the surface of phage particles that carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen-binding domains are expressed as a recombinantly-fused protein to any of the phage coat proteins pIII, pVIII, or pIX. (See, e.g., Shi et al. (2010) JMB 397:385-396.) Examples of phage display methods that can be used to make the immunoglobulins, or fragments thereof, described herein include those disclosed in Brinkman et al. (1995) J. Immunol. Methods 182:41-50; Ames et al. (1995) J. Immunol. Methods 184:177-186; Kettleborough et al. (1994) Eur. J. Immunol. 24:952-958; Persic et al. (1997) Gene 187:9-18; Burton et al. (1994) Advances in Immunology 57:191-280; and PCT publication Nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

In some embodiments, the phage display antibody libraries can be generated using mRNA collected from B cells from the immunized mammals. For example, a splenic cell sample comprising B cells can be isolated from mice immunized with a tumor antigen or NKp30 polypeptide as described above. mRNA can be isolated from the cells and converted to cDNA using standard molecular biology techniques. (See, e.g., Green and Sambrook (2012) Molecular Cloning—A Laboratory Manual, 4th Ed., Cold Spring Harbor Laboratory Press, New York; Harlow and Lane (1988) Antibodies: a Laboratory Manual, Cold Spring Harbor Laboratory Press, Cold Spring Harbor, N.Y; Lo (2004) Antibody Engineering: Methods and Protocols, Springer Science & Business Media; and Borrebaeck (1995) Antibody Engineering, Oxford University Press). The cDNA coding for the variable regions of the heavy chain and light chain polypeptides of immunoglobulins are used to construct the phage display library. Methods for generating such a library are described in, e.g., Merz et al. (1995) J. Neurosci. Methods 62(1-2):213-9; Di Niro et al. (2005) Biochem. J. 388(Pt 3):889-894; and Engberg et al. (1995) Methods Mol. Biol. 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995) J. Immunol. Methods 182(1):41-50.; Ames et al. (1995) J. Immunol. Methods 184(2):177-86.; Kettleborough et al. (1994) Eur. J. Immunol. 24(4):952-8; and Persic et al. (1997) Gene 187(1):9-18. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al. (2006) Nucleic Acids Res. 34(21):e145). Phage produced from the bacteria are recovered and then contacted to, e.g., a target antigen bound to a solid support (immobilized). Phage can also be contacted to antigen in solution, and the complex is subsequently bound to a solid support.

A subpopulation of antibodies screened using the above methods can be characterized for their specificity and binding affinity for a particular antigen (e.g., human tumor antigen or NK activating receptor) using any immunological or biochemical based method known in the art. For example, specific binding of an antibody to the tumor antigen or NK activating receptor can be determined, for example, using immunological or biochemical based methods such as, but not limited to, an ELISA assay, SPR assays, immunoprecipitation assay, affinity chromatography, and equilibrium dialysis as described above. Immunoassays that can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. It is understood that the above methods can also be used to determine if an antibody to the tumor antigen or NK activating receptor does not bind to the human tumor antigen or NK activating receptor proteins.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody. Alternatively, fragments of chemically synthesized nucleic acids, together capable of encoding an antibody, can be joined together using DNA assembly techniques known in the art (e.g. Gibson Assembly).

Any antibody of choice can be further modified to generate an antigen-binding fragment, as described herein, and/or manipulated using known techniques in the art to generate the multispecific antigen-binding constructs as described herein. For example, cross-linking methods can be used to generate a bispecific structure using a heterobifunctional reagent having an amine-reactive group and a sulfhydryl reactive group as described in, e.g., U.S. Pat. No. 4,433,059; bispecific antibody determinants can be generated by recombining half antibodies (heavy-light chain pairs or Fabs) from different antibodies through cycles of reduction and oxidation of disulfide bonds between the two heavy chains, as described in, e.g., U.S. Pat. No. 4,444,878; trifunctional antibodies, e.g., three Fab′ fragments can be cross-linked through sulfhdryl reactive groups, as described in, e.g., U.S. Pat. No. 5,273,743. Methods of generating multispecific constructs include, e.g., methods of generating multispecific constructs having common light chains.

### Expression and Purification of Multispecific Antigen-Binding Constructs

The multispecific antigen-binding constructs disclosed herein can be produced using a variety of techniques known in the art of molecular biology and protein chemistry. For example, a nucleic acid encoding the multispecific antigen-binding construct (as a single multifunctional polypeptide, or as separate molecules of a multimeric complex—e.g., one antigen-binding unit separately from the other antigen-binding unit) can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc. Natl. Acad. Sci. USA 78:2072) or Tn5 neo (Southern and Berg (1982) Mol. Appl. Genet. 1:327). The selectable marker gene can be either linked to the DNA gene sequences to be expressed, or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc. Natl. Acad. Sci. USA, 79:7147), cytomegalovirus, polyoma virus (Deans et al. (1984) Proc. Natl. Acad. Sci. USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr. Opin. Biotechnol. 13(6):625-629; van Kuik-Romeijn et al. (2000) Transgenic Res. 9(2):155-159; and Pollock et al. (1999) J. Immunol. Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, N.Y. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Modification of the Multispecific Antigen-Binding Constructs

The multispecific antigen-binding constructs can be modified as a single multifunctional polypeptide or as separate molecules of a multimeric complex—e.g., one antigen-binding unit separately from the other antigen-binding unit. The modifications can be covalent or non-covalent modifications. Such modifications can be introduced into the antibodies or antigen-binding fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments.

In some embodiments, the antibodies or antigen-binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK) (SEQ ID NO:3), polyhistidine (6-His; HHHHHH) (SEQ ID NO:4), hemagglutinin (HA; YPYDVPDYA) (SEQ ID NO:5), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DYLIGHT™ 488, phycoerythrin (PE), propidium iodide (PI), PerCP, PE-ALEXA FLUOR® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J. Nucl. Med. 38:1221-1229) or chelate (e.g., to DOTA or DTPA), which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., of cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug. Chem. 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisić et al. (2010) Int. J. Pharm. 387(1-2):110-119). The stabilization moiety can improve the stability, or retention of, the antibody (or fragment) by at least, e.g., 1.5 (or at least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J. 10(10):2717-2723; and Co et al. (1993) Mol. Immunol. 30:1361.

### Pharmaceutical Compositions and Formulations

Compositions comprising a multispecific antigen-binding construct or an isolated monoclonal antibody, or antigen-binding portion or fragment thereof, of the present disclosure and a pharmaceutically acceptable carrier are also provided. The compositions can further comprise a diluent, solubilizer, emulsifier, preservative, and/or adjuvant to be used with the methods disclosed herein. Such compositions can be used in a subject having cancer or other condition, such as an autoimmune disorder, that would benefit from the multispecific antigen-binding constructs described herein.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Allen (2012) Remington—The Science and Practice of Pharmacy, 22d Edition, Lloyd V, Allen, ed., The Pharmaceutical Press). In certain embodiments, the formulation comprises PBS; 20 mM NaOAC, pH 5.2, 50 mM NaCl; and/or 10 mM NaOAC, pH 5.2, 9% Sucrose. In certain embodiments, the optimal pharmaceutical composition is determined by one skilled in the art depending upon, for example, the intended route of administration, delivery format and desired dosage. See, for example, Allen (2012) Remington—The Science and Practice of Pharmacy, 22d Edition, Lloyd V, Allen, ed., The Pharmaceutical Press. In certain embodiments, such compositions can influence the physical state, stability, rate of in vivo release and/or rate of in vivo clearance of the multispecific antigen-binding construct.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising the multispecific antigen-binding constructs disclosed herein can be prepared for storage by mixing the selected composition having the desired degree of purity with optional formulation agents (see Allen (2012) Remington—The Science and Practice of Pharmacy, 22d Edition, Lloyd V, Allen, ed., The Pharmaceutical Press) in the form of a lyophilized cake or an aqueous solution. Further, in certain embodiments, a composition comprising the multispecific antigen-binding construct disclosed herein can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments when parenteral administration is contemplated, a therapeutic composition can be in the form of a pyrogen-free, parenterally acceptable aqueous solution comprising a multispecific antigen-binding construct, in a pharmaceutically acceptable vehicle. In certain embodiments, a vehicle for parenteral injection is sterile distilled water in which a multispecific antigen-binding construct is formulated as a sterile, isotonic solution, and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a multispecific antigen-binding construct can be formulated as a dry powder for inhalation. In certain embodiments, an inhalation solution comprising a multispecific antigen-binding construct can be formulated with a propellant for aerosol delivery. In certain embodiments, solutions can be nebulized. Pulmonary administration is further described in PCT Application No. PCT/US94/001875, which describes pulmonary delivery of chemically modified proteins.

In certain embodiments, it is contemplated that formulations can be administered orally. In certain embodiments, a multispecific antigen-binding construct that is administered in this fashion can be formulated with or without carriers customarily used in compounding solid dosage forms, such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of a multispecific antigen-binding construct. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a multispecific antigen-binding construct in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a multispecific antigen-binding construct in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (U.S. Pat. No. 3,773,919 and European Pat. No. EP 058,481), copolymers of L-glutamic acid and gamma ethyl-L-glutamate (Sidman et al. (1993) Biopolymers 22:547-556), poly (2-hydroxyethyl-methacrylate) (Langer et al. (1981) J. Biomed. Mater. Res. 15: 167-277; and Langer (1982) Chem. Tech. 12:98-105), ethylene vinyl acetate (Langer et al.) or poly-D(−)-3-hydroxybutyric acid (European Pat. No, EP 133,988). In certain embodiments, sustained release compositions can also include liposomes, which can be prepared by any of several methods known in the art (See, e.g., Eppstein et al. (1985) Proc. Natl. Acad. Sci. USA 82:3688-3692; European Pat. Nos. EP 036,676; EP 088,046; and EP 143,949).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes are included.

In certain embodiments, the effective amount of a pharmaceutical composition comprising a multispecific antigen-binding construct to be employed therapeutically depend, for example, upon the therapeutic context and objectives. One skilled in the art will appreciate that the appropriate dosage levels for treatment, according to certain embodiments, vary depending, in part, upon the molecule delivered, the indication for which a multispecific antigen-binding construct is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the multispecific antigen-binding construct in the formulation used. In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy can be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a multispecific antigen-binding construct in an ex vivo manner. In such instances, cells, tissues (including, e.g., blood) and/or organs that have been removed from the patient are exposed to a pharmaceutical composition comprising a multispecific antigen-binding construct after which the cells, tissues and/or organs are subsequently implanted back into the patient.

In certain embodiments, the antigen-binding construct can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semipermeable polymeric enclosures or membranes that allow the release of the protein product(s) but prevent the destruction of the cells by the patient's immune system or by other detrimental factors from the surrounding tissues.

### Methods of Use of the Multispecific Antigen-Binding Constructs

As described herein, the present disclosure provides a method of treating a proliferative disorder in a subject in need thereof, comprising administering to the subject a therapeutically effective amount of a multispecific antigen-binding construct (e.g., a bispecific antigen-binding construct) of the present disclosure. In some embodiments, the present disclosure provides a method of enhancing an immune response (e.g., enhanced NK and/or T cell function; enhanced NK and/or T cell-mediated response; increased cytokine, e.g., IFNγ, secretion and/or production from T cells; enhanced NK cell and/or T cell function, including cytolytic activity; enhanced macrophage function; enhanced ADCC function) in a subject in need thereof, comprising administering to the subject a therapeutically effective amount of a multispecific antigen-binding construct or composition comprising the construct of the present disclosure. As exemplified herein, the enhancement of the immune response is greater upon administration of the multispecific antigen-binding construct as compared to an agent that has a single target. In some embodiments, the enhancement of the immune response is greater by at least about 10%, 20%, 30%, 40%, 50%, 60%, 70%, 80%, 90%, 100%, or more as compared to an agent that binds either the tumor or B-cell antigen(s) alone or NKp30 or other NK activating receptor alone. These effects occur regardless of the level of tumor antigen expression (i.e., in target cells having high levels of tumor antigen as well as in target cells having low levels of tumor antigen).

The compositions described herein are useful in, inter alia, methods for treating, delaying progression of, or preventing a variety of cancers or conditions in a subject. The cancer or condition can comprise a CD16 deficient microenvironment. Optionally, the CD16 microenvironment is associated with hematopoietic stem cell transplantation to the subject. The CD16 deficient microenvironment can comprise a population of infiltrating NK cells that have less than 50% expression of CD16 as compared to a control NK cell (e.g., a resting NK cell, a healthy NK cell from the subject, an NK cell from a healthy individual). Optionally, the infiltrating NK cells have less than 45%, less than 40%, less than 35%, less than 30%, less than 25%, less than 20%, less than 15%, less than 10%, less than 5%, less than 4%, less than 3%, less than 2%, or less than 1% expression of CD16 as compared to a control NK cell. The infiltrating NK cells in some subjects do not express CD16. In other embodiments, the CD16 deficient microenvironment comprises a population of infiltrating NK cells in which at least 10% of the infiltrating NK cells have reduced expression of CD16 as compared to a control NK cell. Optionally, the CD16 deficient microenvironment comprises a population of infiltrating NK cells in which at least 15%, at least 20%, at least 25%, at least 30%, at least 35%, at least 40%, at least 45%, or at least 50% of the infiltrating NK cells have reduced expression of CD16 as compared to a control NK cell. In other embodiments, the NK cells have a CD16 copy number less than 150,000. Optionally, the NK cells have a CD16 copy number less than 140,000, less than 130,000, less than 120,000, less than 110,000, less than 100,000, less than 75,000, less than 50,000, or less than 25,000.

The cancer or proliferative disorder or tumor to be treated using the methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, liver cancer, pancreatic cancer, a genitourinary cancer, a bone cancer, renal cancer, and a vascular cancer. Optionally, the cancer is selected from the group consisting of Kaposi's sarcoma, leukemia, acute lymphocytic leukemia (etv6, aml1, cyclophilin b), acute myelocytic leukemia, myeloblasts promyelocyte myelomonocytic monocytic erythroleukemia, chronic leukemia, chronic myelocytic (granulocytic) leukemia, chronic lymphocytic leukemia (cyclophilin b), mantle cell lymphoma, primary central nervous system lymphoma, Burkitt's lymphoma, marginal zone B cell lymphoma (Ig-idiotype), Polycythemia vera Lymphoma, Hodgkin's disease (Imp-1, EBNA-1), non-Hodgkin's disease, mycloma (MUC family, p21ras), multiple myeloma, Waldenstrom's macroglobulinemia, heavy chain disease, solid tumors, sarcoma, carcinoma, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colon carcinoma (p21ras, HER2/neu, c-erbB-2, MUC family), pancreatic cancer, breast cancer (MUC family, HER2/neu, c-erbB-2), ovarian cancer, prostate cancer (Prostate Specific Antigen (PSA) and its antigenic epitopes PSA-1, PSA-2, and PSA-3, PSMA, HER2/neu, c-erbB-2, ga733 glycoprotein), squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma (HER2/neu, c-erbB-2), hepatoma, hepatocellular cancer (α-fetoprotein), bile duct carcinoma, choriocarcinoma, seminoma, embryonal carcinoma, Wilm's tumor, cervical cancer, uterine cancer, testicular tumor (NY-ESO-1), lung carcinoma, small cell lung carcinoma, non-small cell lung carcinoma (HER2/neu, c-erbB-2), bladder carcinoma, epithelial carcinoma, glioma (E-cadherin, α-catenin, β-catenin, γ-catenin, p120ctn), astrocytoma, medulloblastoma, craniopharyngioma, ependymoma, pinealoma, hemangioblastoma, acoustic neuroma, oligodendroglioma, menangioma, melanoma (p5 protein, gp75, oncofetal antigen, GM2 and GD2 gangliosides, Melan-A/MART-1, cdc27, MAGE-3, p21ras, gp100), neuroblastoma, retinoblastoma, nasopharyngeal carcinoma (Imp-1, EBNA-1), esophageal carcinoma, basal cell carcinoma, biliary tract cancer (p21ras), bladder cancer (p21ras), bone cancer, brain and central nervous system (CNS) cancer, cervical carcinoma (p53, p21ras), choriocarcinoma (CEA), colorectal cancers (colorectal associated antigen (CRC)-0017-1A/GA733, APC), connective tissue cancer, cancer of the digestive system, endometrial cancer, esophageal cancer, eye cancer, head and neck cancer, gastric cancer (HER2/neu, c-erbB-2, ga733 glycoprotein), epithelial cell cancer (cyclophilin b), intraepithelial neoplasm, kidney cancer, larynx cancer, liver cancer, lung cancer (small cell, large cell) (CEA, MAGE-3, NY-ESO-1), oral cavity cancer (for example lip, tongue, mouth, and pharynx cancers), ovarian cancer (MUC family, HER2/neu, c-erbB-2), pancreatic cancer, rectal cancer, cancer of the respiratory system, skin cancer, thyroid cancer, and cancer of the urinary system.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), oral, intracranial injection, or intrathecal injection (IT). The injection can be in a bolus or a continuous infusion.

As described herein, multispecific antigen-binding constructs targeting both BCMA and NKp30 can specifically deplete plasma cells in the bone marrow. This indicates that such constructs are useful for treating disorders in which depletion of a target cell population, such as B cells, is required, for example, disorders mediated in whole or in part by autoreactive B cells or antibodies. Accordingly, in yet another aspect, the disclosure features a method for depleting target cells (e.g., tumor cells or B-lineage cells) from a subject, the method comprising administering to a subject in need thereof a multispecific antigen-binding construct described herein in an amount sufficient to deplete one or more target cells expressing a target antigen, wherein the multispecific binding construct comprises at least two linked antigen-binding units, wherein a first antigen-binding unit specifically binds the target antigen, and wherein a second antigen-binding unit specifically binds an NKp30 antigen. In some embodiments, the multispecific antigen-binding construct comprises a third antigen-binding unit. In some embodiments, the multispecific antigen-binding construct comprises a third antigen-binding unit and a fourth antigen-binding unit. In some embodiments, the target antigen is a tumor antigen, such as any of the tumor antigens described herein or known in the art. In some embodiments, the target cells are B cells, such as autoreactive B cells, and, in such cases, the target antigen is a B cell-specific, B cell-restricted, or B cell-enriched antigen, such as CD20 or BCMA. In some embodiments, the subject is a human. In some embodiments, the subject is afflicted with an autoimmune condition. In some embodiments, the autoimmune condition is mediated, in whole or in part, by autoreactive B cells. In some embodiments, the subject being depleted of target cells has or is at risk for red-cell aplasia. The red-cell aplasia can be due, for example, to ABO blood-group incompatibility following an allogenic stem-cell transplant. In some such embodiments, the subject has blood group O and the allogenic stem-cell transplant was from a blood group A donor. In those embodiments where the subject being depleted of target cells has or is at risk for red-cell aplasia, the target cells are, for example, target plasma B cells. Accordingly, in some embodiments, the target antigen is an antigen found on plasma B cells but not on other B cells, for example.

In some embodiments of the methods described herein, a subject being treated with the multispecific constructs provided herein has a disease or disorder in which therapeutic plasma exchange is a primary standard of care or a first-line adjunct, such as, for example, in Guillain-Barre Syndrome (GB S), myasthenia gravis, SLE, ANCA-associated vasculitis, inflammatory myopathies, diffuse scleroderma, autoimmune meningoencephalitis, rheumatoid arthritis, cryoglobulinemia, and primary APS. The American Society for Apheresis (ASFA) guidelines of 2016, define the term plasmapheresis as “a procedure in which blood of the patient or the donor is passed through a medical device which separates plasma from other components of blood and the plasma is removed (i.e., less than 15% of total plasma volume) without the use of colloid replacement solution” and TPE (therapeutic plasma exchange) as “a therapeutic procedure in which blood of the patient is passed through a medical device which separates plasma from other components of blood. The plasma is removed and replaced with a replacement solution such as colloid solution (e.g., albumin and/or plasma) or a combination of crystalloid/colloid solution.” Thus, as used herein, TPE is an extracorporeal blood purification technique for the removal of high molecular weight substances (>15,000 Da), such as pathogenic autoantibodies, immune complexes, cryoglobulins, myeloma light chains, endotoxins, and lipoproteins that contain cholesterol. Accordingly, in some embodiments, the multispecific antigen-binding constructs described herein can be used in those indications where TPE is used to deplete components from the plasma, such as, for example, any of the autoimmune diseases listed in Table 1 of “Therapeutic Plasma Exchange as Management of Complicated Systemic Lupus Erythematosus and Other Autoimmune Diseases,” D. Aguirre-Valencia et al., Autoimmune Diseases (2019) Article ID 5350960, the contents of which are herein incorporated by reference in their entireties.

In another aspect, the disclosure features a method for treating a subject having an inflammatory condition (e.g., an autoimmune condition) that is mediated in whole or in part by autoreactive B cells, the method comprising administering to the subject a multispecific antigen-binding construct described herein in an amount sufficient to deplete one or more autoreactive B cells expressing the target antigen. Also provided is a method of treating a subject with an autoreactive-B cell inflammatory condition by administering to the subject an effective amount of a multispecific antigen-binding construct described herein. Optionally, the effective amount of the multispecific antigen-binding construct or of the pharmaceutical composition thereof reduces bone marrow levels of plasma cells expressing IgG, IgM, or IgA. As used herein, an autoreactive-B cell inflammatory condition is an autoimmune disease mediated by autoreactive B cells and examples include autoimmune disease mediated by autoreactive B cells. The methods described herein can include administering to the subject an anti-inflammatory agent, such as anti-inflammatory agent (corticosteroid, DMARDs, or as anti-cytokine agent) or other agents used to treat inflammation or autoimmune diseases or conditions. Optionally, the target antigen is a B cell-specific, B cell-restricted, or B cell-enriched antigen, such as CD20, CD19, or BCMA. In some embodiments, the autoimmune disease mediated in whole or in part by autoreactive B cells can be one selected from the group consisting of systemic lupus erythematosus (SLE), Sjogren's syndrome, Type 1 diabetes, Addison disease, Pernicious anemia, autoimmune hepatitis, multiple sclerosis, rheumatoid arthritis, myasthenia gravis, primary biliary cholangitis, Goodpasture's disease, primary membranous nephropathy, ovarian insufficiency, pemphigus vulgaris, and autoimmune orchitis. Other such autoimmune diseases are known in the art and described in, e.g., Ludwig et al. (2017) Frontiers in Immunol 8: Article 603 and Hofmann et al. (2018) Frontiers in Immunol 9: Article 835. Other indications involving abnormal B cells that can be treated using the constructs and methods described herein include light-chain amyloidosis, pemphigus vulgaris, and immune thrombocytopenia.

For example, myasthenia gravis is a chronic autoimmune neuromuscular disease that causes weakness in the skeletal muscles, including the arms and legs. In myasthenia gravis, antibodies (immune proteins) block, alter, or destroy the receptors for acetylcholine at the neuromuscular junction, which prevents the muscle from contracting. The prevalence of this disease is about 20 cases per 100,000, and, in most individuals with myasthenia gravis, the disease is caused by antibodies to the acetylcholine receptor itself. However, antibodies to other proteins, such as MuSK (Muscle-Specific Kinase) protein, can also lead to impaired transmission at the neuromuscular junction. Corticosteroids and immunosuppressants typically used to treat myasthenia gravis are associated with a number of significant side effects. For example, use of Soliris only blocks the activity of complement recruited by the pathogenic IgGs directed against the ACh receptor. It does not address the blocking of the Ach receptor by pathogenic IgGs, nor the receptor cross-linking and internalization by these IgGs.

Light-chain amyloidosis or AL amyloidosis is a disorder in which a group of plasma cells make too many light chains, which misfold and clump together to form amyloid fibrils. The fibrils are then deposited in organs. The most common organs affected are the heart and kidneys. Light chain amyloidosis can also affect the stomach, large intestine, liver, nerves and skin. The prevalence of light-chain amyloidosis is about 40 cases per 1,000,000. Up to 80% of patients are ineligible for autologous stem cell transplant (ASCT), and plasma cell directed chemotherapy has been shown to fall short in addressing organ dysfunction caused by amyloid deposition.

Pemphigus vulgaris, which has a prevalence of about 1-10 patients per 100,000, is an uncommon, potentially fatal, autoimmune disorder characterized by intraepidermal blisters and extensive erosions on apparently healthy skin and mucous membranes. Pemphigus vulgaris is characterized by IgG autoantibodies directed against the calcium-dependent cadherins desmoglein 1 and desmoglein 3. These transmembrane glycoproteins affect cell-cell adhesion and signaling between epidermal cells. Acantholysis (loss of intercellular adhesion with consequent epidermal blister formation) results from either direct inhibition of desmoglein function by autoantibody binding or from autoantibody-induced cell signaling that results in down-regulation of cell-cell adhesion. The autoantibodies are present in both serum and skin during active disease. Any area of stratified squamous epithelium can be affected, including mucosal surfaces. Patients who do not respond to corticosteroids and immunosuppressants are treated with IV Ig or Rituxan. Even with IV Ig and Rituxan, complete remission can take several months, and some patients do not respond.

Immune thrombocytopenia (ITP), which has a prevalence of about 9.5 cases per 100,000, occur when autoantibodies are produced against normal platelets. The platelets are destroyed and eliminated from the body, resulting in a shortage of these cells in affected individuals. Some of these antibodies also affect the cells in the bone marrow that produce platelets (megakaryocytes), which leads to a decrease in platelet production, further reducing the number of platelets in the blood. Treatment for ITP is typically focused on either reducing the autoimmune destruction of the platelets, or directly stimulating platelet production with specific growth factors. Use of IV Ig and plasmapheresis can lead to serious complications, and while thrombopoietin receptor agonists lead to increases in blood platelet counts, they do not address the underlying destruction of the platelets.

Accordingly, in some embodiments of the methods described herein, a subject being treated with the multispecific constructs described herein has a disease selected from myasthenia gravis, light-chain amyloidosis, pemphigus vulgaris, and immune thrombocytopenia.

In some embodiments, such multispecific constructs can be combined with other therapies for inflammatory disorders (e.g., autoimmune diseases), such as corticosteroids, DMARDs, or anti-cytokine therapies.

Thus, provided herein is a method of treating a subject with an autoreactive-B cell inflammatory condition by administering to the subject an effective amount of a multispecific antigen-binding construct comprising at least two linked antigen-binding units, wherein a first antigen-binding unit specifically binds a first target antigen expressed by a B-lineage cell and wherein a second antigen-binding unit specifically binds a NKp30 antigen or a pharmaceutical composition comprising the construct. The term autoreactive-B cell inflammatory condition includes conditions driven in part or fully by autoreactive B cells or plasma cells. More specifically, autoreactive-B cell mediated inflammatory conditions include numerous autoimmune conditions as described herein.

The effective amount of a multispecific antigen-binding construct or pharmaceutical composition reduces, for example, bone marrow levels of plasma cells expressing IgG, IgM, or IgA. The method optionally further comprises administering to the subject an anti-inflammatory agent (e.g., corticosteroid, DMARDs, or as anti-cytokine agent) or other agents used in the treatment of inflammatory and autoimmune conditions.

As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have a disease or condition that can be treated with a multispecific antigen-binding construct provided herein. In some embodiments, the disease or condition is a cancer. In some embodiments, the subject is a human with a cancer that can be treated with a multispecific antigen-binding construct provided herein. In some embodiments, the subject is a human that is suspected of having cancer that can be treated with a multispecific antigen-binding construct provided herein.

Treating or treatment of any disease or disorder refers to ameliorating a disease or disorder that exists in a subject. The term ameliorating refers to any therapeutically beneficial result in the treatment of a disease state, e.g., cancer, lessening in the severity or progression, promoting remission or durations of remission, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying or preventing metastasis or progression.

As used herein, administer or administration refers to the act of injecting or otherwise physically delivering a substance as it exists outside the body (e.g., a multispecific antigen-binding construct provided herein) into a patient, such as by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Pat. Nos. EP488401 and EP 430539. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems. In some embodiments, a multispecific antigen-binding construct of the present disclosure is therapeutically delivered to a subject by way of local administration.

As used herein, the term enhanced T cell function or activation of T cells refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation typically requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof. In some embodiments, enhanced T cell function also encompasses enhanced survival and/or enhanced proliferation of the T cell. Methods for measuring such activities are routine and known in the art, and exemplary methods are described herein, such as in the Examples.

As used herein, the term T cell-mediated response refers to any response mediated by T cells, including, but not limited to, effector T cells (e.g., CD8⁺ cells, effector γδ T cells) and helper T cells (e.g., CD4⁺ cells, including subbsets thereof, such as TH1, TH2, TH3, TH17, TH9, and TFH cells). T cell-mediated responses include, for example, T cell cytotoxicity, T cell cytokine secretion, and proliferation. In some embodiments, the T cell-mediated response enhanced by the multispecific antigen-binding constructs described herein includes responses mediated by effector γδ T cells, such as cytolytic activity through the perforin-granzyme pathway and/or through the TRAIL/FAS-L pathways; ADCC-mediated cytolysis or cytokine secretion; and cytokine secretion of IFN-γ and TNF-α through TCR stimulation, NKG2D stimulation, and/or IL-12 or IL-18 induction. See, C. Zou et al., Oncotarget. (2017) 8(5):8900-8909, the contents of which are herein incorporated by reference in their entireties.

Also provided herein is a method for treating or delaying progression of a cancer or reducing or inhibiting tumor growth in a subject by administering to the subject an effective amount of a multispecific antigen-binding construct, an antibody or antigen-binding fragment thereof, a pharmaceutical composition, or a protein conjugate as described herein. Optionally the administration enhances γδ T cell-mediated cytotoxicity or cytokine production.

As used herein, the term therapeutically effective amount or effective amount refers to an amount of a multispecific antigen-binding construct that, when administered to a subject, is effective to treat a disease or disorder or to achieve another desired effect (e.g., effective to enhance the killing of a target cell (such as a cancer cell or B-lineage cell) by NK cells or other NKp30-expressing effector cells, such as effector γδ T cells). A suitable dose of an antibody or fragment thereof described herein, which dose is capable of treating or preventing cancer in a subject or capable of enhancing the killing of a target cell by an NK cell in vitro or in vivo, can depend on a variety of factors including the particular construct used and whether it is used concomitantly with other therapeutic agents. For example, a different dose of a whole multispecific antigen-binding construct may be required to treat a subject with cancer as compared to the dose of a fragment of the multispecific antigen-binding construct (e.g., single antigen-binding unit) required to treat the same subject. Other factors affecting the dose administered to the subject include, e.g., the type or severity of the cancer or autoreactive-B cell mediated condition. For example, a subject having metastatic melanoma may require administration of a different dosage of multispecific antigen-binding construct than a subject with glioblastoma. Similarly, a subject with myasthenia gravis may need a different dosage than a subject with systemic lupus erythematosus. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects.

A pharmaceutical composition can include a therapeutically effective amount of a multispecific antigen-binding construct described herein. Such effective amounts can be readily determined by one of ordinary skill in the art as described above. Considerations include the effect of the administered multispecific antigen-binding construct or the combinatorial effect of the multispecific antigen-binding construct with one or more additional active agents, if more than one agent is used in or with the pharmaceutical composition.

Suitable human doses of any of the multispecific antigen-binding constructs described herein can further be evaluated in, e.g., Phase I dose escalation studies. See, e.g., van Gurp et al. (2008) Am. J. Transplantation 8(8):1711-1718; Hanouska et al. (2007) Clin. Cancer Res. 13(2, part 1):523-531; and Hetherington et al. (2006) Antimicrobial Agents and Chemotherapy 50(10): 3499-3500.

Toxicity and therapeutic efficacy of such multispecific antigen-binding constructs can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A multispecific antigen-binding construct that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of an antigen-binding construct described herein lies generally within a range of circulating concentrations of the multispecific antigen-binding construct that include the ED₅₀ with little or no toxicity. The dosage can vary within this range depending upon the dosage form employed and the route of administration utilized. For antigen-binding constructs described herein, the therapeutically effective dose can be estimated initially from cell culture assays. A dose can be formulated in animal models to achieve a circulating plasma concentration range that includes the EC₅₀ (i.e., the concentration of the construct—e.g., antibody—which achieves a half-maximal inhibition of symptoms) as determined in cell culture. Such information can be used to more accurately determine useful doses in humans. Levels in plasma can be measured, for example, by high performance liquid chromatography. In some embodiments, e.g., where local administration (e.g., to the eye or a joint) is desired, cell culture or animal models can be used to determine a dose required to achieve a therapeutically effective concentration within the local site.

In some embodiments, the multispecific antigen-binding construct described herein can be administered to a subject as a monotherapy. Alternatively, the antigen-binding construct can be administered in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, inflammatory or autoimmune disease therapy (e.g., anti-inflammatory agents or anti-cytokine agents) or other pharmacotherapy.

Chemotherapeutic agents suitable for co-administration with compositions of the present disclosure include, for example, taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide. In some embodiments, the multispecific antigen-binding construct and the one or more additional active agents are administered at the same time. Optionally, the multispecific antigen-binding construct is administered first in time and the one or more additional active agents are administered second in time. In some embodiments, the one or more additional active agents are administered first in time and the multispecific antigen-binding construct is administered second in time. Optionally, the multispecific antigen-binding construct and the one or more additional agents are administered simultaneously in the same or different routes. For example, a composition comprising the antigen-binding construct optionally contains one or more additional agents.

A multispecific antigen-binding described herein can replace or augment a previously or currently administered therapy. For example, upon treating with a multispecific antigen-binding construct, administration of the one or more additional active agents can cease or diminish, e.g., be administered at lower levels or dosages. In some embodiments, administration of the previous therapy can be maintained. In some embodiments, a previous therapy is maintained until the level of the multispecific antigen-binding construct reaches a level sufficient to provide a therapeutic effect.

Monitoring a subject (e.g., a human patient) for an improvement in a cancer or autoreactive-B cell mediated inflammatory condition, as defined herein, means evaluating the subject for a change in a disease parameter, e.g., a reduction in tumor growth or size. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks, or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer described herein.

In some embodiments, a therapeutically effective amount of a multispecific antigen-binding construct, or a composition comprising the construct, described herein is administered to a subject to modulate or enhance an immune response in a subject in need thereof. In some embodiments, the enhanced immune response includes one or more of enhanced T cell function, enhanced NK cell function, or enhanced macrophage function. In certain aspects, the multispecific antigen-binding construct enhances the subject's immune response by agonizing NKp30 function by bridging an immune cell that expresses NKp30 with a second cell (e.g., a tumor cell or B-lineage cell) that expresses a tumor or B-lineage cell antigen.

Also provided herein are methods for activating or sustaining activation of an NK cell that has reduced expression of CD16 as compared to the expression of CD16 by a control NK cell. The method includes contacting the NK cell with an effective amount of the multispecific antigen-binding construct described herein. The multispecific binding construct, for example, comprises at least two linked antigen-binding units, wherein a first antigen-binding unit specifically binds a tumor antigen (e.g., BCMA or HER2/neu), wherein a second antigen-binding unit specifically binds an NKp30 antigen. The contacting of the NK cell with the multispecific antigen-binding construct can be in vitro or in vivo.

In some embodiments, the methods are used to activate or sustain activation of an NK cell in a subject having cancer. The cancer cells may express the target antigen to which the multispecific antigen-binding construct specifically binds. Optionally, the tumor antigen to which the multispecific antigen-binding construct specifically binds is BCMA or HER2. The contacted NK cell can be a tumor infiltrating NK cell.

The activation or sustained activation of the NK cell occurs regardless of whether the NK cell is in a CD16 deficient microenvironment. The CD16 deficient microenvironment may comprise a population of infiltrating NK cells that have less than 50% expression of CD16 as compared to a control NK cell (e.g., a resting NK cell, a healthy NK cell from the subject, an NK cell from a healthy individual). Optionally, the infiltrating NK cells have less than less than 45%, less than 40%, less than 35%, less than 30%, less than 25%, less than 20%, less than 15%, less than 10%, less than 5%, less than 4%, less than 3%, less than 2%, or less than 1% expression of CD16 as compared to a control NK cell. The infiltrating NK cells in some subjects do not express CD16. In other aspects, the CD16 deficient microenvironment comprises a population of infiltrating NK cells in which at least 10% of the infiltrating NK cells have reduced expression of CD16 as compared to a control NK cell. Optionally, the CD16 deficient microenvironment comprises a population of infiltrating NK cells in which at least 15%, at least 20%, at least 25%, at least 30%, at least 35%, at least 40%, at least 45%, or at least 50% of the infiltrating NK cells have reduced expression of CD16 as compared to a control NK cell. In other aspects, the NK cells have a CD16 copy number less than 150,000. Optionally, the NK cells have a CD16 copy number less than 140,000, less than 130,000, less than 120,000, less than 110,000, less than 100,000, less than 75,000, less than 50,000, or less than 25,000.

As used herein, the term enhanced NK cell function or activation of NK cells refers to a cellular process by which NK cells respond to cognate ligands or to the multipspecific antigen-binding constructs decsribed herein by entering the cell cycle (i.e., proliferating), increasing cell-surface expression of one or more activation markers, secreting or increasing secretion of cytokines or chemokines (including IFN-γ, TNF-α, IL-17A, and IL-22) or lytic enzymes (e.g., perforin and granzymes), and initiating or becoming competent to perform cell-based effector functions. Methods for measuring such activities are routine and known in the art, and exemplary methods are described herein, such as in the Examples. For example, in those embodiments where the enhanced NK cell function is increased or enhanced cytokine production by the NK cells, a cytokine assay, such as an ELISA or cytokine bead array assay, can be used to determine the increase. In some embodiments, an increase in cytokine production in the presence of the multispecific antigen-binding constructs is at least 1-fold, at least 2-fold, at least 3-fold, at least 4-fold, at least 5-fold, or more compared to a control or reference antibody. In those embodiments where the enhanced NK cell function is increased or enhanced expression of one or more activation markers, the assay comprises detecting an increase in surface expression of at least one activation marker on the NK cells, for example using flow cytometry. In some embodiments, “increase in surface expression” refers to at least a 5%, 10%, 15%, 20%, 25%, 30%, 35%, 40%, 45%, 50%, 55%, 60%, 65%, 70%, 75%, 80%, 85%, 90%, 95% or 100% increase in surface expression relative to surface expression in the presence of a control antibody or reference antibody.

The multispecific antigen-binding constructs disclosed herein can be used as described in any of the methods herein for the treatment of cancer and, optionally, in a method further comprising administering an anticancer therapy to the subject. Such anticancer therapy (e.g., an immunotherapy, chemotherapy, etc.) as described herein can be administered prior to, concurrently with, or after treatment with the multispecific antigen-binding construct. By way of example, anticancer therapy can be administered one or more times minutes, hours, days, or weeks prior to administration of the multispecific antigen-binding construct. In such cases, the anticancer therapy can be administered alone until the tumor cells downregulate tumor antigen expression and, at such time, the administration of the multispecific antigen-binding construct can begin along with or instead of the anticancer therapy. In certain embodiments of the method, the anticancer therapy and the multispecific antigen-binding construct are administered entirely or partially in overlapping periods. Thus, both can be administered during the same period of minutes, hours, days, weeks, or months. During such overlapping time periods, the anticancer therapy and the multispecific antigen-binding construct can be administered at the same time or at approximately the same time (for example, in the same composition or by the same administration means), or one can be administered prior to or after the other (by minutes, hours, days, etc.). Optionally, the anticancer therapy is an immunotherapy and the cancer in the subject is refractory to the immunotherapy, at least in the absence of treatment with the multispecific antigen-binding construct.

Also provided herein are methods of treating cancer in a subject, wherein the cancer comprises a low level of tumor antigen. The method comprises administering to the subject an effective amount of the multispecific antigen-binding construct described herein. The multispecific binding construct, for example, comprises at least two linked antigen-binding units, wherein a first antigen-binding unit specifically binds a tumor antigen (e.g., BCMA or HER2/neu), wherein a second antigen-binding unit specifically binds an NKp30 antigen and can be any multispecific antigen-binding construct described herein. The cancer to be treated comprises a low level of the tumor antigen. As used herein, a low level of a tumor antigen is any level not considered high by one of skill in the art. By way of example, BCMA expression levels of greater than about 130,000-150,000 copies of BCMA per tumor cell are considered high, whereas BCMA expression levels of less than about 130,000-150,000 are considered medium or low. Thus, low levels of BCMA, as used herein, include less than about 100,000, less than about 90,000, less than about 75,000, and less than 50,000 copies of BCMA per tumor cell. Thus, the method of treating cancer in a subject, wherein the cancer comprises a low level of tumor antigen, includes a level of the tumor antigen less than about 100,000 tumor antigen copies per cancer cell, less than 130 tumor antigen copies per cell, less than about 75,000 tumor antigen copies per cancer cell, less than about 60,000 tumor antigen copies per cancer cell, less than 50,000 tumor antigen copies per cancer cell, less than 40,000 tumor antigen copies per cancer cell, less than 35,000 tumor antigen copies per cell, or any number of fewer copies per cell. The low level of tumor antigen expression may be downregulated over time (e.g., as the tumor cells escape cell death by infiltrating immune cells, such as NK cells), may be a characteristic of a particular tumor cell, or both.

Despite the low level of tumor antigen to which the first antigen-binding unit specifically binds, the multispecific antigen-binding construct can enhance the subject's immune response by agonizing NKp30 function, can enhance NK cell mediated cancer cell lysis, promote production of cytokines (e.g., IFNγ), and enhance ADCC functions. Thus, the multispecific antigen-binding construct can be used as described in any of the methods herein for the treatment of various cancer types and, optionally, in a method further comprising administering an anticancer therapy to the subject. Such anticancer therapy (e.g., an immunotherapy) as described herein can be administered prior to, concurrently with, or after treatment with the multispecific antigen-binding construct. By way of example, anticancer therapy can be administered one or more times minutes, hours, days, or weeks prior to administration of the multispecific antigen-binding construct. In such cases, the anticancer therapy can be administered alone until the tumor cells downregulate tumor antigen expression and, at such time, the administration of the multispecific antigen-binding construct can begin along with or instead of the anticancer therapy. In certain embodiments of the methods described herein, the anticancer therapy and the multispecific antigen-binding construct are administered entirely or partially in overlapping periods. Thus, both can be administered during the same period of minutes, hours, weeks, or months. During such overlapping time periods, the anticancer therapy and the multispecific antigen-binding construct can be administered at the same time or at approximately the same time (for example, in the same composition or by the same administration means) or one can be administered prior to or after the other (by minutes, hours, days, etc.). Optionally, the anticancer therapy is an immunotherapy and the cancer in the subject is refractory to the immunotherapy, at least in the absence of treatment with the multispecific antigen-binding construct.

Also provided herein is a method for enhancing killing of a target cell by a natural killer (NK) cell. The method includes contacting the target cell with an effective amount of a multispecific antigen-binding construct (i.e., enough to enhance killing of the target cell) in the presence of the NK cell, wherein the target cell expresses a tumor antigen at a copy number of less than about 100,000, wherein the multispecific binding construct comprises at least two linked antigen-binding units, wherein a first antigen-binding unit specifically binds the tumor antigen, and wherein a second antigen-binding unit specifically binds an NKp30 antigen. The contacting step can be in vivo or in vitro. When the contacting step is performed in vitro, the viable cells remaining are optionally transplanted to the same or a different subject from the subject from which the cells were derived. For example, the remaining viable cells could be stem cells, hematopoietic cells, or the like.

A method for enhancing the killing of a cancer or B-lineage cell by an NK cell in a subject is also provided. The method comprises administering to the subject an effective amount of a multispecific antigen-binding construct (i.e., an amount sufficient to enhance killing of a cancer or B cell by an NK cell. Optionally, the cancer cell expresses a tumor antigen at a copy number of less than about 100,000 and the multispecific binding construct comprises at least two linked antigen-binding units, wherein a first antigen-binding unit specifically binds the tumor antigen, and wherein a second antigen-binding unit specifically binds a NKp30 antigen.

As used herein, the term enhanced NK cell function or activation of NK cells refers to a cellular process by which NK cells respond to cognate ligands or to the multispecific antigen-binding constructs described herein by entering the cell cycle (i.e., proliferating), increasing cell-surface expression of one or more activation markers, secreting or increasing secretion of cytokines or chemokines (including IFN-γ, TNF-α, IL-17A, and IL-22) or lytic enzymes (e.g., perforin and granzymes), and initiating or becoming competent to perform cell-based effector functions. Methods for measuring such activities are routine and known in the art, and exemplary methods are described herein, such as in the Examples. For example, in those embodiments where the enhanced NK cell function is increased or enhanced cytokine production by the NK cells, a cytokine assay, such as an ELISA or cytokine bead array assay, can be used to determine the increase. In some embodiments, an increase in cytokine production in the presence of the multispecific antigen-binding constructs is at least 1-fold, at least 2-fold, at least 3-fold, at least 4-fold, at least 5-fold, or more compared to a control or reference antibody. In those embodiments where the enhanced NK cell function is increased or enhanced expression of one or more activation markers, the assay comprises detecting an increase in surface expression of at least one activation marker on the NK cells, for example using flow cytometry. In some embodiments, “increase in surface expression” refers to at least a 5%, 10%, 15%, 20%, 25%, 30%, 35%, 40%, 45%, 50%, 55%, 60%, 65%, 70%, 75%, 80%, 85%, 90%, 95% or 100% increase in surface expression relative to surface expression in the presence of a control antibody or reference antibody.

In some aspects, provided herein are multispecific antigen-binding constructs for use in depleting target cells (e.g., tumor cells or B-lineage cells) from a subject in need thereof, wherein the multispecific binding construct comprises at least two linked antigen-binding units, wherein a first antigen-binding unit specifically binds the target antigen, and wherein a second antigen-binding unit specifically binds an NKp30 antigen. In some embodiments, the multispecific antigen-binding construct comprises a third antigen-binding unit. In some embodiments, the multispecific antigen-binding construct comprises a third antigen-binding unit and a fourth antigen-binding unit. In some embodiments, the target antigen is a tumor antigen, such as any of the tumor antigens described herein or known in the art. In some embodiments, the target cells are B cells, such as autoreactive B cells, and, in such cases, the target antigen is a B cell-specific, B cell-restricted, or B cell-enriched antigen, such as CD20 or BCMA. In some embodiments, the subject is a human. In some embodiments, the subject is afflicted with an autoimmune condition. In some embodiments, the autoimmune condition is mediated, in whole or in part, by autoreactive B cells. In some embodiments, the subject being depleted of target cells has or is at risk for red-cell aplasia. In some such embodiments, the subject has blood group O and the allogenic stem-cell transplant was from a blood group A donor. In those embodiments where the subject being depleted of target cells has or is at risk for red-cell aplasia, the target cells are, for example, target plasma B cells. Accordingly, in some embodiments, the target antigen is an antigen found on plasma B cells but not on other B cells, for example. In some aspects, provided herein are multispecific antigen-binding constructs for use in treating an inflammatory condition (e.g., an autoimmune condition or autoreactive-B cell inflammatory condition) that is mediated in whole or in part by autoreactive B cells in a subject in need thereof. In some embodiments, the subject has a disease selected from myasthenia gravis, light-chain amyloidosis, pemphigus vulgaris, and immune thrombocytopenia. In some embodiments, such multispecific constructs can be combined with other therapies for inflammatory disorders (e.g., autoimmune diseases), such as corticosteroids, DMARDs, or anti-cytokine therapies.

In some aspects, provided herein are multispecific antigen-binding constructs for use in modulating or enhancing an immune response, such as a cancer immune response, in a subject in need thereof. In some embodiments, the enhanced immune response includes one or more of enhanced T cell function, enhanced NK cell function, or enhanced macrophage function. In certain aspects, the multispecific antigen-binding construct enhances the subject's immune response by agonizing NKp30 function by bridging an immune cell that expresses NKp30 with a second cell (e.g., a tumor cell or B-lineage cell) that expresses a tumor or B-lineage cell antigen.

In some aspects, provided herein are multispecific antigen-binding constructs for use in activating or sustaining activation of an NK cell that has reduced expression of CD16 as compared to the expression of CD16 by a control NK cell. In some aspects, provided herein are multispecific antigen-binding constructs for use treating or delaying progression of a cancer having a low level of tumor antigen, in a subject in need thereof. In some aspects, provided herein are multispecific antigen-binding constructs for use in increasing or enhancing killing of a target cell by a natural killer (NK) cell in a subject in need thereof. In some aspects, provided herein are multispecific antigen-binding constructs for use in enhancing the killing of a cancer or B-lineage cell by an NK cell in a subject.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

### Example 1

### Multispecific Antigen-Binding Constructs Targeting BCMA and NKp30 Enhance NK Effector Function

To analyze the effect of multispecific antigen-binding constructs targeting BCMA and NKp30 on NK cells, primary human NK cells are primed with 10 ng/mL IL-15 and incubated overnight (in the presence of 10 IU/mL IL-2) with BCMA-expressing target cells of the multiple myeloma cell line H929 and 10 nM of a multispecific antigen-binding construct described herein having a format comprising a whole antibody (IgG1) in which each arm specifically binds to BCMA and at the C-terminus of each heavy chain is an scFv that specifically binds to human NKp30. The first antigen-binding domain comprises the heavy chain (SEQ ID NO: 2) and light chain variable region (SEQ ID NO: 1) of the exemplary CA8 antibody described in U.S. Pat. No. 9,273,141. The scFv comprises heavy chain and light chain variable regions of the antibody produced by the hybridoma 1-2576 (described in detail in U.S. Pat. No. 7,517,966, the disclosure of which as it relates to this antibody and its sequences are incorporated herein by reference in their entirety) or a humanized version thereof. The multispecific antigen-binding constructs targeting BCMA and NKp30 enhance NK effector function as compared to control BCMA antibody alone.

### Additional Constructs

Construct 1, a multispecific antigen-binding construct that specifically binds human BCMA and human NKp30, was generated using standard molecular biology techniques. The construct contains an anti-BCMA IgG1 antibody (mAb1), in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO: 22) linker. The light chains for the anti-BCMA portion and the anti-NKp30 portion of the construct are identical. Construct 1, the structure for which is represented by the illustration of FIG. 1, comprises the heavy chain sequence depicted in SEQ ID NO: 9 and the light chain sequence depicted in SEQ ID NO: 8.

Construct 2, which is also a multispecific antigen-binding construct that specifically binds human BCMA and human NKp30, was generated using standard molecular biology techniques. The construct contains an anti-BCMA IgG1 antibody (mAb1), in which the heavy chain of the antibody is a fusion protein further comprising at its c-terminus the heavy chain variable region of an anti-NKp30 antibody, which is connected to the Fc region of the anti-BCMA antibody by way of a poly-GGGS (SEQ ID NO:22) linker. The light chains for the anti-BCMA portion and the anti-NKp30 portion of the construct are identical. Construct 2, the structure for which is represented by the illustration of FIG. 1, comprises the same anti-BCMA IgG1 antibody portion as Construct 1, and the same light chain as Construct 1, but differs by the variable region sequence of the anti-NKp30 antibody portion (mAb9) of the construct. Construct 2 comprises the heavy chain sequence depicted in SEQ ID NO: 24 and the light chain sequence depicted in SEQ ID NO: 8.

Aglycosylated versions of Constructs 1 and 2 (“Construct 1 aglyco”/Construct 3 or “Construct 2 aglyco”/Construct 4) were also created in which the Fc portion of the heavy chain of each construct contained the N297A amino acid substitution (numbered according to EU numbering). For example, Construct 3 comprises a heavy chain having the amino acid sequence depicted in SEQ ID NO: 26 and a light chain having the amino acid sequence depicted in SEQ ID NO: 8. Construct 4 comprises a heavy chain having the amino acid sequence depicted in SEQ ID NO: 27 and a light chain having the amino acid sequence depicted in SEQ ID NO: 8.

Construct 5 comprises an affinity matured anti-BCMA IgG1 antibody (mAb3) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 5 comprises a heavy chain having the amino acid sequence depicted in SEQ ID NO: 65 and a light chain having the amino acid sequence depicted in SEQ ID NO: 8.

Construct 6 comprises an affinity matured anti-BCMA IgG1 antibody (mAb2) in which the heavy chain of the antibody is a fusion protein further comprising at its C-terminus the heavy chain variable region of an anti-NKp30 antibody (mAb8), which is connected to the Fc region of the anti-BCMA antibody by way of a poly-GGGS (SEQ ID NO:22) linker. The light chains for the anti-BCMA portion and the anti-NKp30 portion of the construct are identical. Construct 6 comprises a heavy chain having the amino acid sequence depicted in SEQ ID NO: 66 and a light chain having the amino acid sequence depicted in SEQ ID NO: 8.

Construct 7 is an afucosylated construct that comprises an anti-BCMA IgG1 antibody (mAb1) in which the heavy chain of the antibody is a fusion protein further comprising at its C-terminus the heavy chain variable region of an anti-NKp30 antibody (mAb8), which is connected to the Fc region of the anti-BCMA antibody by way of an extended poly-GGGS (SEQ ID NO:22) linker. The light chains for the anti-BCMA portion and the anti-NKp30 portion of the construct are identical. Construct 66 comprises a heavy chain having the amino acid sequence depicted in SEQ ID NO: 67 and a light chain having the amino acid sequence depicted in SEQ ID NO: 8.

Construct 8 is an aglycosylated version of construct 5 that comprises an anti-BCMA IgG1 antibody (mAb3) in which the heavy chain of the antibody is a fusion protein further comprising at its C-terminus the heavy chain variable region of an anti-NKp30 antibody (mAb8), which is connected to the Fc region of the anti-BCMA antibody by way of an extended poly-GGGS (SEQ ID NO:22) linker. The light chains for the anti-BCMA portion and the anti-NKp30 portion of the construct are identical. Construct 8 comprises a heavy chain having the amino acid sequence depicted in SEQ ID NO: 68 and a light chain having the amino acid sequence depicted in SEQ ID NO: 8.

### Example 2

### Multispecific Antigen-Binding Constructs Targeting BCMA and NKp30 Enhance NK Effector Function in the Absence of CD16 Expression by the NK Cells

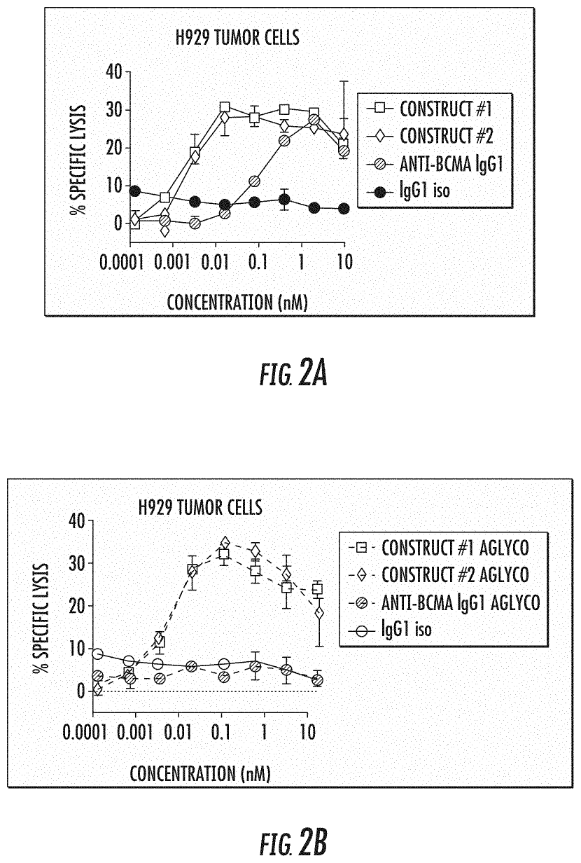

To analyze the effect of multispecific antigen-binding constructs targeting BCMA and NKp30 on NK cells in the absence of CD16 expression, antibodies that cannot bind CD16 were studied (FIG. 2A and FIG. 2B). Glycosylated multispecific antigen-binding constructs targeting BCMA and NKp30 retain the ability to bind CD16, whereas aglycosylated antibodies lack the ability to bind CD16.

Peripheral blood mononuclear cells (PBMC) were isolated from human peripheral blood buffy coats using density gradient centrifugation. NK cells (CD3−CD56+) were isolated from PBMC using negative selection with magnetic beads, and the purity of isolated NK cells was typically greater than 90%. Isolated NK cells were cultured overnight in media supplemented with IL-2 and IL-15 before they were used in cytotoxicity or IFNγ release assays. The following day NK cells were mixed with target cells and various concentrations of Constructs 1 and 2, or anti-BCMA and control antibodies.

As shown in FIG. 2A, the glycosylated bispecific antibodies, Constructs 1 and 2, exhibit superior ADCC activity by peripheral blood NK cells against H929 tumor cells, as compared to the activity of the parent anti-BCMA monoclonal antibody alone. Moreover, Constructs 1 and 2 are still able to mediate potent ADCC when binding to the CD16A receptor on NK cells is abrogated, as demonstrated in FIG. 2B using an aglycosylated version of the constructs that do not bind to the CD16A receptor. The results show that the present multispecific antigen-binding constructs enhanced ADCC by peripheral blood (pb) NK cells and retained this activity in the absence of binding to CD16A.

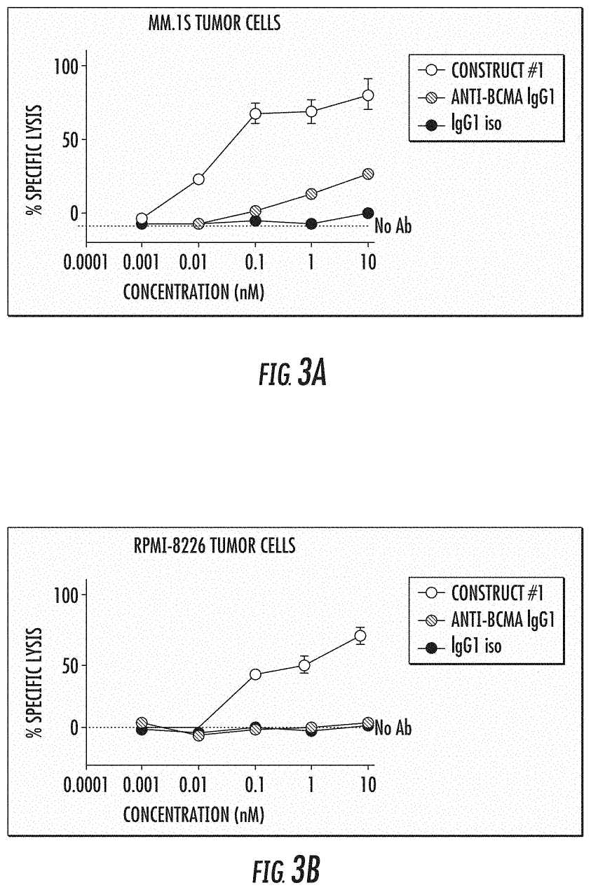

FIG. 3A and FIG. 3B show that Construct 1 enhanced ADCC in the presence of CD16A expression. FIG. 3A shows results using MM.1S tumor cells, which express the tumor antigen BCMA at medium levels, and FIG. 3B shows results using RPMI-8226 tumor cells, which express BCMA at low levels. When cells of the CD16A-expressing NK cell line were used as effector cells, Construct 1 demonstrated increased ADCC compared to the BCMA monoclonal antibody or negative control.

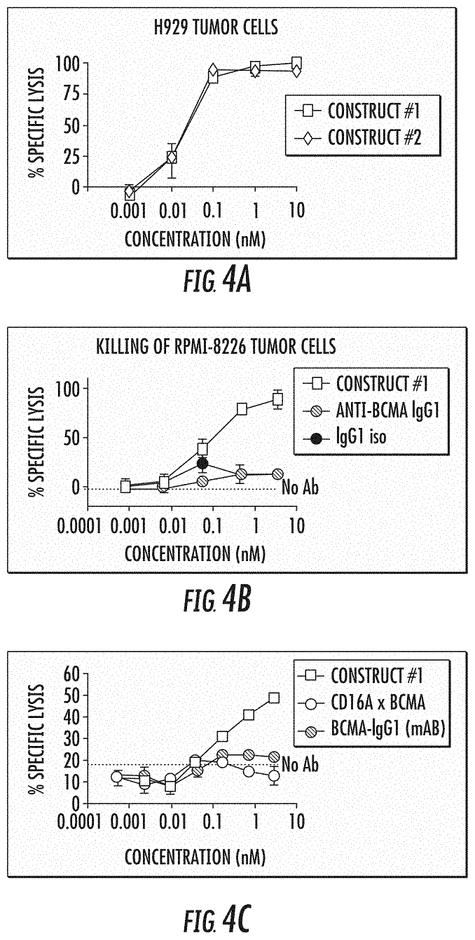

Finally, NK cell lines that do not express CD16 were used as effector cells. FIG. 4A and FIG. 4B show that the present multispecific antigen-binding constructs targeting BCMA and NKp30 induced ADCC in the absence of CD16A expression. FIG. 4A and FIG. 4C show results using H929 tumor cells, which express the tumor antigen BCMA at high levels, and FIG. 4B shows results using RPMI-8226 tumor cells, which express BCMA at low levels. When CD16A-negative NK cell lines (KHYG-1 in FIG. 4A and FIG. 4C, NK cell line in FIG. 4B) were used as effector cells, multispecific antigen-binding constructs targeting BCMA and NKp30 induced tumor-cell killing by CD16-negative NK cells compared to the BCMA monoclonal antibody (FIG. 4B and FIG. 4C), IgG1 isotype control (FIG. 4B), or CD16A-BCMA bispecific construct (FIG. 4C).

### Example 3

### Multispecific Antigen-Binding Constructs Targeting BCMA and NKp30 Exhibit Superior Activity

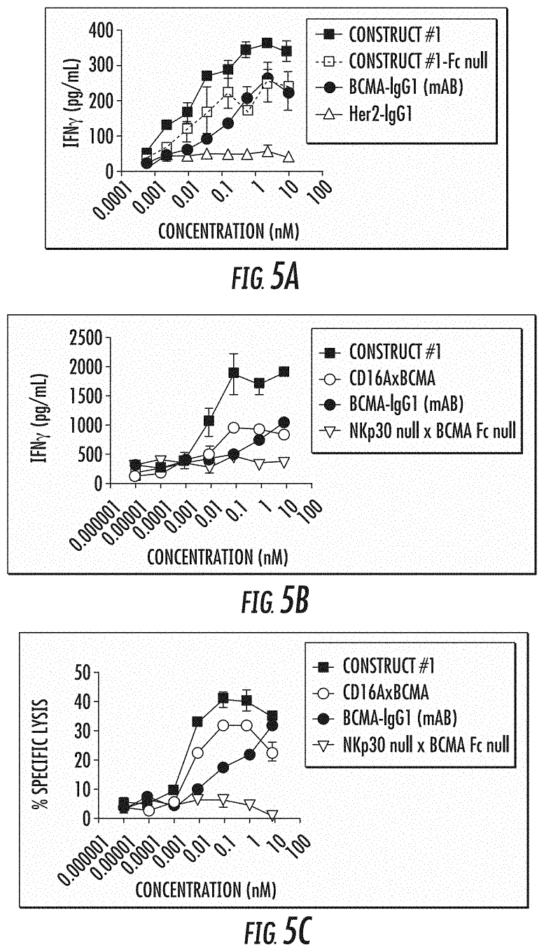

To analyze the effect of the multispecific antigen-binding constructs targeting BCMA and NKp30 that target both NKp30 and CD16, various constructs were studied (FIG. 5A, FIG. 5B, and FIG. 5C). The multispecific antigen-binding constructs targeting BCMA and NKp30 that target both NKp30 and CD16 were shown to exhibit superior activity as measured by the amount of IFNγ produced or the percentage of specific lysis.

FIG. 5A shows results using H929 tumor cells, and FIG. 5B and FIG. 5C show results using MM.1S tumor cells, which express BCMA at low levels. The multispecific antigen-binding constructs targeting BCMA and NKp30 exhibited superior activity to an NKp30-BCMA Fc-null construct (FIG. 4A), the BCMA monoclonal antibody (FIG. 5A, FIG. 5B, and FIG. 5C), a Her2 IgG1 isotype control (in FIG. 4A), a CD16-BCMA bispecific construct (FIG. 5B and FIG. 5C), and an NKp30 null-BCMA Fc null construct (FIG. 5C).

### Example 4

### Multispecific Antigen-Binding Constructs Targeting BCMA and NKp30 Retain IFNγ Production and ADCC Function in the Presence of High, Medium, or Low BCMA-Expressing Tumor Cells

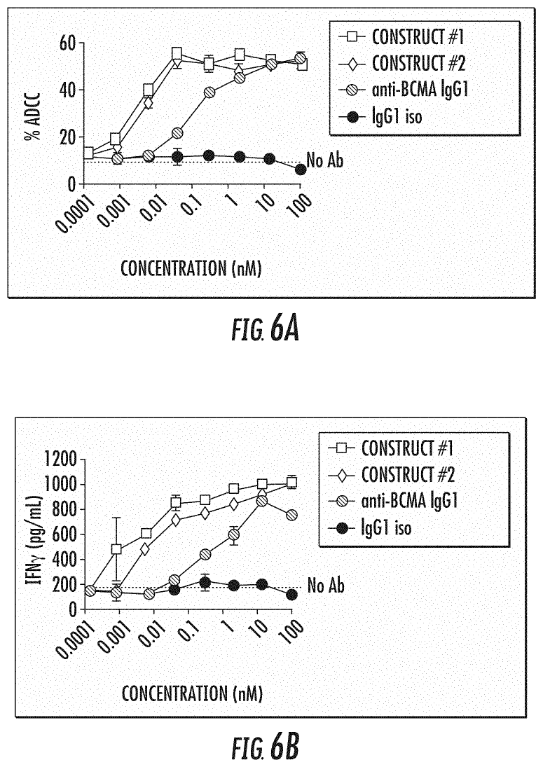

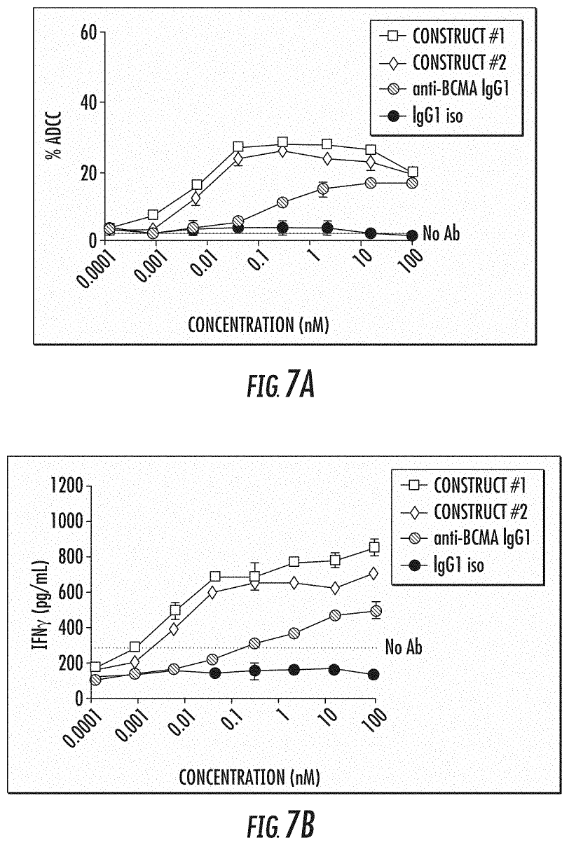

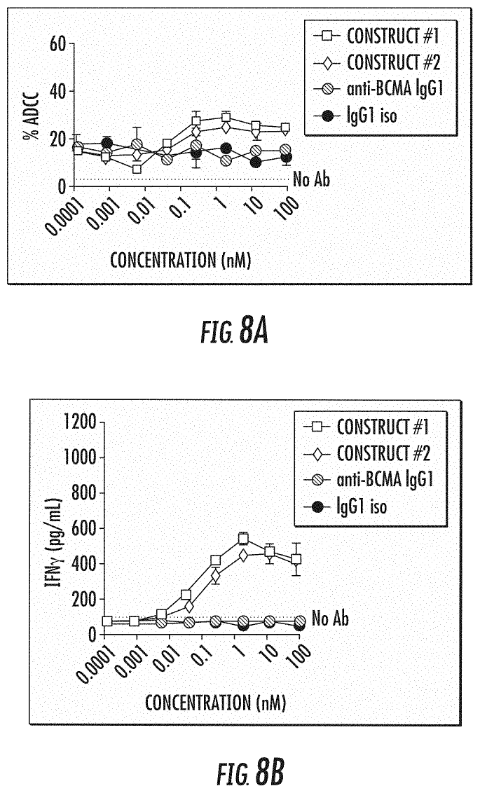

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytotoxicity or IFNγ release assays. The following day NK cells were mixed with target cells H929, which were determined by flow cytometry to express BCMA at about 130,000-150,000 copies per cell (FIG. 6A and FIG. 6B); MM.1S, which were determined by flow cytometry to express BCMA at about 90,000-100,000,000 copies per cell (FIG. 7A and FIG. 7B); or RPMI 8226, which were determined by flow cytometry to express BCMA at about 30,000-40,000 copies per cell (FIG. 8A and FIG. 8B). NK cells and target cells were used at an effector:target (E:T) ratio of 5:1 with an antibody dilution range starting from 10 nM with 1/5 dilution (n=8 concentrations). Cytotoxicity assays were carried out in 96 U bottom well plates for 4 hours at 37° C., whereas the IFNγ assay was carried out in 96 U bottom well plates for 18 hours at 37° C.

After incubation, killing of target cells was measured by release of lactate dehydrogenase (LDH) into the media from damaged cells as a biomarker for cellular cytotoxicity and cytolysis using the PIERCE™ LDH Cytotoxicity Assay Kit (ThermoFisher Scientific, Waltham, Mass.) according to manufacturer's instructions. All experimental conditions were analyzed in triplicate and the percentage of specific lysis was determined as follows: 100×(experimental value −effector cells spontaneous control−target cells spontaneous control)/target cell maximum control−target cells spontaneous control). IFNγ released by NK cells into the media was measured by Elisa.

The results show that, in the presence of multispecific binding constructs having a domain that binds NKp30 and a domain that binds BCMA, NK cells have superior capacity to cause cell lysis and to promote release of the cytokine IFNγ at most concentrations as compared to NK cells in the presence of a BCMA monoclonal antibody or in the presence of an hIgG1 isotype control antibody. The effect is shown in three types of cells, H929 (FIG. 6A and FIG. 6B), MM.1S (FIG. 7A and FIG. 7B), and RPMI-8226 (FIG. 8A and FIG. 8B), which were determined using flow cytometry to express about 130,000-150,000 copies of BCMA per target cell, about 90,000-100,000 copies of BCMA per target cell, and about 30,000-40,000 copies of BCMA per target cell, respectively. Taken together, the data show that the multispecific binding constructs are effective, even at low levels of BCMA on the target cells.

### Example 5

### Multispecific Antigen-Binding Constructs do not Activate NK Cells in Absence of Tumor Cells

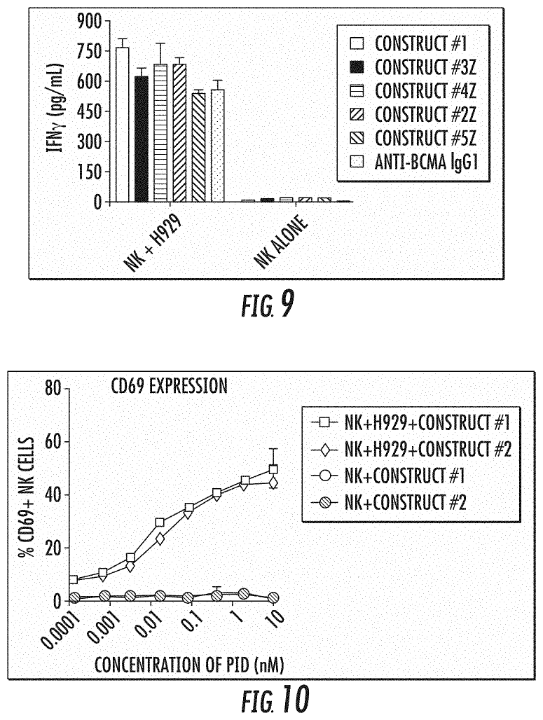

NK cells activated with IL-2 and IL-15 cytokines (FIG. 9) or resting NK cells (FIG. 10 and FIG. 11) were incubated overnight with or without H929 cancer cells (effector:target ratio 1:1) in the presence of several multispecific antigen-binding constructs (such as Construct 1) or the anti-BCMA IgG1 monoclonal antibody control at a concentration of 10 nM (FIG. 9) or at various concentrations of antibodies (FIG. 10 and FIG. 11). After incubation, supernatant was collected and IFNγ was measured by Elisa (Biolegend) (FIG. 9). Expression of CD69 (FIG. 10) and CD137 (FIG. 11) by NK cells was assessed in flow cytometry. In the absence of cancer cells, the multispecific antibody binding constructs were unable to activate NK cells, as measured by IFNγ release, CD69 and CD137 expression.

### Example 6

Multispecific Antigen-Binding Constructs Described Herein Activate NK Cells Even without Prior Stimulation with Cytokines

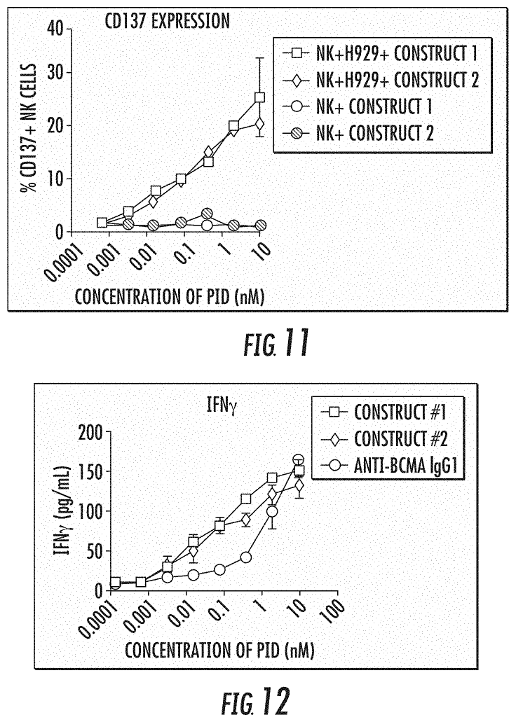

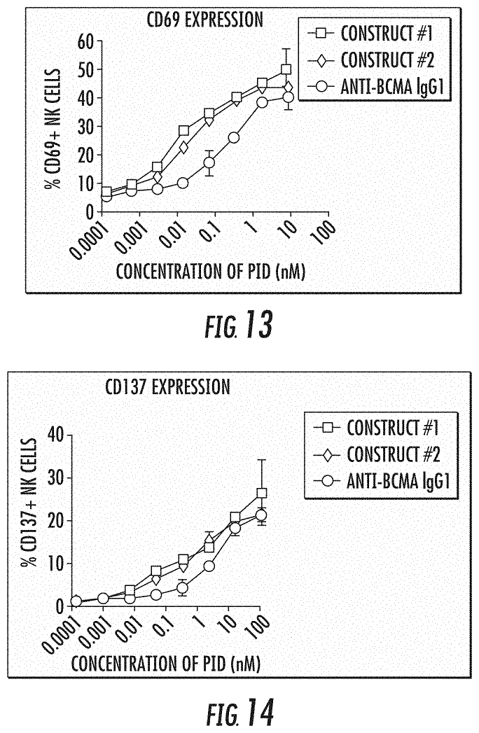

Resting NK cells were incubated overnight at 37° C. with BCMA positive target cells H929 at effector:target (E:T) ratio 1:1 in the presence of one of several multispecific antigen-binding constructs (such as Construct 1) or the anti-BCMA IgG1 monoclonal antibody control at various concentrations. After incubation, supernatant was collected and IFNγ was measured by Elisa (Biolegend) (FIG. 12). Expression of CD69 and CD137 by NK cells (FIG. 13 and FIG. 14, respectively) was assessed by flow cytometry. These data show that the BCMA-NKp30 antigen-binding constructs activate NK cells, even without prior stimulation with cytokines.

### Example 7

### Multispecific Antigen-Binding Constructs Described Herein Potently Induce Target Cell Lysis

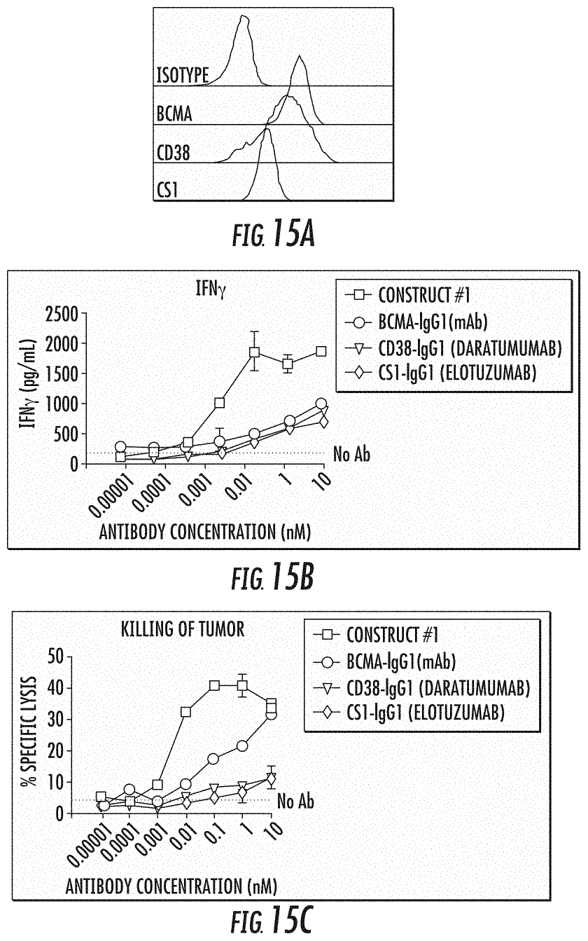

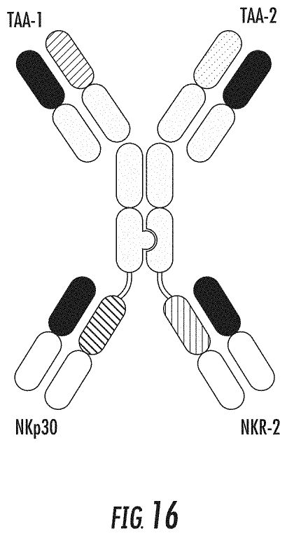

To further analyze the effect of the NKp30-BCMA multispecific antigen-binding constructs, the constructs were used in comparison to certain conventional antibodies that are currently undergoing clinical testing (FIG. 15A, FIG. 15B, and FIG. 15C). FIG. 15A reflects the surface expression of tumor antigens BCMA, CD38, and CS1 on MM.1S tumor cells. The multispecific construct exhibited increased IFNγ production (FIG. 15B) and superior ADCC activity (FIG. 15C) against tumor cells, when tested alongside an anti-BCMA IgG1 antibody, daratumumab which targets CD38, and elotuzumab which targets CS1. These data demonstrate that the NKp30-BCMA multispecific antigen-binding constructs described herein have better efficacy than the conventional antibodies.

### Example 8

### Effect of BCMA NKp30 Bispecific Antibodies in an Immunodeficient Xenograft Mouse Model

NSG™ mice (Jackson Labs, Bar Harbor, Me.) are inoculated with 8×10⁶ BCMA expressing target cells (e.g., H929, MM.iS, or RPMI-8226). Every 4-5 days after the inoculation, irradiated NK cells genetically modified to express CD16 are administered to the mice. Animal survival, body weight, and plasma are monitored following administration of multispecific binding constructs and monospecific binding constructs that have a BCMA binding domain and an NKp30 binding domain.

### Example 9

### Effect of BCMA NKp30 Bispecific Antibodies in Non-Human Primates

In Cynomolgus monkeys, BCMA is a B cell marker, present on plasma cells and the majority of peripheral blood B cells, unlike in humans, in which BCMA is present on plasma cells but not peripheral B cells. Thus, the efficacy of BCMA binding constructs can be assessed by detecting the depletion of B cells in peripheral blood and plasma cells in bone marrow. To assess efficacy, PK, PD, and non-GLP toxicity, Cynomolgus monkeys are administered 50 mg/Kg to 0.1 mg/Kg of the multispecific binding construct having a BCMA binding domain and an NKp30 binding domain or a corresponding monospecific binding construct that binds BCMA. B-cell depletion is assessed 24 h-72 h after treatment by monitoring B-cell count and serum IgG levels.

### Example 10

### Multispecific Antigen-Binding Constructs Targeting Her2 and NKp30 Exhibit Superior Activity

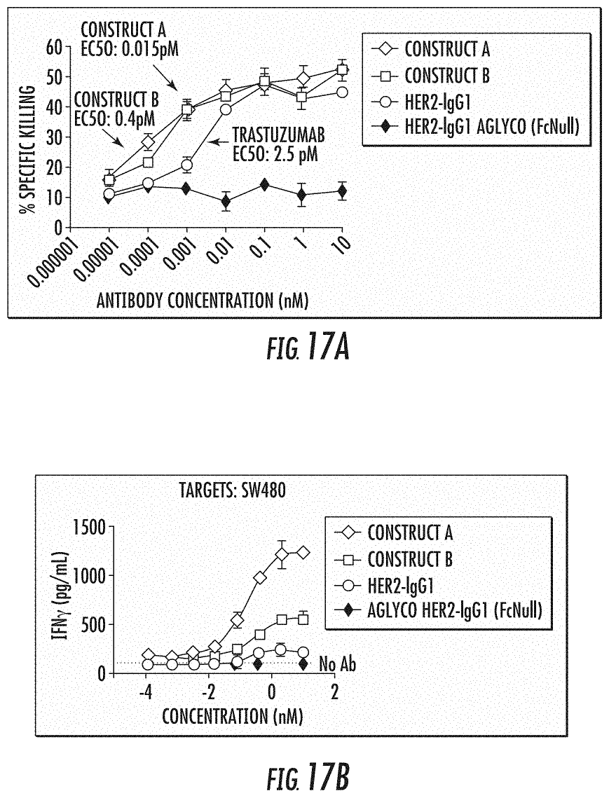

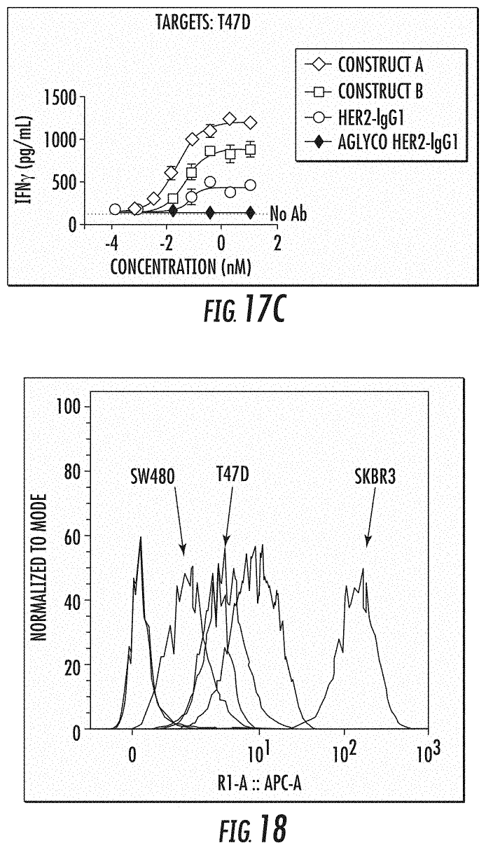

To analyze the effect of the multispecific antigen-binding constructs targeting Her2 and NKp30, various constructs were studied (FIG. 17A, FIG. 17B, and FIG. 17C). Constructs A and B, each a multispecific antigen-binding construct that specifically binds human Her2 and human NKp30, were generated using standard molecular biology techniques. The constructs contain an anti-Her2 IgG1 antibody, in which the heavy chain of the antibody is a fusion protein further comprising at its C-terminus the heavy chain variable region of an anti-NKp30 antibody, which is connected to the Fc region of the anti-Her2 antibody by way of a poly-GGGS (SEQ ID NO:22) linker. The light chains for the anti-Her2 portion and the anti-NKp30 portion of the construct are identical. Construct A, the structure for which is represented by the illustration of FIG. 1, comprises the same heavy chain and light chain region of the anti-NKp30 antibody portion of Constructs 1 and 2.

The multispecific antigen-binding constructs targeting Her2 and NKp30 were shown to exhibit superior activity as measured by the percentage of specific lysis.

FIG. 17A shows results using SKBR3 tumor cells. The multispecific antigen-binding constructs targeting Her2 and NKp30 (Construct A and Construct B) exhibited superior activity to an anti-Her2 monoclonal antibody (trastuzumab, FIG. 17A) and an aglycosylated version of the anti-Her2 monoclonal antibody (FIG. 17A).

### Example 11

### Multispecific Antigen-Binding Constructs Targeting Her2 and NKp30 Retain IFNγ Production Activity in the Presence of High, Medium, or Low Her2-Expressing Tumor Cells

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytotoxicity or IFNγ release assays. The following day NK cells were mixed with target cells SKBR3 (high expression of Her2), SW40 (low level of expression of Her2), or T47D (intermediate to low expression of Her2), the Her2 expression levels for which were determined by flow cytometry (see FIG. 18). NK cells and target cells were used at an effector:target (E:T) ratio of 5:1 with an antibody dilution range starting from 10 nM with 1/10 or 1/5 dilution (n=8 concentrations). After incubation, IFNγ released by NK cells into the media was measured by ELISA.

The results show that, in the presence of multispecific binding constructs having a domain that binds NKp30 and a domain that binds Her2, NK cells have superior capacity to promote release of the cytokine IFNγ at most concentrations as compared to NK cells in the presence of a Her2 monoclonal antibody or in the presence of any one of several anti-Her2 IgG1 monoclonal antibodies (FIG. 17B and FIG. 17C). The effect is shown in all three cell types above. Taken together, the data show that the multispecific binding constructs are effective, even at low levels of Her2 on the target cells.

### Example 12

### Multispecific Antigen-Binding Construct 1 Targeting Both NKp30 and BCMA Depletes Plasma Cells in the Bone Marrow of Cynomolgus Macaques

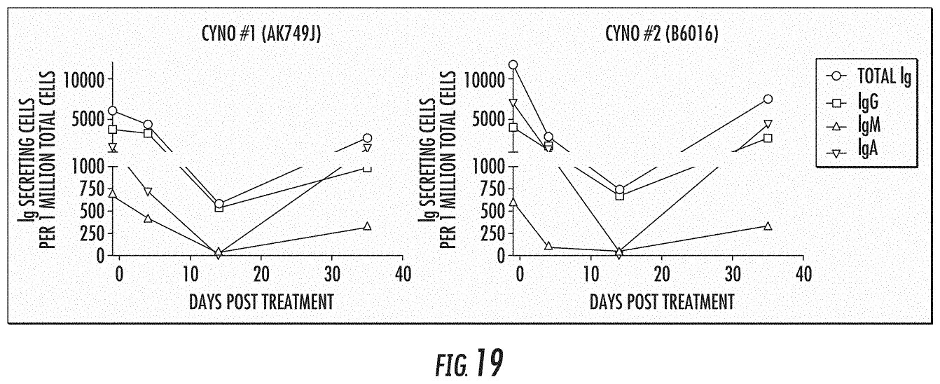

Adult cynomolgus monkeys (n=2) received a single intravenous injection of construct 1 at 30.25 mg/kg. The number of immunoglobulin-secreting cells in the bone marrow of treated monkeys was measured over time using ELISPOT assays specific for IgM, IgG, and IgA. It was assumed that the major part of immunoglobulin-secreting cells in bone marrow are plasma cells, as an estimate of the number of plasma cells in bone marrow. As shown in FIG. 19, a strong decrease in bone marrow plasma cells (>80%) at 2 weeks post-treatment was observed, followed by a rebound 3 weeks later in both treated animals.

### Example 13

### Multispecific Antigen-Binding Construct 1 Targeting Both NKp30 and BCMA Decreases Serum IgM in the Plasma of Cynomolgus Macaques

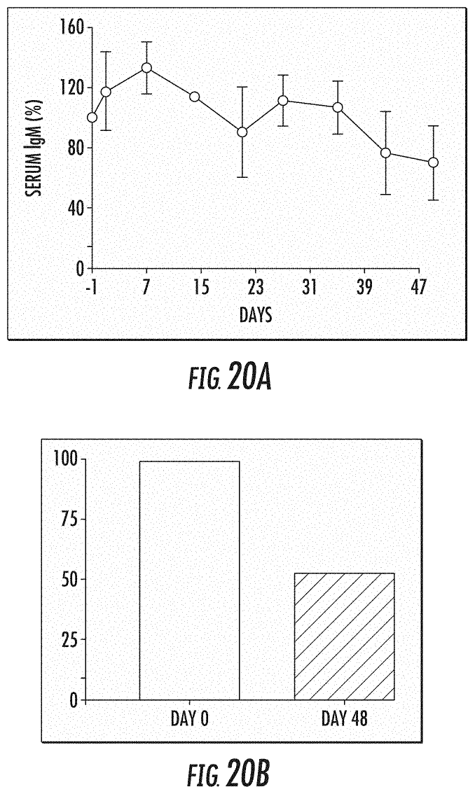

A single dose of construct 1 was given intravenously to cynomolgus macaques at 30.25 mg/kg. Using ELISA, the level of plasma IgM in the peripheral blood of treated cynomolgus macaques was measured over time. This assay was specific for cynomolgus IgM, and a standard cynomolgus IgM was used to calculate the IgM concentration in blood of treated monkeys. A strong decrease in plasma IgM starting was observed 5 weeks post-treatment. Data representative of 3 independent experiments. As shown in FIG. 20, multispecific antigen-binding construct 1 targeting both NKp30 and BCMA, induces a decrease of serum IgM in plasma of treated cynomolgus macaques.

### Example 14

### Multispecific Antigen-Binding Construct 1 Targeting Both NKp30 and BCMA Induces In-Vivo NK-Cell Expansion in Treated Monkeys

Using flow cytometry, the absolute number of NK cells in the peripheral blood of treated monkeys was measured over time, including cell-blood count for absolute-number calculation in cynomolgus monkeys administered intravenous construct 1 at 30.25 mg/kg at day 0. Cynomolgus NK cells were gated on CD45+/CD3−/CD14−/CD20− cell population with exclusion of dead cells. Using flow cytometry, the percentage of NK cells among the leukocyte population in bone marrow of the same treated monkeys was measured over time.

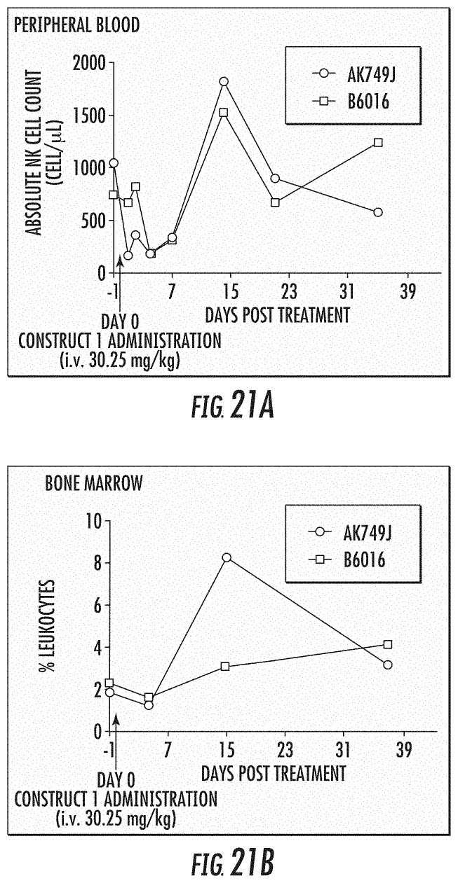

As indicated in FIG. 21A, NK cells expanded in the blood of treated monkeys with a maximum peak at about 14 days post-treatment. NK cells also expanded in bone marrow of treated monkeys (FIG. 21B), although this expansion was less for monkey B6016 than for AK749J.

### Example 15

### Multispecific Antigen-Binding Construct 1 Targeting Both NKp30 and BCMA Induces NK-Cell Activation in Monkey AK749J

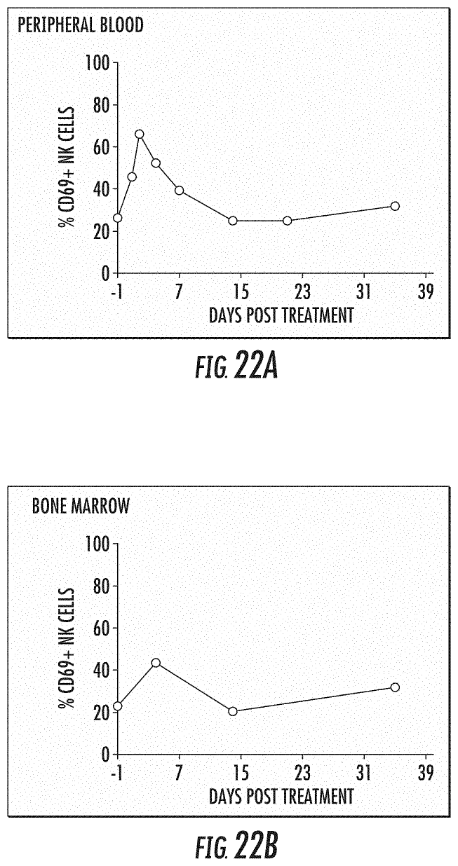

Using flow cytometry, the level of CD69 expression on NK cells in the peripheral blood or bone marrow of cynomolgus monkeys administered intravenous construct 1 at 30.25 mg/kg at day 0 was measured over time. Cynomolgus NK cells were gated on CD45+/CD3−/CD14−/CD20− cell population with exclusion of dead cells. As indicated by CD69 expression, NK cells were activated in the blood (FIG. 22A) and bone marrow (FIG. 22B) of monkey AK749J. It is noted that monkey B6016 had a high CD69 expression (>70%) before treatment and maintained a high CD69 expression during the course of treatment.

### Example 16

### Multispecific Antigen-Binding Construct 1 Targeting Both NKp30 and BCMA Displays a Typical IgG1-Like Pharmacokinetic Profile

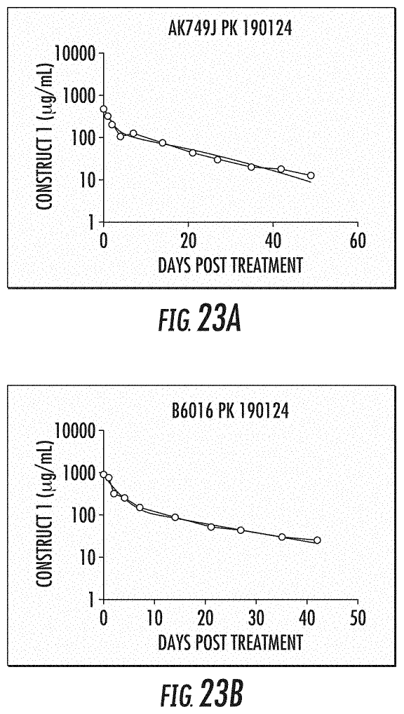

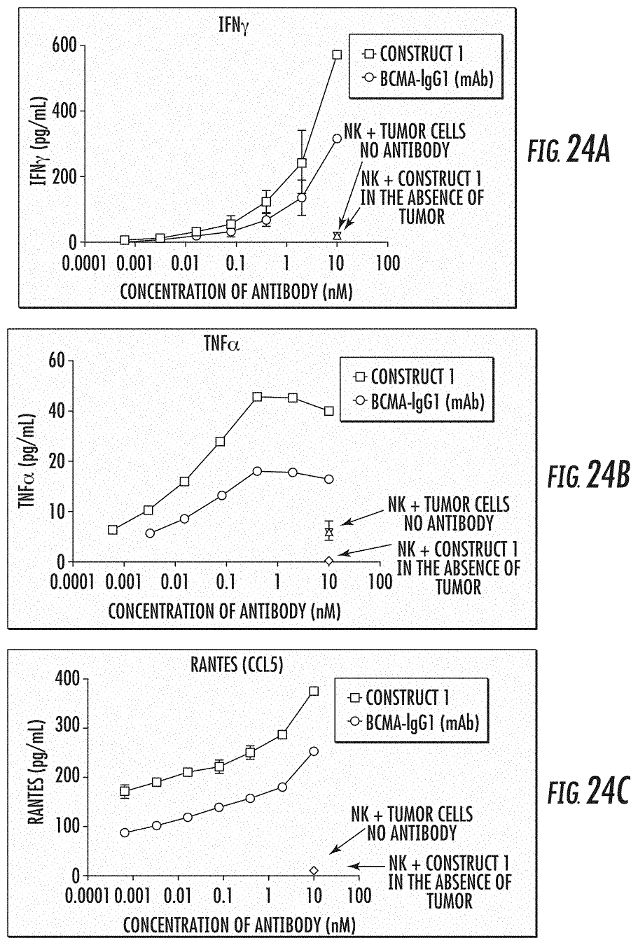

Plasma was collected after a single IV injection of construct 1, and levels of construct 1 in blood of treated monkeys was measured using an antigen-specific ELISA. Using a two-phase decay model, it was estimated that the β-phase half-life was ˜16 days for both monkeys, as shown in FIG. 23.

### Example 17

### Multispecific Antigen-Binding Construct Targeting NKp30 and BCMA Promotes Higher Production of IFNγ, TNFα, and Rantes by NK Cells Only in the Presence of Tumor Cells.

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Freshly isolated NK cells were mixed with target cells H929 at an effector:target (E:T) ratio of 1:1 with an antibody dilution range starting from 10 nM with 1/5 dilution (n=7 concentrations). The following day supernatants from the cultures were collected, and IFNγ, TNFα, and Rantes concentrations were measured by ELISA. As demonstrated herein in FIG. 24, construct 1 promotes higher production of cytokines by fresh resting NK cells as compared to BCMA-IgG1 monoclonal, and does not induce production of cytokines by NK cells in the absence of tumor cells.

### Example 18

Multispecific Antigen-Binding Construct Targeting NKp30 and BCMA Induces Activation and Cytotoxicity Towards MM Tumor Cells by Peripheral NK Cells from Myeloma Patients

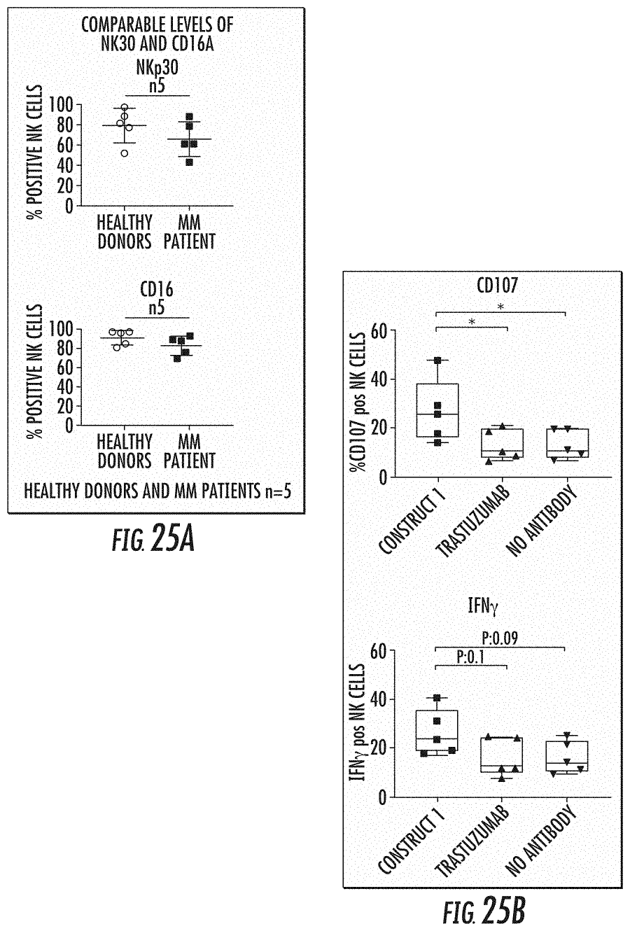

The frequencies of NKp30^{pos} or CD16^{pos} NK cells were assessed by flow cytometry using PBMCs isolated from healthy (n=5) or multiple myeloma patients (n=5) with different disease status (FIG. 25A). PBMCs from the same MM patients used in FIG. 25A were tested against MM.1S tumor cells in the presence of 10 nM of construct 1, or Trastuzumab (used as an isotype control), or no antibody in the presence of monensin and brefeldin A. NK cells degranulation and IFNγ production were measured using flow cytometry by gating on NK (CD56P″, CD3″g) CD107P″ cells and NK (CD56^{pos}, CD3^{neg}) IFNγ positive cells. As shown in FIG. 25A and FIG. 25B expression of NKp30 and Cd16A on PBMC from MM patients is comparable to healthy individuals, and NK cells from MM patients display high functionality in response to construct 1.

### Example 19

Multispecific Antigen-Binding Construct Targeting NKp30 and BCMA Induces NK-Cell Killing of Autologous Myeloma Cells from Bone Marrow of a Multiple Myeloma Patient

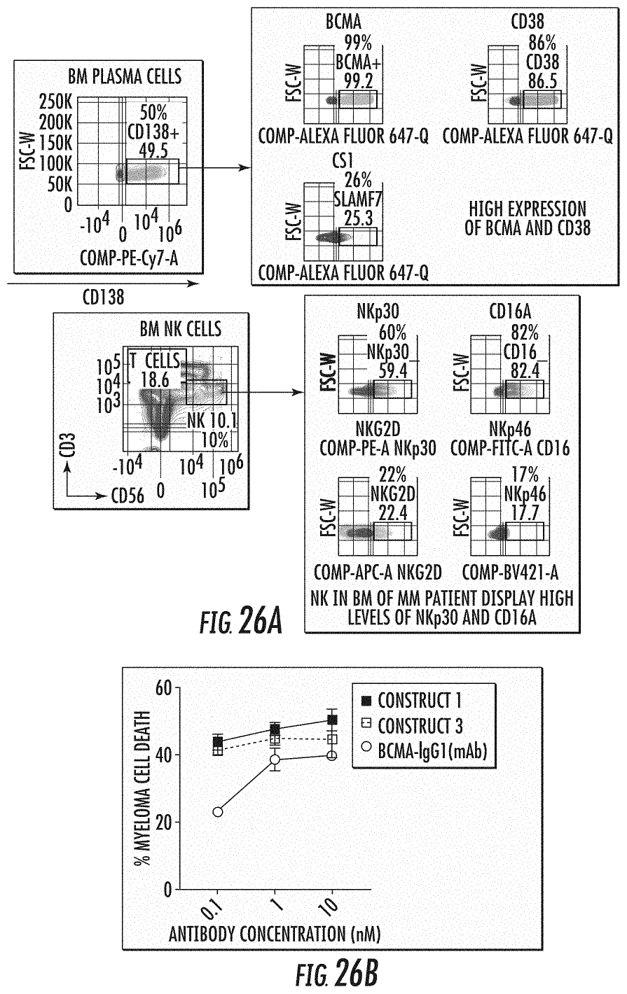

Bone marrow cells were stained with a panel of antibodies to measure expression of BCMA, CS1, and CD38 on bone marrow plasma cells (CD138^{pos}) from a patient newly diagnosed with multiple myeloma. Frequencies of NK cells in the BM of this patient, as well as expression of NKp30, CD16A, NKG2D and NKp46 on NK cells, was assessed via flow cytometry by gating on lymphocytes and CD56^{pos},CD3^{neg} cells, as shown in FIG. 26A. FIG. 26B shows death of autologous bone marrow plasma cells in the presence of construct 1, construct 3, an aglycosylated (Fc null) version of construct 1, and BCMA-IgG1 mAb as measured by flow cytometry as decreased frequency of CD138^{pos} multiple myeloma cells. Data was normalized to control wells without antibody. When bone marrow cells were tested in the presence of construct 1 or construct 3 or BCMA-IgG1 mAb, death of malignant bone marrow cells was observed over no antibody control at a higher extent with construct 1 and its aglycosylated version, construct 3, as compared to a BCMA-IgG1 monoclonal control.

### Example 20

### An Affinity Matured Multispecific Antigen-Binding Construct Targeting NKp30 and BCMA has Improved Binding in the Presence of the Soluble BCMA Ligand, APRIL

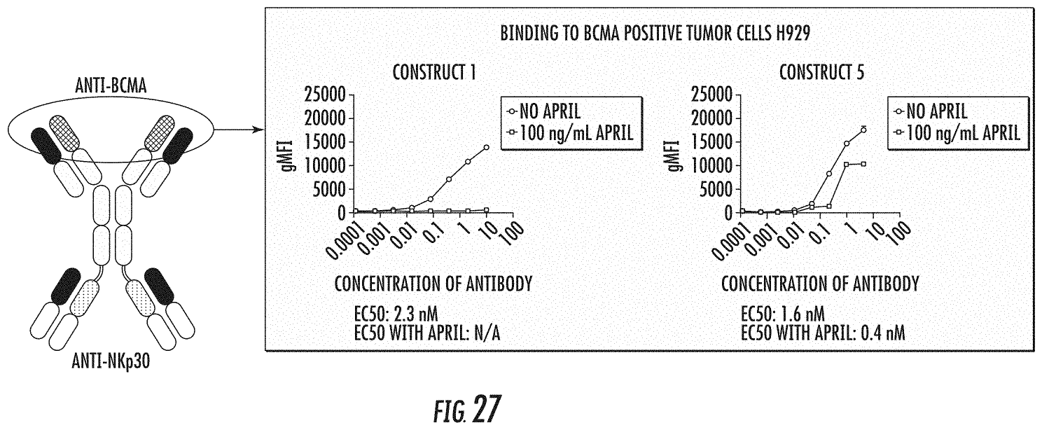

Anti-BCMA-IgG1, construct 5 (a variant of multispecific antigen-binding construct 1 having an affinity matured BCMA arm) and construct 1 were mixed with 100 ng/ml of recombinant human APRIL and incubated with H929 (BCMA^{pos}) cancer cells for 45 minutes. Cells were washed and stained with anti-human IgG F(ab)₂ for 15 minutes. Binding data was assessed by flow cytometry. As shown in FIG. 27, multispecific antigen-binding construct 5, a variant of multispecific antigen-binding construct 1 having an affinity matured BCMA arm, can bind to BCMA positive tumor cells, even in the presence of soluble APRIL.

### Example 21

### Activity of an Affinity Matured Multispecific Antigen-Binding Construct Targeting NKp30 and BCMA is not Impacted by the Presence of the Soluble BCMA Ligands, APRIL and BAFF

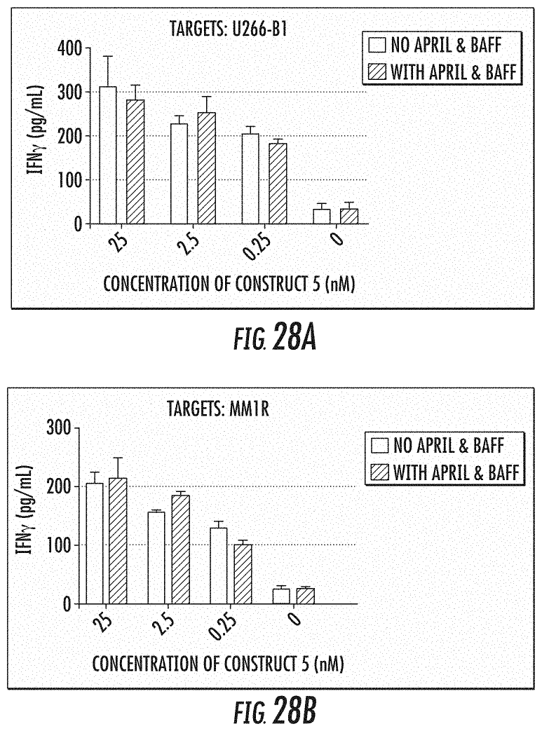

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. The following day NK cells were mixed with target cells U266-B1 and MM1R. NK cells and target cells were used at an effector:target (E:T) ratio of 2:1 with an antibody dilution range starting from 25 nM with 1/10 dilution (n=3 concentrations). ELISA assays for IFNγ were carried out in 96 U bottom well plates after 48 hours at 37° C. As demonstrated herein in FIG. 28, activity of construct 5 (a variant of multispecific antigen-binding construct 1 having an affinity matured BCMA arm) is not affected by the presence of soluble April and BAFF (BCMA ligands), both of which are found in the serum of multiple myeloma patients.

### Example 22

### Activity of an Affinity Matured Multispecific Antigen-Binding Construct Targeting NKp30 and BCMA is not Impacted by the Presence of Soluble BCMA

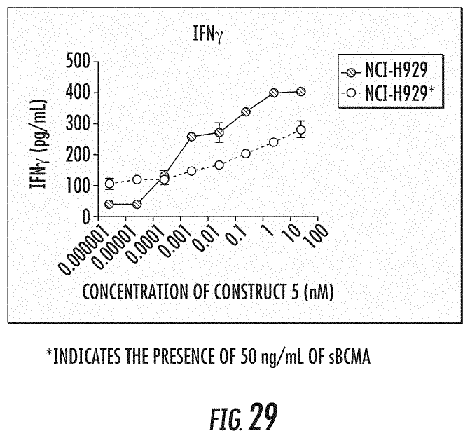

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. The following day NK cells were mixed with target H929 cells at an effector:target (E:T) ratio of 1:1 with an antibody dilution range starting from 25 nM with 1/10 dilution (n=8 concentrations) with or without 50 ng/ml soluble BCMA. ELISA assays for IFNγ were carried out in 96 U bottom well plates after 48 hours at 37° C. As shown in FIG. 29, construct 5, having an affinity matured BCMA arm, shows activity in the presence of high levels of soluble BCMA

### Example 23

### Activity of an Affinity Matured Multispecific Antigen-Binding Construct Targeting NKp30 and BCMA Towards BCMA^{low} Target Cells is Retained

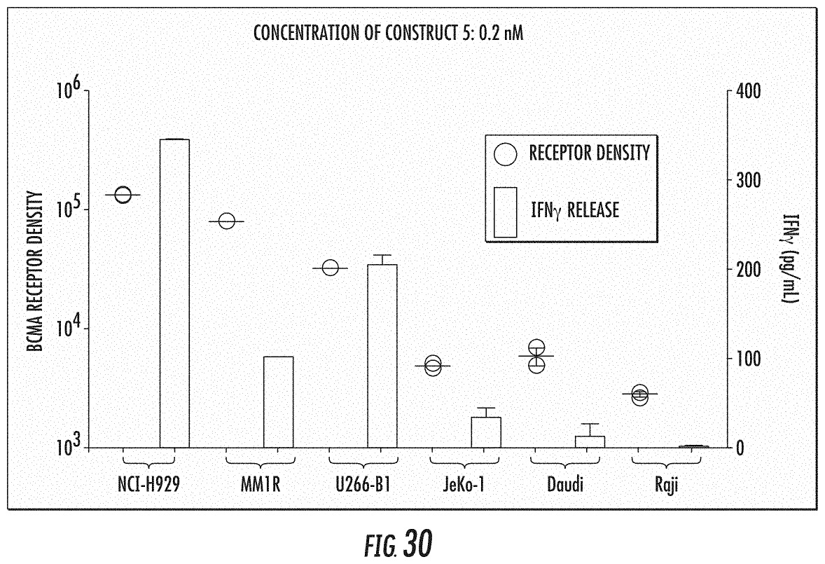

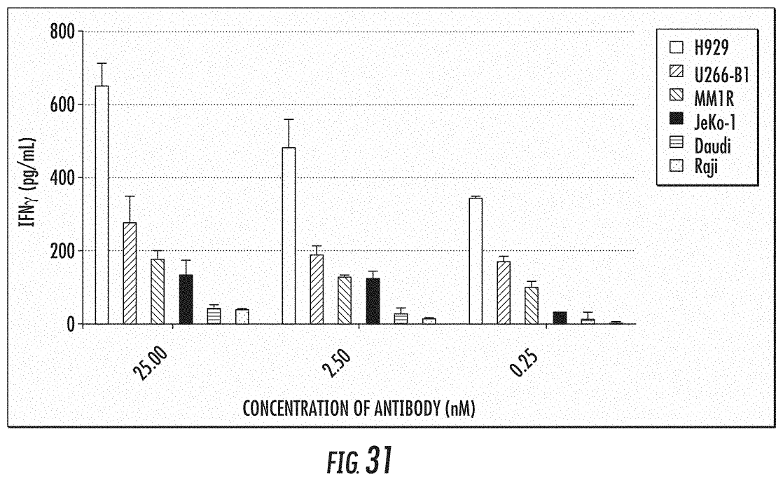

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. The following day NK cells were mixed with target tumor cells expressing different levels of BCMA copies/cell at an effector:target (E:T) ratio of 2:1. ELISA assays for IFNγ were carried out in 96 U bottom well plates after 48 hours at 37° C. Data shows that construct 5 shows selectivity towards BCMA^{high} expressing tumor cells and retained activity towards BCMA^{low} expressing tumor cells but did not show activity towards tumor cells (e.g., Raji) expressing less than 2,000 BCMA copies/cells. As shown in FIG. 30, construct 5 shows selectivity towards BCMA^{high} expressing tumor cells and retained activity towards BCMA^{low} expressing tumor cells, but did not show activity towards tumor cells (e.g., Raji) expressing less than 2,000 BCMA copies/cell.

### Example 24

### An Affinity Matured Multispecific Antigen-Binding Construct Targeting NKp30 and BCMA Shows Selectivity Towards Higher Expressing BCMA Tumor Cells and Retains Activity in Low BCMA-Expressing Cells

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. The following day NK cells were mixed with different target tumor cells expressing different levels of BCMA copies/cell at an effector:target (E:T) ratio of 2:1 with an antibody dilution range of construct 5 starting from 25 nM with 1/10 dilution (n=3 concentrations). ELISA assays for IFNγ were carried out in 96 U bottom well plates after 48 hours at 37° C. As shown in FIG. 31, construct 5 showed activity even at picomolar concentrations in inducing IFNγ production by NK cells co-cultured with a range of cell lines varying in levels of BCMA expression.

### Example 25

### An Affinity Matured Multispecific Antigen-Binding Construct Targeting NKp30 and BCMA Activates NK Cells in the Presence of Lymphoma Cells Expressing Low Copies/Cells of BCMA

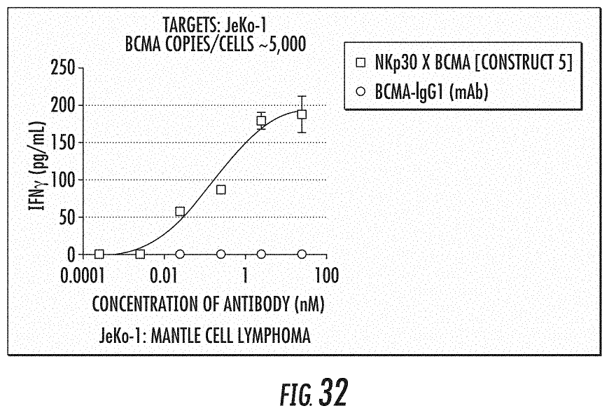

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. The following day NK cells were mixed with JeKo-1 target lymphoma cells expressing 5000 BCMA copies/cell at an effector:target (E:T) ratio of 2:1 with an antibody dilution range of construct X starting from 25 nM with 1/10 dilution (n=6 concentrations). ELISA assays for IFNγ were carried out in 96 U bottom well plates after 48 hours at 37° C. As shown in FIG. 32, construct 5 activates NK cells in the presence of lymphoma cells expressing low copies/cells of BCMA, whereas BCMA-IgG1 monoclonal does not show any activity.

### Example 26

### The Proliferative Signal Induced by an Affinity Matured Multispecific Antigen-Binding Construct Targeting NKp30 and BCMA is Mainly Dependent on NKp30 Engagement

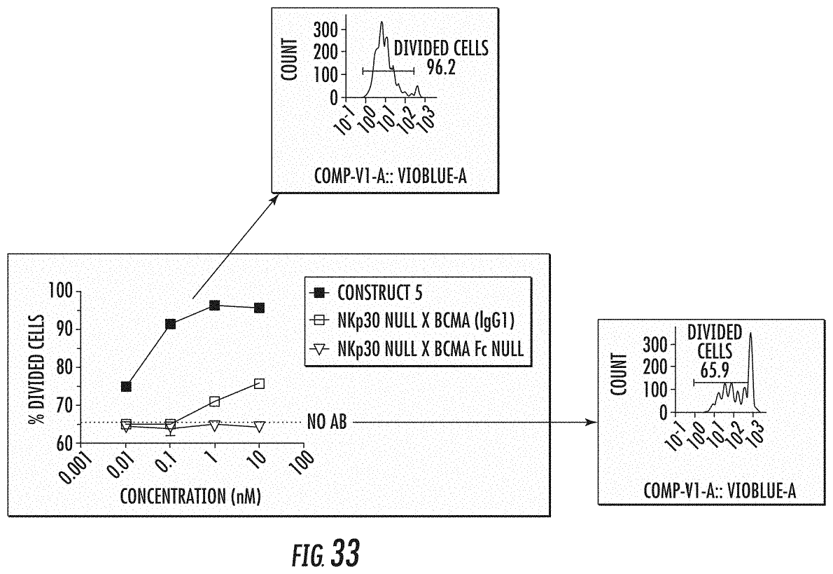

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in cytokine release assays. Primary NK cells were labeled with CELLTRACE™ Violet (CTV) and incubated with H929 cancer cells in the presence of IL-2 and construct 5, a bispecific that binds BCMA, CD16 (through the Fc), but not NKp30 [NKp30 null×BCMA IgG1], or a bispecific that binds BCMA, but not CD16 or NKp30 [NKp30 null×BCMA Fc null]. NK cell proliferation was measured by flow cytometry assessing CTV dilution after 5 days. As shown in FIG. 33, construct 5 induces NK cells proliferation at higher extent than bispecifics that bind CD16A but not NKp30 [NKp30 null×BCMA (IgG1)], or those that do not bind CD16A and NKp30 [NKp30 null×BCMA Fc null] indicating that the proliferative signal of construct 5 is mainly dependent on NKp30 engagement.

### Example 27

### Afucosylation of Fc Improves Activity of Multispecific Antigen-Binding Constructs Targeting NKp30 and BCMA

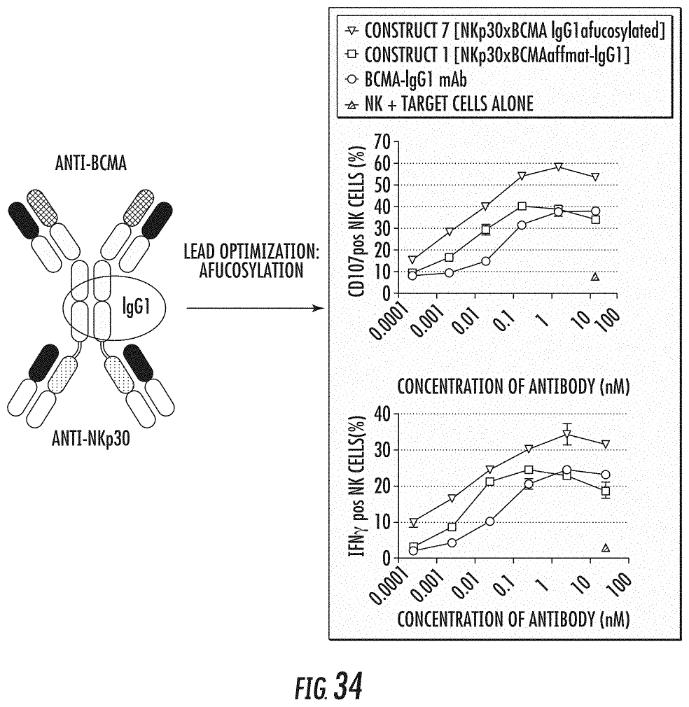

Peripheral blood mononuclear cells (PBMC) were isolated from human peripheral blood buffy coats using density gradient centrifugation. NK cells (CD3−CD56+) were isolated from PBMC using negative selection with magnetic beads. The purity of isolated NK cells typically achieved with the method was greater than 90%. Isolated NK cells were cultured overnight in media supplemented with IL-2 and IL-15 before they were used in degranulation assays. The following day NK cells were mixed with target H929 cells at an effector:target (E:T) ratio of 1:1 with an antibody dilution range starting from 25 nM with 1/10 dilution (n=6 concentrations) CD107 expression on NK cells, as well as intracellular IFNγ, were measured by flow cytometry, and percentage of NK cells expressing CD107 and intracellular IFNγ. As shown in FIG. 34, using construct 7, having an afucosylated Fc on the multispecific antigen-binding construct 1, improves activity.

### Example 28

Nkp30 is expressed on γδ T cells in the bone marrow of multiple myeloma patients

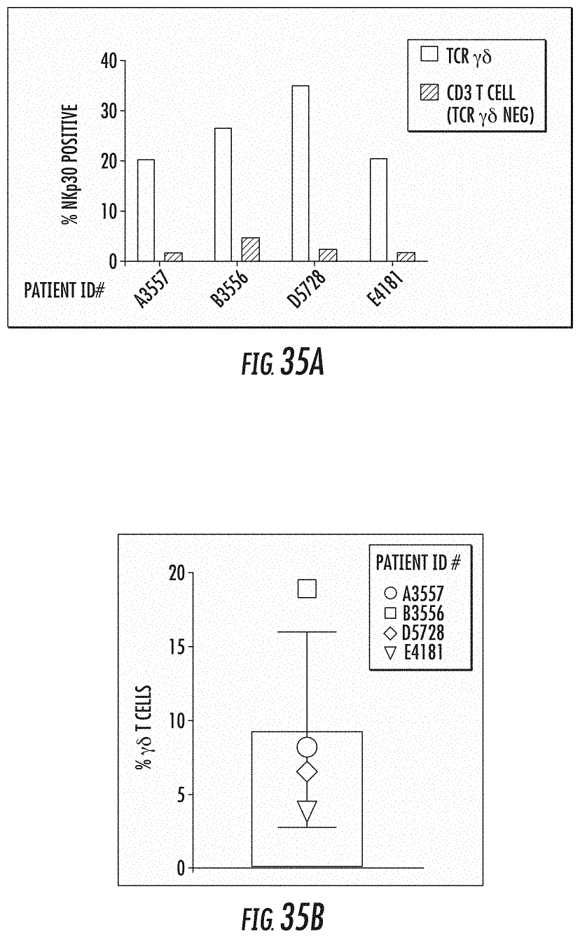

Bone marrow cells were stained with a panel of antibodies, and frequencies of γδ T cells in the BM of four multiple myeloma patients, as well as expression of NKp30 by γδ T cells, were assessed via flow cytometry by gating on lymphocytes, CD3^{pos},TCR γδ^{pos} cells, as shown in FIG. 35A. FIG. 35B shows the frequency of TCR γδ T cells in the bone marrow aspirates of each patient. These data indicate that NKp30 expressing γδ T cells in multiple myeloma patients can also be activated using the multispecific antigen-binding constructs targeting NKp30 and BCMA described herein.

### Example 29

### Multispecific Antigen-Binding Construct 5 Blocks Proliferation of BCMA Positive Tumor Cells

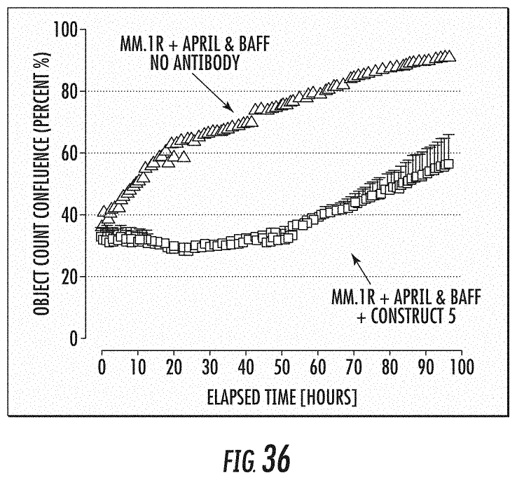

Construct 5 was added at a concentration of 10 pM in culture with BCMA positive tumor cells, MM.1R, with and without 100 ng/ml of APRIL and 10 ng/ml of BAFF, in the absence of NK cells. Tumor cell proliferation was monitored over a time period of 100 hours by fluorescence imaging using an INCUCYTE® Live Cell analysis system. As shown in FIG. 36, multispecific antigen-binding construct 5, a variant of multispecific antigen-binding construct 1 having an affinity matured BCMA arm, blocked proliferation of BCMA positive tumor cells, MM.1R, even in the presence of 100 ng/ml APRIL and 1 ng/ml of BAFF and in the absence of NK cells.

### Example 30

### Multispecific Antigen-Binding Construct 5 Induces Both Greater Proliferation and Cell Division of NK Cells

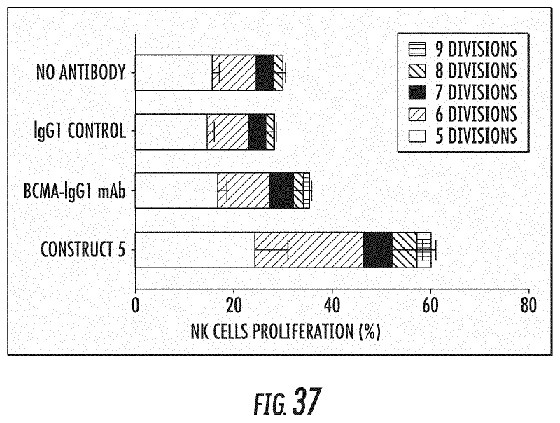

NK cells were cultured for 5 days with H929 labeled with CELLTRACE™ Violet (CTV) in the presence of IL-2 and Construct 5 (1 nM), a BCMA-IgG1 antibody (1 nM), IgG1 control antibody (trastuzumab) (1 nM), or no antibody. Proliferation was measured by dilution of CELLTRACE™ Violet, and the number of cell divisions that NK cells had undergone was calculated using FLOWJO software (FlowJo, LLC; Ashland, Oreg.). As shown in FIG. 37, multispecific antigen-binding construct 5, a variant of multispecific antigen-binding construct 1 having an affinity matured BCMA arm, induced proliferation and cell division of NK cells to a higher extent than a monoclonal BCMA-IgG1 antibody, as measured by dilution of CELLTRACE™ Violet.

### Example 31

### Multispecific Antigen-Binding Construct 5 Induces Potent NK Cell Killing of MM.1R Tumor Cells Even in the Presence of BCMA Ligands, APRIL and BAFF

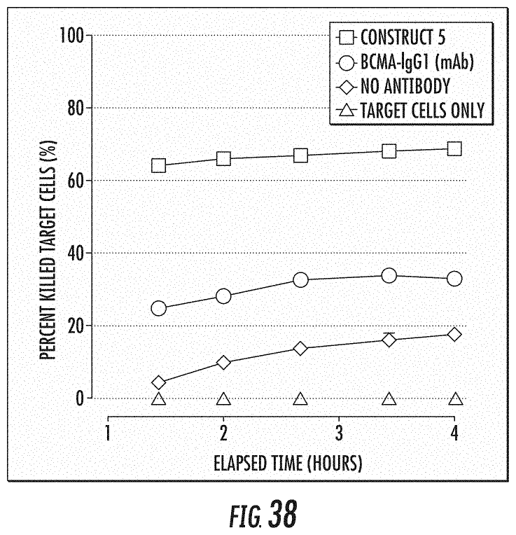

Primary NK cells were co-cultured with BCMA positive tumor cells, MM.1R, that were transduced with Lentiviral NucLight green, at an Effector:Target ratio of 2:1 in the presence or absence of Construct 5 [100 pM], a BCMA-IgG1 mAb [100 pM] or no antibody. Tumor cell killing by NK cells was monitored by fluorescence imaging using an INCUCYTE® Live Cell analysis system over a period of 4 hours. Percent killing was calculated by normalizing to the number of target cells only control group. As shown in FIG. 38, multispecific antigen-binding construct 5, a variant of multispecific antigen-binding construct 1 having an affinity matured BCMA arm, induced potent killing by NK cells of BCMA positive tumor cells, MM.1R, in the presence of 100 ng/ml APRIL and 1 ng/ml of BAFF.

### Example 32

### Multispecific Antigen-Binding Construct 1 Induces Potent Killing of BCMA Tumor Cells Expressing a Wide Range of Antigen Expression

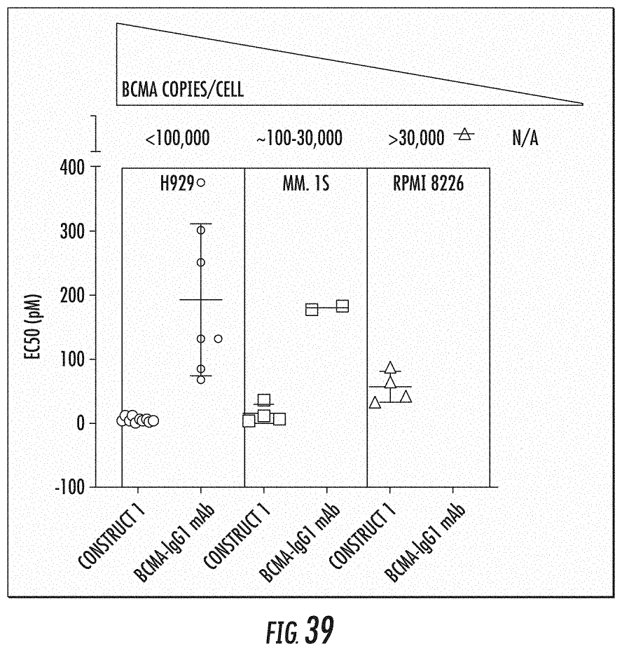

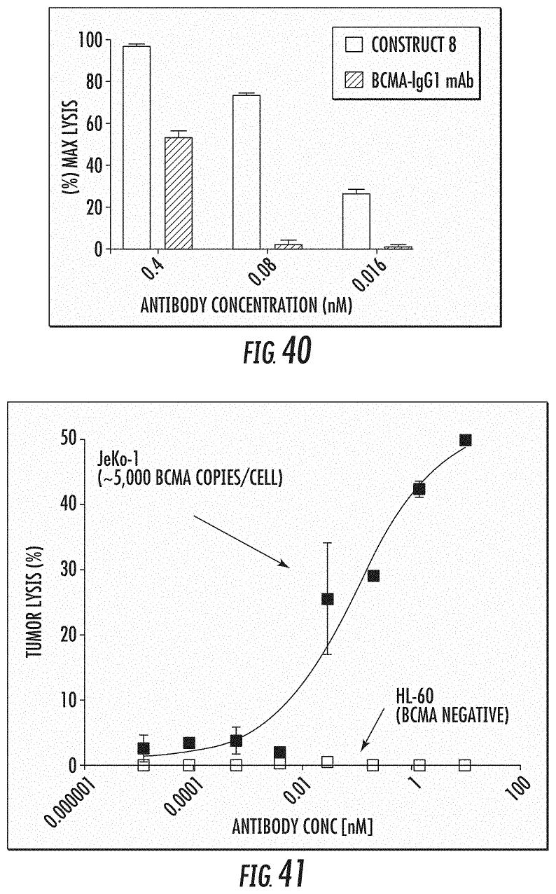

Primary NK cells from different donors were co-cultured for 4 hours with multiple myeloma tumor cell lines that express different levels of BCMA in the presence of serially diluted Construct 1 or BCMA-IgG1 monoclonal antibody. Killing of tumor cells was assessed by a 4-hour LDH (lactate dehydrogenase) release assay that was performed using an effector:target ratio of 5:1. EC50 values for Construct 1 or BCMA-IgG1 monoclonal antibody-induced lysis of target H929, MM.1S and RPMI 8226 tumor cells with effector NK cells from different donors are shown. Data show that Construct 1 was much more potent than the monoclonal BCMA-IgG1 antibody. EC50 value of BCMA-IgG1 tested on RPMI 8226 is shown as not applicable (n/a), because the BCMA-IgG1 did not activate NK cells toward BCMA. As shown in FIG. 39, multispecific antigen-binding construct 1, targeting both NKp30 and BCMA, induces potent killing of BCMA tumor cells expressing a wide range of antigen expression, compared to a monoclonal BCMA-IgG1 antibody.

### Example 33

### Multispecific Antigen-Binding Construct 8 Shows Activity in the Absence of CD16A Engagement

A 4-hour LDH (lactate dehydrogenase) killing assay using primary NK cells was performed. Killing of H929 tumor cells in the presence of different doses of antibodies is shown as a percentage of maximum lysis of H929 tumor cells induced by Construct 8 [an Fc null version of Construct 5] or BCMA-IgG1 mAb. As shown in FIG. 40, multispecific antigen-binding construct 8, an aglycosylated (Fc null) variant of multispecific antigen-binding construct 5, shows activity even in the absence of CD16A engagement.

### Example 34

### Multispecific Antigen-Binding Construct 5 Induces NK Cell Killing of BCMA-Low JeKo-1 Tumor Cells, but not BCMA-Negative HL-60 Tumor Cells

Primary NK cells were co-cultured with BCMA-low (positive) JeKo-1 tumor cells or BCMA-negative HL-60 tumor cells for 4 hours in the presence of Construct 5 serially diluted (5-fold). As shown in FIG. 41, killing of JeKo-1 tumor cells in the presence of Construct 5 was observed in a dose-dependent manner, whereas no killing of BCMA-negative HL-60 tumor cells was detected.

### Example 35

### Multispecific Antigen Binding Constructs Targeting Her2 and NKp30 Exhibit Superior Cytotoxic Activity

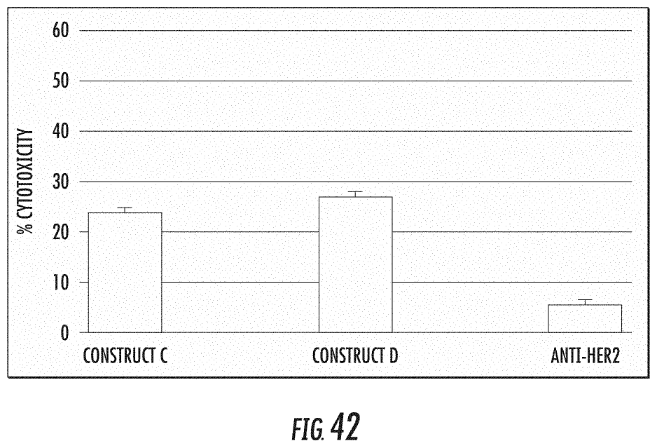

To analyze the effects of multispecific antigen binding constructs targeting Her2 and NKp30, various constructs were studied (FIG. 42). Constructs C and D, each a multispecific antigen binding construct that specifically binds human Her2 and human NKp30, were generated using standard molecular biology techniques. For example, Construct D comprises an anti-Her2 IgG1 antibody, in which the heavy chain of the antibody is a fusion protein further comprising at its C-terminus the heavy chain variable region of an anti-NKp30 antibody, which is connected to the Fc region of the anti-Her2 antibody by way of a poly-GGGS (SEQ ID NO:22) linker. Briefly, the Her2-positive SK-BR3 cell line was used with the effector KHYG-1 NK cell line. The target cells and NK cells were mixed with 1 nM of Construct C, Construct D, or trastuzumab, and incubated for 4 hours at 37° C. in a 5% CO2 incubator. LDH (lactate dehydrogenase) release from the target SK-BR3 cells was measured as the read-out. The multispecific antigen binding constructs targeting Her2 and NKp30 were shown to exhibit superior activity as measured by the percentage of specific lysis, when compared to the anti-Her2 monoclonal antibody, trastuzumab, alone.

### Example 36

### Multispecific Antigen Binding Constructs Targeting BCMA and NKp30 Promote Target Cell Killing by γδ T-Cells

Gamma Delta (γδ) T-cells were sorted using a FACsARIA according to positive expression of CD3 and γδ TCR from human peripheral blood mononuclear cells (PBMCs) isolated from a fresh buffy coat, yielding about 2.5×10⁴ total enriched γδ T-cells. γδ T-cells were cultured at 1×10⁶/mL with 1 μg/mL of anti-CD3, 0.5 μg/mL of anti-CD28, 100-IU of IL-2, and 1 μg/mL of PHA for approximately 12 days. The final yield of γδ T-cells was ˜6×10⁶.

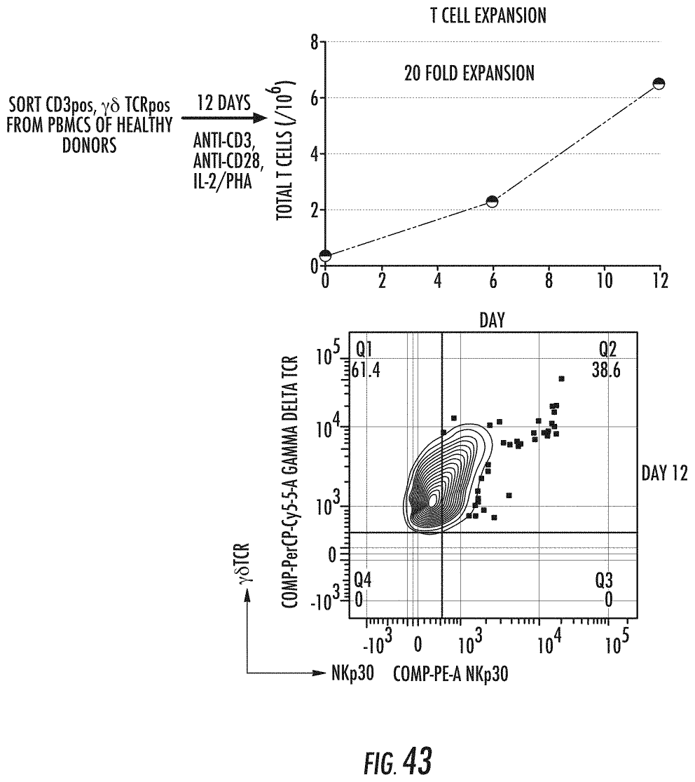

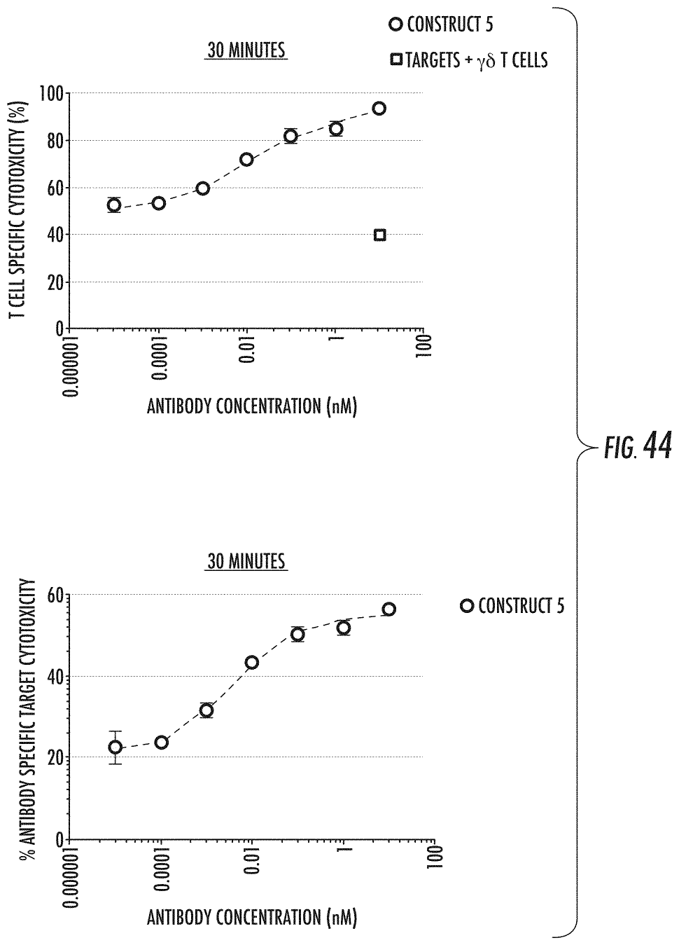

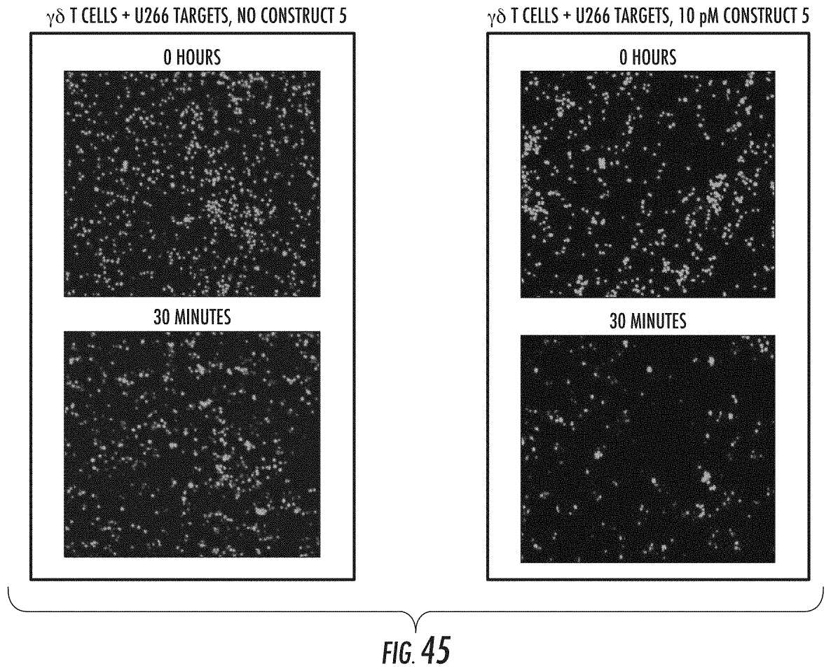

Phenotypic and functional assessment of the expanded γδ T-cells is shown at FIG. 43. As shown, roughly 40% of the expanded γδ T-cells are positive for NKp30 expression.

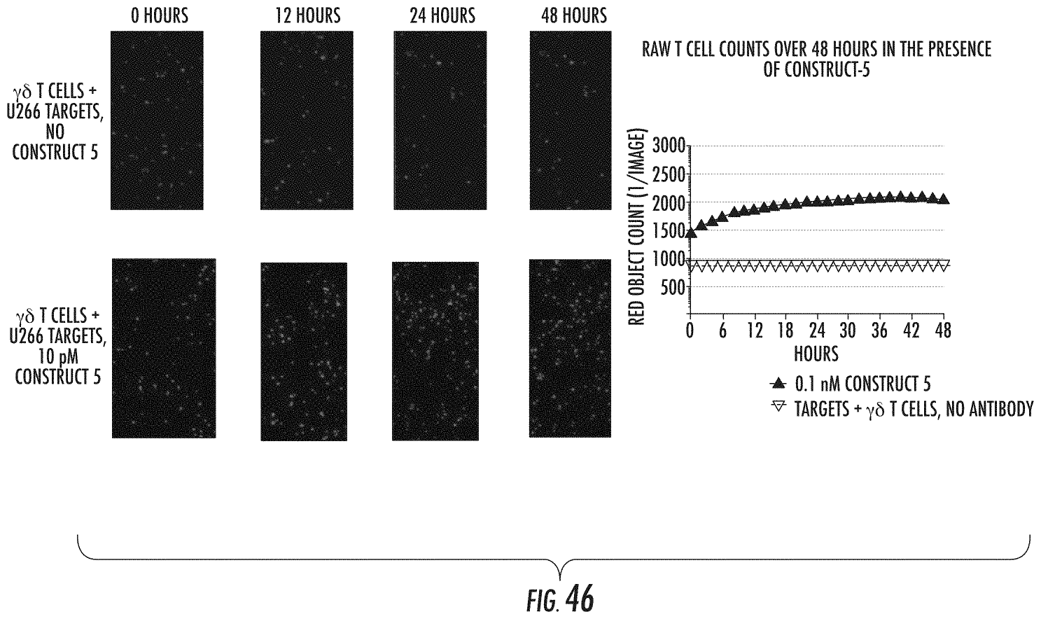

Further phenotypic and functional assessments of expanded γδ T-cells was performed. γδ T-cells were counted and stained with CFSE Far Red at a 10 nM concentration, and plated at 100,000 cells per well in RP10 plus 10 IU of IL-2. U266 Target cells were stained with cell trace CFSE and plated at 50,000 target cells per well in RP10, resulting in a final 2:1 Effector:Target ratio. Serial dilutions of construct 5 were prepared in RP10 media starting at 10 nM and serially diluted 10-fold, to give a total of seven data points. Plates were incubated in the Incucyte Zoom with images taken every 2 hours in the green and red channel. As shown in FIG. 44, T-cell specific cytotoxicity of target U266 cells shows a dose-dependent effect in the presence of Construct 5. Furthermore, only in the presence of Construct 5 is killing of target U266 cells observed after 30 minutes, as demonstrated in FIG. 45. In both the left and right panels, target U266 cells are labeled in green with Invitrogen Cell Trace CFSE. After 30 minutes, in the presence of only 10 pM Construct 5, there is a noticeable decrease in target U266 cells (right panel) demonstrating that engagement of NKp30 on γδ T-cells by Construct 5 promotes target cell killing. In addition to the decrease in the target U266 cells, there is an increase in γδ T-cells after 48 hours, as shown in FIG. 46. In both the upper and lower panels, γδ T-cells are labeled with CFSE Far Red, but only in the presence of 10 pM Construct 5 (bottom panels) is an increase seen in the number of γδ T-cells, demonstrating that engagement of NKp30 on γδ T-cells by Construct 5 increases γδ T-cell proliferation.

### Example 37

### Identification of Critical Binding Residues on NKp30

Antibody binding of NKp30 mutants was performed on a Forte Bio Octet Red384 system (Pall Forte Bio Corporation, Menlo Park, Calif.). His-tagged NKp30 mutants were captured directly from the conditioned medium onto HIS1K Octet sensor. The sensor was subsequently exposed to 100 nM test antibody. Data were processed using ForteBio's Data Analysis Software 7.0 and the binding responses were reported in the table.

To determine which amino acid residues within NKp30 are critical for the binding of mAb8, mAb10, and mAb11 to human NKp30, a combined alanine and arginine scanning mutagenesis analysis was performed to identify residues important for antibody binding to NKp30. NKp30 mutants were generated with single point mutations at selected amino acid residue positions known to be important for binding of an NKp30 ligand, B7-H6, to NKp30. These His-tagged NKp30 mutants were then tested for their ability to bind to mAb8, mAb10, and mAb11.

For mAb8, 150, S82, and L113 were identified as important amino acid residues for binding to NKp30, as mutating these residues resulted in a loss of binding to NKp30. For mAb10 and mAb11, I50 and L113 were identified as important amino acid residues for binding to NKp30, as mutating these residues resulted in a loss of binding to NKp30. FIG. 47A shows a Table with a partial amino acid sequence of NKp30 where residues comprising an epitope bound by mAb8, mAb10, and mAb11 are indicated in bold text. FIG. 47B also depicts X-ray crystallography images of human NKp30, with residues I50, S82, and L113 shown as spheres (left panel) and X-ray crystallography images of human NKp30 bound to B7-H6 with residues I50, S82, and L113 shown as spheres (right panel). These figures indicate that the NKp30 antigen-binding proteins described herein bind to a conformational or non-linear epitope of NKp30, thereby blocking ligand binding.

TABLE 1
Summary of Sequences

| SEQ | | |
|---|---|---|
| ID | | |
| NO. | Description | Sequence* |
| 1 | Variable light | DIQMTQSPSSLSASVGDRVTITCSASQDISNYLNWYQQKPGKA |
| | chain amino | PKLLIYYTSNLHSGVPSRFSGSGSGTDFTLTISSLQPEDFATYY |
| | acid sequence | CQQYRKLPWTFGQGTKLEIKR |
| | of a First | |
| | Antigen | |
| | Binding Unit | |
| | (anti-BCMA | |
| | antibody) | |
| 2 | Variable heavy | QVQLVQSGAEVKKPGSSVKVSCKASGGTFSNYWMHWVRQA |
| | chain amino | PGQGLEWMGATYRGHSDTYYNQKFKGRVTITADKSTSTAYM |
| | acid sequence | ELSSLRSEDTAVYYCARGAIYDGYDVLDNWGQGTLVTVSS |
| | of a First | |
| | Antigen | |
| | Binding Unit | |
| | (anti-BCMA | |
| | antibody) | |
| 3 | FLAG | DYKDDDDK |
| | antigenic tag | |
| 4 | Polyhistidine | HHHHHH |
| | antigenic tag | |
| 5 | Hemagglutinin | YPYDVPDYA |
| | antigenic tag | |
| 6 | Human BCMA | MLQMAGQCSQNEYFDSLLHACIPCQLRCSSNTPPLTCQRYCN |
| | amino acid | ASVTNSVKGTNAILWTCLGLSLIISLAVFVLMFLLRKINSEPLK |
| | sequence | DEFKNTGSGLLGMANIDLEKSRTGDEIILPRGLEYTVEECTCE |
| | | DCIKSKPKVDSDHCFPLPAMEEGATILVTTKTNDYCKSLPAAL |
| | | SATEIEKSISAR |
| 7 | Full-length | MAWMLLLILIIVIVHPGSCALWVSQPPEIRTLEGSSAFLPCSFNA |
| | Human NKp30 | SQGRLAIGSVTWFRDEVVPGKEVRNGTPEFRGRLAPLASSRFL |
| | amino acid | HDHQAELHIRDVRGHDASIYVCRVEVLGLGVGTGNGTRLVV |
| | sequence | EKEHPQLGAGTVLLLRAGFYAVSFLSVAVGSTVYYQGKCLT |
| | (including | WKGPRRQLPAVVPAPLPPPCGSSAHLLPPVPGG |
| | signal | |
| | sequence) | |
| 8 | Light chain | DIQMTQSPSSLSASVGDRVTITCRASQSISSYLNWYQQKPGKAPKL |
| | amino acid | LIYAASSLQSGVPSRFSGSGSGTDFTLTISSLQPEDFATYYCQQSYS |
| | sequence of | TPLTFGGGTKVEIKRTVAAPSVFIFPPSDEQLKSGTASVVCLL |
| | mAb 1-mAb10; | NNFYPREAKVQWKVDNALQSGNSQESVTEQDSKDSTYSLS |
| | Constructs 1-9 | STLTLSKADYEKHKVYACEVTHQGLSSPVTKSFNRGEC |
| 9 | Heavy chain | QVQLVQSGAEVKKPGASVKVSCKASGYTFSSHYVHWVRQAPGQG |
| | amino acid | LEWMGIIDPSGGSTSYAQKFQGRVTMTRDTSTSTVYMELSSLRSE |
| | sequence of | DTAVYYCARGRYDYGDYLGWFDPWGQGTLVTVSSASTKGPSVF |
| | Construct 1 | PLAPSSKSTSGGTAALGCLVKDYFPEPVTVSWNSGALTSG |
| | | VHTFPAVLQSSGLYSLSSVVTVPSSSLGTQTYICNVNHKPS |
| | | NTKVDKKVEPKSCDKTHTCPPCPAPELLGGPSVFLFPPKP |
| | | KDTLMISRTPEVTCVVVDVSHEDPEVKFNWYVDGVEVHN |
| | | AKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFL |
| | | YSKLTVDKSRWQQGNVFSCSVMHEALHNHYTQKSLSLSP |
| | | GGGGSGGGGSQVQLVQSGAEVKKPGASVKVSCKASGHTFTSYF |
| | | MHWVRQAPGQGLEWMGIINPSDDYANYAQKFQGRVTMTRDTST |
| | | STVYMELSSLRSEDTAVYYCATAIFDYWGQGTLVTVSSASTKGPS |
| | | VFPLAPSSKSTSGGTAALGCLVKDYFPEPVTVSWNSGALTS |
| | | GVHTFPAVLQSSGLYSLSSVVTVPSSSLGTQTYICNVNHKP |
| | | SNTKVDKKVEPKSCDKTHT |
| 10 | VH amino acid | QVQLVQSGAEVKKPGASVKVSCKASGYTFSSHYVHWVRQA |
| | sequence of | PGQGLEWMGIIDPSGGSTSYAQKFQGRVTMTRDTSTSTVYM |
| | anti-BCMA | ELSSLRSEDTAVYYCARGRYDYGDYLGWFDPWGQGTLVTV |
| | antibody | SS |
| | (mAb1; | |
| | Constructs 1, 2, | |
| | 3, and 4) | |
| 11 | CDRH1 amino | YTFSSHYVH |
| | acid sequence | |
| | of anti-BCMA | |
| | antibody | |
| | (mAb1, mAb4, | |
| | and mAb5; | |
| | Constructs 1, | |
| | 2, 3, and 4) | |
| 12 | CDRH2 amino | GIIDPSGGSTSYA |
| | acid sequence | |
| | of anti-BCMA | |
| | antibody | |
| | (mAb1; | |
| | Constructs 1, | |
| | 2, 3, and 4) | |
| 13 | CDRH3 amino | CARGRYDYGDYLGWFDPW |
| | acid sequence | |
| | of anti-BCMA | |
| | antibody | |
| | (mAb1, mAb5, | |
| | mAb6, and | |
| | mAb7; | |
| | Constructs 1, | |
| | 2, 3, 4, and 7) | |
| 14 | VH amino acid | QVQLVQSGAEVKKPGASVKVSCKASGHTFTSYFMHWVRQA |
| | sequence of | PGQGLEWMGIINPSDDYANYAQKFQGRVTMTRDTSTSTVYM |
| | anti-NKp30 | ELSSLRSEDTAVYYCATAIFDYWGQGTLVTVSS |
| | antibody | |
| | (mAb8; | |
| | Constructs 1, | |
| | 2, 3, and 4) | |
| 15 | CDRH1 amino | HTFTSYFMH |
| | acid sequence | |
| | of anti-NKp30 | |
| | antibody | |
| | (mAb8; | |
| | Constructs 1, | |
| | 2, 3, 4, 5, 6, | |
| | and 7) | |
| 16 | CDRH2 amino | GIINPSDDYANYA |
| | acid sequence | |
| | of anti-NKp30 | |
| | antibody | |
| | (mAb8; | |
| | Constructs 1, | |
| | 2, 3, 4, 5, 6, | |
| | and 7) | |
| 17 | CDRH amino | CATAIFDYW |
| | acid sequence | |
| | of anti-NKp30 | |
| | antibody | |
| | (mAb8; | |
| | Constructs 1, | |
| | 2, 3, 4, 5, 6, | |
| | and 7) | |
| 18 | VL amino acid | DIQMTQSPSSLSASVGDRVTITCRASQSISSYLNWYQQKPGKA |
| | sequence | PKLLIYAASSLQSGVPSRFSGSGSGTDFTLTISSLQPEDFATYY |
| | (mAb1- | CQQSYSTPLTFGGGTKVEIK |
| | mAb10; | |
| | Constructs 1, | |
| | 2, 3, 4, 5, 6, | |
| | and 7) | |
| 19 | CDRL1 amino | RASQSISSYLN |
| | acid sequence | |
| | (mAb1- | |
| | mAb10; | |
| | Constructs 1, | |
| | 2, 3, 4, 5, 6, | |
| | and 7) | |
| 20 | CDRL2 amino | AASSLQS |
| | acid sequence | |
| | (mAb1-mAb10; | |
| | Constructs | |
| | 1, 2, 3, 4, 5, 6, | |
| | and 7) | |
| 21 | CDRL3 amino | CQQSYSTPLTF |
| | acid sequence | |
| | (mAb1- | |
| | mAb10; | |
| | Constructs 1, | |
| | 2, 3, 4, 5, 6, | |
| | and 7) | |
| 22 | Exemplary | GGGGS |
| | linker amino | |
| | acid sequence | |
| 23 | Exemplary | GGGS |
| | linker amino | |
| | acid sequence | |
| 24 | Heavy chain | QVQLVQSGAEVKKPGASVKVSCKASGYTFSSHYVHWVRQAPGQG |
| | amino acid | LEWMGIIDPSGGSTSYAQKFQGRVTMTRDTSTSTVYMELSSLRSE |
| | sequence of | DTAVYYCARGRYDYGDYLGWFDPWGQGTLVTVSSASTKGPSVF |
| | Construct 2 | PLAPSSKSTSGGTAALGCLVKDYFPEPVTVSWNSGALTSG |
| | (Alternative | VHTFPAVLQSSGLYSLSSVVTVPSSSLGTQTYICNVNHKPS |
| | Heavy Chain | NTKVDKKVEPKSCDKTHTCPPCPAPELLGGPSVFLFPPKP |
| | of Construct 1) | KDTLMISRTPEVTCVVVDVSHEDPEVKFNWYVDGVEVHN |
| | | AKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFL |
| | | YSKLTVDKSRWQQGNVFSCSVMHEALHNHYTQKSLSLSP |
| | | GGGGSGGGGSQVQLVQSGAEVKKPGASVKVSCKASGHTFTSYF |
| | | MHWIRQAPGQGLEWMGIINPSDDYANYAQKFQGRVTMTRDTST |
| | | STVYMELSSLRSEDTAVYYCATAIFDYWGQGTPVTVSSASTKGPS |
| | | VFPLAPSSKSTSGGTAALGCLVKDYFPEPVTVSWNSGALTS |
| | | GVHTFPAVLQSSGLYSLSSVVTVPSSSLGTQTYICNVNHKP |
| | | SNTKVDKKVEPKSCDKTHT |
| 25 | Alternative VH | QVQLVQSGAEVKKPGASVKVSCKASGHTFTSYFMHWVRQAP |
| | amino acid | GQGLEWMGIINPSDDYANYAQKFQGRVTMTRDTSTSTVYME |
| | sequence of | LSSLRSEDTAVYYCATAIFDYWGQGTPVTVSS |
| | anti-NKp30 | |
| | antibody | |
| | mAb8 (mAb9; | |
| | Constructs 2 | |
| | and 4) | |
| 26 | Aglycosylated | QVQLVQSGAEVKKPGASVKVSCKASGYTFSSHYVHWVRQAPGQG |
| | heavy chain | LEWMGIIDPSGGSTSYAQKFQGRVTMTRDTSTSTVYMELSSLRSE |
| | amino acid | DTAVYYCARGRYDYGDYLGWFDPWGQGTLVTVSSASTKGPSVF |
| | sequence of | PLAPSSKSTSGGTAALGCLVKDYFPEPVTVSWNSGALTSG |
| | Construct 1 | VHTFPAVLQSSGLYSLSSVVTVPSSSLGTQTYICNVNHKPS |
| | (Construct 3) | NTKVDKKVEPKSCDKTHTCPPCPAPELLGGPSVFLFPPKP |
| | | KDTLMISRTPEVTCVVVDVSHEDPEVKFNWYVDGVEVHN |
| | | AKTKPREEQYASTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFL |
| | | YSKLTVDKSRWQQGNVFSCSVMHEALHNHYTQKSLSLSP |
| | | GGGGSGGGGSQVQLVQSGAEVKKPGASVKVSCKASGHTFTSYF |
| | | MHWIRQAPGQGLEWMGIINPSDDYANYAQKFQGRVTMTRDTST |
| | | STVYMELSSLRSEDTAVYYCATAIFDYWGQGTLVTVSSASTKGPS |
| | | VFPLAPSSKSTSGGTAALGCLVKDYFPEPVTVSWNSGALTS |
| | | GVHTFPAVLQSSGLYSLSSVVTVPSSSLGTQTYICNVNHKP |
| | | SNTKVDKKVEPKSCDKTHT |
| 27 | Aglycosylated | QVQLVQSGAEVKKPGASVKVSCKASGYTFSSHYVHWVRQAPGQG |
| | alternative | LEWMGIIDPSGGSTSYAQKFQGRVTMTRDTSTSTVYMELSSLRSE |
| | heavy chain | DTAVYYCARGRYDYGDYLGWFDPWGQGTLVTVSSASTKGPSVF |
| | amino acid | PLAPSSKSTSGGTAALGCLVKDYFPEPVTVSWNSGALTSG |
| | sequence of | VHTFPAVLQSSGLYSLSSVVTVPSSSLGTQTYICNVNHKPS |
| | Construct 1 | NTKVDKKVEPKSCDKTHTCPPCPAPELLGGPSVFLFPPKP |
| | (Construct 4) | KDTLMISRTPEVTCVVVDVSHEDPEVKFNWYVDGVEVHN |
| | | AKTKPREEQYASTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFL |
| | | YSKLTVDKSRWQQGNVFSCSVMHEALHNHYTQKSLSLSP |
| | | GGGGSGGGGSQVQLVQSGAEVKKPGASVKVSCKASGHTFTSYF |
| | | MHWIRQAPGQGLEWMGIINPSDDYANYAQKFQGRVTMTRDTST |
| | | STVYMELSSLRSEDTAVYYCATAIFDYWGQGTPVTVSSASTKGPS |
| | | VFPLAPSSKSTSGGTAALGCLVKDYFPEPVTVSWNSGALTS |
| | | GVHTFPAVLQSSGLYSLSSVVTVPSSSLGTQTYICNVNHKP |
| | | SNTKVDKKVEPKSCDKTHT |
| 28 | Human NKp46 | MSSTLPALLCVGLCLSQRISAQQQTLPKPFIWAEPHFMVPKEK |
| | amino acid | QVTICCQGNYGAVEYQLHFEGSLFAVDRPKPPERINKVKFYIP |
| | sequence | DMNSRMAGQYSCIYRVGELWSEPSNLLDLVVTEMYDTPTLS |
| | | VHPGPEVISGEKVTFYCRLDTATSMFLLLKEGRSSHVQRGYG |
| | | KVQAEFPLGPVTTAHRGTYRCFGSYNNHAWSFPSEPVKLLVT |
| | | GDIENTSLAPEDPTFPADTWGTYLLTTETGLQKDHALWDHTA |
| | | QNLLRMGLAFLVLVALVWFLVEDWLSRKRTRERASRASTWE |
| | | GRRRLNTQTL |
| 29 | Human 2B4 | MLGQVVTLILLLLLKVYQGKGCQGSADHVVSISGVPLQLQPN |
| | (CD244) | SIQTKVDSIAWKKLLPSQNGFHHILKWENGSLPSNTSNDRFSFI |
| | Isoform 1 | VKNLSLLIKAAQQQDSGLYCLEVTSISGKVQTATFQVFVFESL |
| | amino acid | LPDKVEKPRLQGQGKILDRGRCQVALSCLVSRDGNVSYAWY |
| | sequence | RGSKLIQTAGNLTYLDEEVDINGTHTYTCNVSNPVSWESHTL |
| | | NLTQDCQNAHQEFRFWPFLVIIVILSALFLGTLACFCVWRRKR |
| | | KEKQSETSPKEFLTIYEDVKDLKTRRNHEQEQTFPGGGSTIYS |
| | | MIQSQSSAPTSQEPAYTLYSLIQPSRKSGSRKRNHSPSFNSTIYE |
| | | VIGKSQPKAQNPARLSRKELENFDVYS |
| 30 | Human 2B4 | MLGQVVTLILLLLLKVYQGKGCQGSADHVVSISGVPLQLQPN |
| | (CD244) | SIQTKVDSIAWKKLLPSQNGFHHILKWENGSLPSNTSNDRFSFI |
| | Isoform 2 | VKNLSLLIKAAQQQDSGLYCLEVTSISGKVQTATFQVFVFDK |
| | | VEKPRLQGQGKILDRGRCQVALSCLVSRDGNVSYAWYRGSK |
| | | LIQTAGNLTYLDEEVDINGTHTYTCNVSNPVSWESHTLNLTQ |
| | | DCQNAHQEFRFWPFLVIIVILSALFLGTLACFCVWRRKRKEKQ |
| | | SETSPKEFLTIYEDVKDLKTRRNHEQEQTFPGGGSTIYSMIQSQ |
| | | SSAPTSQEPAYTLYSLIQPSRKSGSRKRNHSPSFNSTIYEVIGKS |
| | | QPKAQNPARLSRKELENFDVYS |
| 31 | Human | MDYPTLLLALLHVYRALCEEVLWHTSVPFAENMSLECVYPS |
| | CD226 amino | MGILTQVEWFKIGTQQDSIAIFSPTHGMVIRKPYAERVYFLNS |
| | acid sequence | TMASNNMTLFFRNASEDDVGYYSCSLYTYPQGTWQKVIQVV |
| | | QSDSFEAAVPSNSHIVSEPGKNVTLTCQPQMTWPVQAVRWE |
| | | KIQPRQIDLLTYCNLVHGRNFTSKFPRQIVSNCSHGRWSVIVIP |
| | | DVTVSDSGLYRCYLQASAGENETFVMRLTVAEGKTDNQYTL |
| | | FVAGGTVLLLLFVISITTIIVIFLNRRRRRERRDLFTESWDTQKA |
| | | PNNYRSPISTSQPTNQSMDDTREDIYVNYPTFSRRPKTV |
| 32 | Human | MGWIRGRRSRHSWEMSEFHNYNLDLKKSDFSTRWQKQRCPV |
| | NKG2D amino | VKSKCRENASPFFFCCFIAVAMGIRFIIMVAIWSAVFLNSLFNQ |
| | acid sequence | EVQIPLTESYCGPCPKNWICYKNNCYQFFDESKNWYESQASC |
| | | MSQNASLLKVYSKEDQDLLKLVKSYHWMGLVHIPTNGSWQ |
| | | WEDGSILSPNLLTIIEMQKGDCALYASSFKGYIENCSTPNTYIC |
| | | MQRTV |
| 33 | Human | MGNSCYNIVATLLLVLNFERTRSLQDPCSNCPAGTFDNNRNQI |
| | CD137 amino | CSPCPPNSFSSAGGQRTCDICRQCKGVFRTRKECSSTSNAECD |
| | acid sequence | CTPGFHCLGAGCSMCEQDCKQGQLKKGCKDCCFGTFNDQKR |
| | | GICRPWTNCSLDGKSVLVNGTKERDVVCGPSPADLSPGASSV |
| | | TPPAPAREPGHSPQIISFFLALTSTALLFLLFFLTLRFSVVKRGR |
| | | KKLLYIFKQPFMRPVQTTQEEDGCSCRFPEEEEGGCEL |
| 34 | Human CD16a | MWQLLLPTALLLLVSAGMRTEDLPKAVVFLEPQWYRVLEKD |
| | amino acid | SVTLKCQGAYSPEDNSTQWFHNESLISSQASSYFIDAATVDDS |
| | sequence | GEYRCQTNLSTLSDPVQLEVHIGWLLLQAPRWVFKEEDPIHL |
| | | RCHSWKNTALHKVTYLQNGKGRKYFHHNSDFYIPKATLKDS |
| | | GSYFCRGLFGSKNVSSETVNITITQGLAVSTISSFFPPGYQVSFC |
| | | LVMVLLFAVDTGLYFSVKTNIRSSTRDWKDHKFKWRKDPQD |
| | | K |
| 35 | Human CD2 | MSFPCKFVASFLLIFNVSSKGAVSKEITNALETWGALGQDINL |
| | amino acid | DIPSFQMSDDIDDIKWEKTSDKKKIAQFRKEKETFKEKDTYKL |
| | sequence | FKNGTLKIKHLKTDDQDIYKVSIYDTKGKNVLEKIFDLKIQER |
| | | VSKPKISWTCINTTLTCEVMNGTDPELNLYQDGKHLKLSQRVI |
| | | THKWTTSLSAKFKCTAGNKVSKESSVEPVSCPEKGLDIYLIIGI |
| | | CGGGSLLMVFVALLVFYITKRKKQRSRRNDEELETRAHRVAT |
| | | EERGRKPHQIPASTPQNPATSQHPPPPPGHRSQAPSHRPPPPGH |
| | | RVQHQPQKRPPAPSGTQVHQQKGPPLPRPRVQPKPPHGAAEN |
| | | SLSPSSN |
| 36 | Human NKp44 | MAWRALHPLLLLLLLFPGSQAQSKAQVLQSVAGQTLTVRCQ |
| | amino acid | YPPTGSLYEKKGWCKEASALVCIRLVTSSKPRTMAWTSRFTI |
| | sequence | WDDPDAGEFTVTMTDLREEDSGHYWCRIYRPSDNSVSKSVRE |
| | | YLVVSPASASTQTSWTPRDLVSSQTQTQSCVPPTAGARQAPES |
| | | PSTIPVPSQPQNSTLRPGPAAPIALVPVFCGLLVAKSLVLSALL |
| | | VWWGDIWWKTMMELRSLDTQKATCHLQQVTDLPWTSVSSP |
| | | VEREILYHTVARTKISDDDDEHTL |
| 37 | Human HER2 | MKLRLPASPETHLDMLRHLYQGCQVVQGNLELTYLPTNASLS |
| | isoform b | FLQDIQEVQGYVLIAHNQVRQVPLQRLRIVRGTQLFEDNYAL |
| | amino acid | AVLDNGDPLNNTTPVTGASPGGLRELQLRSLTEILKGGVLIQR |
| | sequence | NPQLCYQDTILWKDIFHKNNQLALTLIDTNRSRACHPCSPMC |
| | | KGSRCWGESSEDCQSLTRTVCAGGCARCKGPLPTDCCHEQC |
| | | AAGCTGPKHSDCLACLHFNHSGICELHCPALVTYNTDTFESM |
| | | PNPEGRYTFGASCVTACPYNYLSTDVGSCTLVCPLHNQEVTA |
| | | EDGTQRCEKCSKPCARVCYGLGMEHLREVRAVTSANIQEFA |
| | | GCKKIEGSLAFLPESEDGDPASNTAPLQPEQLQVFETLEEITGY |
| | | LYISAWPDSLPDLSVFQNLQVIRGRILHNGAYSLTLQGLGISW |
| | | LGLRSLRELGSGLALIHEINTHLCFVHTVPWDQLFRNPHQALL |
| | | HTANRPEDECVGEGLACHQLCARGHCWGPGPTQCVNCSQFL |
| | | RGQECVEECRVLQGLPREYVNARHCLPCHPECQPQNGSVTCF |
| | | GPEADQCVACAHYKDPPFCVARCPSGVKPDLSYMPIWKFPDE |
| | | EGACQPCPINCTHSCVDLDDKGCPAEQRASPLTSIISAVVGILL |
| | | VVVLGVVEGILIKRRQQKIRKYTMIRRLLQETELVEPLTPSGAM |
| | | PNQAQMRILKETELRKVKVLGSGAFGTVYKGIWIPDGENVKI |
| | | PVAIKVLRENTSPKANKEILDEAYVMAGVGSPYVSRLLGICLT |
| | | STVQLVTQLMPYGCLLDHVRENRGRLGSQDLLNWCMQIAKG |
| | | MSYLEDVRLVHRDLAARNVLVKSPNHVKITDFGLARLLDIDE |
| | | TEYHADGGKVPIKWMALESILRRRFTHQSDVWSYGVTVWEL |
| | | MTFGAKPYDGIPAREIPDLLEKGERLPQPPICTIDVYMIMVKC |
| | | WMIDSECRPRFRELVSEFSRMARDPQRFVVIQNEDLGPASPLD |
| | | STFYRSLLEDDDMGDLVDAEEYLVPQQGFFCPDPAPGAGGM |
| | | VHHRHRSSSTRSGGGDLTLGLEPSEEEAPRSPLAPSEGAGSDV |
| | | FDGDLGMGAAKGLQSLPTHDPSPLQRYSEDPTVPLPSETDGY |
| | | VAPLTCSPQPEYVNQPDVRPQPPSPREGPLPAARPAGATLERP |
| | | KTLSPGKNGVVKDVFAFGGAVENPEYLTPQGGAAPQPHPPPA |
| | | FSPAFDNLYYWDQDPPERGAPPSTFKGTPTAENPEYLGLDVP |
| | | V |
| 38 | Consensus | YTFX₁X₂X₃YX₄H, where X₁ is T or S, X₂ is N or S, X₃ is Y or H, |
| | BCMA | and X₄ is M or V |
| | CDRH1 amino | |
| | acid sequence | |
| 39 | Consensus | GX₅IDPSX₆GX₇TX₈YA, where X₅ is V or I, X₆ is G or D, X₇ is G, Y |
| | BCMA | or S, and X₈ is N or S |
| | CDRH2 amino | |
| | acid sequence | |
| 40 | Consensus | ARGRYDYX₉DYLGWFDX₁₀, where X₉ is G or S, X₁₀ is P or G |
| | BCMA | |
| | CDRH3 amino | |
| | acid sequence | |
| 41 | CDRH3 amino | ARGRYDYGDYLGWFDP |
| | acid sequence | |
| | anti-BCMA | |
| | antibody | |
| | (mAb1, mAb5, | |
| | mAb6, mAb7 | |
| | and Constructs | |
| | 1, 2, 3, 4, 7, | |
| | and 8) | |
| 42 | CDRH3 amino | ATAIFDY |
| | acid sequence | |
| | anti-NKp30 | |
| | antibody | |
| | (mAb8, mAb9; | |
| | Constructs 1, | |
| | 2, 3, 4, 5, 6, | |
| | and 7) | |
| 43 | CDRL3 amino | QQSYSTPLT |
| | acid sequence | |
| | (mAb1- | |
| | mAb10; | |
| | Constructs 1, | |
| | 2, 3, 4, 5, 6, | |
| | and 7) | |
| 44 | CDRH1 amino | YTFTNYYMH |
| | acid sequence | |
| | of anti-BCMA | |
| | antibody | |
| | (mAb2, mAb6, | |
| | mAb7; | |
| | Construct 6) | |
| 45 | CDRH2 amino | GVIDPSGGYTNYA |
| | acid sequence | |
| | of anti-B CMA | |
| | antibody | |
| | (mAb2, mAb4; | |
| | Construct 6) | |
| 46 | CDRH3 amino | ARGRYDYGDYLGWFDG |
| | acid sequence | |
| | of anti-BCMA | |
| | antibody | |
| | (mAb2, mAb3; | |
| | Constructs 5 | |
| | and 6) | |
| 47 | CDRH1 amino | YTFTSYYMH |
| | acid sequence | |
| | of anti-BCMA | |
| | antibody | |
| | (mAb3, | |
| | Construct 5) | |
| 48 | CDRH2 amino | GVIDPSGGSTNYA |
| | acid sequence | |
| | of anti-BCMA | |
| | antibody | |
| | (mAb3; | |
| | Construct 5) | |
| 49 | CDRH3 amino | ARGRYDYSDYLGWFDP |
| | acid sequence | |
| | of anti-BCMA | |
| | antibody | |
| | (mAb4) | |
| 50 | CDRH2 amino | GVIDPSDGGTNYA |
| | acid sequence | |
| | of anti-BCMA | |
| | antibody | |
| | (mAb5, mAb6, | |
| | and mAb7) | |
| 51 | mAb1 | GTQVQLVQSGAEVKKPGASVKVSCKASGYTFSSHYVHWVRQ |
| | aglycosylated | APGQGLEWMGIIDPSGGSTSYAQKFQGRVTMTRDTSTSTVYM |
| | IgG1 heavy | ELSSLRSEDTAVYYCARGRYYGDYLGWFDWPWGQGTLVTV |
| | chain amino | SASTKGPSVFPLAPSSKSTSGGTAALGCLVDKYFPEPVTVSWN |
| | acid sequence | SGALTSGVHTFPAVLQSSGLYSLSSVVTVPSSSLGTQTYICNV |
| | | NHKPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYASTYRVVSVLTVLHQDWLNGKEYKCKVSNK |
| 52 | mAb2 VH | QVQLVQSGAEVKKPGASVKVSCKASGYTFTNYYMHWVRQA |
| | amino acid | PGQGLEWMGVIDPSGGYTNYAQKFQGRVTMTRDTSTSTVYM |
| | sequence | ELSSLRSEDTAVYYCARGRYDYGDYLGWFDGWGQGTLVTV |
| | | SS |
| 53 | mAb3 VH | QVQLVQSGAEVKKPGASVKVSCKASGYTFTSYYMHWVRQA |
| | amino acid | PGQGLEWMGVIDPSGGSTNYAQKFQGRVTMTRDTSTSTVYM |
| | sequence | ELSSLRSEDTAVYYCARGRYDYGDYLGWFDGWGQGTLVTV |
| | | SS |
| 54 | mAb4 VH | QVQLVQSGAEVKKPGASVKVSCKASGYTFSSHYVHWVRQAP |
| | amino acid | GQGLEWMGVIDPSGGYTNYAQKFQGRVTMTRDTSTSTVYM |
| | sequence | ELSSLRSEDTAVYYCARGRYDYSDYLGWFDPWGQGTLVTVS |
| | | S |
| 55 | mAb5 VH | QVQLVQSGAEVKKPGASVKVSCKASGYTFSSHYVHWVRQAP |
| | amino acid | GQGLEWMGVIDPSDGGTNYAQKFQGRVTMTRDTSTSTVYM |
| | sequence | ELSSLRSEDTAVYYCARGRYDYGDYLGWFDPWGQGTLVTVS |
| | | S |
| 56 | mAb6 VH | QVQLVQSGAEVKKPGASVKVSCKASGYTFTNYYMHWVRQA |
| | amino acid | PGQGLEWMGVIDPSDGGTNYAQKFQGRVTMTRDTSTSTVYM |
| | sequence | ELSSLRSEDMAVYYCARGRYDYGDYLGWFDPWGQGTLVTV |
| | | SS |
| 57 | mAb7 VH | QVQLVQSGAEVKKPGASVKVSCKASGYTFTNYYMHWVRQA |
| | amino acid | PGQGLEWMGVIDPSDGGTNYAQKFQGRVTMTRDTSTSTVYM |
| | sequence | ELSSLRSEDTAVYYCARGRYDYGDYLGWFDPWGQGTLVTVS |
| | | S |
| 58 | mAb2 IgG1 | QVQLVQSGAEVKKPGASVKVSCKASGYTFTNYYMHWVRQA |
| | heavy chain | PGQGLEWMGVIDPSGGYTNYAQKFQGRVTMTRDTSTSTVYM |
| | amino acid | ELSSLRSEDTAVYYCARGRYDYGDYLGWFDGWGQGTLVTV |
| | sequence | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| 59 | mAb3 IgG1 | QVQLVQSGAEVKKPGASVKVSCKASGYTFTSYYMHWVRQA |
| | heavy chain | PGQGLEWMGVIDPSGGSTNYAQKFQGRVTMTRDTSTSTVYM |
| | amino acid | ELSSLRSEDTAVYYCARGRYDYGDYLGWFDGWGQGTLVTV |
| | sequence | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| 60 | mAb4 IgG1 | QVQLVQSGAEVKKPGASVKVSCKASGYTFTSYYMHWVRQA |
| | heavy chain | PGQGLEWMGVIDPSGGSTNYAQKFQGRVTMTRDTSTSTVYM |
| | amino acid | ELSSLRSEDTAVYYCARGRYDYGDYLGWFDGWGQGTLVTV |
| | sequence | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| 61 | mAb 5 IgG1 | QVQLVQSGAEVKKPGASVKVSCKASGYTFSSHYVHWVRQAP |
| | heavy chain | GQGLEWMGVIDPSDGGTNYAQKFQGRVTMTRDTSTSTVYM |
| | amino acid | ELSSLRSEDTAVYYCARGRYDYGDYLGWFDGWGQGTLVTVS |
| | sequence | SASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPG |
| 62 | mAb6 IgG1 | QVQLVQSGAEVKKPGASVKVSCKASGYTFTNYYMHWVRQA |
| | heavy chain | PGQGLEWMGVIDPSDGGTNYAQKFQGRVTMTRDTSTSTVYM |
| | amino acid | ELSSLRSEDMAVYYCARGRYDYGDYLGWFDPWGQGTLVTV |
| | sequence | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| 63 | mAb7 IgG1 | QVQLVQSGAEVKKPGASVKVSCKASGYTFTNYYMHWVRQA |
| | heavy chain | PGQGLEWMGVIDPSDGGTNYAQKFQGRVTMTRDTSTSTVYM |
| | amino acid | ELSSLRSEDTAVYYCARGRYDYGDYLGWFDPWGQGTLVTVS |
| | sequence | SASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPG |
| 64 | mAb8 | GTQVQLVQSGAEVKKPGASVKVSCKASGHTFTSYFMHWVR |
| | IgG1 heavy | QAPGQGLEWMGIINPSDDYANYAQKFQGRVTMTRDTSTSTV |
| | chain amino | YMELSSLRSEDTAVYYCATAIFDYWGQGTPVTVSSASTKGPS |
| | acid sequence | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHTCPPCPAPELLGGPSVFLFPPKPKDTLM |
| | | ISRTPEVTCVVVDVSHEDPEVKFNWYVDGVEVHNAKTKPRE |
| | | EQYASTYRVVSVLTVLHQDWLNGKEYKCKVSNKALPAPIEK |
| | | TISKAKGQPREPQVYTLPPSRDELTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPG |

| | | |
|---|---|---|
| 65 | Heavy chain | QVQLVQSGAEVKKGPASVKVSCKASGYTFTSYYMHWVRQA |
| | amino acid | PGQGLEWMGVIDPSGGSTNYAQKFQGRVTMTRDTSTSTVYM |
| | sequence of | ELSSLRSEDTAVYYCARGRYDYGDYLGWFDGWGQGTLVTV |
| | Costruct 5 | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPGGGGSGG |
| | | GGSQVQLVQSGAEVKKPGASVKVSCKASGHTFTSYFMHWVR |
| | | QAPGQGLEWMGIINPSDDYANYAQKFQGRVTMTRDTSTSTV |
| | | YMELSSLRSEDTAVYYCATAIFDYWGQGTLVTVSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHT |
| 66 | Heavy chain | QVQLVQSGAEVKKGPASVKVSCKASGYTFTSYYMHWVRQA |
| | amino acid | PGQGLEWMGVIDPSGGSTNYAQKFQGRVTMTRDTSTSTVYM |
| | sequence of | ELSSLRSEDTAVYYCARGRYDYGDYLGWFDGWGQGTLVTV |
| | Construct 6 | SSASTKGPSVFPLAPSSKSTSGGTAALGCLVKDYFPEPVTVSW |
| | | NSGALTSGVHTFPAVLQSSGLYSLSSVVTVPSSSLGTQTYICN |
| | | VNHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPGGGGSGG |
| | | GGSQVQLVQSGAEVKKPGASVKVSCKASGHTFTSYFMHWVR |
| | | QAPGQGLEWMGIINPSDDYANYAQKFQGRVTMTRDTSTSTV |
| | | YMELSSLRSEDTAVYYCATAIFDYWGQGTLVTVSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHT |
| 67 | Alternative | QVQLVQSGAEVKKPGASVKVSCKASGYTFSSHYVHWVRQAP |
| | heavy chain | GQGLEWMGIIDPSGGSTSYAQKFQGRVTMTRDTSTSTVYMEL |
| | amino acid | SSLRSEDTAVYYCARGRYDYGDYLGWFDPWGQGTLVTVSSA |
| | sequence of | STKGPSVFPLAPSSKSTSGGTAALGCLVKDYFPEPVTVSWNSG |
| | Construct 1 | ALTSGVHTFPAVLQSSGLYSLSSVVTVPSSSLGTQTYICNVNH |
| | (Construct 7) | KPSNTKVDKKVEPKSCDKTHTCPPCPAPELLGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFFLYSKLTVDKS |
| | | RWQQGNVFSCSVMHEALHNHYTQKSLSLSPGGGGSGGGGSG |
| | | GGGSGGGGSQVQLVQSGAEVKKPGASVKVSCKASGHTFTSY |
| | | FMHWVRQAPGQGLEWMGIINPSDDYANYAQKFQGRVTMTR |
| | | DTSTSTVYMELSSLRSEDTAVYYCATAIFDYWGQGTLVTVSS |
| | | ASTKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKKVEPKSCDKTHT |
| 68 | Aglycosylated | QVQLVQSGAEVKKGPASVKVSCKASGYTFTSYYMHWVRQA |
| | heavy chain | PGQGLEWMGVIDPSGGSTNYAQKFQGRVTMTRDTSTSTVYM |
| | amino acid | ELSSLRSEDTAVYYCARGRYDYGDYLGWFDGWGQGTLVTV |
| | sequence of | SSASTKGPSVFPLAPSSKSTSGGTAALGCLVKDYFPEPVTVSW |
| | Construct 5 | NSGALTSGVHTFPAVLQSSGLYSLSSVVTVPSSSLGTQTYICN |
| | (Construct 8) | VNHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPGGGGSGG |
| | | GGSQVQLVQSGAEVKKPGASVKVSCKASGHTFTSYFMHWVR |
| | | QAPGQGLEWMGIINPSDDYANYAQKFQGRVTMTRDTSTSTV |
| | | YMELSSLRSEDTAVYYCATAIFDYWGQGTLVTVSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKKVEPKSCDKTHT |
| 69 | CDRH1 amino | GTFTSYSVS |
| | acid sequence | |
| | of mAb10 | |
| 70 | CDRH2 amino | GGIVPIFGTADYA |
| | acid sequence | |
| | of mAb10 | |
| 71 | CDRH3 amino | ARGYSYGQTFDY |
| | acid sequence | |
| | of mAb10 | |
| 72 | VH amino acid | QVQLVQSGAEVKKPGSSVKVSCKASGGTFTSYSVSWVRQAP |
| | sequence of | GQGLEWMGGIVPIFGTADYAQKFQGRVTITADESTSTAYMEL |
| | mAb10 | SSLRSEDTAVYYCARGYSYGQTFDYWGQGTLVTVSS |
| 73 | aglycosylated | QVQLVQSGAEVKKPGSSVKVSCKASGGTFTSYSVSWVRQAP |
| | IgG1 heavy | GQGLEWMGGIVPIFGTADYAQKFQGRVTITADESTSTAYMEL |
| | chain amino | SSLRSEDTAVYYCARGYSYGQTFDYWGQGTLVTVSSASTKG |
| | acid sequence | PSVFPLAPSSKSTSGGTAALGCLVKDYFPEPVTVSWNSGALTS |
| | of mAb10 | GVHTFPAVLQSSGLYSLSSVVTVPSSSLGTQTYICNVNHKPSN |
| | | TKVDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYASTYRVVSVLTVLHQDWLNGKEYKCKVSNKALPAPIE |
| | | KTISKAKGQPREPQVYTLPPSRDELTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPG |
| 74 | IgG1 heavy | GQVQLVQSGAEVKKPGASVKVSCKASGYTFSSHYVHWVRQ |
| | chain amino | APGQGLEWMGIIDPSGGSTSYAQKFQGRVTMTRDTSTSTVYM |
| | acid sequence | ELSSLRSEDTAVYYCARGRYDYGDYLGWFDPWGQGTLVTVS |
| | of Construct 9 | SASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLTV |
| | | DKSRWQQGNVFSCSVMHEALHNHYTQKSLSLSPGGGGSGGG |
| | | GSQVQLVQSGAEVKKPGSSVKVSCKASGGTFTSYSVSWVRQ |
| | | APGQGLEWMGGIVPIFGTADYAQKFQGRVTITADESTSTAYM |
| | | ELSSLRSEDTAVYYCARGYSYGQTFDYWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHT |
| 75 | CDRH1 amino | HTFTNYYMH |
| | acid sequence | |
| | of mAb11 | |
| 76 | CDRH2 amino | GVINPNGGDTSYA |
| | acid sequence | |
| | of mAb11 | |
| 77 | CDRH3 amino | ARDRAWDYGGNVRAFDI |
| | acid sequence | |
| | of mAb11 | |
| 78 | VH amino acid | QVQLVQSGAEVKKPGASVKVSCKASGHTFTNYYMHWVRQA |
| | sequence of | PGQGLEWMGVINPNGGDTSYAQKFQGRVTMTRDTSTSTVYM |
| | mAb11 | ELSSLRSEDTAVYYCARDRAWDYGGNVRAFDIWGQGTMVT |
| | | VSS |
| 79 | aglycosylated | QVQLVQSGAEVKKPGASVKVSCKASGHTFTNYYMHWVRQA |
| | IgG1 heavy | PGQGLEWMGVINPNGGDTSYAQKFQGRVTMTRDTSTSTVYM |
| | chain amino | ELSSLRSEDTAVYYCARDRAWDYGGNVRAFDIWGQGTMVT |
| | acid sequence | VSSASTKGPSVFPLAPSSKSTSGGTAALGCLVKDYFPEPVTVS |
| | of mAb11 | WNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVEV |
| | | HNAKTKPREEQYASTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSKL |
| | | TVDKSRWQQGNVFSCSVMHEALHNHYTQKSLSLSPG |
| 80 | Variable light | DIVMTQSPLSLPVTPGEPASISCRSSQSLLHSNGYNYLDWYLQ |
| | chain amino | KPGQSPQLLIYLGSNRASGVPDRFSGSGSGTDFTLKISRVEAED |
| | acid sequence | VGVYYCMQALQTPYTFGGGTKVEIK |
| | of mAb11 | |
| 81 | Light chain | DIVMTQSPLSLPVTPGEPASISCRSSQSLLHSNGYNYLDWYLQ |
| | amino acid | KPGQSPQLLIYLGSNRASGVPDRFSGSGSGTDFTLKISRVEAED |
| | sequence of | VGVYYCMQALQTPYTFGGGTKVEIKRTVAAPSVFIFPPSDEQL |
| | mAb11 | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| 82 | Human NKp30 | LWVSQPPEIRTLEGSSAFLPCSFNASQGRLAIGSVTWFRDEVV |
| | amino acid | PGKEVRNGTPEFRGRLAPLASSRFLHDHQAELHIRDVRGHDA |
| | sequence | SIYVCRVEVLGLGVGTGNGTRLVVEKEHPQLGAGTVLLLRAG |
| | (without signal | FYAVSFLSVAVGSTVYYQGKCLTWKGPRRQLPAVVPAPLPPP |
| | sequence) | CGSSAHLLPPVPGG |
| 83 | Variable light | GATATCCAGATGACACAGTCTCCATCCTCTCTGAGTGCTAG |
| | chain nucleic | TGTGGGTGACCGCGTTACGATAACCTGCCGCGCAAGTCAG |
| | acid sequence | TCCATATCATCATATCTGAACTGGTATCAGCAAAAACCCGG |
| | (mAb1-mAb9; | CAAAGCACCTAAACTATTGATCTACGCCGCATCGTCACTTC |
| | Constructs 1-9) | AGAGTGGTGTGCCGAGTCGTTTTAGTGGTTCCGGAAGCGGT |
| | | ACCGATTTTACCCTGACTATTAGCAGCCTCCAGCCCGAAGA |
| | | TTTCGCGACCTACTATTGCCAGCAGAGCTACTCGACGCCAC |
| | | TAACTTTCGGCGGAGGGACCAAGGTGGAGATCAAA |
| 84 | Heavy chain | CAGGTACAGCTGGTGCAATCTGGTGCGGAGGTGAAGAAACCG |
| | nucleic acid | GGTGCCAGTGTGAAAGTTAGTTGTAAGGCAAGCGGTTACACCT |
| | sequence of | TCTCTTCTCACTACGTTCACTGGGTGCGGCAAGCGCCGGGTCA |
| | Construct 1 | GGGCTTGGAATGGATGGGTATCATCGATCCGTCTGGTGGTTCT |
| | | ACCTCTTACGCACAGAAATTTCAAGGTCGCGTGACCATGACCC |
| | | GAGATACCTCCACGAGTACTGTGTACATGGAATTGTCGTCCCT |
| | | GCGCTCCGAAGACACGGCCGTGTATTACTGTGCGAGAGGACG |
| | | GTATGACTACGGTGACTACTTGGGGTGGTTCGACCCCTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACAACAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGAGGCGGAAGCG |
| | | GAGGTGGCGGATCCCAGGTACAGCTGGTGCAATCTGGTGCG |
| | | GAGGTGAAGAAACCGGGTGCCAGTGTGAAAGTTAGTTGTAAGG |
| | | CAAGCGGTCACACCTTCACCTCTTACTTCATGCACTGGGTGCG |
| | | GCAAGCGCCGGGTCAGGGCTTGGAATGGATGGGTATCATCAA |
| | | CCCGTCTGATGATTACGCAAACTACGCACAGAAATTTCAAGGTC |
| | | GCGTGACCATGACCCGAGATACCTCCACGAGTACTGTGTACAT |
| | | GGAATTGTCGTCCCTGCGCTCCGAAGACACGGCCGTGTATTAC |
| | | TGTGCCACAGCGATCTTTGACTACTGGGGCCAGGGAACCCTG |
| | | GTCACCGTCTCCTCAGCTAGCACTAAAGGACCCAGTGTAT |
| | | TTCCTTTGGCCCCTAGCAGCAAATCTACATCTGGCGGC |
| | | ACAGCCGCTCTGGGTTGTCTGGTGAAAGACTACTTTCC |
| | | TGAACCCGTGACTGTTTCATGGAACAGTGGGGCACTCA |
| | | CGAGCGGAGTGCATACTTTCCCCGCTGTGCTTCAGAGT |
| | | TCTGGACTCTATTCGCTTAGCTCTGTCGTAACCGTCCCT |
| | | AGTTCCAGCCTGGGCACCCAAACATATATATGCAACGT |
| | | TAACCATAAACCCTCAAATACAAAGGTTGATAAGAAGG |
| | | TGGAGCCGAAGAGTTGTGACAAGACCCACACC |
| 85 | Heavy chain | CAGGTACAGCTGGTGCAATCTGGTGCGGAGGTGAAGAAACCG |
| | nucleic acid | GGTGCCAGTGTGAAAGTTAGTTGTAAGGCAAGCGGTTACACCT |
| | sequence of | TCTCTTCTCACTACGTTCACTGGGTGCGGCAAGCGCCGGGTCA |
| | Construct 3 | GGGCTTGGAATGGATGGGTATCATCGATCCGTCTGGTGGTTCT |
| | (aglycosylated | ACCTCTTACGCACAGAAATTTCAAGGTCGCGTGACCATGACCC |
| | Construct 1) | GAGATACCTCCACGAGTACTGTGTACATGGAATTGTCGTCCCT |
| | | GCGCTCCGAAGACACGGCCGTGTATTACTGTGCGAGAGGACG |
| | | GTATGACTACGGTGACTACTTGGGGTGGTTCGACCCCTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACGCCAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGAGGCGGAAGCG |
| | | GAGGTGGCGGATCCCAGGTACAGCTGGTGCAATCTGGTGCG |
| | | GAGGTGAAGAAACCGGGTGCCAGTGTGAAAGTTAGTTGTAAGG |
| | | CAAGCGGTCACACCTTCACCTCTTACTTCATGCACTGGGTGCG |
| | | GCAAGCGCCGGGTCAGGGCTTGGAATGGATGGGTATCATCAA |
| | | CCCGTCTGATGATTACGCAAACTACGCACAGAAATTTCAAGGTC |
| | | GCGTGACCATGACCCGAGATACCTCCACGAGTACTGTGTACAT |
| | | GGAATTGTCGTCCCTGCGCTCCGAAGACACGGCCGTGTATTAC |
| | | TGTGCCACAGCGATCTTTGACTACTGGGGCCAGGGAACCCTG |
| | | GTCACCGTCTCCTCAGCTAGCACTAAAGGACCCAGTGTAT |
| | | TTCCTTTGGCCCCTAGCAGCAAATCTACATCTGGCGGC |
| | | ACAGCCGCTCTGGGTTGTCTGGTGAAAGACTACTTTCC |
| | | TGAACCCGTGACTGTTTCATGGAACAGTGGGGCACTCA |
| | | CGAGCGGAGTGCATACTTTCCCCGCTGTGCTTCAGAGT |
| | | TCTGGACTCTATTCGCTTAGCTCTGTCGTAACCGTCCCT |
| | | AGTTCCAGCCTGGGCACCCAAACATATATATGCAACGT |
| | | TAACCATAAACCCTCAAATACAAAGGTTGATAAGAAGG |
| | | TGGAGCCGAAGAGTTGTGACAAGACCCACACC |
| 86 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCTCTTCTCACTACGTTCACTGGGTGCGGCAAGCGC |
| | of anti-BCMA | CGGGTCAGGGCTTGGAATGGATGGGTATCATCGATCCGTCT |
| | antibody | GGTGGTTCTACCTCTTACGCACAGAAATTTCAAGGTCGCGT |
| | (mAb1, | GACCATGACCCGAGATACCTCCACGAGTACTGTGTACATG |
| | Constructs 1, 2, | GAATTGTCGTCCCTGCGCTCCGAAGACACGGCCGTGTATTA |
| | 3, and 4) | CTGTGCGAGAGGACGGTATGACTACGGTGACTACTTGGGG |
| | | TGGTTCGACCCCTGGGGCCAGGGAACCCTGGTCACCGTCTC |
| | | CTCA |
| 87 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTCA |
| | acid sequence | CACCTTCACCTCTTACTTCATGCACTGGGTGCGGCAAGCGC |
| | of anti-NKp30 | CGGGTCAGGGCTTGGAATGGATGGGTATCATCAACCCGTC |
| | antibody | TGATGATTACGCAAACTACGCACAGAAATTTCAAGGTCGC |
| | (mAb8; | GTGACCATGACCCGAGATACCTCCACGAGTACTGTGTACAT |
| | Constructs 2 | GGAATTGTCGTCCCTGCGCTCCGAAGACACGGCCGTGTATT |
| | and 4) | ACTGTGCCACAGCGATCTTTGACTACTGGGGCCAGGGAAC |
| | | CCCGGTCACCGTCTCCTCA |
| 88 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCACCAACTACTACATGCACTGGGTGCGGCAAGCG |
| | of anti-BCMA | CCGGGTCAGGGCTTGGAATGGATGGGTGTTATCGATCCGTC |
| | antibody | TGGTGGTTACACCAACTACGCACAGAAATTTCAAGGTCGC |
| | (mAb2) | GTGACCATGACCCGAGATACCTCCACGAGTACTGTGTACAT |
| | | GGAATTGTCGTCCCTGCGCTCCGAAGACACGGCCGTGTATT |
| | | ACTGTGCAAGAGGACGGTATGACTACGGTGACTACTTGGG |
| | | GTGGTTCGACGGTTGGGGCCAGGGAACCCTGGTCACCGTC |
| | | TCCTCA |
| 89 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACGTTCACGTCATATTACATGCATTGGGTGCGGCAAGCGC |
| | of anti-BCMA | CGGGTCAGGGCTTGGAATGGATGGGTGTTATCGATCCGTCT |
| | antibody | GGTGGTTCTACCAACTACGCACAGAAATTTCAAGGTCGCGT |
| | (mAb3) | GACCATGACCCGAGATACCTCCACGAGTACTGTGTACATG |
| | | GAATTGTCGTCCCTGCGCTCCGAAGACACGGCCGTGTATTA |
| | | CTGTGCAAGAGGACGGTATGACTACGGTGACTACTTGGGG |
| | | TGGTTCGACGGGTGGGGCCAGGGAACCCTGGTCACCGTCT |
| | | CCTCA |
| 90 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCTCTTCTCACTACGTTCACTGGGTGCGGCAAGCGC |
| | of anti-BCMA | CGGGTCAGGGCTTGGAATGGATGGGTGTTATCGATCCGTCT |
| | antibody | GGTGGTTACACCAACTACGCACAGAAATTTCAAGGTCGCG |
| | (mAb4) | TGACCATGACCCGAGATACCTCCACGAGTACTGTGTACATG |
| | | GAATTGTCGTCCCTGCGCTCCGAAGACACGGCCGTGTATTA |
| | | CTGTGCAAGAGGACGGTATGACTACAGTGACTACTTGGGG |
| | | TGGTTCGACCCCTGGGGCCAGGGAACCCTGGTCACCGTCTC |
| | | CTCA |
| 91 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCTCTTCTCACTACGTTCACTGGGTGCGGCAAGCGC |
| | of anti-BCMA | CGGGTCAGGGCTTGGAATGGATGGGTGTTATCGATCCGTCT |
| | antibody | GATGGTGGTACCAACTACGCACAGAAATTTCAAGGTCGCG |
| | (mAb5) | TGACCATGACCCGAGATACCTCCACGAGTACTGTGTACATG |
| | | GAATTGTCGTCCCTGCGCTCCGAAGACACGGCCGTGTATTA |
| | | CTGTGCAAGAGGACGGTATGACTACGGTGACTACTTGGGG |
| | | TGGTTCGACCCCTGGGGCCAGGGAACCCTGGTCACCGTCTC |
| | | CTCA |
| 92 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCACCAACTACTACATGCACTGGGTGCGGCAAGCG |
| | of anti-BCMA | CCGGGTCAGGGCTTGGAATGGATGGGTGTTATCGATCCGTC |
| | antibody | TGATGGTGGTACCAACTACGCACAGAAATTTCAAGGTCGC |
| | (mAb6) | GTGACCATGACCCGAGATACCTCCACGAGTACTGTGTACAT |
| | | GGAATTGTCGTCCCTGCGCTCCGAAGACATGGCCGTGTATT |
| | | ACTGTGCAAGAGGACGGTATGACTACGGTGACTACTTGGG |
| | | GTGGTTCGACCCCTGGGGCCAGGGAACCCTGGTCACCGTCT |
| | | CCTCA |
| 93 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTTA |
| | acid sequence | CACCTTCACCAACTACTACATGCACTGGGTGCGGCAAGCG |
| | of anti-BCMA | CCGGGTCAGGGCTTGGAATGGATGGGTGTTATCGATCCGTC |
| | antibody | TGATGGTGGTACCAACTACGCACAGAAATTTCAAGGTCGC |
| | (mAb7) | GTGACCATGACCCGAGATACCTCCACGAGTACTGTGTACAT |
| | | GGAATTGTCGTCCCTGCGCTCCGAAGACACGGCCGTGTATT |
| | | ACTGTGCAAGAGGACGGTATGACTACGGTGACTACTTGGG |
| | | GTGGTTCGACCCCTGGGGCCAGGGAACCCTGGTCACCGTCT |
| | | CCTCA |
| 94 | Heavy chain | CAGGTACAGCTGGTGCAATCTGGTGCGGAGGTGAAGAAACCG |
| | nucleic acid | GGTGCCAGTGTGAAAGTTAGTTGTAAGGCAAGCGGTTACACGT |
| | sequence of | TCACGTCATATTACATGCATTGGGTGCGGCAAGCGCCGGGTCA |
| | Construct 5 | GGGCTTGGAATGGATGGGTGTTATCGATCCGTCTGGTGGTTCT |
| | | ACCAACTACGCACAGAAATTTCAAGGTCGCGTGACCATGACCC |
| | | GAGATACCTCCACGAGTACTGTGTACATGGAATTGTCGTCCCT |
| | | GCGCTCCGAAGACACGGCCGTGTATTACTGTGCAAGAGGACG |
| | | GTATGACTACGGTGACTACTTGGGGTGGTTCGACGGGTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACAACAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGAGGCGGAAGCG |
| | | GAGGTGGCGGATCCCAGGTACAGCTGGTGCAATCTGGTGCG |
| | | GAGGTGAAGAAACCGGGTGCCAGTGTGAAAGTTAGTTGTAAGG |
| | | CAAGCGGTCACACCTTCACCTCTTACTTCATGCACTGGGTGCG |
| | | GCAAGCGCCGGGTCAGGGCTTGGAATGGATGGGTATCATCAA |
| | | CCCGTCTGATGATTACGCAAACTACGCACAGAAATTTCAAGGTC |
| | | GCGTGACCATGACCCGAGATACCTCCACGAGTACTGTGTACAT |
| | | GGAATTGTCGTCCCTGCGCTCCGAAGACACGGCCGTGTATTAC |
| | | TGTGCCACAGCGATCTTTGACTACTGGGGCCAGGGAACCCTG |
| | | GTCACCGTCTCCTCAGCTAGCACTAAAGGACCCAGTGTAT |
| | | TTCCTTTGGCCCCTAGCAGCAAATCTACATCTGGCGGC |
| | | ACAGCCGCTCTGGGTTGTCTGGTGAAAGACTACTTTCC |
| | | TGAACCCGTGACTGTTTCATGGAACAGTGGGGCACTCA |
| | | CGAGCGGAGTGCATACTTTCCCCGCTGTGCTTCAGAGT |
| | | TCTGGACTCTATTCGCTTAGCTCTGTCGTAACCGTCCCT |
| | | AGTTCCAGCCTGGGCACCCAAACATATATATGCAACGT |
| | | TAACCATAAACCCTCAAATACAAAGGTTGATAAGAAGG |
| | | TGGAGCCGAAGAGTTGTGACAAGACCCACACC |
| 95 | Heavy chain | CAGGTACAGCTGGTGCAATCTGGTGCGGAGGTGAAGAAACCG |
| | nucleic acid | GGTGCCAGTGTGAAAGTTAGTTGTAAGGCAAGCGGTTACACCT |
| | sequence of | TCACCAACTACTACATGCACTGGGTGCGGCAAGCGCCGGGTC |
| | Construct 6 | AGGGCTTGGAATGGATGGGTGTTATCGATCCGTCTGGTGGTTA |
| | | CACCAACTACGCACAGAAATTTCAAGGTCGCGTGACCATGACC |
| | | CGAGATACCTCCACGAGTACTGTGTACATGGAATTGTCGTCCC |
| | | TGCGCTCCGAAGACACGGCCGTGTATTACTGTGCAAGAGGAC |
| | | GGTATGACTACGGTGACTACTTGGGGTGGTTCGACGGTTGGG |
| | | GCCAGGGAACCCTGGTCACCGTCTCCTCAGCTAGCACCAAG |
| | | GGTCCGAGCGTCTTCCCCCTGGCACCCTCCTCCAAGAG |
| | | CACCTCTGGGGGCACAGCAGCCCTGGGCTGCCTGGTCA |
| | | AGGACTACTTCCCCGAACCGGTGACGGTGTCGTGGAAC |
| | | TCAGGCGCCCTGACCAGCGGCGTGCACACCTTCCCGGC |
| | | TGTCCTACAGTCCTCAGGACTCTACTCCCTCAGCAGCG |
| | | TGGTGACCGTGCCCTCCAGCAGCTTGGGCACCCAGACC |
| | | TACATCTGCAACGTGAATCACAAGCCCAGCAACACCAA |
| | | GGTGGACAAGAAAGTTGAGCCCAAATCTTGTGACAAAA |
| | | CTCACACATGCCCACCGTGCCCAGCACCTGAACTCCTG |
| | | GGGGGACCGTCAGTCTTCCTCTTCCCCCCAAAACCCAA |
| | | GGACACCCTCATGATCTCCCGGACCCCTGAGGTCACAT |
| | | GCGTGGTGGTGGACGTGAGCCACGAAGACCCTGAGGT |
| | | CAAGTTCAACTGGTACGTGGACGGCGTGGAGGTGCATA |
| | | ATGCCAAGACAAAGCCGCGGGAGGAGCAGTACAACAG |
| | | CACGTACCGTGTGGTCAGCGTCCTCACTGTCCTGCACC |
| | | AGGACTGGCTGAATGGCAAGGAGTACAAGTGCAAGGTC |
| | | TCCAACAAAGCCCTCCCAGCCCCCATCGAGAAAACCAT |
| | | CTCCAAAGCCAAAGGGCAGCCCCGAGAACCACAGGTGT |
| | | ACACCCTGCCCCCATCCCGGGATGAGCTGACCAAGAAC |
| | | CAGGTCAGCCTGACCTGCCTGGTCAAAGGCTTCTATCC |
| | | CAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCTACAGCAAGCTCACCGT |
| | | GGACAAGAGCAGGTGGCAGCAGGGGAACGTCTTCTCAT |
| | | GCTCCGTGATGCATGAGGCTCTGCACAACCACTACACA |
| | | CAAAAGAGCCTCTCCCTGTCTCCGGGTGGAGGCGGAAGC |
| | | GGAGGTGGCGGATCCCAGGTACAGCTGGTGCAATCTGGTGC |
| | | GGAGGTGAAGAAACCGGGTGCCAGTGTGAAAGTTAGTTGTAAG |
| | | GCAAGCGGTCACACCTTCACCTCTTACTTCATGCACTGGGTGC |
| | | GGCAAGCGCCGGGTCAGGGCTTGGAATGGATGGGTATCATCA |
| | | ACCCGTCTGATGATTACGCAAACTACGCACAGAAATTTCAAGGT |
| | | CGCGTGACCATGACCCGAGATACCTCCACGAGTACTGTGTACA |
| | | TGGAATTGTCGTCCCTGCGCTCCGAAGACACGGCCGTGTATTA |
| | | CTGTGCCACAGCGATCTTTGACTACTGGGGCCAGGGAACCCTG |
| | | GTCACCGTCTCCTCAGCTAGCACTAAAGGACCCAGTGTAT |
| | | TTCCTTTGGCCCCTAGCAGCAAATCTACATCTGGCGGC |
| | | ACAGCCGCTCTGGGTTGTCTGGTGAAAGACTACTTTCC |
| | | TGAACCCGTGACTGTTTCATGGAACAGTGGGGCACTCA |
| | | CGAGCGGAGTGCATACTTTCCCCGCTGTGCTTCAGAGT |
| | | TCTGGACTCTATTCGCTTAGCTCTGTCGTAACCGTCCCT |
| | | AGTTCCAGCCTGGGCACCCAAACATATATATGCAACGT |
| | | TAACCATAAACCCTCAAATACAAAGGTTGATAAGAAGG |
| | | TGGAGCCGAAGAGTTGTGACAAGACCCACACC |
| 96 | Variable heavy | CAAGTGCAGCTTGTTCAGAGCGGAGCTGAAGTGAAGAAAC |
| | chain nucleic | CCGGTTCTAGTGTTAAAGTGTCATGTAAAGCCAGTGGCGGT |
| | acid sequence | ACCTTCACCTCTTACTCTGTTTCTTGGGTGCGCCAGGCTCCG |
| | of mAb10 | GGCCAGGGCCTGGAGTGGATGGGTGGTATCGTTCCAATCTT |
| | | CGGTACCGCAGATTATGCCCAAAAATTTCAAGGAAGAGTG |
| | | ACGATAACCGCTGATGAATCTACAAGCACTGCATATATGG |
| | | AATTGTCGAGTTTACGGTCCGAGGATACGGCCGTGTATTAC |
| | | TGTGCGAGAGGATACAGCTATGGTCAGACCTTTGACTACTG |
| | | GGGCCAGGGAACCCTGGTCACCGTCTCCTCA |
| 97 | Alternative | CAGGTACAGCTGGTGCAATCTGGTGCGGAGGTGAAGAAACCG |
| | heavy chain | GGTGCCAGTGTGAAAGTTAGTTGTAAGGCAAGCGGTTACACCT |
| | nucleic acid | TCTCTTCTCACTACGTTCACTGGGTGCGGCAAGCGCCGGGTCA |
| | sequence of | GGGCTTGGAATGGATGGGTATCATCGATCCGTCTGGTGGTTCT |
| | Construct 1 | ACCTCTTACGCACAGAAATTTCAAGGTCGCGTGACCATGACCC |
| | (Construct 7) | GAGATACCTCCACGAGTACTGTGTACATGGAATTGTCGTCCCT |
| | | GCGCTCCGAAGACACGGCCGTGTATTACTGTGCGAGAGGACG |
| | | GTATGACTACGGTGACTACTTGGGGTGGTTCGACCCCTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACAACAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGCGGAGGGAGTG |
| | | GAGGTGGAGGCAGTGGTGGAGGCGGAAGCGGAGGTGGCG |
| | | GATCCCAGGTACAGCTGGTGCAATCTGGTGCGGAGGTGAAGA |
| | | AACCGGGTGCCAGTGTGAAAGTTAGTTGTAAGGCAAGCGGTCA |
| | | CACCTTCACCTCTTACTTCATGCACTGGGTGCGGCAAGCGCCG |
| | | GGTCAGGGCTTGGAATGGATGGGTATCATCAACCCGTCTGATG |
| | | ATTACGCAAACTACGCACAGAAATTTCAAGGTCGCGTGACCAT |
| | | GACCCGAGATACCTCCACGAGTACTGTGTACATGGAATTGTCG |
| | | TCCCTGCGCTCCGAAGACACGGCCGTGTATTACTGTGCCACAG |
| | | CGATCTTTGACTACTGGGGCCAGGGAACCCTGGTCACCGTCTC |
| | | CTCAGCTAGCACTAAAGGACCCAGTGTATTTCCTTTGGC |
| | | CCCTAGCAGCAAATCTACATCTGGCGGCACAGCCGCTC |
| | | TGGGTTGTCTGGTGAAAGACTACTTTCCTGAACCCGTG |
| | | ACTGTTTCATGGAACAGTGGGGCACTCACGAGCGGAGT |
| | | GCATACTTTCCCCGCTGTGCTTCAGAGTTCTGGACTCTA |
| | | TTCGCTTAGCTCTGTCGTAACCGTCCCTAGTTCCAGCCT |
| | | GGGCACCCAAACATATATATGCAACGTTAACCATAAAC |
| | | CCTCAAATACAAAGGTTGATAAGAAGGTGGAGCCGAAG |
| | | AGTTGTGACAAGACCCACACC |
| 98 | Variable heavy | CAGGTACAGCTGGTGCAATCTGGTGCGGAGGTGAAGAAAC |
| | chain nucleic | CGGGTGCCAGTGTGAAAGTTAGTTGTAAGGCAAGCGGTCA |
| | acid sequence | CACCTTCACCAACTACTACATGCACTGGGTGCGGCAAGCG |
| | of mAb11 | CCGGGTCAGGGCTTGGAATGGATGGGTGTTATCAACCCGA |
| | | ACGGTGGTGATACCTCTTACGCACAGAAATTTCAAGGTCGC |
| | | GTGACCATGACCCGAGATACCTCCACGAGTACTGTGTACAT |
| | | GGAATTGTCGTCCCTGCGCTCCGAAGACACGGCCGTGTATT |
| | | ACTGTGCGAGAGATCGGGCCTGGGACTACGGTGGTAACGT |
| | | GCGTGCTTTTGATATCTGGGGCCAAGGGACAATGGTCACC |
| | | GTCTCTTCA |
| 99 | Aglycosylated | CAGGTACAGCTGGTGCAATCTGGTGCGGAGGTGAAGAAACCG |
| | heavy chain | GGTGCCAGTGTGAAAGTTAGTTGTAAGGCAAGCGGTTACACGT |
| | nucleic acid | TCACGTCATATTACATGCATTGGGTGCGGCAAGCGCCGGGTCA |
| | sequence of | GGGCTTGGAATGGATGGGTGTTATCGATCCGTCTGGTGGTTCT |
| | Construct 5 | ACCAACTACGCACAGAAATTTCAAGGTCGCGTGACCATGACCC |
| | (Construct 8) | GAGATACCTCCACGAGTACTGTGTACATGGAATTGTCGTCCCT |
| | | GCGCTCCGAAGACACGGCCGTGTATTACTGTGCAAGAGGACG |
| | | GTATGACTACGGTGACTACTTGGGGTGGTTCGACGGGTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACGCCAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGAGGCGGAAGCG |
| | | GAGGTGGCGGATCCCAGGTACAGCTGGTGCAATCTGGTGCG |
| | | GAGGTGAAGAAACCGGGTGCCAGTGTGAAAGTTAGTTGTAAGG |
| | | CAAGCGGTCACACCTTCACCTCTTACTTCATGCACTGGGTGCG |
| | | GCAAGCGCCGGGTCAGGGCTTGGAATGGATGGGTATCATCAA |
| | | CCCGTCTGATGATTACGCAAACTACGCACAGAAATTTCAAGGTC |
| | | GCGTGACCATGACCCGAGATACCTCCACGAGTACTGTGTACAT |
| | | GGAATTGTCGTCCCTGCGCTCCGAAGACACGGCCGTGTATTAC |
| | | TGTGCCACAGCGATCTTTGACTACTGGGGCCAGGGAACCCTG |
| | | GTCACCGTCTCCTCAGCTAGCACTAAAGGACCCAGTGTAT |
| | | TTCCTTTGGCCCCTAGCAGCAAATCTACATCTGGCGGC |
| | | ACAGCCGCTCTGGGTTGTCTGGTGAAAGACTACTTTCC |
| | | TGAACCCGTGACTGTTTCATGGAACAGTGGGGCACTCA |
| | | CGAGCGGAGTGCATACTTTCCCCGCTGTGCTTCAGAGT |
| | | TCTGGACTCTATTCGCTTAGCTCTGTCGTAACCGTCCCT |
| | | AGTTCCAGCCTGGGCACCCAAACATATATATGCAACGT |
| | | TAACCATAAACCCTCAAATACAAAGGTTGATAAGAAGG |
| | | TGGAGCCGAAGAGTTGTGACAAGACCCACACC |
| 100 | aglycosylated | CAGGTACAGCTGGTGCAATCTGGTGCGGAGGTGAAGAAACCG |
| | IgG1 heavy | GGTGCCAGTGTGAAAGTTAGTTGTAAGGCAAGCGGTTACACCT |
| | chain nucleic | TCTCTTCTCACTACGTTCACTGGGTGCGGCAAGCGCCGGGTCA |
| | acid sequence | GGGCTTGGAATGGATGGGTATCATCGATCCGTCTGGTGGTTCT |
| | of Construct 9 | ACCTCTTACGCACAGAAATTTCAAGGTCGCGTGACCATGACCC |
| | | GAGATACCTCCACGAGTACTGTGTACATGGAATTGTCGTCCCT |
| | | GCGCTCCGAAGACACGGCCGTGTATTACTGTGCGAGAGGACG |
| | | GTATGACTACGGTGACTACTTGGGGTGGTTCGACCCCTGGGG |
| | | CCAGGGAACCCTGGTCACCGTCTCCTCAGCTAGCACCAAGG |
| | | GTCCGAGCGTCTTCCCCCTGGCACCCTCCTCCAAGAGC |
| | | ACCTCTGGGGGCACAGCAGCCCTGGGCTGCCTGGTCAA |
| | | GGACTACTTCCCCGAACCGGTGACGGTGTCGTGGAACT |
| | | CAGGCGCCCTGACCAGCGGCGTGCACACCTTCCCGGCT |
| | | GTCCTACAGTCCTCAGGACTCTACTCCCTCAGCAGCGT |
| | | GGTGACCGTGCCCTCCAGCAGCTTGGGCACCCAGACCT |
| | | ACATCTGCAACGTGAATCACAAGCCCAGCAACACCAAG |
| | | GTGGACAAGAAAGTTGAGCCCAAATCTTGTGACAAAAC |
| | | TCACACATGCCCACCGTGCCCAGCACCTGAACTCCTGG |
| | | GGGGACCGTCAGTCTTCCTCTTCCCCCCAAAACCCAAG |
| | | GACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACAACAGCA |
| | | CGTACCGTGTGGTCAGCGTCCTCACTGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATCCCGGGATGAGCTGACCAAGAACCA |
| | | GGTCAGCCTGACCTGCCTGGTCAAAGGCTTCTATCCCA |
| | | GCGACATCGCCGTGGAGTGGGAGAGCAATGGGCAGCC |
| | | GGAGAACAACTACAAGACCACGCCTCCCGTGCTGGACT |
| | | CCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACAC |
| | | AAAAGAGCCTCTCCCTGTCTCCGGGTGGAGGCGGAAGCG |
| | | GAGGTGGCGGATCCCAAGTGCAGCTTGTTCAGAGCGGAGCTG |
| | | AAGTGAAGAAACCCGGTTCTAGTGTTAAAGTGTCATGTAAAGCC |
| | | AGTGGCGGTACCTTCACCTCTTACTCTGTTTCTTGGGTGCGCC |
| | | AGGCTCCGGGCCAGGGCCTGGAGTGGATGGGTGGTATCGTTC |
| | | CAATCTTCGGTACCGCAGATTATGCCCAAAAATTTCAAGGAAGA |
| | | GTGACGATAACCGCTGATGAATCTACAAGCACTGCATATATGGA |
| | | ATTGTCGAGTTTACGGTCCGAGGATACGGCCGTGTATTACTGT |
| | | GCGAGAGGATACAGCTATGGTCAGACCTTTGACTACTGGGGCC |
| | | AGGGAACCCTGGTCACCGTCTCCTCAGCTAGCACTAAAGGA |
| | | CCCAGTGTATTTCCTTTGGCCCCTAGCAGCAAATCTACA |
| | | TCTGGCGGCACAGCCGCTCTGGGTTGTCTGGTGAAAGA |
| | | CTACTTTCCTGAACCCGTGACTGTTTCATGGAACAGTG |
| | | GGGCACTCACGAGCGGAGTGCATACTTTCCCCGCTGTG |
| | | CTTCAGAGTTCTGGACTCTATTCGCTTAGCTCTGTCGTA |
| | | ACCGTCCCTAGTTCCAGCCTGGGCACCCAAACATATAT |
| | | ATGCAACGTTAACCATAAACCCTCAAATACAAAGGTTG |
| | | ATAAGAAGGTGGAGCCGAAGAGTTGTGACAAGACCCAC |
| | | ACC |
| 101 | mAb8 | QVQLVQSGAEVKKPGASVKVSCKASGHTFTSYFMEIWVRQAP |
| | IgG1 heavy | GQGLEWMGIINPSDDYANYAQKFQGRVTMTRDTSTSTVYME |
| | chain amino | LSSLRSEDTAVYYCATAIFDYWGQGTLVTVSSASTKGPSVFPL |
| | acid sequence | APSSKSTSGGTAALGCLVKDYFPEPVTVSWNSGALTSGVHTF |
| | (version | PAVLQSSGLYSLSSVVTVPSSSLGTQTYICNVNHKPSNTKVDK |
| | without GT | KVEPKSCDKTHTCPPCPAPELLGGPSVFLFPPKPKDTLMISRTP |
| | cloning site) | EVTCVVVDVSHEDPEVKFNWYVDGVEVHNAKTKPREEQYA |
| | | STYRVVSVLTVLHQDWLNGKEYKCKVSNKALPAPIEKTISKA |
| | | KGQPREPQVYTLPPSRDELTKNQVSLTCLVKGFYPSDIAVEW |
| | | ESNGQPENNYKTTPPVLDSDGSFFLYSKLTVDKSRWQQGNVF |
| | | SCSVMHEALHNHYTQKSLSLSPG |
| 102 | mAb1 | QVQLVQSGAEVKKPGASVKVSCKASGYTFSSHYVHWVRQAP |
| | aglycosylated | GQGLEWMGIIDPSGGSTSYAQKFQGRVTMTRDTSTSTVYMEL |
| | IgG1 heavy | SSLRSEDTAVYYCARGRYDYGDYLGWFDPWGQGTLVTVSSA |
| | chain amino | STKGPSVFPLAPSSKSTSGGTAALGCLVKDYFPEPVTVSWNSG |
| | acid sequence | ALTSGVHTFPAVLQSSGLYSLSSVVTVPSSSLGTQTYICNVNH |
| | (version | KPSNTKVDKKVEPKSCDKTHTCPPCPAPELLGGPSVFLFPPKP |
| | without GT | KDTLMISRTPEVTCVVVDVSHEDPEVKFNWYVDGVEVHNAK |
| | cloning site) | TKPREEQYASTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFFLYSKLTVDKS |
| | | RWQQGNVFSCSVMHEALHNHYTQKSLSLSPG |

*For table entries containing annotated sequences: italicized sequences are variable regions; bolded regions are constant region sequences; and underlined sequences correspond to linker amino acid sequences.

1. A multispecific antigen-binding construct comprising at least two linked antigen-binding units, wherein a first antigen-binding unit specifically targets a tumor cell by binding a first tumor antigen or specifically targets a B cell by binding a first target antigen expressed by a B cell, and wherein a second antigen-binding unit specifically binds a NKp30 antigen.

2-17. (canceled)

18. A multispecific antigen-binding construct comprising at least four linked antigen-binding units, wherein (i) a first antigen-binding unit specifically targets a tumor cell by binding a first tumor antigen or targets a B cell by binding a first target antigen expressed by a B cell, a second antigen-binding unit specifically binds a first NK cell activating receptor, a third antigen-binding unit binds a second NK cell activating receptor, and a fourth antigen-binding unit binds to a second tumor antigen or second target antigen expressed by a B cell, and wherein (ii) the first NK receptor is NKp30 and the second NK receptor is not NKp30.

19-73. (canceled)

74. An antibody or antigen-binding fragment thereof that specifically binds human BCMA, wherein the antibody or antigen-binding fragment thereof comprises a heavy chain variable region comprising:
(a) a CDRH1 of SEQ ID NO: 38 (YTFX₁X₂X₃YX₄H, wherein X₁ is T or S, X₂ is N or S, X₃ is Y or H, and X₄ is M or V);
(b) a CDRH2 of SEQ ID NO: 39 (GX₅IDPSX₆GX₇TX₈YA, wherein X₅ is V or I, X₆ is G or D, X₇ is G, Y or S, and X₈ is N or S); and
(c) a CDRH3 of SEQ ID NO: 40 (ARGRYDYX₉DYLGWFDX₁₀, wherein X₉ is G or S, X₁₀ is P or G).

75-89. (canceled)

90. The antibody or antigen-binding fragment thereof of claim 74, that specifically binds to human BCMA, wherein, when bound to human BCMA, the antibody or antigen-binding fragment thereof binds to at least one of the amino acid residues bound by an anti-human BCMA antibody comprising the heavy chain variable region sequence of any one of SEQ ID NO:10, SEQ ID NO: 52, SEQ ID NO: 53, SEQ ID NO: 54, SEQ ID NO: 55, SEQ ID NO: 56, and SEQ ID NO: 57 and a light chain variable sequence of SEQ ID NO:18.

91. The antibody or antigen-binding fragment thereof of claim 111, wherein the antibody or antigen-binding fragment thereof comprises a heavy chain variable region comprising (a) a CDRH1 of SEQ ID NO: 15, a CDRH2 of SEQ ID NO: 16, and a CDRH3 of SEQ ID NO: 42 or (b) a CDRH1 of SEQ ID NO: 69, a CDRH2 of SEQ ID NO: 70, and a CDRH3 of SEQ ID NO: 71.

92-95. (canceled)

96. The antibody or antigen-binding fragment thereof of claim 111, wherein, when bound to human NKp30, the antibody or antigen-binding fragment thereof cross blocks the binding of an anti-human NKp30 antibody comprising (a) the heavy chain variable region sequence of SEQ ID NO: 14, SEQ ID NO: 25, or SEQ ID NO: 72 and the light chain variable region sequence of SEQ ID NO: 18, or (b) the heavy chain variable region sequence of SEQ ID NO: 78 and the light chain variable region sequence of SEQ ID NO: 80.

97-110. (canceled)

111. An antibody or antigen-binding portion thereof that binds to an epitope of human NKp30, wherein the epitope is within or overlapping with amino acids 50-113 of SEQ ID NO: 7, and wherein one or more of substitutions at I50, S82, and L113 disrupts binding of the antibody or antigen-binding portion to human NKp30.

112-129. (canceled)

130. A nucleic acid encoding the multispecific antigen-binding construct or isolated monoclonal antibody or antigen-binding fragment thereof according to any one of claims Error! Reference source not found, 18, 74, 90, 91, 96, or 111.

131. An expression vector comprising the nucleic acid of claim Error! Reference source not found.

132. (canceled)

133. A cell comprising the expression vector of claim Error! Reference source not found.

134-136. (canceled)

137. A method for producing a multispecific antigen-binding construct or an antibody or antigen-binding fragment thereof, the method comprising culturing the cell of claim Error! Reference source not found. under conditions suitable for expression of the multispecific antigen-binding construct or antibody or antigen-binding fragment thereof from the expression vector by the cell.

138. (canceled)

139. A pharmaceutical composition comprising the multispecific antigen-binding construct or the antibody or antigen-binding fragment thereof according to any one of claims Error! Reference source not found, 18, 74, 90, 91, 96, or 111 and a pharmaceutically acceptable carrier.

140. A protein conjugate molecule comprising:
(a) the multispecific antigen-binding construct or the antibody or antigen-binding fragment thereof according to any one of claims Error! Reference source not found, 18, 74, 90, 91, 96, or 111 and
(b) a heterologous moiety, wherein the heterologous moiety is conjugated to the multispecific antigen-binding construct or the isolated antibody or antigen-binding fragment thereof of (a).

141. (canceled)

142. A method for enhancing an immune response against a cancer cell in a subject, the method comprising administering to the subject an effective amount of the multispecific antigen-binding constructs, the antibody or antigen-binding fragment thereof, the pharmaceutical composition, or the protein conjugate of any one of claims Error! Reference source not found, 18, 74, 90, 91, 96, 111, 139, or 140, thereby enhancing an immune response against a cancer cell in the subject.

143-146. (canceled)

147. A method for enhancing an immune response against a B cell in a subject, the method comprising administering to the subject an effective amount of the multispecific antigen-binding constructs, the antibody or antigen-binding fragment thereof, the pharmaceutical composition, or the protein conjugate of any one of claims Error! Reference source not found, 18 74, 90, 91, 96, 111, 139, or 140, thereby enhancing an immune response against a B cell in the subject.

148-151. (canceled)

152. A method for treating cancer in a subject, the method comprising administering to the subject an effective amount of the multispecific antigen-binding constructs, the antibody or antigen-binding fragment thereof of, the pharmaceutical composition, or the protein conjugate any one of claims Error! Reference source not found, , 18, 74, 90, 91, 96, 111, 139, or 140.

153-176. (canceled)

177. A method of treating a subject with an autoimmune disease, the method comprising administering to the subject the multispecific antigen-binding constructs, the antibody or antigen-binding fragment thereof of, the pharmaceutical composition, or the protein conjugate of any one of claims Error! Reference source not found, 18, 74, 90, 91, 96, 111, 139, or 140.

178-185. (canceled)

186. A method of activating or sustaining activation of a natural killer (NK) cell comprising contacting the NK cell with an effective amount of the multispecific antigen-binding construct, the antibody or antigen-binding fragment thereof of, the pharmaceutical composition, or the protein conjugate of any one of claims Error! Reference source not found, 18, 74, 90, 91, 96, 111, 139, or 140.

187-188. (canceled)

189. A method of activating or sustaining activation of a natural killer (NK) cell in a subject, comprising contacting the NK cell with an effective amount of the multispecific antigen-binding construct, the antibody or antigen-binding fragment thereof of, the pharmaceutical composition, or the protein conjugate of any one of claims Error! Reference source not found, 18, 74, 90, 91, 96, 111, 139, or 140, wherein the NK cell has reduced expression of CD16 as compared to a control NK cell.

190-198. (canceled)

199. A method for enhancing the killing of a target cell by a natural killer (NK) cell, comprising contacting the target cell with the multispecific antigen-binding construct, isolated monoclonal the antibody or antigen-binding fragment thereof of, the pharmaceutical composition, or the protein conjugate of any one of claims Error! Reference source not found, 18, 74, 90, 91, 96, 111, 139, or 140 in the presence of the NK cell.

200-203. (canceled)

204. A method for enhancing the killing of a cancer cell by an NK cell in a subject, the method comprising administering to the subject an effective amount of the multispecific antigen-binding construct, the antibody or antigen-binding fragment thereof of, the pharmaceutical composition, or the protein conjugate of any one of claims Error! Reference source not found, 18, 74, 90, 91, 96, 111, 139, or 140, wherein the cancer cell expresses a tumor antigen to which the construct or isolated monoclonal antibody or antigen-binding fragment thereof binds at a copy number of less than 100,000.

205-207. (canceled)

208. A method of treating a subject with an autoreactive-B cell inflammatory condition, comprising administering to the subject an effective amount of the multispecific antigen-binding constructs, the antibody or antigen-binding fragment thereof of, the pharmaceutical composition, or the protein conjugate of any one of claims Error! Reference source not found, 18, 74, 90, 91, 96, 111, 139, or 140.

209-283. (canceled)
